(19) United States
(12) Patent Application Publication
(10) Pub. No.: US 2020/0079825 A1
(43) Pub. Date: Mar. 12, 2020

(54) RARE PHOSPHORYLATED HIGH MOLECULAR WEIGHT (HMW) TAU SPECIES THAT ARE INVOLVED IN NEURONAL UPTAKE AND PROPAGATION AND APPLICATIONS THEREOF

(71) Applicant: THE GENERAL HOSPITAL CORPORATION, Boston, MA (US)

(72) Inventors: Shuko TAKEDA, Boston, MA (US); Bradley T. HYMAN, Swampscott, MA (US)

(73) Assignee: The General Hostital Corporation, Boston, MA (US)

(21) Appl. No.: 15/743,787

(22) PCT Filed: Jul. 13, 2016
(86) PCT No.: PCT/US2016/042094

§ 371 Date: Jan. 11, 2018

Related U.S. Application Data
(60) Provisional application No. 62/222,845, filed on Sep. 24, 2015.
(60) Provisional application No. 62/194,978, filed on Jul. 21, 2015.
(60) Provisional application No. 62/191,769, filed on Jul. 13, 2015.

(51) Int. Cl. C07K 14/47; C07K 16/18; G01N 33/68

(52) U.S. Cl. CPC C07K 14/4711; C07K 16/18; G01N 2800/2814; G01N 33/6896; G01N 33/6875

(57) ABSTRACT

The disclosure provides novel forms of phosphorylated tau species and applications thereof as well as methods of diagnosing and/or treating tau-associated neurodegeneration.

### CROSS-REFERENCE TO RELATED APPLICATION

This application claims benefit under 35 U.S.C. § 119(c) of U.S. Provisional Application Nos. 62/191,769, filed Jul. 13, 2015, 62/194,978, filed Jul. 21, 2015, and 62/222,845, filed Sep. 24, 2015, the contents of which are incorporated herein by reference in their entireties.

### GOVERNMENT SUPPORT

This invention was made with Government Support under Contract No. AG026249 awarded by the National Institutes of Health (NIH). The Government has certain rights in the invention.

### TECHNICAL FIELD

The technology described herein relates generally to novel forms of tau species and applications thereof as well as methods of treating and/or diagnosing tau-associated neurodegeneration in a subject.

### SEQUENCE LISTING

The instant application contains a Sequence Listing which has been submitted electronically in ASCII format and is hereby incorporated by reference in its entirety. Said ASCII copy, created on Jul. 11, 2016, is named 030258-085413-PCT_SL.txt and is 4,274 bytes in size.

### BACKGROUND

Accumulation and aggregation of microtubule-associated protein tau (Mandelkow et al. (1995) Neurobiology of aging, 16(3):355-362; discussion 362-353), as intracellular inclusions known as neurofibrillary tangles (NFTs), is a pathological hallmark of neurodegenerative diseases including Alzheimer's disease (AD) (Iqbal et al. (2010) Current Alzheimer research, 7(8): 656-664). Cognitive deficits in AD are most closely linked with progression of NFTs in a hierarchical pattern, starting in the entorhinal cortex (EC) and marching throughout the brain during disease progression (Hyman et al. (1984) Science, 225 (4667): 1168-1170). One theory posits a “prion-like” spreading of tau: misfolded tau travels between neurons and provides a template for aggregation of naive endogenous tau in recipient neurons, which becomes neurotoxic. Although the precise mechanisms for this characteristic tau pathology spread remain unknown, it has been previously discussed that the tau pathology spread can occur by a trans-synaptic transfer of tau proteins between neurons (Pooler et al. (2013) Alzheimer's research & therapy, 5(5): 49; Walker et al. (2013) JAMA neurology, 70(3): 304-310). However, the tau species involved in inter-neuron propagation remains unclear.

Better understanding of the molecular basis of tau propagation can allow preventing progression from early mild memory impairment to full cognitive deterioration and dementia. Accordingly, there is a need to identify specific tau species responsible for inter-neuron propagation, which can be used as a more effective target for therapeutic intervention and biomarker development.

### SUMMARY

Described herein are low-abundance, soluble high molecular weight (HMW) tau species present in postmortem brain cortical extracts from tau-transgenic mice and AD patients, and particularly specific phosphorylated forms of such species that are involved in neuronal uptake and propagation between neurons. Thus, various aspects described herein stem from, at least in part, discovery of specific phosphorylation forms of the phosphorylated soluble HMW tau species, e.g., soluble HMW tau species phosphorylated at one or more of the following amino acid residues: serine 396, serine 199, and serine 404 that are important in neuronal uptake and propagation between neurons, wherein the locations of the phosphorylation sites (e.g., S396, S199, and S404) are based on a full-length tau reference sequence as defined in SEQ ID NO: 1. It is also demonstrated herein that neuronal uptake of phosphorylated soluble HMW tau species, not low-molecular-weight (LMW) tau fractions, was detected in pre-tangle stage and wild-type (control) mouse models when the HMW tau species or LMW tau fractions were injected into the frontal cortex of the mice. Thus, in one aspect, the discovery of specific phosphorylated forms of rare soluble HMW tau species involved in inter-neuron-propagation provides an effective target for therapeutic intervention and biomarker development. Accordingly, embodiments of various aspects described herein relate to compositions comprising phosphorylated soluble HMW tau species that are responsible for inter-neuron propagation, and applications thereof. Methods of treating and diagnosing tau-associated neurodegeneration in a subject are also provided herein.

In one aspect, a composition comprising phosphorylated soluble high molecular weight (HMW) tau species is provided herein. The phosphorylated soluble HMW tau species in the composition is non-fibrillar and has a molecular weight of at least about 500 kDa, and the composition is substantially free of soluble low molecular weight (LMW) tau species. In this composition, the phosphorylated soluble HMW tau species that is phosphorylated at amino acid residue serine 422 is present at a lower amount than that of the soluble HMW tau species phosphorylated at one or more of the following amino acid residues: serine 396, serine 199, and serine 404, wherein the locations of the phosphorylation sites (e.g., S396, S199, and S404) are based on a full-length tau reference sequence as defined in SEQ ID NO: 1.

In some embodiments, the phosphorylated soluble HMW tau species can have a molecular weight of at least about 669 kDa. In some embodiments, the phosphorylated soluble HMW tau species can have a molecular weight of about 669 kDa to about 1000 kDa.

In some embodiments, the non-fibrillar, phosphorylated soluble HMW tau species can be in a form of globular particles. The particle size can vary with the molecular weight of the tau species. In some embodiments, the particle size can range from about 10 nm to about 30 nm.

In some embodiments, the phosphorylated soluble HMW tau species can be positive for Alz50 and negative for Thioflavin-S(ThioS).

The phosphorylated soluble HMW tau species can be soluble in an aqueous and/or buffered solution. For example, in some embodiments, the phosphorylated soluble HMW tau species can be soluble in phosphate-buffered saline. In some embodiments, the phosphorylated soluble HMW tau species can be soluble in a biological fluid, e.g., a brain interstitial fluid or cerebrospinal fluid.

In some embodiments, the phosphorylated soluble HMW tau species can be preferentially taken up by a neuron and axonally transported from the neuron to a synaptically-connected neuron, as compared to neuron uptake and neuron-to-neuron transport of the soluble LMW tau species. The soluble LMW tau species has a lower molecular weight than that of the phosphorylated soluble HMW tau species. In some embodiments, the soluble LMW tau species can have a molecular weight of no more than 200 kDa.

In some embodiments, the compositions described herein can comprise an agent to suit the need of a selected application. For example, where the phosphorylated soluble HMW tau is to be used as an antigen to raise an antibody, purified phosphorylated soluble HMW tau can be combined with saline or phosphate-buffered saline. Alternatively, or in addition, the HMW tau antigen can be admixed with or conjugated to an adjuvant or carrier, e.g., a carrier peptide, to enhance its antigenicity.

Accordingly, another aspect described herein provides an isolated antibody or antigen-binding portion thereof that specifically binds soluble HMW tau species bearing phosphate at particular locations. For example, in one embodiment, the isolated antibody or antigen-binding portion thereof specifically binds soluble HMW tau species phosphorylated at serine 396, and does not bind soluble low molecular weight (LMW) tau species. In one embodiment, the antibody or antigen-binding portion thereof contacts serine 396 when it is phosphorylated, but not when it lacks phosphorylation. As noted, the phosphorylated soluble HMW tau species to which the antibody binds is non-fibrillar and has a molecular weight of at least about 500 kDa, and the LMW tau species has a molecular weight of no more than 200 kDa. In some embodiments, the phosphorylated soluble HMW tau species can have a molecular weight of at least about 669 kDa or more. In some embodiments, the phosphorylated soluble HMW tau species can have a molecular weight of about 669 kDa to about 1000 kDa.

In some embodiments, the isolated antibody or antigen-binding portion thereof specifically binds soluble HMW tau species phosphorylated at serine 404, and does not bind soluble low molecular weight (LMW) tau species. In one embodiment, the antibody or antigen-binding portion thereof contacts serine 404 when it is phosphorylated, but not when it lacks phosphorylation.

In some embodiments, the isolated antibody or antigen-binding portion thereof specifically binds soluble HMW tau species phosphorylated at serine 199, and does not bind soluble low molecular weight (LMW) tau species. In one embodiment, the antibody or antigen-binding portion thereof contacts serine 199 when it is phosphorylated, but not when it lacks phosphorylation.

The inventors have shown that blocking the phosphorylation site at serine 396 of the phosphorylated soluble HMW tau species can significantly reduce neuronal uptake of the HMW tau species and thus inter-neuron propagation. Thus, in some embodiments, the isolated antibody or antigen-binding portion described herein can reduce the phosphorylated soluble HMW tau species being taken up by a neuron, and/or reduce the phosphorylated soluble HMW tau species being axonally transported from a neuron to a synaptically-connected neuron, e.g., by at least 10% or more.

The inventors have shown that a relatively low level of phosphorylated soluble HMW tau species was released from the neurons and found in brain interstitial fluid and cerebrospinal fluid. The inventors have also shown that the phosphorylated soluble HMW tau species, which accounts for only a small fraction of all tau in the samples, was robustly taken up by neurons, and was involved in inter-neuron propagation, whereas uptake of soluble LMW tau species (e.g., monomer/dimer size) or even non-phosphorylated soluble HMW tau species was very inefficient. By blocking specific phosphorylation site(s), e.g., at serine 396, of HMW tau species, and/or removing such specific phosphorylated form, neuronal uptake of the HMW tau species was significantly reduced. Thus, a method of preventing propagation of pathological tau protein between synaptically-connected neurons is also provided herein. The method comprises selectively reducing the extracellular level of a phosphorylated soluble HMW tau species in contact with a synaptically-connected neuron, wherein the phosphorylated soluble HMW tau species is non-fibrillar, has a molecular weight of at least about 500 kDa, and is phosphorylated at least at serine 396 (S396). A reduced level of the phosphorylated soluble HMW tau species results in reduced propagation of pathological tau protein between synaptically-connected neurons.

In some embodiments, the method can further comprise selectively reducing the extracellular level of an additional phosphorylated soluble HMW tau species in contact with a synaptically-connected neuron, wherein the additional phosphorylated soluble HMW tau species is non-fibrillar, has a molecular weight of at least about 500 kDa, and is phosphorylated at least at serine 199 (S199), and/or serine 404 (S404).

In some embodiments, the extracellular level of a soluble HMW tau species phosphorylated at serine 422 is not substantially reduced during said selective reduction. In some embodiments, the extracellular level of a soluble HMW tau species phosphorylated at serine 409 is not substantially reduced during said selective reduction. In some embodiments, the extracellular level of a soluble HMW tau species phosphorylated at serine 400 is not substantially reduced during said selective reduction. In some embodiments, the extracellular level of a soluble HMW tau species phosphorylated at serine 262 is not substantially reduced during said selective reduction. In some embodiments, the extracellular level of a soluble HMW tau species phosphorylated at threonine 205 is not substantially reduced during said selective reduction.

In some embodiments, the extracellular level of soluble LMW tau species is not substantially reduced during the selective reduction.

Methods for selectively reducing the extracellular level of soluble HMW tau species can be based on physical removal and/or molecular interactions between the phosphorylated soluble HMW tau species and a proper antagonist. In some embodiments, the phosphorylated soluble HMW tau species can be selectively reduced by contacting the extracellular space or fluid in contact with the synaptically-connected neurons with an antagonist of the soluble HMW tau species phosphorylated at serine 396, serine 199, and/or serine 404. Examples of an antagonist of such phosphorylated soluble HMW tau species include, without limitations, an antibody, a nuclease (e.g., but not limited to, a zinc finger nuclease (ZFN)), transcription activator-like effector nuclease (TALEN), a gene-editing composition (e.g., a CRISPR/Cas system), a transcriptional repressor, a nucleic acid inhibitor (e.g., RNAi, siRNA, anti-miR, antisense oligonucleotides, ribozymes, and a combination of two or more thereof), a small organic molecule, an aptamer, and a combination of two or more thereof.

Tau pathology is known to spread in a hierarchical pattern in Alzheimer's disease (AD) brain during disease progression, e.g., by trans-synaptic transfer of pathological forms of tau between neurons to facilitate propagation of neurofibrillary tangles (insoluble and fibrillar tau aggregates). Since the soluble HMW tau species phosphorylated at least at S396 is identified herein to be involved in neuron-to-neuron propagation, intervention to deplete such phosphorylated soluble HMW tau species can inhibit tau propagation and hence disease progression in tauopathies. Accordingly, a method of reducing tau-associated neurodegeneration in a subject is provided herein. Examples of tau-associated neurodegeneration include, but are not limited to, Alzheimer's disease, Parkinson's disease, or frontotemporal dementia. The method of treatment comprises selectively reducing the level of a phosphorylated soluble HMW tau species in the brain (e.g., in the brain interstitial fluid) or cerebrospinal fluid (CSF) of a subject determined to have, or be at risk for, tau-associated neurodegeneration, wherein the phosphorylated soluble HMW tau species is non-fibrillar, has a molecular weight of at least about 500 kDa, and is phosphorylated at least at serine 396, wherein a reduced level of the phosphorylated soluble HMW tau species results in reduced tau-associated neurodegeneration.

In some embodiments, the method can further comprise selectively reducing the level of an additional phosphorylated soluble HMW tau species in the brain (e.g., in the brain interstitial fluid) or cerebrospinal fluid (CSF) of the subject, wherein the additional phosphorylated soluble HMW tau species is non-fibrillar, has a molecular weight of at least about 500 kDa, and is phosphorylated at least at serine 199 (S199), and/or serine 404 (S404).

In some embodiments, the level of a soluble HMW tau species phosphorylated at serine 422 is not substantially reduced in the brain (e.g., in the brain interstitial fluid) or cerebrospinal fluid (CSF) during the treatment. In some embodiments, the level of a soluble HMW tau species phosphorylated at serine 409 is not substantially reduced in the brain (e.g., in the brain interstitial fluid) or cerebrospinal fluid (CSF) during the treatment. In some embodiments, the level of a soluble HMW tau species phosphorylated at serine 400 is not substantially reduced in the brain (e.g., in the brain interstitial fluid) or cerebrospinal fluid (CSF) during the treatment. In some embodiments, the level of a soluble HMW tau species phosphorylated at serine 262 is not substantially reduced in the brain (e.g., in the brain interstitial fluid) or cerebrospinal fluid (CSF) during the treatment. In some embodiments, the level of a soluble HMW tau species phosphorylated at threonine 205 is not substantially reduced in the brain (e.g., in the brain interstitial fluid) or cerebrospinal fluid (CSF) during the treatment.

In some embodiments, the level of soluble LMW tau species in the subject is not substantially reduced in the brain (e.g., in the brain interstitial fluid) or cerebrospinal fluid (CSF) during the treatment.

In some embodiments, at least a portion of the target soluble HMW tau species population (e.g., soluble HMW tau species phosphorylated at least at serine 396) present in brain interstitial fluid of the subject is removed or rendered inactive for propagation. In some embodiments, at least a portion of the target soluble HMW tau species population (e.g., soluble HMW tau species phosphorylated at least at serine 396) present in cerebrospinal fluid of the subject is removed or rendered inactive for propagation.

Methods for selectively reducing the level of the target soluble HMW tau species (e.g., soluble HMW tau species phosphorylated at least at serine 396) in the brain of a subject can be based on physical removal and/or molecular interactions between the target soluble HMW tau species and a proper antagonist. In some embodiments, the phosphorylated soluble HMW tau species present in the brain interstitial fluid and/or cerebrospinal fluid of the subject can be selectively reduced by administering to the brain or CSF of the subject an antagonist of soluble HMW tau species phosphorylated at serine 396, serine 199, and/or serine 404. Examples of an antagonist of such phosphorylated soluble HMW tau species include, without limitations, an antibody, a nuclease (e.g., but not limited to, a zinc finger nuclease (ZFN), transcription activator-like effector nuclease (TALEN), a gene-editing composition (e.g., CRISPR/Cas system), a transcriptional repressor, a nucleic acid inhibitor (e.g., RNAi, siRNA, anti-miR, antisense oligonucleotides, ribozymes, and a combination of two or more thereof), a small organic molecule, an aptamer, and a combination of two or more thereof.

In some embodiments, the method can further comprise selecting a subject determined to have the target soluble HMW tau species (e.g., soluble HMW tau species phosphorylated at least at serine 396) present in the brain or CSF at a level above a reference level. A reference level can represent a level of target soluble HMW tau species (e.g., soluble HMW tau species phosphorylated at least at serine 396) present in healthy subject(s).

In a further aspect, a method of diagnosing tau-associated neurodegeneration based on the presence and/or levels of a soluble HMW tau species phosphorylated at least at serine 396 is also 79-297-82 provided herein. Exemplary tau-associated neurodegeneration includes, but is not limited to, Alzheimer's disease, Parkinson's disease, or frontotemporal dementia. The inventors have shown that the cerebrospinal fluid (CSF) (e.g., ventricular or lumbar CSF) from AD brain extract contained significantly higher levels of phosphorylated soluble HMW tau species, when compared to that of the control brain. Therefore, a method of diagnosing tau-associated neurodegeneration can comprise (a) fractionating a sample of brain interstitial fluid or cerebrospinal fluid from a subject; and (b) detecting a phosphorylated soluble HMW tau species in the sample such that the presence and amount of the phosphorylated soluble HMW tau species is determined, wherein the phosphorylated soluble HMW tau species is non-fibrillar, has a molecular weight of at least about 500 kDa. and is phosphorylated at least at serine 396; and (c) identifying the subject to have, or be at risk for tau-associated neurodegeneration when the level of the phosphorylated soluble HMW tau species in the sample is above a reference level; or identifying the subject to be less likely to have tau-associated neurodegeneration when the level of the phosphorylated soluble HMW tau species is the same as or below a reference level.

In some embodiments, a reference level can represent a level of soluble HMW tau species phosphorylated at least at serine 396 present in healthy subject(s).

The level of soluble HMW tau species phosphorylated at least at serine 396 is generally much lower in healthy subject(s) than in AD subject(s). In some embodiments, the level of soluble HMW tau species phosphorylated at least at serine 396 is about 33 times lower in healthy subject(s) than in AD subject(s). Accordingly, in some embodiments, the subject is identified to have, or be at risk for tau-associated neurodegeneration when the level of the phosphorylated soluble HMW tau species in the sample is at least about 20 times or higher (including, e.g., at least about 25 times, at least about 30 times, at least about 35 times, at least about 40 times or greater) above a reference level (e.g., the level of soluble HMW tau species phosphorylated at least at serine 396 in healthy subject(s)).

In one embodiment, the level of soluble HMW tau species phosphorylated at least at serine 396 in healthy subject(s) is about 0.4 ng per ml of cerebrospinal fluid or brain interstitial fluid. Accordingly, in some embodiments, the subject is identified to have, or be at risk for tau-associated neurodegeneration when the level of the phosphorylated soluble HMW tau species in the sample (e.g., cerebrospinal fluid or brain interstitial fluid) is at least about 10 ng/mL or higher, including, e.g., at least about 11 ng/mL, at least about 12 ng/mL, at least about 13 ng/mL, at least about 14 ng/mL, at least about 15 ng/mL, at least about 20 ng/mL, at least about 25 ng/mL, at least about 30 ng/mL, at least about 35 ng/mL, at least about 40 ng/mL, or higher.

In some embodiments, the method can further comprise detecting an additional phosphorylated soluble HMW tau species in the sample such that the presence and amount of the additional phosphorylated soluble HMW tau species is determined, wherein the additional phosphorylated soluble HMW tau species is non-fibrillar, has a molecular weight of at least about 500 kDa. and is phosphorylated at least at serine 199 (S199), and/or serine 404 (S404).

In some embodiments, the sample, prior to the fractionating of (a), can be substantially free of soluble LMW tau species, wherein the soluble LMW tau species has a molecular weight of no more than 200 kDa. For example, a sample of brain interstitial fluid or cerebrospinal fluid can be obtained from a subject to be diagnosed by microdialysis, e.g., using a proper filter molecular-weight cut-off, which would allow only molecules with a molecular weight of at least about 600 kDa to be collected.

In alternative embodiments, the sample, prior to the fractionating of (a), can comprise soluble LMW tau species, wherein the soluble LMW tau species has a molecular weight of no more than 200 kDa. By fractionating the sample, one can isolate the phosphorylated soluble HMW tau species from the rest of the sample (e.g., a portion including soluble LMW tau species) to determine a diagnostic level. Without limitations, fractionation can be based on size exclusion and/or antibody-based methods.

In some embodiments where soluble LMW tau species is present in the sample, the method can further comprise detecting the amount of the soluble LMW tau species phosphorylated at serine 396 in the sample. In these embodiments, the subject can be identified to have, or be at risk for tau-associated neurodegeneration if a ratio of the S396-phosphorylated soluble HMW tau species to the S396-phosphorylated soluble LMW tau species is the same as or above a reference level ratio; or the subject is identified to be less likely to have tau-associated neurodegeneration if the ratio of the S396-phosphorylated soluble HMW tau species to the S396-phosphorylated soluble LMW tau species is below the reference level ratio. A reference level ratio can represent a level ratio of S396-phosphorylated soluble HMW tau species to S396-phosphorylated soluble LMW tau species present in healthy subject(s).

In some embodiments, the method can further comprise administering to the brain of the subject identified to have, or be at risk for tau-associated neurodegeneration an antagonist of the soluble HMW tau species phosphorylated at serine 396, serine 199, and/or serine 404. Examples of an antagonist of such phosphorylated soluble HMW tau species include, without limitations, an antibody, a nuclease (e.g., but not limited to, a zinc finger nuclease (ZFN), transcription activator-like effector nuclease (TALEN), a gene-editing composition (e.g., CRISPR/Cas system), a transcriptional repressor, a nucleic acid inhibitor (e.g., RNAi, siRNA, anti-miR, antisense oligonucleotides, ribozymes, and a combination of two or more thereof), a small organic molecule, an aptamer, and a combination of two or more thereof. Methods known for effectively delivering an agent to the brain of a subject can be used to perform the treatment methods of various aspects described herein. In some embodiments of this aspect and other aspects described herein, the agent can be administered to the brain via a carrier. An exemplary carrier can be a virus or viral vector (e.g., but not limited to, retrovirus, adenovirus, adeno-associated virus (AAV), recombinant AAV expression vector), a nanoparticle, and/or a liposome.

In some embodiments of this aspect and other aspects described herein, the brain of the subject can be further determined to have an amyloid beta plaque and the administration can reduce neurotoxicity (and/or increase neuron survival) in the presence of amyloid beta.

Not only does the discovery of specific phosphorylated forms of soluble HMW tau species (e.g., phosphorylated at serine 396, serine 404, and serine 199) provide one or more therapeutic targets and biomarkers for tau-associated neurodegeneration as described herein, the specific phosphorylated forms of soluble HMW tau species can also be used in vitro to induce inter-neuron propagation, a phenotypic feature of progression in neurodegeneration, and thus develop an in vitro model to screen for effective agents that reduce cross-synaptic spread of misfolded tau proteins to treat tau-associated neurodegeneration. Accordingly, a further aspect provided herein relates to a method of identifying an agent that is effective to reduce cross-synaptic spread of misfolded tau proteins. The method comprises (a) contacting a first neuron in a first chamber of a neuron culture device with a composition comprising a phosphorylated soluble HMW tau species, the phosphorylated soluble HMW tau species being phosphorylated at serine 396, wherein the first neuron is axonally connected with a second neuron in a second chamber of the neuron culture device, and wherein the second neuron is not contacted with the phosphorylated soluble HMW tau species; (b) contacting the first neuron from (a) in the first chamber with a candidate agent; and (c) detecting transport of the phosphorylated soluble HMW tau species from the first neuron to the second neuron. An effective agent for reducing cross-synaptic spread of misfolded tau proteins can be identified based on detection of the presence or absence of the phosphorylated soluble HMW tau species in an axon and/or soma of the second neuron.

While any neuron culture device suitable for monitoring axonal extension and/or transport can be used in the methods described herein, in some embodiments, the neuron culture device is a microfluidic device. In some embodiments, the microfluidic device can comprise a first chamber for placing a first neuron and a second chamber for placing a second neuron, wherein the first chamber and the second chamber are interconnected by at least one microchannel exclusively sized to permit axon growth.

The S396-phosphorylated soluble HMW tau species described herein can also be used in screening assays to identify agents that modulate the formation or activity of the HMW tau species itself (e.g., by blocking the formation or stability of the HMW tau species, or, for example, by blocking post-translational modifications or by destabilizing the HMW tau structure, or by reducing or inhibiting phosphorylation of the phosphorylated soluble HMW tau species at least at serine 396). For example, aptamers, small organic molecules or other agents can be applied to neuronal cell cultures and the presence or amount of S396-phosphorylated soluble HMW tau or level of phosphorylation at serine 396 of the HMW tau species can be monitored. An agent so identified that blocks the formation or accumulation of S396-phosphorylated soluble HMW tau species and/or reduces the level of phosphorylation at serine 396 of the HMW tau species would be of interest as a potential therapeutic.

Another aspect described herein relates to a solid support comprising phosphorylated soluble high molecular weight (HMW) tau species immobilized thereon, wherein substantially all of the phosphorylated soluble HMW tau species are phosphorylated at one, two or all of the following amino acid residues: serine 396, serine 199, and serine 404. The solid support can substantially lack LMW tau.

A further aspect described herein relates to a solid support comprising phosphorylated soluble HMW tau species antagonists immobilized hereon, wherein substantially all of the phosphorylated soluble HMW tau species antagonists specifically bind soluble HMW tau species bearing phosphate at serine 396, serine 199, or serine 404. The solid support can substantially lack antagonists to LMW tau. In some embodiments, the solid support can comprise an antagonist to a non-tau molecule.

A preparation of S396-phosphorylated soluble HMW tau polypeptide comprising covalent cross-links between one or more tau polypeptide monomers is also described herein.

It should be noted that the location of phosphorylation sites on soluble HMW tau species as described herein, e.g., S396, S199, S404, S422, T205, and S262, is based on a tau reference sequence of SEQ ID NO: 1, and will shift or change accordingly when a different tau sequence is used, for example, when a fragment of the tau reference sequence of SEQ ID NO: 1, or a different tau isoform is used. One of skill in the art can readily identify phosphorylated sites on different human tau isoforms or functional variants thereof that correspond to the ones based on SEQ ID NO: 1 (e.g., S396, S199, S404, S422, T205, and S262). For example, by aligning the tau reference sequence of SEQ ID NO: 1 and a tau sequence of interest using any art-recognized sequence alignment tool, e.g., NCBI Protein BLAST, one can correspond the phosphorylation sites from SEQ ID NO: 1 to a different tau sequence of interest.

Further embodiments of the technology described herein are described and encompassed in; Takeda, S. et al. Nat Commun. October 13; 6:8490 (2015): Takeda, S. et al. Ann Neurol. 2016 Jun. 28; Wegmann, S. et al. EMBO J. 2015 Dec. 14; 34(24):3028-41; the contents of which are incorporated herein in their entireties.

### BRIEF DESCRIPTION OF THE DRAWINGS

FIGS. 1A-1L show neuronal uptake of HMW tau from brain extract of rTg4510 tau-transgenic mouse. (FIG. 1A) Primary neurons were incubated with PBS-soluble brain extracts (3,000 g-150,000 g centrifugation supernatant, 500 ng/ml human tau) from a 12-month-old rTg4510 mouse. (FIG. 1A, left) Immunostaining with human tau specific antibody (green) and total (human and mouse) tau antibody (red, as a neuronal marker). (FIG. 1A, right) Quantification of human tau uptake. (n=9-12) (FIG. 1B) Neurons were incubated with brain extracts (500 ng/ml human tau) for 2 and 5 days. (FIG. 1C) Tau uptake assay in HEK-tau-biosensor cells. Brain extracts (1 μg protein) were applied to the cells (lipofectamine (−)). (n=4) Mann-Whitney U-test. (FIGS. 1D-1E) Size exclusion chromatography (SEC) of PBS-soluble brain extracts. (FIG. 1D) Representative graph of human tau levels (ELISA) in SEC-separated samples. (FIG. 1E) Mean human tau levels of HMW (Frc.2-4), MMW (Frc.9-10), and LMW (Frc.13-16) SEC fractions. (n=3) HMW, high molecular weight; MMW, middle molecular weight; LMW, low molecular weight. (FIG. 1F, left) Neurons were incubated with SEC fractions (100 ng/ml human tau) from 3,000 g extract and immunostained. (FIG. 1F, right) Quantification of human tau uptake. (n=3-5) (FIG. 1G) Tau uptake assay in HEK-tau-biosensor cells. HMW (Frc.2)/LMW (Frc. 14) fractions were applied without lipofectamine. (n=4) (FIG. 1H) AFM analysis of HMW tau isolated from rTg4510 brain (10,000 g total extract, SEC Frc. 3). Full color range corresponds to a vertical scale of 20 nm. Scale bar: 100 nm. (FIG. 1H, right) Size (AFM heights) distribution histogram of HMW tau. (FIG. 1I) Human tau taken by neurons was co-stained with Alz50 antibody or ThioS. Brain sections from rTg4510 mouse were used as positive controls for each staining. (FIGS. 1J-1L) HMW tau uptake into neurons in vivo. (FIG. 1J) HMW (Frc.2-3)/LMW (Frc. 13-14) SEC fractions (rTg4510, PBS-3,000 g, 100 ng/ml human tau) or PBS were injected into the left hippocampus of pre-tangle stage rTg4510 mice (2-3 months). (FIG. 1K) Three weeks after the injection, the brains were collected and immunostained for tau (AT8). Scale bar: 500 μm. (FIG. 1L) Quantification of AT8-positive neurons in the ipsilateral dentate gyrus (Kruskal-Wallis test). Scale bar: 25 μm, except for (FIG. 1H) and (FIG. 1K). *P<0.05, **P<0.01.

FIGS. 2A-2H show that lack of PBS-soluble phosphorylated soluble HMW tau species is associated with low tau uptake in primary neurons. (FIG. 2A, top) Uptake of human tau from brain extracts from rTg4510 and rTg21221 mice by primary neurons (PBS-3,000 g, 500 ng/ml human tau). Neurons were immunostained with human tau specific antibody (green) and total (human and mouse) tau antibody (red). (FIG. 2A, bottom) Tau uptake assay in HEK-tau-biosensor cells. Brain extracts (10 μg protein) were applied to the cells (lipofectamine (−)). (n=4) Scale bar: 50 μm. (FIG. 2B) Human tau levels in brain extracts (ELISA). (FIG. 2C) Immunoblot analysis of PBS-soluble extracts with total tau antibody (DA9). Up-shifted bands in rTg4510 brain suggest phosphorylation of tau (arrow). (FIG. 2D) Brain extracts were immunoblotted with phospho-tau specific antibodies recognizing different epitopes. Representative immunoblot and quantification of phospho-tau levels at each epitope. (n=3-4) (FIGS. 2E-2F) SEC analysis of PBS-soluble tau. (FIG. 2E) Representative graph of human tau levels (ELISA) in SEC-separated samples (FIG. 2F) Mean human tau levels of HMW (Frc.2-4) and LMW (Frc. 13-16) SEC fractions. (n=3-6) (FIG. 2G) Immunoblot analysis (SDS-PAGE) of SEC-separated fractions from brain extracts (total tau, DAKO). Quantification of band density is also shown (right graphs) (n=4). (FIG. 2H) Dot blot analysis of PBS-soluble brain extracts with tau oligomer-specific antibody (T22), human tau-specific antibody (Tau13), and total tau antibody. Quantification of dot blot signals is also shown (right) (n=4). 11-13 months old animals were used. *P<0.05, **P<0.01.

FIGS. 3A-3C show a three-chambered microfluidic device for modeling dual-layered neurons. (FIG. 3A) Schematics of a microfluidic device for culturing neurons in three distinct chambers. Mouse primary neurons are plated into the 1st and 2nd chambers (100 μm in thickness) and axon growth is guided through microgrooves (3 μm in thickness, 600 μm in length) connecting each chamber. (FIG. 3B, left) Axons from the 1st chamber neuron (green, DA9 as axonal marker) extend into the 2nd chamber within 4 days (neurons were plated only in the 2nd chamber). No MAP2 positive dendrites (red) were found in the 2nd chamber, indicating that a 600 μm microgroove is sufficiently long to isolate axon terminals from soma and dendrites. (FIG. 3B, middle) Most axons from the 2nd chamber neuron extend into the 3rd chamber (neurons were plated only in the 2nd chamber). (FIG. 3B, right) Two sets of neurons were plated into the 1st and 2nd chamber and established synaptic contact in the 2nd chamber. (FIG. 3C) Neurons in the 1st and 2nd chambers were transfected with green fluorescent protein (GFP) and red fluorescent protein (RFP), respectively. GFP positive axon from the 1st chamber neuron extended into the 2nd chamber, connecting to RFP positive 2nd chamber neuron, which projected its axon into the 3rd chamber. Scale bar: 50 μm.

FIGS. 4A-4E show neuron-to-neuron transfer of rTg4510 mouse brain-derived human tau species in a three-chambered microfluidic device. (FIG. 4A) PBS-soluble extract from rTg4510 brain (12 months old, 500 ng/ml human tau) was added to the 1st chamber of a 3-chamber microfluidic device. Diffusion of brain extract from the 1st to the 2nd chamber was blocked by a hydrostatic pressure barrier. (FIG. 4B) Immunostaining for human tau (green) and total (human and mouse) tau (red) at day 5. Human tau positive neurons were detected in the 2nd chamber (white arrow). Neurons in the side reservoir of the 2nd chamber were negative for human tau staining (bottom). (FIG. 4C) A human tau positive axon (arrow) and dendrite (arrow head) extending from the 2nd chamber neuron. (FIG. 4D) Concentration dependency of tau uptake and propagation. rTg4510 brain extract (PBS-3,000 g) was diluted in culture medium to obtain three different concentrations (6, 60, and 600 ng/ml) of human tau and added into the 1st chamber. Neurons were immunostained for human tau and total (human and mouse) tau at day 5. (FIG. 4D, right) Quantification of fluorescence intensity of human tau staining in the 2nd chamber. (n=4-7) (FIG. 4E) Time course of neuron-to-neuron transfer of rTg4510 brain derived human tau. The rTg4510 brain extract (PBS-3,000 g, 500 ng/ml human tau) was added to the 1st chamber and incubated for up to 14 days. Neurons were immunostained at different time points. Human tau positive 2nd chamber neurons (arrow head) and axons from the 1st chamber neuron (arrow) were detected after 5 days of incubation. Human tau positive axons were detected in the 3rd chamber after 8 days (arrow). Scale bar: 50 μm. *P<0.05.

FIGS. 5A-5C show that rTg4510 brain derived human tau was stable and propagated even after removal of brain extract from the chamber. (FIG. 5A) rTg4510 brain extract (12 months old, PBS-3,000 g) was diluted in culture medium (500 ng/ml human tau in final concentration) and added to the 1st chamber of 3-chamber microfluidic neuron device. After 2 days (before tau propagation occurs) or 5 days (after tau propagation occurred, but not yet progressed to the 3rd chamber) of incubation, brain extract was washed out from the 1st chamber and replaced with fresh culture medium. (FIGS. 5B-5C) Neurons were immunostained for human tau (green) and total (human and mouse) tau (red) at designated time points. (FIG. 5B) Human tau positive neuron was detected in the 2nd chamber (day 8, arrow) even after Tg brain extract was washed out from the 1st chamber at day 2. (FIG. 5C) Human tau was detected in the 3rd chamber axons (arrow) even after Tg brain extract was washed out from the 1st chamber at day 5. Human tau taken up by the 1st chamber neuron was still detectable at day 14 (9 days after removal of Tg brain extract). Scale bar: 50 μm.

FIGS. 6A-6M show neuronal uptake of PBS-soluble HMW tau derived from human AD brain. (FIGS. 6A-6B) Primary neurons were incubated with AD or control brain extracts (cases were matched for age and postmortem interval (Table 2)) and immunostained at day 2 (FIG. 6A). (FIG. 6B) Quantification of fluorescence intensity of human tau staining. (FIGS. 6C-6D) Tau uptake (FIG. 6C) and seeding activity (FIG. 6D) assay in HEK-tau-biosensor cells. (Mann-Whitney U-test) (FIG. 6E) Subcellular localization of human tau taken up by neurons (PBS-3,000 g, 500 ng/ml human tau). (FIG. 6F) Neuron-to-neuron transfer of tau in a 3-chamber microfluidic device. AD brain extract (PBS-3,000 g, 500 ng/ml human tau) was added to the 1st chamber. Human tau positive neurons were detected in both the 1st and 2nd chamber at day 7 (arrow). (FIGS. 6G-6H) Quantification of total-tau (FIG. 6G) and phospho-tau (FIG. 6H) levels in AD and control brain extract (ELISA). (FIG. 6I) Brain extracts were immunoblotted with phospho-tau specific antibodies recognizing different epitopes. Representative immunoblot and quantification of phospho-tau levels at each epitope. (FIGS. 6J-6K) SEC analysis of PBS-soluble tau from AD and control brain. (FIG. 6J) Representative graph of total tau levels (ELISA) in SEC-separated samples. Small peaks for HMW fractions were detected in both groups (right panel). (FIG. 6K) Mean total tau levels of HMW SEC fractions. (FIG. 6L) Tau uptake from each SEC fraction (5 or 500 ng/ml human tau) by primary neurons. (FIG. 6M) Phospho-tau levels in each SEC fraction (ELISA). Scale bar: 25 μm. *P<0.05, **P<0.01.

FIGS. 7A-7J show that tau phosphorylation correlates with cellular uptake (FIGS. 7A-7C) Non-phosphorylated soluble HMW tau was not taken up by neurons. (FIG. 7A) Tau oligomer mixture solution was prepared from recombinant human tau, followed by SEC and tau ELISA. (FIG. 7B) Phospho-tau levels in SEC fractions and brain extracts (pS396 tau ELISA). (FIG. 7C) Each SEC fraction was incubated with primary neurons. Neurons were immunostained at day 2. (FIGS. 7D-7F) Dephosphorylation reduced tau uptake. (FIG. 7D) Immunoblot analysis of total-tau (Tau13) and phospho-tau (pS396) levels in rTg4510 (12 months old) brain extracts treated with lambda phosphatase. (FIG. 7E) SDD-AGE analysis of brain extracts treated with phosphatase. (FIG. 7F) Tau uptake assay. Phosphatase-treated brain extract was applied to HEK-tau-biosensor cells. (n=3, **P<0.01) (FIGS. 7G-7J) Immunodepletion of phospho-tau reduced neuronal tau uptake. rTg4510 (12 months old) brain extracts were immunodepleted with total-tau or phospho-tau specific antibodies. (n=5) (FIG. 7G) Total tau levels in tau-immunodepleted samples (ELISA). **P<0.01 vs. control IgG. (FIG. 7H) Tau uptake in primary neurons (day 2). *P<0.05 vs. control IgG. (FIG. 7I) Blocking efficiency was defined as the percentage of tau-uptake reduction (vs. control-IgG) multiplied by tau levels in the immunodepleted brain extracts (% control-IgG). *P<0.05 vs. total tau (HT7). (FIG. 7J) Representative images of tau uptake in primary neurons. Scale bar: 50 μm. SDD-AGE, Semi-denaturing detergent agarose gel electrophoresis.

FIGS. 8A-8E show that extracellular tau species from rTg4510 mouse brain can be taken up by primary neurons. (FIG. 8A) A large-pore probe in vivo microdialysis with push-pull perfusion system. ISF samples were collected from freely-moving rTg4510 and control mice (seven months old) using a 1,000 kDa cut-off probe. (FIG. 8B) Representative probe placement. Horizontal brain sections were obtained after ISF collection (24 hours after probe insertion) and stained for human tau (green) and DAPI. Dotted line depicts probe location (top). The probe was briefly perfused with Texas red dye (70 kDa, 1 mg/ml) to locate the site of microdialysis. There was no morphological evidence of substantial neuronal loss. There was no apparent difference in the number of human tau positive neurons between ipsilateral (probe-implanted side) and contralateral hippocampal sections (b, bottom). Hip, hippocampus. (FIG. 5C) Representative graph of human tau levels in SEC-separated ISF sample from rTg4510 mouse. 400 ul of microdialysate was loaded on SEC column and tau levels in each fraction were measured by ELISA. (FIG. 8D) ISF samples were incubated with primary neurons, which were then immunostained for human tau and total (human and mouse) tau. ISF from rTg4510 was diluted to a final concentration of 40 ng/ml human tau and the same volume of ISF from a control mouse was used for incubation. (FIG. 8E) Concentration dependency of ISF tau uptake by primary neurons. rTg4510 brain ISF was diluted in culture medium to obtain three different concentrations (10, 20, and 40 ng/ml) of human tau. HMW, high molecular weight. Scale bar: 50 μm.

FIGS. 9A-9D show tau seeding activity assay in HEK-tau-biosensor cells. (FIG. 9A) Representative image of intracellular tau aggregate induced by rTg4510 brain extract (PBS-soluble, 3,000 g, 10 μg protein). Single confocal (top) and z-stack (3D, bottom) images were taken at 12 hours. Scale bar: 10 μm. (FIG. 9B) Time-course of tau seeding. PBS-soluble 3,000 g or 150,000 g brain extracts from rTg4510 mice were applied to HEK-tau-biosensor cells with lipofectamine (1%/). Time-lapse confocal images (FRET channel; ex. 458 nm, em. 500-550 nm) were taken every 10 min and fluorescence intensity of the FRET images was measured. (FIG. 9B, left) Representative graph of FRET intensity. (FIG. 9B, right) Confocal images (FRET channel) at 0, 6, 12, and 24 hours time points are shown. 3,000 g brain extracts have higher seeding activity than 150,000 g extracts. Scale bar: 50 μm. (FIGS. 9C-9D) Seeding activities of 3,000 g and 150,000 g brain extracts (n=4/group) were compared at 3 and 17 hours time points. (FIG. 9C) Representative confocal images of tau aggregates. 3,000 g brain extract induced intracellular tau aggregation as early as 3 hours (arrow head). (FIG. 9D) Quantification of FRET density. *P<0.05. 12-month-old rTg4510 mice were used.

FIGS. 10A-10C show dot blot and SDS-PAGE analysis of HMW tau from rTg4501 brain extracts. (FIGS. 10A-10B) PBS-3,000 g brain extracts from rTg4510 (12 months old) were incubated with 8 M urea (FIG. 10A) or 10% SDS (FIG. 10B) for 24 hours at 37° C. and analyzed by dot blot using tau oligomer-specific (T22) and total tau (Tau13) antibodies. Representative images of dot blot (left) and quantification of immunoreactivities of each antibody (right) are shown. Immunoreactivity of the tau oligomer-specific antibody (T22) significantly decreased after exposure to 8 M urea. (n=5-7) **P<0.01 (paired t-test). (FIG. 10C) SDS-PAGE analysis of the SEC HMW fraction (Frc.2) from rTg4510 brain extracts (PBS-3,000 g). The SEC HMW fraction was incubated with 8 M urea for 24 hours at 37° C. and analyzed by SDS-PAGE using total tau (DAKO) antibody. Representative blot (left) and quantification (right) are shown. (n=3) **P<0.01 (paired t-test). The HMW smear disappeared after exposure to 8 M urea, indicating the existence of a multimeric tau assembly in the HMW fraction.

FIG. 11 is a panel of fluorescent images showing subcellular localization of tau taken up by neurons. Mouse primary neurons were incubated with PBS-soluble brain extracts (3,000 g, 500 ng/ml human tau) from a 12-month-old rTg4510 mouse and immunostained with human tau specific antibody (Tau13, green) and subcellular markers (red) on day 3. Scale bar: 25 μm

FIG. 12 is a panel of fluorescent images showing concentration dependency of tau uptake in vitro. Primary neurons were incubated with rTg4510 brain extracts (12 months old, PBS-3,000 g, 0.1-50 ng/ml human tau) and immunostained with human tau specific antibody (Tau13, green) and total (human and mouse) tau antibody (red). Scale bar: 25 μm

FIGS. 13A-13B show neuronal tau uptake and propagation in the absence of astrocytes. (FIGS. 13A-13B) Mouse primary neurons were incubated with rTg4510 (12 months old) brain extracts (PBS-3,000 g, 500 ng/ml human tau) in a normal culture dish (FIG. 13A) or 3-chamber microfluidic device (FIG. 13B), and immunostained with human tau specific antibody (Tau13, green), MAP2 antibody (red, neuronal marker), and GFAP antibody (blue, astrocyte marker). (FIG. 13A) Neuronal tau uptake in the absence of astrocyte. GFAP positive astrocytes, although infrequent, were found in the primary neuron culture dish (FIG. 13A, 1: Astrocyte (+), arrow). Neuronal tau uptake (arrow head) was detected in both the presence (FIG. 13A, 1: Astrocyte (+)) and absence (FIG. 13A, 2: Astrocyte (−)) of astrocytes. (FIG. 13B) Neuron-to-neuron transfer of tau in the absence of astrocytes. There was no detectable astrocyte contamination in the 2nd chamber of the microfluidic device. rTg4510 brain extract was added to the 1st chamber and human tau positive neuron was detected in the 2nd chamber (day 8) in the absence of astrocyte. Scale bar: 50 μm.

FIGS. 14A-14C show HMW tau uptake into neurons in vivo. (FIG. 14A) HMW (Frc.2-3)/LMW (Frc. 13-14) SEC fractions from rTg4510 brain extract (12 months old, PBS-3,000 g, 100 or 500 ng/ml human tau) or PBS were injected into the left frontal cortex of 3-month-old WT mice. (FIG. 14B) 48 hours after injection, brains were collected and immunostained with human tau specific antibody (Tau13, green), anti-NeuN antibody (red, neuronal marker), and DAPI (blue). Human tau positive neurons were detected from mice injected with HMW fraction (arrow heads). (FIG. 14C) Semi-quantitative analysis of human tau positive neurons. Scale bar: 10.μm.

FIGS. 15A-15C are graphs showing correlations of tau uptake by primary neurons with human tau levels in each indicated SEC-separated fraction. The degree of neuronal tau uptake correlated with HMW tau levels, but not with MMW or LMW tau levels. HMW, high molecular weight; MMW, middle molecular weight; LMW, low molecular weight. 11-13 months old rTg4510 mice were used (n=7). Pearson correlation analysis.

FIG. 16 is a gel image showing SDD-AGE analysis of PBS-soluble brain extracts. SDD-AGE of brain extracts (PBS-3,000 g) from rTg4510 and rTg21221 mice (12 months old) shows lack of HMW tau species in rTg21221 mice, although the rTg4510 brain has both HMW and LMW tau. Rabbit polyclonal anti-total tau antibody (# ab64193, Abcam) was used as primary antibody. HMW, high molecular weight; LMW, low molecular weight. SDD-AGE, Semi-denaturing detergent agarose gel electrophoresis.

FIGS. 17A-17D show anterograde and retrograde tau propagation in a three-chambered microfluidic device. (FIGS. 17A-17B) Anterograde tau propagation in a 3-chamber microfluidic device. rTg4510 brain extract (12 months old, PBS-3,000 g, 500 ng/ml human tau) was added to the 1st chamber. (FIG. 17A) Human tau-positive neurons were detected in the 2nd chamber on day 5 (arrow). (FIG. 17B) Quantification of human tau levels in the culture media collected from the 1st, 2nd, and 3rd chambers on day 5 (human total tau ELISA). There was no detectable level of human tau in the 2nd or 3rd chamber. (n=3) (FIGS. 17C-17D) Retrograde tau propagation in a 3-chamber microfluidic device. rTg4510 brain extract (12 months old, PBS-3,000 g, 500 ng/ml human tau) was added to the 2nd chamber. (FIG. 17C) Human tau-positive neurons and axons were detected in the 1st and 3rd chambers on day 5 (arrow). (FIG. 17D) Quantification of human tau levels in the culture media collected from the 1st, 2nd, and 3rd chambers on day 5 (human total tau ELISA). There was no detectable level of human tau in the 1st or 3rd chamber. (n=3) Diffusion of brain extract between chambers was blocked by a hydrostatic pressure barrier. Scale bar: 50 μm.

FIGS. 18A-18B show results of a cell viability assay with ethidium homodimer-1 (EthD-1) staining, indicating that tau uptake and intracellular aggregation do not cause acute cell death. rTg4510 brain extract (12 months old, PBS-3,000 g, 10 μg protein) was transduced into HEK-tau-biosensor cells in the absence of lipofectamine. Cells were stained with EthD-1 (4 μM), which stains the dead cells due to their compromised cell membranes and leaves the healthy cells unstained, and Hoechst 33342 (1 μg/ml) at the time points of 16 hours and day 4. (FIG. 18A) Confocal images at 16 hours (left) and on day 4 (right) shows: tau sensor cells with intracellular tau aggregates, but negative for EthD-1 staining (1), EthD-1 positive dead cells without tau aggregates (2), and EthD-1 positive dead cells with tau aggregates (3). (FIG. 18B) Percentage of EthD-1 positive dead cells in total, tau aggregates negative (Tau (−)), and tau aggregates positive (Tau (+)) cells (day 4). Total number of cells was obtained by Hoechst staining. There was no difference in the percentage of dead cells among groups (n=4/group, one-way ANOVA, F(2, 9)=0.099; P=0.906). Scale bar: 50 μm.

FIGS. 19A-19D show effect of HMW tau on neuronal viability (MTT assay). (FIG. 19A) Mouse primary neurons were incubated with HMW (Frc.2)/LMW (Frc. 14) SEC fractions from rTg4510 brain extracts (12 months old, PBS-3,000 g, 10 ng/ml human tau). Neuronal viability was measured by MTT assay at 48 hours. There was no difference in MTT-reducing activity between groups (n=4/group, P=0.829, Student's t-test). (FIGS. 19B-19D) Effect of immunodepletion of HMW tau on neuronal tau uptake and viability. (FIG. 19B) HMW SEC fraction from rTg4510 brain extracts (12 months old, PBS-3,000 g, 10 ng/ml human tau) were immunodepleted with total (HT7) or phospho (pS396) tau antibodies (n=3/group, **P<0.01, one-way ANOVA and a subsequent Tukey-Kramer test). (FIG. 19C) Mouse primary neurons were incubated with immunodepleted HMW fractions for 48 hours and immunostained with human tau specific antibody (green) and total (human and mouse) tau antibody (red). Immunodepletion with total (HT7) and phospho (pS396) tau antibodies reduced neuronal tau uptake. Scale bar: 20 μm. (FIG. 19D) Immunodepletion with total (HT7) and phospho (pS396) tau antibodies did not alter neuronal MTT-reducing activity at 48 hours (n=3/group, P=0.575, one-way ANOVA).

FIGS. 20A-20E show seeding and uptake activity of high-molecular-weight (HMW) extracellular tau derived from brain interstitial fluid (ISF) or cerebrospinal fluid (CSF) of tau-transgenic rTg4510 mice and control rTg21221 mice. FIG. 20A is a schematic diagram showing in vivo brain microdialysis using a probe with a 1000 kDa molecular weight cutoff to collect HMW tau from brain interstitial fluid (ISF), which is then applied to HEK-tau-biosensor cells as described in Holmes et al. (Ref. #23). FIGS. 20B-20C show brain ISF (FIG. 20B) and CSF (FIG. 20C) derived from tau-transgenic rTg4510 mice had higher seeding activity that those from the control brain. FIGS. 20D-20E show brain ISF (FIG. 20D) and CSF (FIG. 20E) derived from tau-transgenic rTg4510 mice had higher cellular uptake activity that those from the control brain.

FIGS. 21A-21H show that human AD postmortem ventricular CSF contains bioactive HMW tau. Ventricular CSF collected from postmortem AD subjects is applied to HEK-tau biosensor cells as described in Holmes et al. PNAS (2014) 111:E4376-4385, e.g., FRET biosensor HEK293 cell Tau(P301S)-RD-CFP/YFP, for tau seeding assay (with addition of lipofectamine) or tau uptake assay (without addition of lipofectamine). FIG. 21A is a graph showing the total tau levels in the ventricular CSF of each indicated AD subject, as measured using the human tau-specific ELISA. FIG. 21B is a graph showing molecular-weight size distribution of tau present in the AD human CSF samples assessed by size-exclusion chromatography (SEC). FIG. 21C is a graph showing the amount of HMW tau (fraction 1 from SEC) in the AD human CSF samples as measured using the human tau-specific ELISA. FIG. 21D is a set of fluorescent images showing seeding activity (upper row) and cellular uptake activity (lower row) of tau derived from the AD human CSF samples as measured using HEK-tau-biosensor cells. FIG. 21E contains a set of fluorescent images and quantitative data comparing seeding activity of tau derived from either AD human total CSF or HMW tau-comprising fractionated CSF in various concentrations, as measured using HEK-tau-biosensor cells. FIG. 21F shows amount of tau left in the AD human CSF samples after immunodepletion with various indicated antibodies, namely control IgG, anti-total tau antibody (HT7), and anti-pS395 tau antibody.

FIG. 21G shows seeding activity (left) and blocking efficiency (right) of tau derived from the AD human CSF samples after immunodepletion with the indicated antibodies. Immunodepletion of phospho-tau reduced seeding activity. FIG. 21H is a gel image of semi-denaturing detergent agarose gel electrophoresis (SDD-AGE) showing that tau is hyperphosphorylated in the AD human CSF samples.

FIGS. 22A-22C show that HMW tau accumulates in the lumbar CSF of AD human subjects. FIG. 22A is a set of graphs showing total tau levels (left), phospho-tau levels (middle), and Aβ1-42 levels (right) in the lumbar CSF of AD and control human subjects. FIG. 22B shows the correlation of total tau and Aβ1-42 levels in the lumbar CSF of AD and control human subjects. FIG. 22C shows total tau levels measured in each indicated fraction of the lumbar CSF collected from control subjects and AD subjects. CSF collected from the lumbar is subjected to SEC for separation into fractions of different molecular weight ranges, and the total tau in each indicated faction is measured by human tau-specific ELISA. The left graph shows the data of all 13 control subjects. The middle graph shows the data of all 8 AD subjects. Each line corresponds to a human patient. The right graph shows the average total tau levels in each indicated fraction based on the measured data shown in the left and middle graphs.

FIG. 23 shows quantification of tau Protein on Invitrogen ELISA, AD vs. Control, HMW vs. LMW.

FIG. 24 shows quantification of tau Protein on tau5/HT7 ELISA, AD vs. Control, HMW vs. LMW. HMW fraction from human AD brain contains more tau as detected by Ht7/tau5 ELISA compared to commercial standard Invitrogen ELISA (FIG. 23)

FIG. 25 shows another potential screening mechanism. The Carboxy terminus (tau 46 detectable) is difficult to detect in HMW tau, indicating that it is hidden or truncated.

FIGS. 26A-26B show sensitivity and specificity of the FRET-based tau seeding assay in vitro. FIG. 26A shows HEK-tau-biosensor cells were treated with AD brain extracts (PBS-soluble, 10,000 g spin) from the entorhinal cortex or cerebellum (1% lipofectamine). Total tau levels were measured with ELISA. The number of tau aggregate-positive cells was quantified at 48 h. (n=3/group) *P<0.05, Mann-Whitney U-test. FIG. 26B shows HEK-tau-biosensor cells were treated with AD brain extracts (entorhinal cortex or cerebellum) or recombinant full-length human tau (441 aa) at designated tau concentrations (1% lipofectamine). Representative confocal images (FRET channel) of intracellular tau aggregates at 48 h are shown. Scale bar: 10 μm. The assay was performed in a 384-well plate.

FIGS. 27A-27D show seeding and uptake activity are present in mouse extracellular tau. FIG. 27A shows Protein fractionation and concentration using ultrafiltration spin column. (FIG. 27A, top left) Three molecular weight markers (MWMs), 669 kDa (thyroglobulin), 75 kDa (conalbumin), and 13.7 kDa (ribonuclease), were fractionated using an ultrafiltration spin column with a 100 kDa cut-off membrane (3,000 g spin, 10 min). The original MWM mixture solution, concentrate, and filtrate were analyzed by using SEC. SEC patterns of the original MWM mixture (FIG. 27A, bottom left), concentrate (top right), and filtrate (bottom right) demonstrate that high-molecular-weight proteins (669 kDa) were retained in the concentrate and most lower-molecular-weight proteins (75 kDa and 13.7 kDa MWMs) were fractionated into the filtrate. FIGS. 27B-27D show seed-competent CSF tau was present in the high-molecular-weight fraction. FIG. 27B shows CSF samples from rTg4510 mice were fractionated by sequential ultrafiltration using 100 kDa and 10 kDa cut-off membranes. Pooled CSF samples (180 μl total volume) were used for each run of fractionation. FIG. 27C shows human tau levels in each fraction (100 kDa cut-off concentrate, 10 kDa cut-off concentrate, and 10 kDa cut-off filtrate) were measured using human tau-specific ELISA. (n=3) FIG. 27D shows tau seeding assay in HEK-tau-biosensor cells. Each fraction was applied to the cells with lipofectamine (1%). Confocal images (FRET channel) at day 3 are shown. Scale bar: 10 μm. Tau seeding/uptake assays were performed in 384-well plates.

FIGS. 28A-28J show human AD postmortem ventricular CSF contains bioactive HMW tau species. FIG. 28A shows total tau levels in human postmortem ventricular CSF samples (ELISA). FIGS. 28B-D show AD ventricular CSF contains HMW tau species. FIG. 28B shows SEC analysis of ventricular CSF tau. Total tau levels in each SEC fraction were measured using ELISA.

FIG. 28C shows total tau values in HMW SEC fraction (Frc. 1). FIG. 28D shows SDS-PAGE analysis demonstrates the presence of HMW tau species in postmortem ventricular CSF. Recombinant full-length tau was loaded as a molecular weight marker (30 ng/lane). Total tau antibody (Tau13) was used. FIGS. 28E-F show tau seeding (FIG. 28E) and uptake (FIG. 28F) assay in HEK-tau-biosensor cells. Ventricular CSF samples were applied to the cells with (seeding assay) or without (uptake assay) lipofectamine in 384-well plates. Confocal images (FRET channel) at 48 or 72 h are shown. White arrowhead indicates intracellular tau aggregate. FIG. 28G shows the ventricular CSF HMW tau displays higher seeding activity. The HMW tau species (SEC Frc.1) was isolated from ventricular CSF using SEC and applied to HEK-tau-biosensor cells (384-well plates) at different concentrations of total tau (0.04, 0.2 and 1.0 ng/ml). (FIG. 28G, left) Representative confocal images of tau aggregates induced by HMW tau (white arrowhead). (FIG. 28G, right) Quantification of intracellular tau aggregates. (n=4-7) **P<0.01, Mann-Whitney U-test. FIGS. 28H-J show the immunodepletion of phospho-tau from ventricular CSF efficiently reduced tau seeding activity. Ventricular CSF (#1319, #1223 and #1226) were immunodepleted with total (HT7) or phospho (pS396) tau specific antibodies.

FIG. 28H shows total tau levels in tau-immunodepleted samples (ELISA). (n=3) **P<0.01, One-way ANOVA and a subsequent Dunnett test. FIG. 28I shows tau seeding assay in HEK-tau-biosensor cells. (FIG. 28I, left) Representative confocal images of tau aggregates (white arrowhead). (FIG. 28I, right) Quantification of intracellular tau aggregates (384-well plates). (n=3) **P<0.01, One-way ANOVA and a subsequent Dunnett test. FIG. 28J shows blocking efficiency was defined as the percentage of tau-uptake reduction (vs. control-IgG) multiplied by tau levels in the immunodepleted ventricular CSF (% control-IgG). (n=3) *P<0.05, Student's t-test. Scale bar: 10 μm. HMW, high molecular weight; LMW, low molecular weight; SDS-PAGE, Semi-denaturing detergent agarose gel electrophoresis.

FIGS. 29A-29F show HMW tau species accumulates in the lumbar CSF of AD patients. FIGS. 29A-C show lumbar CSF total tau (FIG. 29A), phospho-tau (FIG. 29B) and Aß1-42 (FIG. 29C) levels in control (n=19), AD (n=15), and FTD (n=10) patients. AD patients had significantly increased total and phospho tau (pT181) levels (vs. control and FTD) and lower Aß1-42 levels (vs. control). *P<0.05, **P<0.01, Kruskal-Wallis test followed by Steel-Dwass multiple comparison test. Bars indicate mean values. FIG. 29D shows scatterplot of CSF Aß1-42 and total tau levels in control, AD, and FTD patients. The cut-points of 133 pg/ml for total tau (red line) and 370 pg/ml for Aß1-42 (blue line) generate a sensitivity and specificity of >80%. FIGS. 29E-F show SEC analysis of lumbar CSF tau. FIG. 29E shows the thin dashed lines represent total tau levels for individual subjects, and the thick solid line represents the averaged value for each group. FIG. 29E shows total tau levels in each SEC fraction. AD patients had significantly higher tau levels in the HMW SEC fraction (Frc.1) compared with control subjects. *P<0.05, Kruskal-Wallis test followed by Steel-Dwass multiple comparison test.

FIGS. 30A-30I show potentially seed-competent HMW tau is present in lumbar CSF of AD patients. FIGS. 30A-B show lumbar CSF samples (1.5 ml) from eight subjects were fractionated and concentrated using an ultrafiltration spin column with a 100 kDa cut-off membrane. Tau seeding activities in the HMW fraction (100 kDa cut-off concentrate) were assessed using HEK-tau-biosensor cells (1% lipofectamine) in 384-well plates. FIG. 30B shows tau seeding assay in HEK-tau-biosensor cells. Confocal images (FRET channel) at day 3 are shown. FIGS. 30C-E show sensitive and quantitative detection of tau seeding activity in human lumbar CSF using FRET flow cytometry. FIG. 30C shows large-volume (8 ml) lumbar CSF samples were fractionated and concentrated using an ultrafiltration spin column with a 100 kDa cut-off membrane. Concentrates were applied to HEK-tau-biosensor cells (1% lipofectamine) in 96-well plates, followed by confocal image analysis and FRET flow cytometry. FIG. 30D shows confocal images (FRET channel) at day 3. FIG. 30E shows tau seeding activity measured by FRET flow cytometry. High HMW tau CSF from an AD patient (# Z09683) produced a high FRET signal. **P<0.01 vs. NT, One-way ANOVA and a subsequent Dunnett test. NT, no treatment. Values in parentheses indicate the levels of HMW tau in SEC Frc. 1 measured in FIG. 28. FIGS. 30F-I show ultrafiltration does not generate a seed-competent HMW tau species from recombinant tau. 1.5 ml of recombinant full-length human tau (441 aa) at 1 μg/ml was concentrated using an ultrafiltration spin column with a 10 kDa cut-off membrane. FIG. 30F shows total tau levels of pre- and post-ultrafiltration samples (ELISA). FIG. 30G shows SEC analysis of recombinant tau. FIG. 30H shows SDD-AGE analysis. Thirty ng of tau from pre-/post-ultrafiltration samples were loaded per lane. FIG. 30I shows tau seeding assay in the HEK-tau-biosensor cells (384-well plates). Ten ng/ml of tau (pre-/post-ultrailtration) were applied to the cells with 1% lipofectamine. Confocal images (FRET channel) at day 3 are shown. AD brain extract (PBS-soluble, 10,000 g spin, 10 ng/ml total tau) from entorhinal cortex was used as a positive control. White arrowhead indicates intracellular tau aggregates. Scale bar: 20 μm. HMW, high molecular weight; SDD-AGE, Semi-denaturing detergent agarose gel electrophoresis.

FIGS. 31A-31G show trans-synaptic propagation of human tau in ECrTgTau mice in the absence of endogenous mouse tau. FIG. 31 A shows 3D brain model, horizontal brain section illustrating transgenic human P301Ltau expression in the entorhinal cortex (EC) of the ECrTgTau mouse lines, and the propagation of transgenic tau to the dentate gyrus (DG). Tau composition in ECrTgTau and control mouse lines investigated FIG. 31 B shows immunostained horizontal sections show the expression of human P301Ltau in EC neurons in the absence of endogenous mouse tau (ECrTgTau-Mapt0/0). Fluorescence in situ hybridization of human tau mRNA combined with immunofluorescence labeling (immuno-FISH) of human tau protein (huTau) verifies P301Ltau transgene expression in the EC. Scale bars, 50 μm. FIG. 31 C shows propagation of human tau protein to neurons in the DG (white arrowheads) in ECrTgTau-Mapt^{0/0} mice. Close-ups show DG neurons from three ECrTgTau-Mapt^{0/0} mice (DG I-III). Immuno-FISH proofs the absence of human tau expression in DG neurons, which have huTau protein but no human tau mRNA. Scale bars, 50 μm. FIG. 31 D shows immunostained horizontal sections of ECrTgTau mice show the expression of human P301Ltau in EC neurons in the presence of endogenous mouse tau. Immuno-FISH proofs the absence of human tau expression in these DG neurons. Scale bars, 50 μm FIG. 31 E shows human P301Ltau propagation to DG neurons (white arrowheads) in the presence of endogenous mouse tau in ECrTgTau mice. Close-ups show DG neurons from three ECrTgTau mice (DG I-III). Scale bars, 50 μm. FIG. 31 F shows human (huTau, antibody Tau13) and total tau (hu+moTau, DAKO) levels in entorhinal cortex (EC) extracts from 18-month-old mice show equal human P301Ltau expression in ECrTgTau and ECrTgTau-Mapt0/0 mice (Mean±SEM, P=0.201, n=3 mice/group, one-way ANOVA with Bonferroni correction). FIG. 31 G shows the number of human tau-positive cell bodies in the DG (Mean±SEM, P=0.58, n=4 sections and 3 mice/group) and human tau in hippocampal (HPC) extracts (P=0.14, n=3 mice/group) were similar in ECrTgTau-Mapt^{0/0} and ECrTgTau mice (two-tailed Student's t-test).

FIGS. 32A-32E show P301Ltau propagation after viral expression in the entorhinal cortex. FIG. 32A shows Adeno-associated virus (AAV) construct designed for expression of eGFP and human P301Ltau as individual proteins, separated by the self-cleaving 2a peptide, under the CBA promoter (AAV8 CBA-eGFP-2a-huTauP301L). AAV-transduced “donor neurons” express eGFP and huTauP301L, and tau “recipient neurons” are identified after immunostaining for human tau as huTau+ but GFP-neurons. FIG. 32B shows primary cortical neuron cultures that were transduced with AAV eGFP-2a-P301Ltau at 7 DIV, and fixed and immunostained for GFP and human tau (Tau13 antibody) at 14 DIV, show tau donor (GFP+, huTau+; ˜10% neurons) and a small number of tau recipient neurons (GFP−, huTau+; ˜1% neurons). Western blot of whole cell lysates verified efficient cleavage (˜95%) of eGFP and P301Ltau by the 2a peptide (n=3). FIG. 32C shows eight weeks after AAV injection into right EC of aged Mapt0/0 mice (n=3), immunostained brain sections showed that huTauP301L (red) propagated to a few DG “recipient neurons” (white arrowheads). Scale bar, 50 μm. FIG. 32D shows unilateral AAV-mediated human P301L tau expression in the EC and DG of age-matched WT mice (n=3). Representative images of brain sections show donor neurons in the injected EC and DG, and a few tau recipient neurons (white arrowheads) adjacent to the AAV injection site. Scale bar, 50 μm. FIG. 32E shows in the contralateral hemisphere of the same brain section as in (FIG. 32D), some tau recipient neurons (white arrowheads) were also present in the (non-injected) axonal projection areas in the contralateral EC (GFP-filled terminal ends). Scale bar, 100 μm.

FIGS. 33A-33C show tau phosphorylation, misfolding, and gliosis in ECrTgTau(-Mapt^{0/0}) mice. FIG. 33A shows brain sections from 18-month-old ECrTgTau-Mapt0/0 and ECrTgTau mice were co-immunolabeled for human tau and misfolded tau (Alz50). Misfolded tau was only found in EC and DG neurons (white arrowheads) of ECrTgTau, but not ECrTgTau-Mapt^{0/0} animals (n=4 sections/mouse, 3 mice/group). Scale bars, 50 μm. FIGS. 33B-C show immunofluorescence labeling and stereological counting of microglia in entorhinal cortex (FIG. 33B) and astrocytes in hippocampus (FIG. 33C) indicated early signs of neurodegeneration in ECrTgTau mice. The significantly increased number of Iba1-positive microglia in the EC layer II/III of ECrTgtau mice (compared to WT) was partially rescued in ECrTgTau-Mapt^{0/0} mice (non-significant). The number of GFAP-positive astrocytes was similar across all genotypes (non-significant). Mean±SEM, n=4 sections per mouse, 3 mice/group, one-way ANOVA with Bonferroni correction. Scale bars, 100 μm.

FIGS. 34A-34F show tau knockout rescues P301Ltau-induced atrophy and neurodegeneration. FIG. 34 A shows human, mouse, and total tau protein levels in cortical TBS-extracts of rTg4510, rTg4510-Mapt0/0, and control mice: The amount of human tau (Tau13 antibody) was comparable in rTg4510 and rTg4510-Mapt^{0/0} moTau (Tau/5) was comparable in WT and rTg4510, and total tau levels (hu+moTau, DAKO antibody) were (expected) highest in rTg4510 mice. n=3 mice/group, non-significant. FIG. 34 B shows whole brain weights of 9-month-old animals revealed pronounced brain matter loss in rTg4510 compared to WT mice (weight loss >16%), which was rescued in rTg4510-Mapt^{0/0} mice to >96%. n=5 mice/group. FIG. 34 C shows cortical thickness measured adjacent to HPC, from CTX surface to corpus callosum, was decreased in rTg4510 mice by ˜25% compared to WT mice. rTg4510-Mapt^{0/0} showed no CTX thinning compared to Mapt0/0 or WT mice. n=3 mice/group. FIG. 34D shows the number of neurons (NeuN+ cells) in the cortex of rTg4510 mice was significantly reduced to ˜67% compared to both WT and rTg4510-Mapt^{0/0}. n=3 mice/group. FIG. 34E shows the volume of hippocampal region CA1, with CT the most affected regions in rTg4510 mice, was significantly reduced in rTg4510 by ˜70% volume; rTg4510-Mapt^{0/0} had significantly larger CA1 volume left (reduced by only ˜40%). n=3 mice/group. FIG. 34F shows rTg4510 showed strong signs of neuroinflammation with extremely high numbers of activated astroglia (GFAP+) and microglia (Iba1+) in the CTX compared to WT mice. Both astro- and microgliosis were reduced by ˜50% in rTg4510-Mapt^{0/0} mice, n=3 sections/mouse and 5 mice per/group. Scale bars, 100 μm. Mean±SEM. Two-tailed Student's t-test and one-way ANOVA with Bonferroni for multiple comparison. ns, not significant.

FIGS. 35A-35E show reduced P301Ltau and NFT neurotoxicity in the absence of endogenous tau. FIG. 35 A shows cortical extracts from rTg4510-Mapt^{0/0} brains had significantly less phospho-tau (CP13, PHF1, 12E8) than rTg4510 extracts. Compared to WT mice, both transgenic tau lines had high levels of phospho-tau. n=3 mice/group. FIG. 35 B shows representative images of gallyas silver-stained aggregated tau in cortices from 12-month-old mice unravel stunning differences in the degree of tau pathology in rTg4510 compared to rTg4510-Mapt^{0/0} mice. n=3 mice/group. FIG. 35 C shows higher magnification images of silver (12-month-old) and thioflavine-S(9-month-old)-stained cortices show mature tangles (white arrowheads in Thio-S stain) in rTg4510 and rTg4510-Mapt^{0/0} mice; enhanced pathological changes such as neuritic tau accumulation and neuropil vacuolation around NFTs are found only in rTg4510 mice. Stereological counting revealed similar numbers of cortical NFTs between rTg4510 and rTg4510-Mapt^{0/0} mice at 9 and 12 months of age. Because of the pronounced neuronal death only in rTg4510 mice, the percentage of tangle-bearing neurons was ˜1.6- to 1.8-fold higher in 9- and 12-month-old rTg4510 mice. n=3 sections/mouse, 3 mice/group. FIG. 35 D shows immuno-FISH for huTau mRNA and phospho-tau (PHF1) shows obvious differences in the distribution of neurons in cortex layer I/III: 9-month-old rTg4510 mice had more neurons filled with NFT-like phosphorylated tau (PHF1+), and rTg4510-Mapt^{0/0} mice had significantly more huTau mRNA-positive neurons (FISH+). rTg4510-Mapt^{0/0} mice had also more neurons still expressing both PHF1 and huTau mRNA (PHF1+FISH+), suggesting a reduced neurotoxicity of P301Ltau expression in rTg4510-Mapt^{0/0} mice. n=3 sections per mouse, 3 mice/group. FIG. 35 E shows immuno-FISH showing EC neurons having both misfolded somatic tau (Alz50) and human tau mRNA (white arrowheads) in rTg4510-Mapt0/0 but rarely in rTg4510 mice. n=3 sections, 2 mice/group. Mean±SEM. Two-tailed Student's t-test and one-way ANOVA with Bonferroni for multiple comparison. ns, not significant. (FIGS. 35B-D) Scale bars, 100 μm. (FIG. 35E) Scale bar, 50 μm.

FIGS. 36 A-36C show differences in tau oligomers and reduced seeding activity in rTg4510-Mapt^{0/0} mice. FIG. 36A shows extraction of cortices revealed similar human tau (Tau13) in TBS-extracts (not significant) but significantly more human tau in Triton X-100 (TX-100) extracts of rTg4510-Mapt^{0/0} compared to rTg4510 mice. Mean±SEM, n=3 mice/group, two-tailed Student's t-test, ns, non-significant. FIG. 36B shows native gel electrophoresis of cortical TBS-extracts showed small differences in HMW (oligomeric) human tau between rTg4510 and rTg4510-Mapt^{0/0} brains. Western blot lanes were averaged across ˜⅔ of the width (black rectangular and arrow in Tau13 blot). The mean±SEM (n=3 mice/group) of these averages was plotted as longitudinal lane profiles. Differences in HMW tau are indicated arrowheads. FIG. 36C shows TBS-brain extracts were applied to a HEK293 cell tau aggregation seeding assay (Holmes et al, 2014; Sanders et al, 2014), in which TauRDP301S-CFP and TauRDP301S-YFP are co-expressed intracellularly. The formation of intracellular fluorescent TauRDP301S aggregates leads to Foerster resonance energy transfer (FRET) activity between co-aggregated CFP and YFP-tags and correlates with the tau aggregation seeding activity of the applied brain extracts. After 24 h, cells treated with extract (0.5 and 1.0 lg total protein per 96 well) from 9-month-old rTg4510 had significantly more intracellular YFP-positive (white arrowheads) aggregates compared to rTg4510-Mapt^{0/0} extracts; FRET activity of TauRDP301 S aggregates appeared similar for both rTg4510 and rTg4510-Mapt^{0/0}. WT and Mapt^{0/0} extracts never showed seeding activity. Addition of lipofectamine corrected for differences in cellular uptake of tau and led to similar differences in seeding activity between rTg4510 and rTg4510-Mapt^{0/0} mice. Two-tailed Student's t-test, mean±SEM, n=3 replicates. ns, not significant. Insets, 100 μm. Scale bars, 50 μm.

### DETAILED DESCRIPTION OF THE INVENTION

Described herein are low-abundance, soluble phosphorylated high molecular weight (HMW) tau species present in postmortem brain cortical extracts from tau-transgenic mice and AD patients. It has been discovered that these soluble phosphorylated HMW tau species are involved in neuronal uptake and propagation between neurons. In particular, it is demonstrated herein that phosphorylation of specific residue(s) leads to the preferential uptake, axonal transport, and trans-synaptic propagation of this HMW form of tau. Thus, various aspects described herein stem from, at least in part, discovery of specific phosphorylation forms of the HMW tau species, including, for example, HMW tau species phosphorylated at one or more of the following amino acid residues: serine 396, serine 199, and serine 404 that are more important in neuronal uptake and propagation between neurons than non-phosphorylated forms or forms phosphorylated at different residues, wherein the locations of the phosphorylation sites (e.g., S396, S199, and S404) are based on a full-length tau reference sequence as defined in SEQ ID NO: 1. The inventors have also demonstrated that neuronal uptake of phosphorylated soluble HMW tau species, not low-molecular-weight (LMW) tau fractions, was detected in pre-tangle stage and wild-type (control) mouse models when the HMW tau species or LMW tau fractions were injected into the frontal cortex of the mice. Thus, in one aspect, the discovery of specific phosphorylated forms of rare soluble HMW tau species involved in inter-neuron-propagation provides a more effective target for therapeutic intervention and biomarker development. Accordingly, embodiments of various aspects described herein relate to compositions enriched in phosphorylated soluble HMW tau species that are responsible for inter-neuron propagation and applications thereof. Methods of treating and diagnosing tau-associated neurodegeneration in a subject are also provided herein.

As used herein, the term “enriched” with respect to soluble HMW tau species phosphorylated at least at serine 396, serine 199, and/or serine 404 in enriched compositions described herein, means that the concentrations of those phosphorylated soluble HMW tau species are higher in the enriched compositions described herein than what are found in vivo, e.g., higher than the concentrations found in the cerebrospinal fluid (CSF) of a patient with Alzheimer's disease. In addition, the enriched compositions described herein display a higher neuronal uptake, and/or cross-synaptic transport of the phosphorylated soluble HMW tau species between neurons, as compared to naturally-occurring in vivo compositions (e.g., CSF of a patient with Alzheimer's disease), when neurons are contacted with the same amount of total tau in the enriched compositions described herein or in the naturally-occurring in vivo compositions.

### Compositions Comprising Phosphorylated Soluble High Molecular Weight (HMW) Tau Species

In one aspect, a composition comprising at least one or more (e.g., at least two, at least three, at least four, at least five, at least six or more) phosphorylated forms of soluble high molecular weight (HMW) tau species is provided herein. The phosphorylated form(s) of the soluble HMW tau species in the composition is/are non-fibrillar, each has a molecular weight of at least about 500 kDa, and the composition is substantially free of soluble low molecular weight (LMW) tau species. In addition, for the soluble, phosphorylated HMW tau species described herein, the tau species phosphorylated at amino acid residue serine 422 (S422) is/are a lower level (e.g., by weight or by moles) than that of the soluble HMW tau species phosphorylated at one, two, or all of the following amino acid residues: serine 396, serine 199, and serine 404, wherein the locations of the phosphorylation sites (e.g., S396, S199, and S404) are based on a full-length tau reference sequence as defined in SEQ ID NO: 1. In some embodiments, the amount (e.g., by weight or by moles) of the S422-phosphorylated soluble HMW tau species (i.e., soluble HMW tau species phosphorylated at S422) can be lower than that of the soluble HMW tau species phosphorylated at one, two, or all of serine 396, serine 199, and serine 404, by at least about 10% or more, including, e.g., at least about 20%, at least about 30%, at least about 40%, at least about 50%, at least about 60%, at least about 70%, at least about 80%, at least about 90%, at least about 95% or more. In some embodiments, the composition can be substantially free of soluble HMW tau species phosphorylated at S422. In some embodiments, the proportions of various phosphorylated forms of soluble HMW tau species (e.g., serine 422 vs. serine 396, serine 199, and/or serine 404) in the compositions described herein can be substantially different from the proportions found in vivo.

In some embodiments where the composition further comprises a soluble HMW tau species phosphorylated at threonine 205 (T205), the amount (e.g., by weight or by moles) of the T205-phosphorylated soluble HMW tau species can be lower than that of the soluble HMW tau species phosphorylated at one, two, or all of serine 396, serine 199, and serine 404, by at least about 10% or more, including, e.g., at least about 20%, at least about 30%, at least about 40%, at least about 50%, at least about 60%, at least about 70%, at least about 80%, at least about 90%, at least about 95% or more. In some embodiments, the composition can be substantially free of soluble HMW tau species phosphorylated at T205. In some embodiments, the proportions of various phosphorylated forms of soluble HMW tau species (e.g., threonine 205 vs. serine 396, serine 199, and/or serine 404) in the compositions described herein can be substantially different from the proportions found in vivo.

In some embodiments where the composition further comprises a soluble HMW tau species phosphorylated at serine 262 (S262), the amount (e.g., by weight or by moles) of the S262-phosphorylated soluble HMW tau species can be lower than that of the soluble HMW tau species phosphorylated at one, two, or all of serine 396, serine 199, and serine 404, by at least about 10% or more, including, e.g., at least about 20%, at least about 30%, at least about 40%, at least about 50%, at least about 60%, at least about 70%, at least about 80%, at least about 90%, at least about 95% or more. In some embodiments, the composition can be substantially free of soluble HMW tau species phosphorylated at S262. In some embodiments, the proportions of various phosphorylated forms of soluble HMW tau species (e.g., threonine 205 vs. serine 396, serine 199, and/or serine 404) in the compositions described herein can be substantially different from the proportions found in vivo.

In some embodiments of various aspects described herein, the soluble HMW tau species that are phosphorylated at serine 396, serine 199, and/or serine 404 can also have S422, T205, and/or S262 phosphorylated.

In some embodiments of various aspects described herein, the soluble HMW tau species that are phosphorylated at serine 396, serine 199, and/or serine 404 can lack phosphorylation at S422, T205, and/or S262.

In some embodiments, the composition can be substantially free of non-phosphorylated soluble HMW tau species.

As used herein, the term “substantially free of” with respect to a selected tau species (e.g., soluble LMW tau species, a specific phosphorylated form of soluble HMW tau species, or non-phosphorylated soluble HMW tau species) includes the complete absence (i.e., 0%) of a selected tau species and a trace amount of the selected tau species that is not readily detectable by known methods in the art, e.g., size exclusion chromatogram (SEC), immunoassay (e.g., western blot or ELISA), microscopy, and atomic force microscopy.

Where a composition described herein is substantially free of soluble low molecular weight (LMW) tau species, the phrase “substantially free of soluble low molecular weight (LMW) tau species” includes the complete absence (i.e., 0%) of LMW tau species and a trace amount of LMW tau species that is not readily detectable by size exclusion chromatogram and/or immunoassay (e.g., western blot or ELISA). For example, a composition is considered as substantially free of soluble LMW tau species when the LMW fraction of the composition (e.g., after SEC fractionation) does not contain any tau species detectable by an anti-tau antibody (that is, an anti-tau antibody that can bind LMW tau). In some embodiments, the proportions of total soluble HMW (phosphorylated and/or non-phosphorylated) to soluble LMW tau in the compositions described herein are substantially different from the proportions in which these protein forms occur in vivo. Thus, where soluble HMW tau normally makes up only a small proportion of the total tau occurring in vivo, e.g., in humans, described herein are compositions or preparations in which at least 50% of the total tau protein is the soluble HMW form or particularly phosphorylated soluble HMW form, e.g., preparations with at least 50% phosphorylated soluble HMW and 50% or less soluble LMW tau, at least 60% phosphorylated soluble HMW tau and 40% soluble LMW tau or less, at least 70% phosphorylated soluble HMW tau and 30% soluble LMW tau or less, at least 80% phosphorylated soluble HMW tau and 20% or less soluble LMW tau, at least 90% phosphorylated soluble HMW tau and 10% or less soluble LMW tau, or at least 95% phosphorylated soluble HMW tau and 5% or less soluble LMW tau. In some embodiments, the composition can comprise no more than 5% (w/w) soluble LMW tau species, including, e.g., no more than 4%, no more than 3%, no more than 2%, no more than 1%, no more than 0.5% (w/w) soluble LMW tau species. As used herein, the term “substantially lacking LMW tau” means that a given HMW tau preparation has less than 1% LMW tau, and preferably less than 0.1% LMW tau, less than 0.01% tau or lower, by weight.

Where a composition described herein is substantially free of soluble HMW tau species that are phosphorylated at S422, T205, and/or S262, the composition can have 0% of such phosphorylated soluble HMW tau species or a trace amount of such phosphorylated soluble HMW species that is not readily detectable by size exclusion chromatogram and/or immunoassay (e.g., western blot or ELISA). For example, a composition is considered as substantially free of a specific phosphorylated form of soluble HMW tau species when the tau species is not detectable (i.e., below the detectable limit) in HMW portion(s) of the composition (e.g., after SEC fractionation) by an immunoassay using an antibody against the specific phosphorylated form. For illustrative purpose, a composition is substantially free of soluble HMW tau species phosphorylated at S422 when HMW portion(s) of the composition (e.g., after SEC fractionation) does not display any detectable signal upon contact with an anti-phospho S422 tau antibody. Different phospho-specific anti-tau antibodies can be used to detect the presence or absence of various phosphorylated forms in a composition described herein. In some embodiments, the composition can comprise no more than 5% (w/w) soluble HMW tau species that is phosphorylated at S422, T205, and/or S262, including, e.g., no more than 4%, no more than 3%, no more than 2%, no more than 1%, no more than 0.5% (w/w) soluble HMW tau species that is phosphorylated at S422, T205, and/or S262.

Where a composition described herein is substantially free of non-phosphorylated soluble HMW tau species, the composition can have 0% of non-phosphorylated soluble HMW tau species or a trace amount of non-phosphorylated soluble HMW species that is not readily detectable by size exclusion chromatogram and/or immunoassay (e.g., western blot or ELISA). For example, a composition is considered as substantially free of non-phosphorylated soluble HMW tau species when the HMW portion of the composition (e.g., after SEC fractionation) contains substantially the same amount of phosphorylated tau (e.g., detected using an anti-phospho tau antibody) as the total tau (phosphorylated and non-phosphorylated tau) detected using an anti-tau antibody. In some embodiments, the composition can comprise no more than 5% (w/w) non-phosphorylated soluble HMW tau species, including, e.g., no more than 4%, no more than 3%, no more than 2%, no more than 1%, no more than 0.5% (w/w) non-phosphorylated soluble HMW tau species.

As used herein, the term “high molecular weight tau species” or “HMW tau species” or “HMW tau” refers to a complex comprising tau species molecules, which complex is non-fibrillar and oligomeric, and has a non-beta pleated sheet structure. The tau species-comprising complex has a molecular weight of at least about 500 kDa, e.g., as determined by size exclusion chromatography (SEC). As used herein, the “non-beta pleated sheet” refers to a form of tau species molecules that do not form beta sheet structure, but can be taken up by neurons and propagate between neurons. The HMW tau species has a pathologically misfolded conformation (e.g., positive for Alz50 antibody staining) and appears as oligomeric structures (e.g., in atomic force microscopy). In some embodiments, the HMW tau species can be positive for Alz50 and negative for Thioflavin-S(ThioS). The HMW tau species can be intracellular (e.g., inside neurons) or extracellular (e.g., in the brain interstitial fluid and/or cerebrospinal fluid). In some embodiments, the HMW tau species can be produced by fractionating brain extracts and/or brain interstitial fluid or cerebrospinal fluid (e.g., ventricular or lumbar) from tau-transgenic animals (e.g., tau-transgenic mice), e.g., by centrifugation (e.g., ×3000 g or less) and/or size exclusion chromatography as described in the Examples, and selecting the fraction(s) with a molecular weight of at least about 500 kDa or more. In some embodiments, the HMW tau species can be produced by multimerizing recombinant tau proteins. In some embodiments, the HMW tau species can be phosphorylated or hyper-phosphorylated as described in further details below.

As used herein, the term “phosphorylated” when referring to soluble HMW tau species means an HMW tau species molecule with a phosphate group added to at least one or more (e.g., at least two, at least three or more) amino acid residues of the protein molecule. Phosphorylation can occur on serine (S) and/or threonine (T) residues at different sites of the HMW tau species protein molecule, including, but not limited to S199, S202, T205, S262, S396, S400, S404, S409, and S422. Accordingly, as used herein, the term “n-phosphorylated soluble HMW tau species” where n represents a phosphorylation site as described herein, refers to a soluble HMW tau species phosphorylated at least at site n, i.e., either solely at site n or at multiple sites (e.g., at least two more), one of which is site n. By way of example only, the term “S396-phosphorylated soluble HMW tau species” encompasses a soluble HMW tau species that is only phosphorylated at S396, and a soluble HMW tau species that is phosphorylated at two sites or more, one of which is S396. Accordingly, in some embodiments, the S396-phosphorylated soluble HMW tau species can be phosphorylated at S396 as well as, for example, at a site that also promotes neuronal uptake and propagation of the tau protein such as S199 and/or S404; or at a site that does not produce any significant effect on neuronal uptake and propagation of the tau protein such as S422.

In some embodiments, the phosphorylated soluble HMW tau species in the compositions described herein can be hyper-phosphorylated. As used herein, the term “hyper-phosphorylated” or “hyperphosphorylation” refers to the circumstance where the number of phosphorylated sites (i.e., the number of phosphate moieties) on the soluble HMW tau species in a composition is greater than that on the LMW tau species or non-aggregating normal tau proteins. In some embodiments, the total number of phosphate moieties on the soluble HMW tau species that are phosphorylated at least at S396 and optionally at S199 and/or S404 is greater than that on the LMW tau species or non-aggregating normal tau proteins, by at least 50% or more, including, e.g., at least 60%, at least 70%, at least 80%, at least 90%, at least 95% or higher. In some embodiments, the total number of phosphate moieties on the soluble HMW tau species that are phosphorylated at least at S396 and optionally at S199 and/or S404 is greater than that on the LMW tau species or non-aggregating normal tau proteins, by at least 1.1-fold or more, including, e.g., at least 1.25-fold, at least 1.5-fold, at least 2-fold, at least 3-fold, at least 4-fold, or higher. Full length tau isoform is represented by, e.g., NCBI Accession No. NP_005901.2, the information at which is incorporated herein by reference.

In some embodiments, at least 50% or more (including, e.g., at least 60%, at least 70%, at least 80%, at least 90%, at least 95%, at least 98%, or up to 100%) of the phosphorylated soluble HMW tau species in a composition described herein are phosphorylated at least at S396 and optionally at S199 and/or S404. In some embodiments, at least 50% or more (including, e.g., at least 60%, at least 70%, at least 80%, at least 90%, at least 95%, at least 98%, or up to 100%) of the phosphorylated soluble HMW tau species in a composition described herein are phosphorylated at least at both S396 and S199 or S404. In some embodiments, at least 50% or more (including, e.g., at least 60%, at least 70%, at least 80%, at least 90%, at least 95%, at least 98%, or up to 100%) of the phosphorylated soluble HMW tau species in a composition described herein are phosphorylated at least at all of S396, S199, and S404.

As used herein, the term “non-fibrillar” refers to HMW tau species that are not aggregated into insoluble neurofibrillary tangles (NFTs). NFTs are generally formed inside neurons from hyperphosphorylated tau proteins that are assembled into insoluble filaments. In contrast, the soluble, phosphorylated HMW tau species as described herein does not natively form a fibrillar aggregate.

In some embodiments, the phosphorylated soluble HMW tau species can be in a form of an oligomeric tau assembly. As used herein in reference to the phosphorylated soluble HMW tau species described herein, the term “oligomeric” or “oligomers” means a complex or an assembly comprising a finite number of tau monomer or dimer subunits. In some embodiments, the phosphorylated soluble HMW tau species described herein can comprise at least 2 or more tau monomer or dimer subunits, including, e.g., at least 3, at least 4, at least 5, at least 6, at least 7, at least 8, at least 9, at least 10, at least 15, at least 20, at least 30, at least 40 or more, tau monomer or dimer subunits. In some embodiments, the HMW tau species described herein can comprise about 3-100 tau monomer or dimer subunits, about 4-90 tau monomer or dimer subunits, about 5-80 tau monomer or dimer subunits, about 5-70 tau monomer or dimer subunits, about 5-60 tau monomer or dimer subunits, about 5-50 tau monomer or dimer subunits, about 5-40 tau monomer or dimer subunits, about 5-30 tau monomer or dimer subunits, or about 5-20 tau monomer or dimer subunits.

In some embodiments, the non-fibrillar soluble HMW tau species can be present in an assembly of particles. The particles can be of any shape, and are not limited to spherical or globular particles. The particle size of the phosphorylated soluble HMW tau species can vary with their molecule weights. In some embodiments, the phosphorylated soluble HMW tau species can be in a form of globular particles. As used herein, the term “globular” refers to an HMW tau species molecule or particle that is substantially spherical. By “substantially spherical” is meant that the ratio of the lengths of the longest to the shortest perpendicular axis of the particle cross section is less than or equal to about 1.5. Substantially spherical does not require a line of symmetry. Further, substantially spherical particles can have surface irregularities. In some embodiments, the ratio of the lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15, less than or equal to about 1.1.

In some embodiments, the particle size of the phosphorylated soluble HMW tau species can range from about 1 nm to about 50 nm, about 5 nm to about 40 nm, about 10 nm to about 30 nm, or about 15 nm to about 25 nm. In some embodiments, the phosphorylated soluble HMW tau species in the compositions described herein can form a particle size distribution with populations of varying sizes. In some embodiments, the phosphorylated soluble HMW tau species in the compositions described herein can all have substantially the same particle size.

Tau Proteins or Microtubule Associated Protein Tau (MAPT):

Tau proteins belong to the family of microtubule-associated proteins (MAP). They are mainly expressed in neurons where they play an important role in the assembly of tubulin monomers into microtubules to constitute the neuronal microtubules network, although non-neuronal cells (e.g., heart, kidney, lung, muscle, or pancreas cells) can have trace amounts. Microtubules are involved in maintaining the cell shape and serve as tracks for axonal transport. Tau proteins are translated from a single gene located on chromosome 17. Their expression is developmentally regulated by an alternative splicing mechanism and six different isoforms exist in the human adult brain. Buee et al., Brain Research Reviews (2000) 33: 95-130.

Tau can be subdivided into four regions: an N-terminal projection region, a proline-rich domain, a microtubule-binding domain (MBD), and a C-terminal region. Morris et al., Neuron (2011) 70: 410-426. Alternative splicing around the N-terminal region and MBD generates six main isoforms in adult human brain (Goedert et al., Neuron (1989) 3: 519-526), with the range from 352-441 amino acids. They differ in either zero, one or two inserts of 29 amino acids at the N-terminal part (exon 2 and 3), and three or four repeat-regions at the C-terminal part exon 10 missing. Therefore, the longest isoform in the CNS has four repeats (R1, R2, R3 and R4) and two inserts (441 amino acids total), while the shortest isoform has three repeats (R1, R3 and R4) and no insert (352 amino acids total). Tau isoforms are named by how many microtubule binding repeat sequences are expressed (termed R) and by which N-terminal exons are included (termed N). For example, 3R tau has three microtubule binding repeat sequences, while 4R tau has four due to inclusion of exon 10. 0N tau includes no N-terminal exons, 1N tau exon 2, and 2N tau exons 2 and 3 (Lee et al., Annu. Rev. Neurosci. (2001) 24: 1121-1159). Tau mutations are numbered by their location in 4R2N human tau (Lee et al., 2001). Six additional isoforms are formed by alternative splicing around exon 6, resulting in a total of 12 tau isoforms expressed in brain (Wei and Andreadis, J. Neurochem (1998) 70; 1346-1356). The references cited herein are incorporated herein by reference.

In some embodiments, the phosphorylated soluble HMW tau species can be an oligomer enriched in at least one or more (e.g., at least two or more) of the tau isoforms selected from the group consisting of tau isoform 1, tau isoform 2, tau isoform 3, tau isoform 4, tau isoform 5, and tau isoform 6. In some embodiments, the phosphorylated soluble HMW tau species can be an oligomer enriched in at least one or more (e.g., at least two or more) of the tau isoforms selected from the group consisting of (2−3−10−); (2+3−10−); (2+3+10−); (2−3−10+); (2+3−10+); (2+3+10+). All MAP (microtubule-associated protein) tau protein isoforms are known in the art and their nucleotide and protein sequences are available on the world wide web from the NCBI, including, e.g., human. Table 1 below shows exemplary Accession Nos of the nucleotide and amino acid sequences of different human tau isoforms that are available at NCBI.

TABLE 1
Amino acid sequences of different human tau isoforms

| Various human MAPT | | |
|---|---|---|
| (microtubule | Nucleotide | Amino acid |
| associated protein | sequence (NCBI | sequence (NCBI |
| tau) isoforms | Accession No.) | Accession No.) |
| MAPT isoform 1 | NM_016835 | NP_058519 |
| MAPT isoform 2 | NM_005910 | NP_005901 |
| | | (SEQ ID NO: 1) |
| MAPT isoform 3 | NM_016834 | NP_058518 |
| MAPT isoform 4 | NM_016841 | NP_058525 |
| MAPT isoform 5 | NM_001123067 | NP_001116539 |
| MAPT isoform 6 | NM_001123066 | NP_001116538 |
| MAPT isoform 7 | NM_001203251 | NP_001190180 |
| MAPT isoform 8 | NM_001203252 | NP_001190181 |

As used herein, the term “MAPT” or “microtubule-associated protein tau” generally refers to a MAPT polypeptide or a MAPT polynucleotide that is similar or identical to the sequence of a wild-type MAPT. In some embodiments, the term “MAPT polypeptide” refers to a polypeptide having an amino acid sequence that is at least 70% or more (including at least 75%, at least 80%, at least 85%, at least 90%, at least 95%, at least 97%, at least 99%, or 100%) identical to that of a wild-type MAPT, and is capable of modulating the stability of axonal microtubules.

In some embodiments, the term “MAPT polynucleotide” refers to a polynucleotide having a nucleotide sequence that is at least 70% or more (including at least 75%, at least 80%, at least 85%, at least 90%, at least 95%, at least 97%, at least 99%, or 100%) identical to that of a wild-type MAPT or a portion thereof, and encodes a MAPT polypeptide as described herein.

The wild-type MAPT sequences of various isoforms and of different species are available on the world wide web from the NCBI, including human, mouse, rat, and dog. For example, the nucleotide sequences encoding different isoforms of human MAPT and their corresponding amino acid sequences are available at NCBI and their Accession Nos are included in Table 1 shown herein.

Where the term “MAPT” refers to a MAPT polypeptide, a “variant” of a MAPT polypeptide encompasses a portion or fragment of such a MAPT polypeptide that retains at least about 70% or more (including at least 75%, at least 80%, at least 85%, at least 90%, at least 95%, at least 97%, at least 99%, or 100%) of the axonal microtubule-stabilizing activity of the wild-type MAPT polypeptide. The variant also encompasses conservative substitution variants of a MAPT polypeptide that retain at least about 70% or more (including at least 75%, at least 80%, at least 85%, at least 90%, at least 95%, at least 97%, at least 99%, or 100%) of the axonal microtubule-stabilizing activity of the wild-type MAPT polypeptide.

The amino acid identity between two polypeptides can be determined, for example, by first aligning the two polypeptide sequences using an alignment algorithm, such as BLAST® or by other methods well-known in the art.

As used herein, the term “soluble” when referring to the HMW or LMW tau species, means the HMW or LMW tau species dissolves and forms a substantially homogeneous solution in a biological fluid, e.g., CSF, brain interstitial fluid, plasma, etc., e.g., under a physiological condition (e.g., at room temperature or at body temperature such as about 37° C., at the physiological pH of CSF or brain ISF, and under atmospheric pressure), as compared to insoluble neurofibrillary tangles or insoluble fibrillar tau aggregates under the same condition. The HMW tau soluble species as described herein also dissolve and form a substantially homogeneous solution in an aqueous buffer solution (e.g., a phosphate-buffered saline) under a physiological condition (e.g., at room temperature or at body temperature such as about 37° C., at the physiological pH of CSF or brain ISF, and under atmospheric pressure), as compared to insoluble neurofibrillary tangles or insoluble fibrillar tau aggregates under the same condition.

As used herein, the term “molecular weight” refers to the mass of a given molecule (e.g., an HMW or LMW tau species molecule) or the average mass of a population (e.g., two or more) of given molecules (e.g., a population of HMW tau species molecules or LMW tau species molecules). Different average values (e.g., number average molecular weight vs. mass average molecular weight) can be defined depending on the statistical method that is applied. In some embodiments, the molecular weight is number average molecular weight. The molecular weights of HMW or LMW tau species can be generally measured by any methods known in the art, e.g., but not limited to, gel electrophoresis, gel chromatography, size exclusion chromatography, light scattering, and/or mass spectrometry. In some embodiments, the molecular weight of the HMW tau species or LMW tau species is determined by size exclusion chromatography (SEC). For example, standard protein samples of known molecular size are separated on a SEC column and the retention volumes or elution volumes (i.e., the volume of eluting buffer necessary to remove a particular analyte from a packed column) are recorded. A calibration plot of log molecular mass (Y axis) versus elution volume (X axis) is prepared. The calibration plot can be used to estimate the molecular weight distribution of a tau species mixture by separating the mixture on the same column as the standards and recording its retention volume. The molecular weight of the fractionated tau species can then be extrapolated from the calibration plot.

In the compositions described herein, the phosphorylated soluble HMW tau species has a molecular weight of at least about 500 kDa or more. In some embodiments, the phosphorylated soluble HMW tau species can have a molecular weight of at least about 550 kDa, at least about 600 kDa, at least about 650 kDa, at least about 700 kDa, at least about 750 kDa, at least about 800 kDa, at least about 850 kDa, at least about 900 kDa, at least about 950 kDa, or more. In some embodiments, the phosphorylated soluble HMW tau species can have a molecular weight of at least about 669 kDa or more. In some embodiments, the phosphorylated soluble HMW tau species can have a molecular weight of about 500 kDa to about 2000 kDa, about 550 kDa to about 1500 kDa, about 600 kDa to about 1000 kDa, about 650 kDa to about 1000 kDa or about 669 kDa to about 1000 kDa. In some embodiments, the phosphorylated soluble HMW tau species in the compositions described herein can form a molecular weight distribution with populations of varying molecular weights. In some embodiments, the phosphorylated soluble HMW tau species in the compositions described herein can all have substantially the same molecular weight.

In some embodiments, the phosphorylated soluble HMW tau species can consist essentially of, or consist of, tau proteins (e.g., in monomer and/or dimer subunits).

In some embodiments, the phosphorylated soluble HMW tau species can comprise other constituents such as other proteins and/or lipids.

In some embodiments, the soluble HMW tau species phosphorylated at least at one, two, or all of serine 396, serine 199, and serine 404 can be preferentially taken up by a neuron. As used herein, the term “preferentially taken up by a neuron” refers to a neuron, in an in vitro or in vivo assay, showing a higher uptake of soluble HMW tau species phosphorylated at least at one, two, or all of serine 396, serine 199, and serine 404, than of soluble LMW tau species, or soluble HMW tau species without phosphorylation at serine 396, serine 199 or serine 404, or non-phosphorylated soluble HMW tau species (i.e., soluble HMW tau species with no phosphorylation at all), when the neuron is contacted with the same amount of each indicated tau species. To determine the neuronal uptake of a tau species in vitro, one can perform the in vitro tau uptake assay as described in Example 1. For example, mouse neurons can be incubated with a human tau species to be characterized over a period of time, followed by immunostaining of the neurons with a human tau-specific antibody to detect exogenous human tau in mouse neurons. Other in vitro assays, e.g., using tau-biosensor cells as described in Example 1, can also be used to determine neuronal uptake of a specific tau species. In vivo assays such as injection of a human tau species to a frontal cortex of mice and subsequent detection of neuronal uptake of the human tau species by immunostaining of the brain sections from the frontal cortex as described in Example 1, can be performed to determine tau uptake in vivo. Accordingly, in some embodiments, soluble HMW tau species phosphorylated at least at one, two, or all of serine 396, serine 199, and serine 404 show a higher neuronal uptake than soluble LMW tau species, or soluble HMW tau species without phosphorylation at serine 396, serine 199 or serine 404, or non-phosphorylated soluble HMW tau species (i.e., soluble HMW tau species with no phosphorylation at all) by at least about 30% or more (including, e.g., at least about 40%, at least about 50%, at least about 60%, at least about 70%, at least about 80%, at least about 90%, at least about 95% or higher), as measured in an in vitro or in vivo assay described herein. In some embodiments, soluble HMW tau species phosphorylated at least at one, two, or all of serine 396, serine 199, and serine 404 show a higher neuronal uptake than soluble LMW tau species, or soluble HMW tau species without phosphorylation at serine 396, serine 199 or serine 404, or non-phosphorylated soluble HMW tau species (i.e., soluble HMW tau species with no phosphorylation at all) by at least about 1.5-fold or more (including, e.g., at least about 2-fold, at least about 2.5-fold, at least about 3-fold, at least about 3.5-fold, at least about 4-fold, at least about 5-fold, at least about 10-fold, or higher), as measured in an in vitro or in vivo assay described herein.

Once the specific phosphorylated soluble HMW tau species is taken up by a neuron, the phosphorylated soluble HMW tau species can be axonally transported from the neuron to a synaptically-connected neuron. In some embodiments, the neuron-to-neuron tau transfer can occur in the absence of astrocytes. Thus, in some embodiments, a neuron can preferentially axonally transport soluble HMW tau species phosphorylated at least at one, two, or all of serine 396, serine 199, and serine 404 from its cell body to a synaptically-connected neuron. As used herein, the term “preferentially axonally transport phosphorylated soluble HMW tau species from its cell body to a synaptically-connected neuron” refers to a neuron showing a higher rate and/or frequency of axonally transporting soluble HMW tau species phosphorylated at least at one, two, or all of serine 396, serine 199, and serine 404 from its cell body to a synaptically-connected neuron, as compared to axonal transport between synaptically connected neurons of soluble LMW tau species, or soluble HMW tau species without phosphorylation at serine 396, serine 199 or serine 404, or non-phosphorylated soluble HMW tau species (i.e., soluble HMW tau species with no phosphorylation at all), when the neuron is contacted in vitro with the same amount of each indicated tau species for the same period of time. The axonal transport of phosphorylated soluble HMW tau species between synaptically connected neurons can be determined in an in vitro assay using a three-chamber microfluidic device as described in Example 1. Accordingly, in some embodiments, as measured in an in vitro assay described herein, a neuron can have a higher rate and/or frequency of axonally transporting soluble HMW tau species phosphorylated at least at one, two, or all of serine 396, serine 199, and serine 404 from its cell body to a synaptically-connected neuron, by at least about 30% or more (including, e.g., at least about 40%, at least about 50%, at least about 60%, at least about 70%, at least about 80%, at least about 90%, at least about 95% or higher), as compared to axonal transport between synaptically connected neurons of soluble LMW tau species, or soluble HMW tau species without phosphorylation at serine 396, serine 199 or serine 404, or non-phosphorylated soluble HMW tau species (i.e., soluble HMW tau species with no phosphorylation at all). In some embodiments, as measured in an in vitro assay described herein, a neuron can have a higher rate and/or frequency of axonally transporting soluble HMW tau species phosphorylated at least at one, two, or all of serine 396, serine 199, and serine 404 from its cell body to a synaptically-connected neuron, by at least about 1.5-fold or more (including, e.g., at least about 2-fold, at least about 2.5-fold, at least about 3-fold, at least about 3.5-fold, at least about 4-fold, at least about 5-fold, at least about 10-fold, or higher), as compared to axonal transport between synaptically connected neurons of soluble LMW tau species, or soluble HMW tau species without phosphorylation at serine 396, serine 199 or serine 404, or non-phosphorylated soluble HMW tau species (i.e., soluble HMW tau species with no phosphorylation at all).

The term “axonally transport” or “axonal transport” is used herein to refer to directed transport of molecules and/or organelles along an axon of a neuron. The “axonal transport” can be “anterograde” (outward from the cell body) or “retrograde” (back toward the cell body). Thus, anterograde transport of phosphorylated soluble HMW tau species delivers the phosphorylated soluble HMW tau species taken up a neuron from its cell body outwards to distant synapses.

As used herein, the term “synaptically connected” refers to neurons which are in communication with one another via a synapse. A synapse is a zone of a neuron specialized for a signal transfer. Synapses can be characterized by their ability to act as a region of signal transfer as well as by the physical proximity at the synapse between two neurons. Signaling can be by electrical or chemical means.

As used herein, the term “low molecular weight tau species” or “LMW tau species” refers to a population of tau species molecules that are substantially tau monomer subunits and/or tau dimer subunits. The LMW tau species can be intracellular (e.g., inside neurons) or extracellular (e.g., in the brain interstitial fluid and/or cerebrospinal fluid). In some embodiments, the LMW tau species can be produced by fractionating brain extracts and/or brain interstitial fluid from tau-transgenic animals (e.g., tau-transgenic mice), e.g., by centrifugation at higher speed (e.g., 50000×g or more) and/or size exclusion chromatography as described in the Examples, and selecting the fraction(s) with a molecular weight of no more than 200 kDa, or less. In some embodiments, the soluble LMW tau species can have a molecular weight of no more than 200 kDa, or less, including, e.g., no more than 150 kDa, no more than 100 kDa, no more than 50 kDa, or less.

In some embodiments, the compositions described herein can comprise an agent to suit the need of a selected application. For example, in some embodiments, the compositions described herein can be adapted to raise an antibody against one or more phosphorylated forms of the phosphorylated soluble HMW tau species. Where the phosphorylated soluble HMW tau is to be used as an antigen to raise an antibody, purified phosphorylated soluble HMW tau, e.g., purified soluble HMW tau phosphorylated at one, two, or all of serine 396, serine 199, and serine 404, can be combined with saline or phosphate-buffered saline. Alternatively, or in addition, the purified phosphorylated soluble HMW tau antigen can be admixed with or conjugated to an adjuvant or carrier, e.g., a carrier peptide, to enhance its antigenicity. Accordingly, in some embodiments, the compositions described herein can further comprise an adjuvant for raising an antibody against one or more specific phosphorylated forms of the phosphorylated soluble HMW tau species (e.g., soluble HMW tau species phosphorylated at one, two, or all of serine 396, serine 199, and serine 404). The term “adjuvant,” as used herein, refers to molecule(s), compound(s), and/or material(s) which, when administered to an individual or an animal (e.g., mice) in vivo, increase(s) the immune response of the individual or the animal to an antigen (e.g., soluble HMW tau species) administered. Some antigens are weakly immunogenic when administered alone or are toxic to the individual at concentrations which evoke immune responses in the individual or animal. An adjuvant can enhance the immune response of the individual or animal to the antigen by making the antigen more strongly immunogenic, thus enhancing antibody production. The adjuvant effect can also lower the dose of the antigen necessary to achieve an immune response in the individual or animal. Commonly used adjuvants include, but are not limited to, Incomplete Freund's Adjuvant, which consists of a water in oil emulsion, Freund's Complete Adjuvant, which comprises the components of Incomplete Freund's Adjuvant, with the addition of Mycobacterium tuberculosis, and alum. The phosphorylated soluble HMW tau compositions described herein can also be conjugated to an adjuvant. Conjugation to an antigenic carrier such as keyhole limpet hemocyanin can also be used to increase the antigenicity of the phosphorylated soluble HMW tau complexes described herein (e.g., HMW tau phosphorylated at one, two or all of serine 396, serine 199, and serine 404). Other antigenic carrier proteins that can be conjugated to phosphorylated soluble HMW tau (e.g., HMW tau phosphorylated at one, two or all of serine 396, serine 199, and serine 404) include, e.g., Concholepas concholepas, hemocyanin (“Blue Carrier”), bovine serum albumin, cationized bovine serum, nd ovalbumin. It is further contemplated that treatments that stabilize the phosphorylated soluble HMW tau complexes in the HMW and phosphorylated form (e.g., phosphorylated at one, two, or all of serine 396, serine 199, and serine 404) that is preferentially transmitted from neuron to neuron will render the phosphorylated soluble HMW tau more likely to serve as an antigen to raise antibodies specific for a phosphorylated form of the HMW tau species as opposed to either the non-phosphorylated or less-phosphorylated soluble HMW or soluble LMW forms of the tau protein. Various approaches can be used to effect stabilization of the HMW tau structures. For example, HMW tau can be stabilized in the HMW/synaptically transmissible form of tau by cross linking isolated HMW tau. A number of chemical cross-linking reagents are known and available commercially, including homobifunctional and heterobifunctional cross linkers that react, e.g., with amines (e.g., N-hydroxsuccinimide (NHS) ester cross linkers, including disuccinimidyl glutarate, disuccinimidyl suberate, bis[sulfosuccinimidyl] suberate, dithiobis[succinimidyl] propionate, among others, imidoester including dimethyl adipimidate-2HCl, dimethyl suberimidate, etc.) or with sulfhydryls (e.g., maleimide-based cross linkers, e.g., bismaleimidoethane, bismaleimidohexane, dithiobismaleimidoethane, etc.) The cross-linkers can be modulated by tailoring reaction conditions as known in the art. In one embodiment a relatively small degree of cross-linking can provide substantial stabilization relative to non-cross linked HMW, and thereby provide an enhanced activity as, e.g., as an antigen or as a target for screening assays. HMW tau can also be stabilized, e.g., by interacting with a surface, e.g., plastic, nitrocellulose, or nylon membrane. Methods to preserve phosphorylation are known in the art and can be used to preserve phosphorylation of the HMW tau species. For example, phosphatases that may be present in the compositions described herein can be inactivated, e.g., by heat. In these embodiments, stabilized phosphorylated soluble HMW tau can serve as a substrate or target for screening for agents that specifically bind a desired phosphorylation form of HMW tau protein. As but one example, a surface functionalized with HMW tau can be used to pan for, e.g., bacteriophage displaying a binding polypeptide. Libraries of bacteriophage display constructs are well-known in the art. One can enhance the likelihood of obtaining a phage-displayed HMW tau binding protein in a specific phosphorylated form (e.g., S396 phosphorylated soluble HMW tau binding protein) that does not substantially bind LMW tau or HMW tau phosphorylated at other sites by first contacting the phage library with LMW tau, and/or HMW tau phosphorylated at other non-desirable sites, immobilized on a surface to subtract out those library members including LMW-tau binding polypeptides and/or HMW-tau binding polypeptides that bind to non-desirable phosphorylated forms. After subtraction in this manner, the library is then contacted with the desired phosphorylated form of the HMW tau immobilized on a separate surface, followed by isolation and propagation of those phages that stick to the desired phosphorylated form of the HMW tau. Solid supports/surfaces can include, without limitation, nitrocellulose or nylon membranes, affinity column chromatography matrices, nylon or other polymer beads, among others.

In some embodiments, the soluble HMW tau species phosphorylated at least at S396, S404 and/or S199 can be modified. By way of example only, in some embodiments, the soluble HMW tau species phosphorylated at least at S396, S404 and/or S199 can be coupled or conjugated to a detectable label. In some embodiments, the soluble HMW tau species phosphorylated at least at S396, S404 and/or S199 and a detectable label can be fused together to form a fusion protein. The term “detectable label” as used herein refers to a composition capable of producing a detectable signal indicative of the presence of a target with the detectable label attached thereto. Detectable labels include any composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, bioluminescent moieties, and the like. As such, a label is any composition detectable by spectroscopic, photochemical, biochemical, immunochemical, electrical, optical or chemical means needed for the methods and devices described herein.

### Antagonists of Various Phosphorylated Forms of Soluble HMW Tau Species

Unlike insoluble neurofibrillary tangles as intracellular proteins in neurons, the phosphorylated soluble HMW tau species described herein is a novel soluble, non-fibrillar tau complex or assembly, which can be found in the extracellular space, e.g., soluble in brain interstitial fluid and/or cerebrospinal fluid. As described earlier, the inventors have shown that phosphorylated soluble HMW tau species are preferentially taken up by neurons and axonally transported to synaptically-connected neurons, thus progressing tau spreading between neurons. By reducing or blocking neuron uptake of a specific phosphorylated form of soluble HMW tau species (e.g., S396-phosphorylated soluble HMW tau species) described herein, tau spreading can be limited or prevented. Accordingly, provided herein, in various aspects, are compositions comprising phosphorylated soluble HMW tau species antagonist agents, such as antibodies or antigen-binding fragments thereof, nucleic acids, and small organic molecules, for inhibiting or reducing a specific phosphorylated form of soluble HMW tau species (e.g., S396-phosphorylated soluble HMW tau species) being taken up by a neuron and/or axonally transported from the neuron to a synaptically-connected neuron, and methods of use thereof for inhibition or reduction of neuron uptake of a specific phosphorylated form of soluble HMW tau species (e.g., S396-phosphorylated soluble HMW tau species) and pathologies associated with tau propagation.

As used interchangeably herein, the terms “tau propagation” and “tau spreading” refers to the entire biological process of transporting misfolded tau protein or extracellular tau particles between neurons. Tau propagation includes neuronal uptake of extracellular tau protein or particles, seeding of tau protein or particles to induce intracellular tau aggregation, and transfer of the tau protein or particles from one neuron to a synaptically-connected neuron. In some embodiments, the neuron-to-neuron tau transfer can occur in the absence of astrocytes.

As used herein, the term “seeding,” when referring to tau seeding, means that the HMW tau species interacts with intracellular, soluble forms of tau proteins and induces aggregation of the intracellular tau proteins to form insoluble aggregate. In some embodiments, the HMW tau species can cause intracellular tau proteins to transform to a misfolded protein state, thereby inducing tau aggregation.

As used herein, a “phosphorylated soluble HMW tau species antagonist agent” or “an antagonist of a phosphorylated soluble HMW tau species” refer to an agent, such as a small organic molecule, inhibitory nucleic acid, or phosphorylated soluble HMW tau species-specific antibody or antigen-binding fragment thereof, that inhibits or causes or facilitates a qualitative or quantitative inhibition, decrease, or reduction in one or more processes, mechanisms, effects, responses, functions, activities or pathways mediated by one or more phosphorylated forms of soluble HMW tau species to be specifically targeted. Thus, the term “phosphorylated soluble HMW tau species antagonist agent” refers to an agent that inhibits or reduces specific phosphorylation or level of phosphorylation at one or more specific sites (e.g., S396, S199, and/or S404), e.g., by at least about 10% or more (including, e.g., at least about 20%, at least about 30%, at least about 40%, at least about 50%, at least about 60%, at least about 70%, at least about 80%, at least about 90% or more, up to 100%); or one that inhibits formation of the phosphorylated soluble HMW tau species, e.g., from phosphorylated tau proteins; or one that binds to, partially or totally blocks, decreases, or prevents neuron uptake of soluble HMW tau species that is phosphorylated at least at one or more target sites (e.g., S396, S199 and/or S404), e.g., by at least about 10% or more (including, e.g., at least about 20%, at least about 30%, at least about 40%, at least about 50%, at least about 60%, at least about 70%, at least about 80%, at least about 90% or more, up to 100%); and/or blocks, decreases, or prevents inter-neuron propagation of the phosphorylated soluble HMW tau species that is phosphorylated at least at one or more target sites (e.g., S396, S199 and/or S404) upon the neuron uptake, e.g., by at least about 10% or more (including, e.g., at least about 20%, at least about 30%, at least about 40%, at least about 50%, at least about 60%, at least about 70%, at least about 80%, at least about 90% or more, up to 100%).

In some embodiments of these aspects and all such aspects described herein, the phosphorylated soluble HMW tau species antagonist agents do not bind soluble low molecular weight (LMW) tau species. For example, in some embodiments, soluble HMW tau species antagonist agents do not bind soluble LMW tau species that have a molecular weight of no more than 200 kDa, including, e.g., no more than 150 kDa, no more than 100 kDa, or lower.

As used herein, the term “do not bind” refers to an agent with completely no, or minimal binding affinity to a non-target molecule. An agent with minimal binding affinity to a non-target molecule means that the ratio of K_{D} values of the agent for a non-target molecule to a target molecule that it selectively binds (as defined below) is greater than 10 or higher, including, e.g., greater than 100, 10³, 10⁴, 10⁵, 10⁶, or higher. Accordingly, in some embodiments, the phosphorylated soluble HMW tau species antagonist agent does not bind soluble LMW tau species at all. In some embodiments, the phosphorylated soluble HMW tau species antagonist agent has a K_{D} value for soluble LMW tau species that is at least 10 times, including, e.g., at least 100 times, at least 10³ times, at least 10⁴ times, at least 10⁵ times, at least 10⁶ times or more, higher than that for a specific phosphorylated form of soluble HMW tau species. Methods to determine K_{D} values of an agent to a molecule are known to a skilled person in the art.

The term “agent” as used herein in reference to a phosphorylated soluble HMW tau species antagonist means any compound or substance such as, but not limited to, a small organic molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments, an agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. In some embodiments, agents are small organic molecules having a chemical moiety. For example, chemical moieties include unsubstituted or substituted alkyl, aromatic, or heterocyclyl moieties. Compounds can be known to have a desired activity and/or property, e.g., inhibit neuron uptake of phosphorylated soluble HMW tau species and/or optional inter-neuron propagation of the phosphorylated soluble HMW tau species, or can be selected from a library of diverse compounds, using, for example, screening methods.

In some embodiments, the phosphorylated soluble HMW tau species antagonist agents can specifically bind and reduce or inhibit neuron uptake of, non-fibrillar, soluble HMW tau species described herein, for example, soluble HMW tau species with a molecular weight of at least about 500 kDa that is phosphorylated at least at serine 396. In some embodiments, the phosphorylated soluble HMW tau species can have a molecular weight of at least about 669 kDa or more. In some embodiments, the phosphorylated soluble HMW tau species can have a molecular weight of about 669 kDa to about 1000 kDa. In some embodiments, the non-fibrillar soluble HMW tau species can be in a form of particles, e.g., globular particles. The particle size can vary with the molecular weight of the tau species. In some embodiments, the particle size can range from about 10 nm to about 30 nm. In some embodiments, the phosphorylated soluble HMW tau species antagonist agents can specifically bind and reduce or inhibit neuron uptake of, non-fibrillar, soluble HMW tau species described herein, for example, with a molecular weight of at least about 500 kDa and being further phosphorylated at least at serine 404 and/or at serine 199.

In some embodiments, the phosphorylated soluble HMW tau species antagonist agents can specifically bind at, or in close proximity to, a serine 396 (S396) phosphorylation site of soluble HMW tau species described herein, for example, with a molecular weight of at least about 500 kDa, such that the antagonist agent inhibits phosphorylation, or reduces degree of phosphorylation at the S396 phosphorylation site. In some embodiments, the phosphorylated soluble HMW tau species can have a molecular weight of about 669 kDa to about 1000 kDa. In some embodiments, the non-fibrillar soluble HMW tau species can be in a form of particles, e.g., globular particles. The particle size can vary with the molecular weight of the tau species. In some embodiments, the particle size can range from about 10 nm to about 30 nm.

In some embodiments, the phosphorylated soluble HMW tau species antagonist agents can specifically bind at, or in close proximity to, a serine 404 (S404) phosphorylation site of soluble HMW tau species described herein, for example, with a molecular weight of at least about 500 kDa, such that the antagonist agent inhibits phosphorylation, or reduces degree of phosphorylation at the $404 phosphorylation site.

In some embodiments, the phosphorylated soluble HMW tau species antagonist agents can also specifically bind at, or in close proximity to, a serine 119 (S119) phosphorylation site of soluble HMW tau species described herein, for example, with a molecular weight of at least about 500 kDa, such that the antagonist agent also inhibits phosphorylation, or reduces level of phosphorylation at the S199 phosphorylation site.

In some embodiments, the phosphorylated soluble HMW tau species antagonist agents described herein are functional in an aqueous solution and/or a buffered solution. As used herein, the term “functional” in reference to a phosphorylated soluble HMW tau species antagonist agent means that the phosphorylated soluble HMW tau species antagonist agent is able to specifically bind at, or in close proximity to, a target phosphorylation site (e.g., S396, S404, and/or S199) of soluble HMW tau species described herein, and/or is able to specifically bind and reduce or inhibit neuron uptake of soluble HMW tau species that is phosphorylated at a target site (e.g., S396, S404, and/or S199). For example, in some embodiments, the phosphorylated soluble HMW tau species antagonist agents can specifically bind at, or in close proximity to, a target phosphorylation site (e.g., S396, S404, and/or S199) of HMW tau species described herein soluble in an aqueous solution and/or a buffered solution, and/or is able to specifically bind and reduce or inhibit neuron uptake of HMW tau species that is phosphorylated at a target site (e.g., S396, S404, and/or S199) and is soluble in an aqueous solution and/or a buffered solution. In some embodiments, the aqueous solution and/or buffered solution can comprise a phosphate-buffered saline. In some embodiments, aqueous solution and/or buffered solution can comprise a biological fluid, e.g., a brain interstitial fluid or cerebrospinal fluid.

As used herein, “selectively binds” or “specifically binds” refers to the ability of a phosphorylated soluble HMW tau species antagonist agent as described herein to bind to a specific phosphorylated form of a soluble HMW tau species polypeptide or a specific phosphorylation site of the phosphorylated soluble HMW tau species polypeptide, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. For example, if an antagonist (small organic molecule, antibody or other) described herein binds to the specific phosphorylated form of a soluble HMW tau species polypeptide (e.g., S396-, S404-, and/or S199-phosphorylated soluble HMW tau species polypeptide) or the specific phosphorylation site (e.g., S396, S404 and/or S199) of the phosphorylated soluble HMW tau species polypeptide with a K_{D} of 10⁻⁵ M or lower, but not substantially to other molecules, e.g., LMW tau species, HMW tau species without phosphorylation at S396, S404 and/or S199, or a related homologue, or not substantially to other phosphorylation sites of the phosphorylated soluble HMW tau species (e.g., S422), then the agent is said to specifically bind the specific phosphorylated form of the phosphorylated soluble HMW tau species polypeptide. In some embodiments an agent that specifically binds phosphorylated tau, whether HMW or LMW, is contemplated for its effects on blocking propagation of tau pathology. However, it is preferred, as described herein, that the agent or antagonist (whether small organic molecule, antibody or other) binds to a selected phosphorylated form of an HMW tau or a selected phosphorylation site of the HMW tau, and not substantially to LMW tau or HMW tau without the selected phosphorylation or a non-target phosphorylation site (e.g., S422) of HMW tau. By “not substantially” is meant that the K_{D} for a specific phosphorylated form of HMW tau (e.g., S396-, S404-, and/or S199-phosphorylated soluble HMW tau) or a specific phosphorylation site of HMW tau (e.g., S396, S404, and/or S99), as determined, e.g., by competition assay or by other means known in the art, is at least 10²-fold lower than that for other non-target molecules (e.g., LMW tau or HMW tau without the selected phosphorylation or a non-target phosphorylation site (e.g., S422) of HMW tau), and preferably at least 10³-fold lower, at least 10⁴-fold lower, 10-fold lower or less. Specific binding can be influenced by, for example, the affinity and avidity of the antagonist and the concentration of the antagonist used. A person of ordinary skill in the art can determine appropriate conditions under which the antagonists described herein selectively bind using any suitable methods, such as titration of a phosphorylated soluble HMW tau species antagonist agent in a suitable assay and measuring neuron uptake of various forms of phosphorylated soluble HMW tau species, such as those described herein in the Examples.

In some embodiments, the phosphorylated soluble HMW tau species antagonist agents described herein can be modified. By way of example only, in some embodiments, the phosphorylated soluble HMW tau species antagonist agents described herein can be coupled or conjugated to a detectable label as described herein. In some embodiments, the phosphorylated soluble HMW tau species antagonist agents described herein and a detectable label can be fused together to form a fusion protein.

Phosphorylated Soluble HMW Tau Species Antagonist Antibodies and Antigen-Binding Fragments Thereof:

Also provided herein, in some aspects, are compositions comprising phosphorylated soluble HMW tau species antagonist antibodies that specifically bind at, or in close proximity to, a target phosphorylation site (e.g., S396, S404, and/or S199) of soluble HMW tau species described herein, and/or specifically binds and reduces (e.g., by at least 30% or more) or inhibits neuron uptake of soluble HMW tau species that is phosphorylated at a target site (e.g., S396, S404, and/or S199); and does not bind soluble low molecular weight (LMW) tau species. The inventors have shown that blocking the phosphorylation site at serine 396 of the phosphorylated soluble HMW tau species can significantly reduce neuronal uptake of the HMW tau species and thus inter-neuron propagation.

Phosphorylated soluble HMW tau species antibody antagonists for use in the compositions and methods described herein include complete immunoglobulins, antigen binding fragments of immunoglobulins, as well as antigen-binding fragments that comprise antigen binding domains of immunoglobulins. As used herein, “antigen-binding fragments” of immunoglobulins include, for example, Fab, Fab′, F(ab′)2, scFv and dAbs. Modified antibody formats have been developed which retain binding specificity, but have other characteristics that can be desirable, including for example, bispecificity, multivalence (more than two binding sites), and compact size (e.g., binding domains alone).

As antibodies can be modified in a number of ways, the term “antibody” should be construed as covering any specific binding member or substance having an antibody binding domain with the required specificity for a phosphorylated form of soluble HMW tau species. Thus, this term covers antibody fragments, derivatives, functional equivalents and homologues of antibodies, including any polypeptide comprising an immunoglobulin binding domain that is specific for a phosphorylated form of an HMW tau (e.g., S396-, S404-, and/or S199-phosphorylated soluble HMW tau), whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

Accordingly, in some aspects, provided herein are phosphorylated soluble HMW tau species antagonist antibodies or antibody fragment thereof that are specific for a specific phosphorylated form (e.g., S396-, S404, and/or S199-phosphorylated) of soluble HMW tau species, wherein the phosphorylated soluble HMW tau species antagonist antibodies or antibody fragment thereof specifically binds to the specific phosphorylated form of the phosphorylated soluble HMW tau species and reduces or inhibits the biological activity of the specific phosphorylated soluble HMW tau species, e.g., being taken up by neuron(s) and/or inducing inter-neuron propagation. In some embodiments, phosphorylated soluble HMW tau species is human phosphorylated soluble HMW tau species.

As used herein, a “phosphorylated soluble HMW tau species antibody” is an antibody that binds to a specific phosphorylated form of soluble HMW tau species with sufficient affinity and specificity that does not substantially bind soluble LMW tau species or other phosphorylated forms of soluble HMW tau species. The antibody selected will normally have a binding affinity for a specific phosphorylated form of soluble HMW tau species, for example, the antibody can bind a specific phosphorylated form of human soluble HMW tau species with a K_{D} value between 10⁻⁵ M to 10⁻¹⁰ M or lower. Antibody affinities can be determined, for example, by a surface plasmon resonance based assay (such as the BIAcore assay described in PCT Application Publication No. WO2005/012359); enzyme-linked immunoabsorbent assay (ELISA); and competition assays (e.g. RIA's). A phosphorylated soluble HMW tau-specific antibody as described herein will bind soluble HMW tau protein phosphorylated at a specific site (e.g., S396, S404, and/or S199) with a K_{D} at least 100-fold lower than its K_{D} for soluble LMW tau protein or soluble HMW tau protein phosphorylated at other sites, preferably at least 10³-fold lower, 10⁴-fold lower or even 10 fold lower. Relative affinities can also be evaluated, e.g. by competition assays.

In certain aspects described herein, a phosphorylated soluble HMW tau species antibody can be used as a therapeutic agent in targeting and interfering with diseases or conditions where phosphorylated soluble HMW tau species activity is involved. Also, a phosphorylated soluble HMW tau species antibody can be subjected to other biological activity assays, e.g., in order to evaluate its effectiveness as a therapeutic, or its effectiveness as a diagnostic aid, etc. Such assays are known in the art and depend on the target antigen and intended use for the antibody. Examples include measurements of phosphorylated soluble HMW tau species being taken up by neuron(s) as described in Example 1; antibody-dependent cellular cytotoxicity (ADCC) and complement-mediated cytotoxicity (CDC) assays (U.S. Pat. No. 5,500,362); and agonistic activity or hematopoiesis assays (see WO 95/27062). Other biological activity assays that can be used to assess a phosphorylated soluble HMW tau species antibody are described in the Examples section such as measuring inter-neuron propagation of phosphorylated soluble HMW tau species using a 3-chamber microfluidic device.

As used herein, a “blocking” antibody or an antagonist antibody is one which inhibits or reduces biological activity of the antigen it binds. In this context, “reduces” refers to at least a 50% reduction in the relevant biological activity (e.g., interneuron transmission of phosphorylated soluble HMW tau), e.g., at least 60%, at least 70%, at least 80%, at least 90% or more. For example, a phosphorylated soluble HMW tau species antagonist antibody binds a specific phosphorylated form of soluble HMW tau species and inhibits the ability of the phosphorylated soluble HMW tau species to, for example, to be taken up by neuron(s) and/or to get involved in inter-neuron propagation. While 100% inhibition is not necessarily required to achieve a therapeutic benefit, in certain embodiments, blocking antibodies or antagonist antibodies completely inhibit the biological activity of a specific phosphorylated form of soluble HMW tau species described herein.

Thus, phosphorylated soluble HMW tau species antibodies or antibody fragments thereof that are useful in the compositions and methods described herein include any antibodies or antibody fragments thereof that bind with sufficient affinity and specificity to a phosphorylated form of soluble HMW tau species, i.e., are specific for a selected phosphorylated form of soluble HMW tau species, and can reduce or inhibit the biological activity of the phosphorylated soluble HMW tau species, specifically ability of phosphorylated soluble HMW tau species being taken up by neuron(s) and/or inducing inter-neuron propagation.

As described herein, an “antigen” is a molecule that is bound by a hypervariable region binding site of an antibody or antigen-binding fragment thereof. Typically, antigens are bound by antibody ligands and are capable of raising an antibody response in vivo. An antigen can be a polypeptide, protein, nucleic acid or other molecule. In the case of conventional antibodies and fragments thereof, the antigen binding site as defined by the hypervariable loops (L1, L2, L3 and H1, H2, H3) is capable of binding to the antigen.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

In some embodiments of the aspects described herein, a phosphorylated soluble HMW tau species antagonist antibody is a monoclonal antibody. The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with various aspects described herein can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example.

The phosphorylated soluble HMW tau species antagonist monoclonal antibodies described herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

As used herein, a “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous mouse immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody can be prepared via immortalization of human B lymphocytes producing an antibody directed against a target antigen (such B lymphocytes can be recovered from an individual or can have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

In other embodiments of these aspects, the phosphorylated soluble HMW tau species antagonist antibody is a phosphorylated soluble HMW tau species-specific antibody fragment. The term “antibody fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains. (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) the dAb fragment (Ward et al., Nature 341, 544-546 (1989)) which consists of a V_{H} domain; (vii) isolated CDR regions; (viii) F(ab′)2 fragments, a bivalent fragment including two Fab′ fragments linked by a disulfide bridge at the hinge region; (ix) single chain antibody molecules (e.g., single chain Fv; scFv) (Bird e al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870).

Accordingly, in some such embodiments, the phosphorylated soluble HMW tau species antagonist antibody fragment is a Fab fragment comprising V_{L}, C_{L}, V_{H} and C_{H}1 domains. In some embodiments, the phosphorylated soluble HMW tau species antagonist antibody fragment is a Fab′ fragment, which is a Fab fragment having one or more cysteine residues at the C-terminus of the C_{H}1 domain. In some embodiments, the phosphorylated soluble HMW tau species antagonist antibody fragment is a Fd fragment comprising V_{H} and C_{H}1 domains. In some embodiments, the phosphorylated soluble HMW tau species antagonist antibody is a Fd′ fragment comprising V_{H} and C_{H}1 domains and one or more cysteine residues at the C-terminus of the C_{H}1 domain. In some embodiments, the phosphorylated soluble HMW tau species antagonist antibody fragment is a Fv fragment comprising the V_{L} and V_{H} domains of a single arm of an antibody. In some embodiments, the phosphorylated soluble HMW tau species antagonist antibody fragment is a dAb fragment comprising a V_{H} domain. In some embodiments, the phosphorylated soluble HMW tau species antagonist antibody fragment comprises isolated CDR regions. In some embodiments, the human phosphorylated soluble HMW tau species antagonist antibody fragment is a F(ab′)2 fragment, which comprises a bivalent fragment comprising two Fab′ fragments linked by a disulfide bridge at the hinge region. In some embodiments, the phosphorylated soluble HMW tau species antagonist antibody fragment is a single chain antibody molecule, such as a single chain Fv. In some embodiments, the phosphorylated soluble HMW tau species antagonist antibody fragment is a diabody comprising two antigen binding sites, comprising a heavy chain variable domain (V_{H}) connected to a light chain variable domain (V_{L}) in the same polypeptide chain. In some embodiments, the phosphorylated soluble HMW tau species antagonist antibody fragment is a linear antibody comprising a pair of tandem Fd segments (V_{H}-C_{H}1-V_{H}-C_{H}1) which, together with complementary light chain polypeptides, form a pair of antigen binding regions.

Antibodies to various forms of phosphorylated soluble HMW tau species can be raised by one of skill in the art using well known methods. Antibodies are readily raised in animals such as rabbits or mice by immunization with an antigen (e.g., soluble HMW tau species) or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., Eds., Antibodies: A Laboratory Manual, Cold Spring Harbor Laboratory, New York (1988), which is hereby incorporated by reference in its entirety. Both polyclonal and monoclonal antagonistic antibody of phosphorylated soluble HMW tau species can be used in the methods described herein. In some embodiments, a monoclonal antagonistic antibody of phosphorylated soluble HMW tau species is used where conditions require increased specificity for a particular protein.

In some embodiments, the phosphorylated soluble HMW tau species can be bispecific, e.g., one comprising an antigen binding domain specific for HMW tau phosphorylated at S396, and another antigen binding domain specific for HMW tau phosphorylated at S199 and/or S404.

Other Antibody Modifications.

In some embodiments of these aspects, amino acid sequence modification(s) of the antibodies or antibody fragments thereof specific for phosphorylated soluble HMW tau species described herein are contemplated. For example, it can be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also can alter post-translational processes of the antibody, such as changing the number or position of glycosylation sites.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antibody with an N-terminal methionyl residue or the antibody fused to a cytotoxic polypeptide. Other insertional variants of the antibody molecule include the fusion to the N- or C-terminus of the antibody to an enzyme (e.g. for antibody-directed enzyme prodrug therapy (ADEPT)) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated for use in the phosphorylated soluble HMW tau species antagonist antibodies or antibody fragments thereof described herein.

Substantial modifications in the biological properties of the antibodies or antibody fragments thereof specific for various phosphorylated forms of soluble HMW tau species are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (1), Pro (P), Phe (F), Trp (W), Met (M); (2) uncharged polar: Gly (G), Ser (S), Thr (T), Cys (C), Tyr (Y), Asn (N), Gin (Q); (3) acidic: Asp (D), Glu (E); (4) basic: Lys (K), Arg (R), His (H).

Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gin; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Any cysteine residue not involved in maintaining the proper conformation of the phosphorylated soluble HMW tau species antibodies or antibody fragments thereof can be substituted, generally with serine, to improve the oxidative stability of the molecule and prevent aberrant crosslinking. Conversely, cysteine bond(s) can be added to the antibody to improve its stability (particularly where the antibody is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants involves affinity maturation using phage display.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used.

Addition of glycosylation sites to the phosphorylated soluble HMW tau species antibodies or antibody fragments thereof is accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody (for O-linked glycosylation sites).

Where the antibody comprises an Fc region, the carbohydrate attached thereto can be altered. For example, antibodies with a mature carbohydrate structure that lacks fucose attached to an Fc region of the antibody are described in US Pat Appl No US 2003/0157108 A1, Presta, L. See also US 2004/0093621 A1 (Kyowa Hakko Kogyo Co., Ltd). Antibodies with a bisecting N-acetylglucosamine (GlcNAc) in the carbohydrate attached to an Fc region of the antibody are referenced in WO03/011878, Jean-Mairet et al. and U.S. Pat. No. 6,602,684, Umana et al. Antibodies with at least one galactose residue in the oligosaccharide attached to an Fc region of the antibody are reported in WO97/30087, Patel et al. See, also, WO98/58964 (Raju, S.) and WO99/22764 (Raju, S.) concerning antibodies with altered carbohydrate attached to the Fc region thereof.

To increase the serum half-life of phosphorylated soluble HMW tau species antibodies described herein, one can incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Antibodies with improved binding to the neonatal Fc receptor (FcRn), and increased half-lives, are described in WO0/42072 (Presta, L.) and US2005/0014934A1 (Hinton et al.). These antibodies comprise an Fc region with one or more substitutions therein which improve binding of the Fc region to FcRn.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

The phosphorylated soluble HMW tau species antibodies and antibody fragments thereof described herein can also be formulated as immunoliposomes, in some embodiments. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated, for example, by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the phosphorylated soluble HMW tau species antibodies described herein can be conjugated to the liposomes as described in Martin et al. J. Biol. Chem. 257: 286-288 (1982) via a disulfide interchange reaction. A therapeutic agent, e.g., for treatment of tauopathy is optionally contained within the liposome. See Gabizon et al. J. National Cancer Inst. 81(19)1484 (1989).

Nucleic Acid Inhibitors of Phosphorylated Soluble HMW Tau Species.

In some embodiments of the compositions and methods described herein, a phosphorylated soluble HMW tau species antagonist agent is an RNA interference agent that specifically targets (microtubule-associated protein tau (MAPT) and can be used for the inhibition of expression of MAPT in vivo. RNA interference (RNAi) uses small interfering RNA (siRNA) duplexes that target the messenger RNA encoding a target polypeptide for selective degradation and is a powerful approach for inhibiting the expression of selected target polypeptides. siRNA-dependent post-transcriptional silencing of gene expression involves cleaving the target messenger RNA molecule at a site guided by the siRNA. “RNA interference (RNAi),” as used herein, refers to the evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In some embodiments, the RNA interference agent or siRNA is a double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be of at least 10%, at least 20%, at least 30%, at least 40%, at least 50%, at least 60%, at least 70%, at least 80%, at least 90%, at least 95%, or at least 99% or more as compared to the expression of a target gene or the activity or level of the protein encoded by a target gene which has not been targeted by an RNA interfering agent.

As used herein, siRNAs also include small hairpin (also called stem loop) RNAs (shRNAs). In some embodiments, these shRNAs are composed of a short (e.g., about 19 to about 25 nucleotide) antisense strand, followed by a nucleotide loop of about 5 to about 9 nucleotides, and the analogous sense strand. Alternatively, in other embodiments, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). The target gene or sequence of the RNA interfering agent can be a cellular gene or genomic sequence, e.g., the human MAPT genomic sequence. An siRNA can be substantially homologous to the target gene or genomic sequence, or a fragment thereof, i.e., the MAPT gene or mRNA. As used in this context, the term “homologous” is defined as being substantially identical, sufficiently complementary, or similar to the target MAPT mRNA, or a fragment thereof, to effect RNA interference of the target MAPT. In addition to native RNA molecules, RNA suitable for inhibiting or interfering with the expression of a target sequence includes RNA derivatives and analogs. Preferably, the siRNA is identical to its target. The siRNA preferably targets only one sequence.

Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one can also screen the potential target sequences for similar sequences in the sequence databases to identify potential sequences which may have off-target effects. For example, according to Jackson et al. (Id.), 15, or perhaps as few as 11 contiguous nucleotides, of sequence identity are sufficient to direct silencing of non-targeted transcripts. Therefore, one can initially screen the proposed siRNAs to avoid potential off-target silencing using the sequence identity analysis by any known sequence comparison methods, such as BLAST. siRNA sequences are chosen to maximize the uptake of the antisense (guide) strand of the siRNA into RISC and thereby maximize the ability of RISC to target human GGT mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the human MAPT mRNA.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence may be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated. The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

In some embodiments, the RNA interference agent targeting MAPT is delivered or administered in a pharmaceutically acceptable carrier. Additional carrier agents, such as liposomes, can be added to the pharmaceutically acceptable carrier. In another embodiment, the RNA interference agent is delivered by a vector encoding the small hairpin RNA (shRNA) in a pharmaceutically acceptable carrier to the cells in an organ of an individual. The shRNA is converted by the cells after transcription into siRNA capable of targeting MAPT.

In some embodiments, the vector is a regulatable vector, such as tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, the RNA interference agents used in the methods described herein are taken up actively by neurons in vivo following intracranial injection, e.g., hydrodynamic injection, without the use of a vector, illustrating efficient in vivo delivery of the RNA interfering agents. One method to deliver the siRNAs is catheterization of the blood supply vessel of the target organ. Other strategies for delivery of the RNA interference agents, e.g., the siRNAs or shRNAs used in the methods described herein, can also be employed, such as, for example, delivery by a vector, e.g., a plasmid or viral vector, e.g., a lentiviral vector and/or adeno-associated viral (AAV) vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs targeting MAPT described herein, using a basic peptide by conjugating or mixing the RNA interfering agent with a basic peptide, e.g., a fragment of a TAT peptide, mixing with cationic lipids or formulating into particles. The RNA interference agents, e.g., the siRNAs targeting MAPT mRNA, can be delivered singularly, or in combination with other RNA interference agents, e.g., siRNAs, such as, for example siRNAs directed to other cellular genes.

Synthetic siRNA molecules, including shRNA molecules, can be generated using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule for use in the compositions and methods described herein can be selected from a given target gene sequence, e.g., a MAPT coding sequence, beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. Analysis of sequence databases, including but not limited to the NCBI, BLAST, Derwent and GenSeq as well as commercially available oligosynthesis companies such as OLIGOENGINE®, can also be used to select siRNA sequences against EST libraries to ensure that only one gene is targeted.

Delivery of RNA Interfering Agents.

Methods of delivering RNA interference agents, e.g., an siRNA, or vectors containing an RNA interference agent, to the target cells, e.g., lymphocytes or other desired target cells, for uptake include injection of a composition containing the RNA interference agent, e.g., an siRNA targeting MAPT, or directly contacting the cell, e.g., a lymphocyte, with a composition comprising an RNA interference agent, e.g., an siRNA targeting MAPT. In other embodiments, an RNA interference agent, e.g., an siRNA targeting MAPT, can be injected directly into any neuron or the brain of a subject, via, e.g., hydrodynamic injection or catheterization. Administration can be by a single injection or by two or more injections. The RNA interference agent is delivered in a pharmaceutically acceptable carrier. One or more RNA interference agents can be used simultaneously.

In some embodiments, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. A viral-mediated delivery mechanism can also be employed to deliver siRNAs to cells in vitro and in vivo as described in Xia, H. et al. (2002) Nat Biotechnol 20(10): 1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). The RNA interference agents targeting MAPT, e.g., the siRNAs or shRNAs, can be introduced along with components that perform one or more of the following activities: enhance uptake of the RNA interfering agents, e.g., siRNA, by neurons; inhibit annealing of single strands; stabilize single strands; or otherwise facilitate delivery to the target neuron and increase inhibition of the target MAPT. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

Small Organic Molecule Inhibitors of Phosphorylated Soluble HMW Tan Species.

In some embodiments of the compositions and methods described herein, a phosphorylated soluble HMW tau species antagonist agent is a small organic molecule antagonist or agent that specifically targets soluble HMW tau species phosphorylated at S396, alone or in combination with S199 and/or S404, and can be used for the inhibition of the phosphorylated soluble HMW tau species being taken up by neuron(s) and/or inducing inter-neuron propagation.

As used herein, the term “small organic molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments, a small organic molecule antagonist of phosphorylated soluble HMW tau species selectively binds to soluble HMW tau species phosphorylated at S396, alone or in combination with S199 and/or S404, and does not substantially bind soluble low molecular weight (LMW) tau species and/or soluble HMW tau species phosphorylated at other sites.

MAPT-Specific Nucleases:

In some embodiments of the compositions and methods described herein, a phosphorylated soluble HMW tau species antagonist agent is a nuclease that specifically targets MAPT gene and can be used for the inhibition of phosphorylated soluble HMW tau species being taken up by neuron(s) and/or inducing inter-neuron propagation.

As used herein, the term “nuclease” refers to an agent that induces a break in a nucleic acid sequence, e.g., a single or a double strand break in a double-stranded DNA sequence. Nucleases include those which bind a preselected or specific sequence and cut at or near the preselected or specific sequence, e.g., engineered zinc finger nucleases (ZFNs) and engineered TAL effector nucleases. Nucleases are not limited to ZFNs and TAL (transcription activator-like) effector nuclease, but can be any nuclease suitable for use with a targeting vector to achieve improved targeting efficiency. Non-limiting examples include other zinc finger-based nucleases and engineered meganucleases that cut at preselected or specific sequences (e.g., MAPT).

Specifically contemplated herein are active zinc finger nuclease proteins specific for MAPT and fusion proteins, including zinc finger protein transcription factors (ZFP-TFs) or zinc finger nucleases (ZFNs), comprising these MAPT-specific zinc finger proteins. The proteins comprising MAPT-specific zinc finger proteins can be used for therapeutic purposes, including for treatment of tau-associated neurodegeneration or tauopathy. For example, zinc finger nuclease targeting of the MAPT locus in neurons can be used to disrupt or delete the MAPT sequence. Zinc finger nucleases have been used to target different genes, e.g., as described in International Patent Application Nos. WO 2010/076939, WO2010/107493, and WO2011/139336; U.S. Patent Application No. US 2011/0158957; and U.S. Pat. No. 8,563,314 (each hereby incorporated by reference), and can be adapted to disrupt or inhibit expression and/or activity of MAPT gene.

TAL effector nucleases suitable for use in the methods of various aspects described herein include any TAL nucleases known in the art. Examples of suitable TAL nucleases, and methods for preparing suitable TAL nucleases, are disclosed, e.g., in US Patent Application No. 2011/0239315; 2011/0269234; 2011/0145940; 2003/0232410; 2005/0208489; 2005/0026157; 2005/0064474; 2006/0188987 and 2006/0063231 (each hereby incorporated by reference). In various embodiments, TAL effector nucleases are engineered that cut in or near a target nucleic acid sequence (e.g., MAPT) in, e.g., a genome of interest, wherein the target nucleic acid sequence is at or near a sequence to be modified by a targeting vector. TAL effector nucleases are proteins that comprise an endonuclease domain and one or more TAL effector DNA binding domains, wherein the one or more TAL effector DNA binding domains comprise a sequence that recognizes a preselected or specific nucleic acid sequence (e.g., MAPT).

In some embodiments, CRISPR (Clustered Regularly Interspaced Short Palindromic Repeats)/Cas system can be used to induce single or double strand breaks in target nucleic acid sequences (e.g., MAPT). It is based on an adaptive defense mechanism evolved by bacteria and archaea to protect them from invading viruses and plasmids, which relies on small RNAs for sequence-specific detection and silencing of foreign nucleic acids. Methods of using CRISPR/Cas system for gene editing and/or altering expression of gene products are known in the art, e.g., as described in U.S. Pat. No. 8,697,359, and in International Patent Application Nos. WO 2014/131833 and WO 2013/176772 (each incorporated herein by reference), and can be adapted to disrupt and/or inhibit expression level and/or activity of MAPT gene.

### Methods of Treatment Based on Selective Reduction in the Extracellular Level of Soluble HMW Tau Species in a Specific Phosphorylated Form

The inventors have shown that a relatively low level of phosphorylated soluble HMW tau species was released from neurons and found in brain interstitial fluid and cerebrospinal fluid. The inventors have also shown that the phosphorylated soluble HMW tau species, which accounts for only a small fraction of all tau in the samples, was robustly taken up by neurons, and was involved in inter-neuron propagation, whereas uptake of soluble LMW tau species (e.g., monomer/dimer size) or even non-phosphorylated soluble HMW tau species was very inefficient. By blocking specific phosphorylation site(s), e.g., at serine 396, of HMW tau species, and/or removing such specific phosphorylated form, neuronal uptake of the HMW tau species was significantly reduced. Thus, a method of preventing or reducing propagation of pathological tau protein between synaptically-connected neurons is also provided herein. The method comprises selectively reducing the extracellular level of a phosphorylated soluble HMW tau species in contact with a synaptically-connected neuron, wherein the phosphorylated soluble HMW tau species is non-fibrillar, has a molecular weight of at least about 500 kDa, and is phosphorylated at least at serine 396 (S396). A reduced extracellular level of the phosphorylated soluble HMW tau species results in reduced propagation of pathological tau protein between synaptically-connected neurons.

Without wishing to be bound by theory, where the inter-neuron propagation of pathological tau protein is concentration-dependent, by selectively reducing the extracellular level of a phosphorylated soluble HMW tau species (e.g., phosphorylated at least at serine 396) by at least 50% or more, the inter-neuron propagation of the pathological tau protein can be reduced. In some embodiments, by selectively reducing the extracellular level of a phosphorylated soluble HMW tau species (e.g., phosphorylated at least at serine 306) to or below a threshold level, the inter-neuron propagation of the pathological tau protein can be substantially inhibited. In some embodiments, the threshold level of extracellular, phosphorylated soluble HMW tau species can be no more than 500 ng/mL or lower, including, e.g., no more than 450 ng/mL, no more than 400 ng/mL, no more than 350 ng/mL, no more than 300 ng/mL, no more than 250 ng/mL, no more than 200 ng/mL, no more than 150 ng/mL, no more than 100 ng/mL, no more than 50 ng/mL, no more than 40 ng/mL, no more than 30 ng/mL, no more than 20 ng/mL, no more than 10 ng/mL, no more than 5 ng/mL, or lower. In some embodiments, the threshold level of extracellular, phosphorylated soluble HMW tau species can be between 60 ng/mL and 500 ng/mL. In some embodiments, the threshold level of extracellular, phosphorylated soluble HMW tau species can be no more than 60 ng/mL.

As used herein, the term “selectively reducing” means a greater ability to reduce extracellular level of a target phosphorylated soluble HMW tau species described herein than to reduce extracellular level of a soluble LMW tau species described herein, a non-target phosphorylated soluble HMW tau species, and/or non-phosphorylated soluble HMW tau species. In some embodiments, “selectively reducing” refers to reducing at least about 30% or more (including, e.g., at least about 40%, at least about 50%, at least about 60%, at least about 70%, at least about 80%, at least about 90%, at least 95% or more) of the extracellular level of a target phosphorylated soluble HMW tau species (e.g., S396, S404, and/or S199), while the extracellular level of soluble LMW tau species, non-target phosphorylated soluble HMW tau species (e.g., S422, S409, S400, S262, and/or T205), and/or non-phosphorylated soluble HMW tau species is reduced by no more than 30% or less (including, e.g., no more than 20%, no more than 10%, no more than 9%, no more than 8%, no more than 6%, no more than 5%, no more than 4%, no more than 2%, no more than 1% or lower). In some embodiments, “selectively reducing” refers to reducing at least about 30% or more (including, e.g., at least about 40%, at least about 50%, at least about 60%, at least about 70%, at least about 80%, at least about 90%, at least 95% or more) of the extracellular level of a target phosphorylated soluble HMW tau species (e.g., S396, S404, and/or S199), while the extracellular level of soluble LMW tau species, non-target phosphorylated soluble HMW tau species (e.g., S422, S409, S400, S262, and/or T205), and/or non-phosphorylated soluble HMW tau species is not substantially reduced during the selective reduction. For example, no more than 10% or less (including, e.g., no more than 9%, no more than 8%, no more than 6%, no more than 5%, no more than 4%, no more than 2%, no more than 1% or lower) of the extracellular level of soluble LMW tau species, non-target phosphorylated soluble HMW tau species (e.g., S422, S409, S400, S262, and/or T205), and/or non-phosphorylated soluble HMW tau species is reduced during the selective reduction.

As used herein, the term “extracellular level” refers to the level of a soluble molecule (e.g., HMW tau species or LMW tau species) outside of a neuron. Depending on the context of each application, in one embodiment, the extracellular level refers to the level in a cell culture medium. In one embodiment, the extracellular level refers to the level in brain interstitial fluid. In one embodiment, the extracellular level refers to the level in cerebrospinal fluid (e.g., ventricular cerebrospinal fluid or lumbar cerebrospinal fluid).

In some embodiments, the extracellular level of the target phosphorylated soluble HMW tau species (e.g., soluble HMW tau species phosphorylated at least at S396, S404 and/or S199) can be selectively reduced to a concentration of no more than 250 ng/mL, no more than 200 ng % mL, no more than 150 ng/mL, no more than 100 ng/mL, no more than 75 ng/mL, no more than 50 ng/mL, no more than 25 ng/mL, no more than 20 ng/mL, no more than 10 ng/mL, no more than 5 ng/mL, no more than 1 ng/mL or lower. In some embodiments, the extracellular level of the target phosphorylated soluble HMW tau species (e.g., soluble HMW tau species phosphorylated at least at S396, S404 and/or S199) can be selectively reduced to a concentration of about 20 ng/mL to about 90 ng/mL. In one embodiment, the target phosphorylated soluble HMW tau species (e.g., soluble HMW tau species phosphorylated at least at S396, S404 and/or S199) can be completely removed, i.e., the extracellular level is reduced to 0 ng/mL.

Methods for selectively reducing the extracellular level of phosphorylated soluble HMW tau species can be based on physical removal and/or molecular interactions between the phosphorylated soluble HMW tau species and an anti-phosphorylated soluble HMW tau species antagonist described herein. In some embodiments, target phosphorylated soluble HMW tau species (e.g., soluble HMW tau species phosphorylated at least at S396, S404 and/or S199) can be selectively reduced by a combination of microdialysis and molecular interaction between the phosphorylated soluble HMW tau species and a phospho-specific anti-tau antibody. The term “microdialysis” as used herein generally denotes a method of collecting a molecule or substance of interest from a microenvironment to be analyzed or treated, e.g., from a human or animal tissue or fluid, into a collector device (e.g., an interior part of a micro-dialysis probe) through a semi-permeable membrane or a selectively-permeable membrane. For example, a molecule or substance of interest (e.g., soluble HMW tau species) diffuses through the membrane (e.g., a membrane with a particular MW cutoff) and collected by a perfusion fluid flowing in an interior part of a micro-dialysis probe. The collected soluble HMW tau species can then be contacted with a phospho-specific anti-tau antibody (e.g., an antibody that specifically binds tau species phosphorylated at S396) to selectively remove or reduce the soluble HMW tau species phosphorylated at least at S396.

In some embodiments, the phosphorylated soluble HMW tau species (e.g., soluble HMW tau species phosphorylated at least at S396, S404 and/or S199) can be selectively reduced by contacting the extracellular space or fluid in contact with the synaptically-connected neurons with at least one or more antagonist of the phosphorylated soluble HMW tau species, e.g., as described in the section “Antagonists of various phosphorylated forms of soluble HMW tau species” herein. Examples of an antagonist of various phosphorylated forms of soluble HMW tau species include, without limitations, an antibody, a nuclease (e.g., but not limited to, a zinc finger nuclease (ZFN), transcription activator-like effector nuclease (TALEN), a CRISPR/Cas system, a transcriptional repressor, a nucleic acid inhibitor (e.g., RNAi, siRNA, anti-miR, antisense oligonucleotides, ribozymes, and a combination of two or more thereof), a small organic molecule, an aptamer, and a combination of two or more thereof. In some embodiments where the contact is in vitro, the target phosphorylated soluble HMW tau species (e.g., soluble HMW tau species phosphorylated at least at S396, S404 and/or S199) can be selectively reduced by adding at least one or more antagonist of the target phosphorylated soluble HMW tau species described herein into the cell culture medium in which synaptically-connected neurons are cultured. In some embodiments where the contact is in vivo, the target phosphorylated soluble HMW tau species (e.g., soluble HMW tau species phosphorylated at least at S396, S404 and/or S199) can be selectively reduced by introducing at least one or more antagonist of the target phosphorylated soluble HMW tau species described herein into brain interstitial fluid or cerebrospinal fluid, including, e.g., ventricular cerebrospinal fluid and/or lumbar cerebrospinal fluid.

A reduced extracellular level of the target phosphorylated soluble HMW tau species (e.g., soluble HMW tau species phosphorylated at least at S396, S404 and/or S199) using the methods described herein can result in reduced neuron uptake of the phosphorylated soluble HMW tau species by at least by about 10% or more (including, e.g., at least about 20%, at least about 30%, at least about 40%, at least about 50%, at least about 60%, at least about 70%, at least about 80%, at least about 90%, or more), thereby reducing propagation of pathological tau protein between synaptically-connected neurons by at least by about 10% or more (including, e.g., at least about 20%, at least about 30%, at least about 40%, at least about 50%, at least about 60%, at least about 70%, at least about 80%, at least about 90%, or more), as compared to without the selective reduction of the target phosphorylated soluble HMW tau species.

The methods described herein can be used for therapeutic treatment of tau-associated neurodegeneration or tauopathy. Tau pathology is known to spread in a hierarchical pattern in Alzheimer's disease (AD) brain during disease progression, e.g., by trans-synaptic transfer of pathological forms of tau between neurons to facilitate propagation of neurofibrillary tangles (insoluble and fibrillar tau aggregates). Since the soluble HMW tau species phosphorylated at least at S396 is identified herein to be involved in neuron-to-neuron propagation, intervention to deplete such phosphorylated soluble HMW tau species can inhibit tau propagation and hence disease progression in tauopathies. Accordingly, a method of reducing tau-associated neurodegeneration in a subject is provided herein. Examples of tau-associated neurodegeneration include, but are not limited to, Alzheimer's disease, Parkinson's disease, or frontotemporal dementia. The method of treatment comprises selectively reducing the level of a phosphorylated soluble HMW tau species in the brain (e.g., in the brain interstitial fluid) or cerebrospinal fluid (CSF) of a subject determined to have, or be at risk for, tau-associated neurodegeneration, wherein the phosphorylated soluble HMW tau species is non-fibrillar, has a molecular weight of at least about 500 kDa, and is phosphorylated at least at serine 396, wherein a reduced level of the phosphorylated soluble HMW tau species results in reduced tau-associated neurodegeneration.

In some embodiments, the method can further comprise selectively reducing the level of an additional phosphorylated soluble HMW tau species in the brain (e.g., in the brain interstitial fluid) or cerebrospinal fluid (CSF) of the subject, wherein the additional phosphorylated soluble HMW tau species is non-fibrillar, has a molecular weight of at least about 500 kDa, and is phosphorylated at least at serine 199 (S199), and/or serine 404 (S404).

In some embodiments, the level of a soluble HMW tau species phosphorylated at serine 422 is not substantially reduced in the brain (e.g., in the brain interstitial fluid) or cerebrospinal fluid (CSF) during the treatment. In some embodiments, the level of a soluble HMW tau species phosphorylated at serine 409 is not substantially reduced in the brain (e.g., in the brain interstitial fluid) or cerebrospinal fluid (CSF) during the treatment. In some embodiments, the level of a soluble HMW tau species phosphorylated at serine 400 is not substantially reduced in the brain (e.g., in the brain interstitial fluid) or cerebrospinal fluid (CSF) during the treatment. In some embodiments, the level of a soluble HMW tau species phosphorylated at serine 262 is not substantially reduced in the brain (e.g., in the brain interstitial fluid) or cerebrospinal fluid (CSF) during the treatment. In some embodiments, the level of a soluble HMW tau species phosphorylated at threonine 205 is not substantially reduced in the brain (e.g., in the brain interstitial fluid) or cerebrospinal fluid (CSF) during the treatment.

In some embodiments, the level of soluble LMW tau species in the subject is not substantially reduced in the brain (e.g., in the brain interstitial fluid) or cerebrospinal fluid (CSF) during the treatment.

In some embodiments, at least a portion (e.g., at least 30% or more, including, e.g., at least 40%, at least 50%, at least 60%, at least 70%, at least 80%, at least 90%, at least 95% or more) or all (i.e., 100%) of the target soluble HMW tau species population (e.g., soluble HMW tau species phosphorylated at least at serine 396, serine 404 and/or serine 199) present in brain interstitial fluid of the subject is removed or rendered inactive for propagation. In some embodiments, at least a portion (e.g., at least 30% or more, including, e.g., at least 40%, at least 50%, at least 60%, at least 70%, at least 80%, at least 90%, at least 95% or more) or all (i.e., 100%) of the target soluble HMW tau species population (e.g., soluble HMW tau species phosphorylated at least at serine 396, serine 404, and/or serine 199) present in cerebrospinal fluid of the subject is removed or rendered inactive for propagation.

Methods for selectively reducing the extracellular level of target phosphorylated soluble HMW tau species have been described above and can be applied to selectively reduce the level of target phosphorylated soluble HMW tau species (e.g., HMW tau species phosphorylated at least at S396, S404 and/or S199) in the brain of a subject. For example, in some embodiments, the target phosphorylated soluble HMW tau species (e.g., HMW tau species phosphorylated at least at S396, S404 and/or S199) present in the brain interstitial fluid and/or cerebrospinal fluid of the subject can be selectively reduced by brain microdialysis in combination with immunodepletion. For example, brain microdialysis can be used to separate soluble HMW tau species from LMW tau species, and the separated soluble HMW tau species can then be contacted with a phospho-specific anti-tau antibody (e.g., a phospho-S396 anti-tau antibody). In some embodiments, the target phosphorylated soluble HMW tau species (e.g., HMW tau species phosphorylated at least at S396, S404 and/or S199) present in the brain interstitial fluid and/or cerebrospinal fluid of the subject can be selectively reduced by administering to the brain of the subject an antagonist of target phosphorylated soluble HMW tau species, e.g., by intracranial injection, intracortical injection, or intracerebroventricular injection, or via peripheral administration of a molecule that crosses the blood brain barrier in sufficient quantities.

In some embodiments, the method can further comprise selecting a subject determined to have target phosphorylated soluble HMW tau species (e.g., HMW tau species phosphorylated at least at S396, S404 and/or S199) present in the brain (e.g., in brain interstitial fluid or cerebrospinal fluid) at a level above a reference level, or determined to be at risk for, or have tau-associated neurodegeneration or tauopathy.

In some embodiments, the reference level can be at least about 5 ng/mL, at least about 10 ng/mL, at least about 15 ng/mL, at least about 20 ng/mL, at least about 25 ng/mL or higher (including, e.g., at least about 50 ng/mL, at least about 100 ng/mL, at least about 150 ng/mL, at least about 200 ng/mL or higher). In some embodiments, the reference level can be no more than 500 ng/mL, no more than 400 ng/mL, no more than 300 ng/mL, no more than 200 ng/mL, no more than 100 ng/mL, no more than 50 ng/mL, no more than 25 ng/mL, no more than 10 ng/mL, or no more than 5 ng/mL. In some embodiments, the reference level can be no more than 100 pg/mL, no more than 50 pg/mL, no more than 25 pg/mL, no more than 20 pg/mL, no more than 10 pg/mL, or lower.

In some embodiments, a reference level can represent an extracellular level of target phosphorylated soluble HMW tau species (e.g., HMW tau species phosphorylated at least at S396, S404 and/or S199) present in the brain (e.g., in brain interstitial fluid or cerebrospinal fluid, including, e.g., ventricular or lumbar cerebrospinal fluid) of healthy subject(s). The extracellular level of target phosphorylated soluble HMW tau species (e.g., HMW tau species phosphorylated at least at S396, S404 and/or S199) is generally much lower in the brain (e.g., in brain interstitial fluid or cerebrospinal fluid, including, e.g., ventricular or lumbar cerebrospinal fluid) of healthy subject(s) than in the brain of AD subject(s). In some embodiments, the extracellular level of target phosphorylated soluble HMW tau species (e.g., HMW tau species phosphorylated at least at S396, S404 and/or S199) is about 33 times lower in the brain of healthy subject(s) than in the brain of AD subject(s). Accordingly, in some embodiments, the method can further comprise selecting a subject whose extracellular level of target phosphorylated soluble HMW tau species (e.g., HMW tau species phosphorylated at least at S396, S404 and/or S199) present in the brain (e.g., in brain interstitial fluid or cerebrospinal fluid) is determined to be at least about 20 times or higher (including, e.g., at least about 25 times, at least about 30 times, at least about 35 times, at least about 40 times or greater) above a reference level (e.g., the extracellular level of soluble HMW tau species phosphorylated at least at serine 396 in the brain of healthy subject(s)).

In one embodiment, the extracellular level of target phosphorylated soluble HMW tau species (e.g., HMW tau species phosphorylated at least at S396, S404 and/or S199) in the brain of healthy subject(s) is about 0.4 ng per ml of cerebrospinal fluid or brain interstitial fluid. Accordingly, in some embodiments, the subject selected for the method described herein have target phosphorylated soluble HMW tau species (e.g., HMW tau species phosphorylated at least at S396, S404 and/or S199) present in the brain (e.g., in brain interstitial fluid or cerebrospinal fluid) at an extracellular level of at least about 10 ng/mL or higher, including, e.g., at least about 11 ng/mL, at least about 12 ng/mL, at least about 13 ng/mL, at least about 14 ng/mL, at least about 15 ng/mL, at least about 20 ng/mL, at least about 25 ng/mL, at least about 30 ng/mL, at least about 35 ng/mL, at least about 40 ng/mL, or higher.

Methods to diagnose or identify a subject for tau-associated neurodegeneration or tauopathy are known in the art and can be used herein to select a subject amenable to the methods of treatment described herein. Methods of diagnosing tau-associated neurodegeneration as described below and as described in the section “Selection of Subjects in Need Thereof for the Methods of Treatment Described herein” below can also be used to select a subject amenable to the methods of treatment described herein.

### Methods of Diagnosing Tau-Associated Neurodegeneration

In a further aspect, a method of diagnosing tau-associated neurodegeneration based on the presence and/or levels of a soluble HMW tau species phosphorylated at least at serine 396 is also provided herein. Exemplary tau-associated neurodegeneration includes, but is not limited to, Alzheimer's disease, Parkinson's disease, or frontotemporal dementia. The inventors have shown that the cerebrospinal fluid (CSF) (e.g., ventricular or lumbar CSF) from AD brain extract contained significantly higher levels of phosphorylated soluble HMW tau species, when compared to that of the control brain. Therefore, the method of diagnosing tau-associated neurodegeneration can comprise (a) fractionating a sample of brain interstitial fluid or cerebrospinal fluid from a subject; and (b) detecting a phosphorylated soluble HMW tau species in the sample such that the presence and amount of the phosphorylated soluble HMW tau species is determined, wherein the phosphorylated soluble HMW tau species is non-fibrillar, has a molecular weight of at least about 500 kDa, and is phosphorylated at least at serine 396; and (c) identifying the subject to have, or be at risk for tau-associated neurodegeneration when the level of the phosphorylated soluble HMW tau species in the sample is the same as or above a reference level; or identifying the subject to be less likely to have tau-associated neurodegeneration when the level of the phosphorylated soluble HMW tau species is below a reference level. A reference level can represent a level of soluble H-MW tau species phosphorylated at least at serine 396 present in healthy subject(s).

In some embodiments, the method can further comprise detecting an additional phosphorylated soluble HMW tau species in the sample such that the presence and amount of the additional phosphorylated soluble HMW tau species is determined, wherein the additional phosphorylated soluble HMW tau species is non-fibrillar, has a molecular weight of at least about 500 kDa, and is phosphorylated at least at serine 199 (S199), and/or serine 404 (S404). Antibodies to these phosphorylation sites are commercially available, e.g., from Life Technologies.

In some embodiments, the amount of soluble HMW tau species that is phosphorylated at least at S396 and optionally at S404 and/or S199 in the sample can be detected for diagnostic purpose.

Accordingly, in some embodiments, a reference level can represent the amount of soluble HMW tau species that is phosphorylated at least at S396 and optionally at S404 and/or S199 present in the brain of healthy subject(s). In these embodiments, a reference level can represent the amount of soluble HMW tau species phosphorylated at least at S396 and optionally at S404 and/or S199 present in brain interstitial fluid or cerebrospinal fluid of healthy subject(s). In these embodiments, the reference level can be at least about 5 ng/mL, at least about 10 ng/mL, at least about 15 ng/mL, at least about 20 ng/mL, at least about 25 ng/mL or higher (including, e.g., at least about 50 ng/mL, at least about 100 ng/mL, at least about 150 ng/mL, at least about 200 ng/mL or higher). In some embodiments, the reference level can be no more than 500 ng/mL, no more than 400 ng/mL, no more than 300 ng/mL, no more than 200 ng/mL, no more than 100 ng/mL, no more than 50 ng/mL, no more than 25 ng/mL, no more than 10 ng/mL, or no more than 5 ng/mL.

As used herein, the term “fractionating” refers to separating a sample into a plurality of fractions based on a certain parameter, e.g., molecular sizes or molecular weights and/or phosphorylation. In the context of various aspects described herein, the term “fractionating” refers to separating at least one or more phosphorylated forms of soluble HMW tau species from a sample of brain interstitial fluid or cerebrospinal fluid or enriching the sample with a target phosphorylated form of soluble HMW tau species (e.g., soluble HMW tau species phosphorylated at least at S396, S404, and/or S199). In some embodiments, the fractionation is based on molecular size and/or molecular weight of molecules present in the sample. Such size or weight exclusion methods are known in the art, e.g., but not limited to size exclusion chromatography, centrifugation, gel electrophoresis, sucrose density, affinity chromatography, dialysis, or a combination of two or more thereof. Additionally or alternatively, the fractionation can be based on phosphorylation at a specific site. Methods to detect soluble HMW tau species phosphorylated at a specific site are known in the art, including, e.g., immunoprecipitation, and can be used to perform the fractionation step described herein.

In some embodiments, the sample, prior to the fractionating of (a), can be substantially free of soluble LMW tau species, wherein the soluble LMW tau species has a molecular weight of no more than 200 kDa or lower. For example, a sample of brain interstitial fluid or cerebrospinal fluid can be obtained from a subject to be diagnosed by microdialysis, e.g., using a permeable membrane with a proper molecular-weight cut-off, e.g., which would allow only molecules with a molecular weight of at least about 600 kDa to be collected.

In alternative embodiments, the sample, prior to the fractionating of (a), can comprise soluble LMW tau species, wherein the soluble LMW tau species has a molecular weight of no more than 200 kDa. By fractionating the sample, one can isolate the phosphorylated soluble HMW tau species from other low MW molecules in the sample (e.g., soluble LMW tau species) and enrich the sample with the phosphorylated soluble HMW tau species for diagnostic purposes. In some embodiments, the fractionation can be based on size exclusion and/or antibody-based methods.

After fractionation, the target phosphorylated soluble HMW tau species (e.g., soluble HMW tau species phosphorylated at least at S396, S404, and/or S199) in the sample can be detected by any methods typically used to detect tau protein, including, e.g., but not limited to, ELISA, western blot, immunoassay, size exclusion chromatography, a combination of two or more thereof.

In some embodiments where soluble LMW tau species is present in the sample, the method can further comprise detecting the amount of the soluble LMW tau species phosphorylated at serine 396 in the sample. In these embodiments, the subject can be identified to have, or be at risk for tau-associated neurodegeneration if a ratio of the amount of the S396-phosphorylated soluble HMW tau species to that of the S396-phosphorylated soluble LMW tau species is the same as or above a reference level ratio; or the subject is identified to be less likely to have tau-associated neurodegeneration if the ratio of the amount of the S396-phosphorylated soluble HMW tau species to that of the S396-phosphorylated soluble LMW tau species is below the reference level ratio. A reference level ratio can represent a level ratio of the amount of the S396-phosphorylated soluble HMW tau species to that of the S396-phosphorylated soluble LMW tau species present in healthy subject(s).

In some embodiments where soluble LMW tau species is present in the sample, the method can further comprise detecting the level of phosphorylation at serine 396 of the soluble LMW tau species in the sample. In these embodiments, the subject can be identified to have, or be at risk for tau-associated neurodegeneration if a ratio of the S396 phosphorylation level in the phosphorylated soluble HMW tau species to that in the LMW tau species is the same as or above a reference level ratio; or the subject is identified to be less likely to have tau-associated neurodegeneration if the ratio of the S396 phosphorylation level in the phosphorylated soluble HMW tau species to that in the LMW tau species is below the reference level ratio. A reference level ratio can represent a ratio of S396 phosphorylation level in soluble HMW tau species to that in LMW tau species present in healthy subject(s).

In some embodiments where soluble LMW tau species is present in the sample, the method can further comprise detecting the total amount of the soluble LMW in the sample (e.g., regardless of whether it is phosphorylated or not). In these embodiments, the subject can be identified to have, or be at risk for tau-associated neurodegeneration if a ratio of the amount of the S396-phosphorylated soluble HMW tau species to the total amount of the soluble LMW tau species is the same as or above a reference ratio; or the subject is identified to be less likely to have tau-associated neurodegeneration if the ratio of the amount of the S396-phosphorylated soluble HMW tau species to the total amount of the soluble LMW tau species is below the reference ratio. A reference ratio can represent a ratio of the amount of the S396-phosphorylated soluble HMW tau species to the total amount of the soluble LMW tau species present in healthy subject(s). In some embodiments, the reference ratio can represent a ratio of the extracellular amount of the S396-phosphorylated soluble HMW tau species to the total extracellular amount of the soluble LMW tau species present in the brain of healthy subject(s). The level of LMW tau greatly exceeds that of HMW tau, even in individuals with AD. For example, HMW tau generally makes up only ˜1-5% (or less) of total tau proteins. Thus, it is contemplated that HMW tau makes up only ˜1-5% (or less) of total tau proteins. Thus, in some embodiments, a reference ratio can represent a ratio of the amount of target phosphorylated soluble HMW tau species (e.g., soluble HMW tau species phosphorylated at least at S396, S404, and/or S199) to the total amount of soluble LMW tau species present in brain interstitial fluid or cerebrospinal fluid of healthy subject(s). In some embodiments, the reference ratio can range from about 1:10000 to about 1:20, about 1:1000 to about 1:50, or about 1:500 to about 1:50, or about 1:100 to about 1:20.

In some embodiments, the method can further comprise administering to a subject identified to have, or be at risk for tau-associated neurodegeneration a therapeutic treatment, e.g., a pharmaceutical composition comprising one or more antagonist of the soluble HMW tau species phosphorylated at S396, S199, and/or S404. Examples of an antagonist of such phosphorylated soluble HMW tau species include, without limitations, an antibody, a nuclease (e.g., but not limited to, a zinc finger nuclease (ZFN), transcription activator-like effector nuclease (TALEN), a gene-editing composition (e.g., CRISPR/Cas system), a transcriptional repressor, a nucleic acid inhibitor (e.g., RNAi, siRNA, anti-miR, antisense oligonucleotides, ribozymes, and a combination of two or more thereof), a small organic molecule, an aptamer, and a combination of two or more thereof. Methods known for effectively delivering an agent to the brain of a subject can be used to perform the treatment methods of various aspects described herein. In some embodiments of this aspect and other aspects described herein, the agent can be administered to the brain via a carrier. An exemplary carrier can be a virus or viral vector (e.g., but not limited to, retrovirus, adenovirus, adeno-associated virus (AAV), recombinant AAV expression vector), a nanoparticle, and/or a liposome.

In some embodiments of this aspect and other aspects described herein, the brain of the subject can be further determined to have amyloid beta plaques and the administration can reduce neurotoxicity (and/or increase neuron survival) in the presence of amyloid beta. See, e.g., Pooler et al. “Amyloid accelerates tau propagation and toxicity in a model of early Alzheimer's disease” Acta Neuropathologica Communications (2015) 3:14, which reports that tau propagation and/or tau-induced neuronal loss is substantially increased in the presence of amyloid beta. By administering to a subject identified to have concurrent amyloid beta deposition in the brain a pharmaceutical composition comprising one or more antagonists of the soluble HMW tau species phosphorylated at S396, S199, and/or S404, tau propagation and/or tau-induced neuronal loss can be reduced, thereby reducing neurotoxicity (and/or increasing neuron survival) in the presence of amyloid beta.

Methods of Screening for Agents that Reduce Cross-Synaptic Spread of Misfolded Tau Proteins

Not only does the discovery of specific phosphorylated forms of soluble HMW tau species (e.g., phosphorylated at serine 396, serine 404, and serine 199) provide one or more therapeutic targets and biomarkers for tau-associated neurodegeneration as described herein, the target phosphorylated soluble HMW tau species described herein (e.g., soluble HMW tau species phosphorylated at least at S396, S404 and/or S199) can also be used in screening assays to identify agents that modulate the formation or activity of the HMW tau species itself (e.g., by blocking the formation or stability of the HMW tau species, or, for example, by blocking post-translational modifications or by destabilizing the HMW tau structure, or by reducing or inhibiting phosphorylation of the phosphorylated soluble HMW tau species at least at serine 396). For example, a test agent, e.g., aptamers, small organic molecules or other agents, can be applied to neuronal cell cultures and the presence or amount of target phosphorylated soluble HMW tau (e.g., soluble HMW tau phosphorylated at least at S396, S404 and/or S199) or level of phosphorylation at S396, S404 and/or S199 of the HMW tau species can be monitored. An agent so identified that blocks the formation or accumulation of target phosphorylated soluble HMW tau species (e.g., soluble HMW tau phosphorylated at least at S396, S404 and/or S199) and/or reduces the level of phosphorylation at S396, S404, and/or S199 of the HMW tau species can be of interest as a potential therapeutic.

Additionally or alternatively, the specific phosphorylated forms of soluble HMW tau species can also be used in vitro to induce inter-neuron propagation, a phenotypic feature of progression in neurodegeneration, and thus develop an in vitro model to screen for effective agents that reduce cross-synaptic spread of misfolded tau proteins to treat tau-associated neurodegeneration. Accordingly, a further aspect provided herein relates to a method of identifying an agent that is effective to reduce cross-synaptic spread of misfolded tau proteins. The method comprises (a) contacting a first neuron in a first chamber of a neuron culture device with a composition comprising a phosphorylated soluble HMW tau species, the phosphorylated soluble HMW tau species being phosphorylated at serine 396, wherein the first neuron is axonally connected with a second neuron in a second chamber of the neuron culture device, and wherein the second neuron is not contacted with the phosphorylated soluble HMW tau species; (b) contacting the first neuron from (a) in the first chamber with a candidate agent; and (c) detecting transport of the phosphorylated soluble HMW tau species from the first neuron to the second neuron. An effective agent for reducing cross-synaptic spread of misfolded tau proteins can be identified based on detection of the presence or absence of the phosphorylated soluble HMW tau species in an axon and/or soma of the second neuron.

In some embodiments, the first neuron can be contacted with the phosphorylated soluble HMW tau species and the candidate agent concurrently. In some embodiments, the first neuron can be contacted with the phosphorylated soluble HMW tau species prior to contact with the candidate agent. In some embodiments, the first neuron can be contacted with the phosphorylated soluble HMW tau species after contact with the candidate agent.

As used herein, the term “candidate agent” refers to any compound or substance such as, but not limited to, a small organic molecule, nucleic acid, polypeptide, peptide, drug, ion, etc., which is desired to be tested for its ability to reduce or inhibit neuron uptake of a specific phosphorylated form of soluble HMW tau species and/or to reduce inter-neuron propagation of the phosphorylated soluble HMW tau species. A “candidate agent” can be any chemical, entity, or moiety, including, without limitation, synthetic and naturally-occurring proteinaceous and non-proteinaceous entities. In some embodiments, a candidate agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. In some embodiments, agents are small organic molecules having a chemical moiety. For example, chemical moieties include unsubstituted or substituted alkyl, aromatic, or heterocyclyl moieties.

An effective agent for reducing cross-synaptic spread of misfolded tau proteins can be identified based on detection of the presence or absence of, or level of the target phosphorylated soluble HMW tau species (e.g., soluble HMW tau species phosphorylated at S396, S404 and/or S199) in an axon and/or soma of the second neuron. If the target phosphorylated soluble HMW tau species (e.g., soluble HMW tau species phosphorylated at S396, S404 and/or S199) in an axon and/or soma of the second neuron is reduced by at least about 30% or more (including, e.g., at least about 40%, at least about 50%, at least about 60%, at least about 70%, at least about 80%, at least about 90%, at least about 95%, or more, and up to 100%), as compared to when the first neuron in the first chamber is not contacted with the candidate agent, the candidate agent is identified as an effective agent for reducing cross-synaptic spread of misfolded tau proteins or the target phosphorylated soluble HMW tau species described herein.

As used herein, the term “axonally connected” refers to neurons are connected by an axon. As used herein, the term “axon” refers to a long cellular protrusion from a neuron, whereby efferent (outgoing) action potentials are conducted from the cell body towards target cells.

While any neuron culture device suitable for monitoring axonal extension and/or transport can be used in the methods described herein, in some embodiments, the neuron culture device is a microfluidic device. In some embodiments, the microfluidic device can comprise a first chamber for placing at least a first neuron and a second chamber for placing at least a second neuron, wherein the first chamber and the second chamber are interconnected by at least one microchannel exclusively sized to permit axon growth. In some embodiments, the microfluidic device can comprise a first chamber for placing a first population (e.g., at least 2 or more) of neurons and a second chamber for placing a second population (e.g., at least 2 or more) of neurons, wherein the first chamber and the second chamber are interconnected by at least two or more microchannels, each exclusively sized to permit axon growth.

As used herein, the term “exclusively sized to permit axon growth” refers to the dimensions of the interconnecting microchannel(s) being sized to exclusively allow an extension of an axon, originating from the cell body of neuron(s) in the first chamber to enter the second chamber. For example, the length of the microchannel(s) interconnecting the first chamber and the second chamber is optimized such that no MAP2-positive dendrites can enter the second chamber, thus isolating axon terminals from soma and dendrites. In some embodiments, the length of the microchannel(s) can be at least about 400 μm or more, including, e.g., at least about 500 μm, at least about 600 μm, at least about 700 μm, at least about 800 μm, at least about 900 μm, at least about 1000 μm. In one embodiment, the length of the microchannel(s) can be at least about 450 μm or more. In one embodiment, the length of the microchannel(s) can be at least about 600 μm or more.

In some embodiments, the width of the microchannels can be exclusively sized to permit axon growth. In some embodiments, the width of the microchannels can range from about 3 μm to about 15 μm, or from about 5 μm to about 10 μm, or from about 6 μm to about 10 μm.

In some embodiments, the microfluidic device can further comprise a third chamber for placing at least a third neuron, wherein the second chamber and the third chamber are interconnected by at least one microchannel exclusively sized to permit axon growth as described herein.

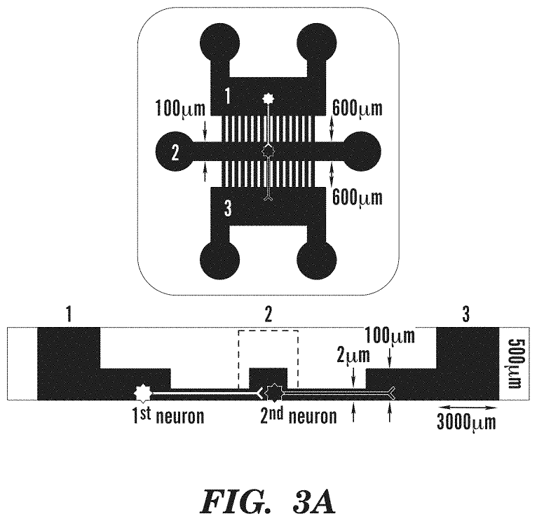

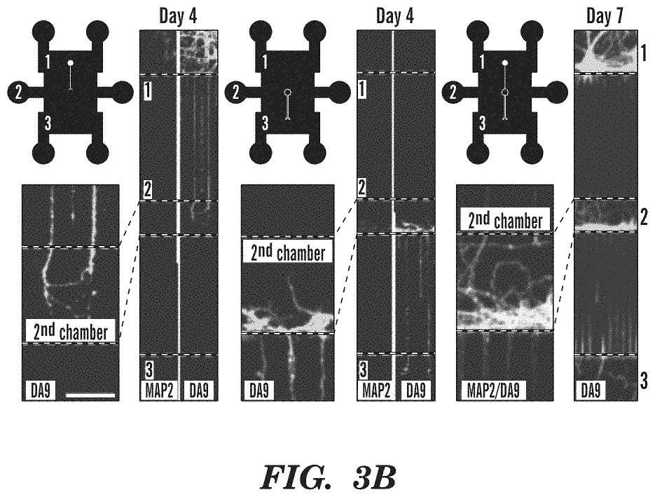

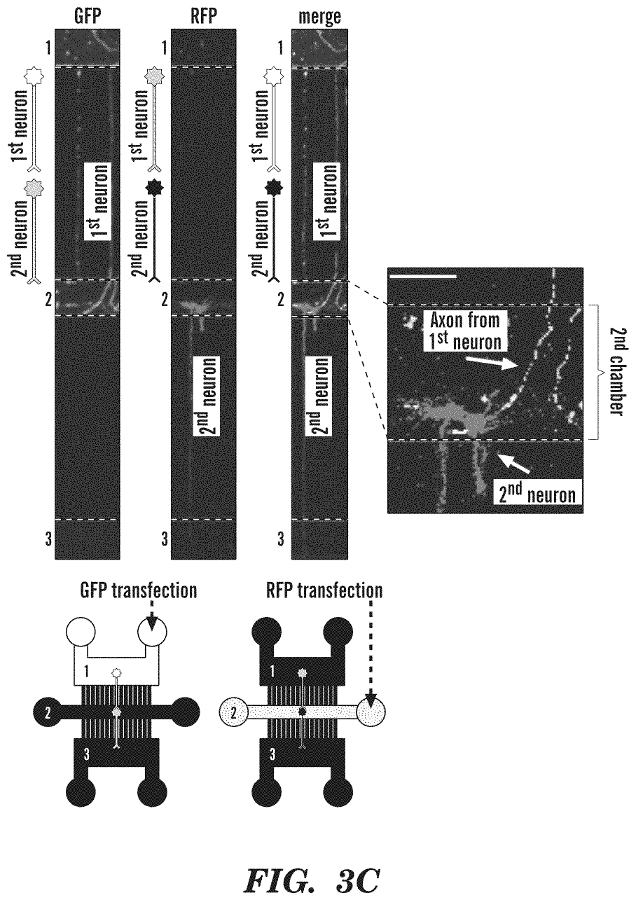

By way of example, FIG. 3A shows a schematic diagram of an exemplary neuron culture device. FIG. 3A shows a microfluidic device 300, which comprises a first chamber 302 for placing at least a first neuron and a second chamber 304 for placing at least a second neuron, wherein the first chamber 302 and the second chamber 304 are interconnected by at least one microchannel 306 exclusively sized to permit axon growth. In some embodiments, more than one microchannel 306 (e.g., at least two or more microchannels) interconnecting the two chambers can be desirable so that multiple axons can be monitored simultaneously. In some embodiments, the microfluidic device 300 can further comprise a third chamber 308 for placing at least a third neuron, wherein the second chamber 304 and the third chamber 308 are interconnected by at least one microchannel 306 exclusively sized to permit axon growth.

To prevent diffusion of the phosphorylated soluble HMW tau species and candidate agent from the first chamber into other chambers (e.g., the second chamber and/or the optional third chamber), the second chamber and/or the optional third chamber can be added with a greater amount of cell culture medium than what is added in the first chamber such that the volume difference between the chambers can result in continuous convection (“hydrostatic pressure barrier”). In some embodiments, the amount of the cell culture medium added into the second and/or the optional third chamber can be greater than that in the first chamber by at least about 1.5-fold, at least about 2-fold, at least about 3-fold, at least about 4-fold, at least about 5-fold, at least about 10-fold or higher. In one embodiment, the amount of the cell culture medium added into the second and/or the optional third chamber can be greater than that in the first chamber by at least about 4-fold or higher.

To detect the transport of target phosphorylated soluble HMW tau species (e.g., soluble HMW tau species phosphorylated at S396, S404 and/or S199) from a first neuron to a second neuron, the neurons can be fixed, immunostained for presence of soluble HMW tau species using anti-tau antibodies or anti-phosphorylated tau antibodies or antibodies that are specific for a specific phosphorylated tau species as described in the Examples or any commercially-available anti-tau antibodies, and examined under a microscope. In some embodiments, in order to distinguish the first neuron from the second neuron, the first neuron and the second neurons can be labeled with a different fluorescent molecule.

### Selection of Subjects in Need of Treatment

The terms “treatment” and “treating” as used herein, with respect to treatment of a disease, means preventing the progression of the disease, or altering the course of the disorder (for example, but are not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of treating a tau-associated neurodegeneration or tauopathy, e.g., AD, therapeutic treatment refers to reduced neurodegenerative morphologies, e.g., reduced inter-neuron propagation after administration of a phosphorylated soluble HMW tau species antagonist agent as described herein. In another embodiment, the therapeutic treatment refers to alleviation of at least one symptom associated with a tau-associated neurodegeneration or tauopathy, e.g., AD. Measurable lessening includes any statistically significant decline in a measurable marker or symptom, such as assessing the cognitive improvement with neuropsychological tests such as verbal and perception after treatment. In one embodiment, at least one symptom of a tau-associated neurodegeneration or tauopathy, e.g., AD, is alleviated by at least about 10%, at least about 15%, at least about 20%, at least about 30%, at least about 40%, or at least about 50%. In another embodiment, at least one symptom is alleviated by more than 50%, e.g., at least about 60%, or at least about 70%. In one embodiment, at least one symptom is alleviated by at least about 80%, at least about 90% or greater, as compared to a control (e.g. in the absence of a soluble HMW tau species antagonist agent as described herein).

In some embodiments, the methods of treatment described herein can further comprise a step of diagnosing a subject with a tau-associated neurodegeneration or tauopathy AD prior to the treatment. Subjects amenable to methods of treatment are subjects that have been diagnosed with a tau-associated neurodegeneration or tauopathy. Exemplary tau associated neurodegeneration or tauopathy includes, but is not limited to Alzheimer's disease, Parkinson's disease, or frontotemporal dementia.

In one embodiment, subjects amenable to methods of treatment are subjects that have been diagnosed with Alzheimer's disease. Methods for diagnosing Alzheimer's disease are known in the art. For example, the stage of Alzheimer's disease can be assessed using the Functional Assessment Staging (FAST) scale, which divides the progression of Alzheimer's disease into 16 successive stages under 7 major headings of functional abilities and losses: Stage 1 is defined as a normal adult with no decline in function or memory. Stage 2 is defined as a normal older adult who has some personal awareness of functional decline, typically complaining of memory deficit and forgetting the names of familiar people and places. Stage 3 (early Alzheimer's disease) manifests symptoms in demanding job situation, and is characterized by disorientation when traveling to an unfamiliar location; reports by colleagues of decreased performance; name- and word-finding deficits; reduced ability to recall information from a passage in a book or to remember a name of a person newly introduced to them; misplacing of valuable objects; decreased concentration. In stage 4 (mild Alzheimer's Disease), the patient may require assistance in complicated tasks such as planning a party or handling finances, exhibits problems remembering life events, and has difficulty concentrating and traveling. In stage 5 (moderate Alzheimer's disease), the patient requires assistance to perform everyday tasks such as choosing proper attire. Disorientation in time, and inability to recall important information of their current lives, occur, but patient can still remember major information about themselves, their family and others. In stage 6 (moderately severe Alzheimer's disease), the patient begins to forget significant amounts of information about themselves and their surroundings and require assistance dressing, bathing, and toileting. Urinary incontinence and disturbed patterns of sleep occur. Personality and emotional changes become quite apparent, and cognitive abulia is observed. In stage 7 (severe Alzheimer's disease), speech ability becomes limited to just a few words and intelligible vocabulary may be limited to a single word. A patient can lose the ability to walk, sit up, or smile, and eventually cannot hold up the head.

Other alternative diagnostic methods for AD include, but not limited to, cellular and molecular testing methods disclosed in US Patent No.: U.S. Pat. Nos. 7,771,937, 7,595,167, 55,580,748, and PCT Application No.: WO2009/009457, the content of which is incorporated by reference in its entirety. Additionally, protein-based biomarkers for AD, some of which can be detected by non-invasive imaging, e.g., PET, are disclosed in U.S. Pat. No. 7,794,948, the content of which is incorporated by reference in its entirety.

Genes involved in AD risk can be used for diagnosis of AD. One example of other AD risk genes is apolipoprotein E-ε4 (APOE-ε4). APOE-ε4 is one of three common forms, or alleles, of the APOE gene; the others are APOE-e2 and APOE-e3. APOE provides the blueprint for one of the proteins that carries cholesterol in the bloodstream. Everyone inherits a copy of some form of APOE from each parent. Those who inherit one copy of APOE-ε4 have an increased risk of developing AD. Those who inherit two copies have an even higher risk, but not a certainty of developing AD. In addition to raising risk, APOE-ε4 may tend to make symptoms appear at a younger age than usual. Other AD risk genes in addition to APOE-e4 are well established in the art. Some of them are disclosed in US Pat. App. No.: US 2010/0249107, US 2008/0318220, US 2003/0170678 and PCT Application No.: WO 2010/048497, the content of which is incorporated by reference in its entirety. Genetic tests are well established in the art and are available, for example for APOE-e4. A subject carrying the APOE-ε4 allele can, therefore, be identified as a subject at risk of developing AD.

In further embodiments, subjects with amyloid beta (Aβ) burden are amenable to the methods of treatment described herein. Such subjects include, but not limited to, the ones with Down syndrome, the unaffected carriers of APP or presenilin gene mutations, and the late onset AD risk factor, apolipoprotein E-ε4.

The term “amyloid beta” or “Aβ” is used herein to refer to a family of peptides that are the principal chemical constituent of the senile plaques and vascular amyloid deposits (amyloid angiopathy) found in the brain, e.g., in patients of Alzheimer's disease (AD), Down's Syndrome, and Hereditary Cerebral Hemorrhage with Amyloidosis of the Dutch-Type (HCHWA-D). Amyloid beta peptides are fragments of beta-amyloid precursor protein (APP) which comprises a variable number of amino acids, typically 38-43 amino acids.

In some embodiments, subjects with a tau-associated neurodegeneration or tauopathy (e.g., AD) who are currently receiving a therapeutic treatment for the tau-associated neurodegeneration or tauopathy can also be subjected to the methods of treatment as described herein.

In some embodiments, a subject who has been diagnosed with an increased risk for developing a tau-associated neurodegeneration or tauopathy (e.g., AD), e.g., using the diagnostic methods described herein or any diagnostic methods (e.g., for AD) known in the art, can be subjected to the methods of treatment as described herein.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

In one embodiment, the subject is a mammal. The mammal can be a human, non-human primate, mouse, rat, dog, cat, horse, or cow, but are not limited to these examples. Mammals other than humans can be advantageously used as subjects that represent animal models of neurodegeneration. In addition, the methods and compositions described herein can be employed in domesticated animals and/or pets. In some embodiments, the subject is a human subject. A human subject can be of any age, gender, race or ethnic group, e.g., Caucasian (white), Asian, African, black, African American, African European, Hispanic, Mideastem, etc.

### Pharmaceutical Compositions and Modes of Administration for Methods of Treatment Described Herein

Another aspect provided herein encompasses pharmaceutical compositions comprising an effective amount of at least one or more (e.g., 1, 2, 3, or more) phosphorylated soluble HMW tau species antagonist agent as described herein. In one embodiment, the composition further comprises at least one or a combination of two or more additional therapeutic agents that inhibit neurodegeneration, e.g., an anti-tau antibody, an antibody against amyloid beta, an AKAP79 peptide, FK506, and/or a NFAT antagonist described in U.S. Patent App. No. 2013/0195866, the content of which is incorporated herein by reference.

In some embodiments, a vector can be used to express and deliver a phosphorylated soluble HMW tau species antagonist agent into neurons. For example, a viral vector as described herein with an expression cassette can encode a MAPT antagonist sequence. The precise determination of an effective dose can be based on individual factors, including their plaque size, age, and amount of time since neurodegeneration. Therefore, dosages can be readily adjusted for each individual patient by those skilled in the art.

Any expression vector known in the art can be used to express the sensor systems described herein. The term “vectors” used interchangeably with “plasmid” refer to a nucleic acid molecule capable of transporting another nucleic acid to which it has been linked. Vectors capable of directing the expression of genes and/or nucleic acid sequence to which they are operatively linked are referred to herein as “expression vectors.” In general, expression vectors of utility in recombinant DNA techniques are often in the form of “plasmids” which refer to circular double stranded DNA loops which, in their vector form are not bound to the chromosome. Other expression vectors can be used in different embodiments described herein, for example, but are not limited to, plasmids, episomes, bacteriophages or viral vectors, and such vectors may integrate into the host's genome or replicate autonomously in the particular cell. Other forms of expression vectors known by those skilled in the art which serve the equivalent functions can also be used. Expression vectors comprise expression vectors for stable or transient expression encoding the DNA.

In some embodiments, the expression vector further comprises a promoter. As used herein, a “promoter” or “promoter region” or “promoter element” used interchangeably herein refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

In some embodiments, the expression vector further comprises a regulatory sequence. The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, an optional operate sequence to control transcription, a sequence encoding suitable mRNA ribosomal binding sites, and sequences to control the termination of transcription and/or translation. Regulatory sequences are selected for the assay to control the expression of split-biomolecular conjugate in a cell-type in which expression is intended.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” or “operatively associated” are used interchangeably herein, and refer to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

In some embodiments, an expression vector is a viral vector. As used herein, the term “viral vector” refers to any form of a nucleic acid derived from a virus and used to transfer genetic material into a cell via transduction. The term encompasses viral vector nucleic acids, such as DNA and RNA, encapsidated forms of these nucleic acids, and viral particles in which the viral vector nucleic acids have been packaged. Examples of a viral vector include, but are not limited to, retroviruses, lentiviruses, adenoviruses, adeno-associated viruses, and combinations thereof.

For administration to a subject in need thereof, e.g., a subject diagnosed with or predisposed to tau-associated neurodegeneration or tauopathy (e.g., AD), a phosphorylated soluble HMW tau species antagonist can be provided in a pharmaceutically acceptable composition. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutically acceptable composition can further comprise one or more pharmaceutically carriers (additives) and/or diluents. As used herein, the term “pharmaceutically-acceptable carrier” means a pharmaceutically-acceptable material, composition or vehicle, such as a liquid, diluent, excipient, manufacturing aid or encapsulating material, for administration of a phosphorylated soluble HMW tau species antagonist described herein. Each carrier must be “acceptable” in the sense of being compatible with the other ingredients of the formulation and not injurious to the patient. Pharmaceutically acceptable carriers include any and all solvents, dispersion media, coatings, antibacterial and antifungal agents, isotonic and absorption delaying agents, and the like which are compatible with the activity of the phosphorylated soluble HMW tau species antagonist and/or the tau antagonist and are physiologically acceptable to the subject. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil: (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation.

For compositions or preparations described herein to be administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutically acceptable carriers can vary in the pharmaceutical compositions described herein, depending on the administration route and formulation. For example, the pharmaceutically acceptable composition described herein can be delivered via injection. These routes for administration (delivery) include, but are not limited to, subcutaneous or parenteral including intravenous, intracortical, intracranial, intracerebroventricular, intramuscular, intraperitoneal, and infusion techniques. In one embodiment, the pharmaceutical acceptable composition is in a form that is suitable for intracortical injection. In another embodiment, the pharmaceutical composition is formulated for intracranial injection. Other forms of administration can be also be employed, e.g., oral, systemic, or parenteral administration.

The pharmaceutical compositions described herein can be specially formulated for administration in solid or liquid form. Additionally, the pharmaceutical compositions can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. Nos. 35 3,270,960.

When administering a pharmaceutical composition described herein parenterally, it will be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), and suitable mixtures thereof. In some embodiments, the pharmaceutical carrier can be a buffered solution (e.g. PBS).

In some embodiments, the pharmaceutical composition can be formulated in an emulsion or a gel. In such embodiments, at least one phosphorylated soluble HMW tau species antagonist described herein can be encapsulated within a biocompatible gel, e.g., hydrogel and a peptide gel. The gel pharmaceutical composition can be implanted to the brain near the degenerating neuronal cells, e.g., the cells in proximity to the amyloid plaque or neurofibrillary tangles, or in the interstitial space of the brain.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to the pharmaceutical compositions described herein, however, any vehicle, diluent, or additive used should have to be biocompatible or inert with the phosphorylated soluble HMW tau species antagonists described herein.

The compositions can be isotonic, i.e., they can have the same osmotic pressure as blood and lacrimal fluid. The desired isotonicity of the pharmaceutical compositions described herein can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

In some embodiments, neurons transduced with a vector encoding a phosphorylated soluble HMW tau species antagonist described herein can be included in the pharmaceutical compositions and stored frozen. In such embodiments, an additive or preservative known for freezing cells can be included in the compositions. A suitable concentration of the preservative can vary from 0.02% to 2% based on the total weight although there may be appreciable variation depending upon the preservative or additive selected. One example of such additive or preservative can be dimethyl sulfoxide (DMSO) or any other cell-freezing agent known to a skilled artisan. In such embodiments, the composition will be thawed before use or administration to a subject, e.g., neuronal stem cell therapy.

Typically, any additives (in addition to the phosphorylated soluble HMW tau species antagonists described herein and/or tau antagonists described herein) can be present in an amount of 0.001 to 50 wt % solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

The pharmaceutical compositions described herein can be prepared by mixing the ingredients following generally-accepted procedures. For example, an effective amount of at least one phosphorylated soluble HMW tau species antagonist described herein can be re-suspended in an appropriate pharmaceutically acceptable carrier and the mixture can be adjusted to the final concentration and viscosity by the addition of water or thickening agent and possibly a buffer to control, pH or an additional solute to control tonicity. An effective amount of at least one phosphorylated soluble HMW tau species antagonist described herein and any other additional agent, e.g., for inhibiting neurodegeneration, can be mixed with the cell mixture. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

Suitable regimes for initial administration and further doses or for sequential administrations can be varied. In one embodiment, a therapeutic regimen includes an initial administration followed by subsequent administrations, if necessary. In some embodiments, multiple administrations of at least one phosphorylated soluble HMW tau species antagonist described herein can be injected to the subject's brain. For example, at least one phosphorylated soluble HMW tau species antagonist described herein can be administered in two or more, three or more, four or more, five or more, or six or more injections. In some embodiments, the same phosphorylated soluble HMW tau species antagonist described herein can be administered in each subsequent administration. In some embodiments, a different phosphorylated soluble HMW tau species antagonist described herein can be administered in each subsequent administration. Injections can be made in cortex, e.g., somatosensory cortex. In other embodiments, injections can be administered in proximity to a plaque, e.g., amyloid-beta plaque or neurofibrillary tangles.

The subsequent injection can be administered immediately after the previous injection, or after at least about 1 minute, after at least about 2 minute, at least about 5 minutes, at least about 15 minutes, at least about 30 minutes, at least about 1 hour, at least about 2 hours, at least about 3 hours, at least about 6 hours, at least about 12 hours, at least about 24 hours, at least about 2 days, at least about 3 days, at least about 4 days, at least about 5 days, at least about 6 days or at least about 7 days. In some embodiments, the subsequent injection can be administered after at least about 1 week, at least about 2 weeks, at least about 1 month, at least about 2 years, at least about 3 years, at least about 6 years, or at least about 10 years.

In various embodiments, a dosage comprising a pharmaceutical composition described herein is considered to be pharmaceutically effective if the dosage reduce degree of neurodegeneration, e.g., indicated by an increased neuron survival or reduced neurotoxicity or improvement in brain or cognitive function, by at least about 5%, at least about 10%, at least about 15%, at least about 20%, at least about 30%, at least about 40%, or at least about 50%. In one embodiment, the brain or cognitive function is improved by more than 50%, e.g., at least about 60%, or at least about 70%. In another embodiment, the brain or cognitive function is improved by at least about 80%, at least about 90% or greater, as compared to a control (e.g. in the absence of the composition described herein).

### Exemplary Articles or Preparations Comprising a Specific Phosphorylated Form of Soluble HMW Tau Species

Another aspect described herein relates to a solid support comprising phosphorylated soluble high molecular weight (HMW) tau species immobilized thereon, wherein substantially all of the phosphorylated soluble HMW tau species are phosphorylated at one, two or all of the following amino acid residues: serine 396, serine 199, and serine 404. The solid support can substantially lack LMW tau, soluble HMW tau polypeptides phosphorylated at sites other than serine 396, serine 199, and serine 404, and/or non-phosphorylated soluble HMW tau polypeptides.

A further aspect described herein relates to a solid support comprising phosphorylated soluble HMW tau species antagonists immobilized hereon, wherein substantially all of the phosphorylated soluble HMW tau species antagonists specifically bind soluble HMW tau species bearing phosphate at serine 396, serine 199, or serine 404. The solid support can substantially lack antagonists to LMW tau, soluble HMW tau polypeptides phosphorylated at sites other than serine 396, serine 199, and serine 404, and/or non-phosphorylated soluble HMW tau polypeptides. In some embodiments, the solid support can comprise an antagonist to a non-tau molecule immobilized thereon. For example, in some embodiments, the solid support can comprise an antagonist to a non-tau molecule that is associated with neurodegenerative diseases or disorders.

As used herein, the term “solid support” refers to a solid structure comprising a surface onto which a target molecule is bound (e.g., covalently or non-covalently), adsorbed, or deposited.

The solid support can be made of any materials, e.g., but not limited to, plastics, glass, cellulose, paper, polymer, metal, hydrogel, and/or a combination of two or more thereof.

The solid support can come in any form to suit the need of an application. Examples of a solid support include, but are not limited to, a dipstick or a test strip, a multi-well plate or a microtiter plate, a microarray, a hollow fiber, a scaffold, a bead, a magnetic bead, a microscope slide, and/or a membrane. For example, in some embodiments, a target phosphorylated soluble HMW tau polypeptide (e.g., soluble HMW tau polypeptide phosphorylated at least at S396, S404, and/or S199) can be immobilized on a solid support, e.g., a microarray or a multi-well plate, and used to identify agents that specifically bind the target phosphorylated soluble HMW tau polypeptide. The identified agents can then be subjected to further functional analysis, e.g., contacting neuronal cells with the identified agent to determine if the agent can reduce or inhibit neuronal uptake of the target phosphorylated soluble HMW tau species as described herein.

To facilitate coupling or immobilization of the target phosphorylated soluble HMW tau polypeptide (e.g., soluble HMW tau polypeptide phosphorylated at least at S396, S404, and/or S199) or an antagonist thereof to a solid support, the molecule to be coupled or immobilized can be modified. Methods for coupling a protein to a solid support are known in the art and can be used herein. For example, in some embodiments, the target phosphorylated soluble HMW tau polypeptide (e.g., soluble HMW tau polypeptide phosphorylated at least at S396, S404, and/or S199) or an antagonist thereof can be biotinylated or conjugated to an art-recognized cross-linking functional group (e.g., but not limited to NHS esters, imidoesters, carbodiimides, maleimides, haloacetyls, pyridyl disulfides, carbonyls, aldehydes, hydrazides, and/or a combination of two or more thereof) for attachment to a surface of a solid support.

A preparation of target phosphorylated soluble HMW tau polypeptide (e.g., soluble HMW tau polypeptide phosphorylated at least at S396, S404, and/or S199) comprising covalent cross-links between one or more tau polypeptide monomers and/or dimers is also described herein.

As used herein, the term “covalent cross-link” refers to a covalent chemical bond linking a molecule to another molecule. Crosslinking reactions and compositions to crosslink two protein or peptide molecules are known in the art, including, e.g., NHS ester reactions, malemide reactions, hydrazide reactions, and/or ECD coupling reactions. A number of chemical cross-linking reagents are known and available commercially, including homobifunctional and heterobifunctional cross linkers that react, e.g., with amines (e.g., N-hydroxsuccinimide (NHS) ester cross linkers, including disuccinimidyl glutarate, disuccinimidyl suberate, bis[sulfosuccinimidyl] suberate, dithiobis[succinimidyl] propionate, among others, imidoester including dimethyl adipimidate-2HCl, dimethyl suberimidate, etc.) or with sulfhydryls (e.g., maleimide-based cross linkers, e.g., bismaleimidoethane, bismaleimidohexane, dithiobismaleimidoethane, etc.). The cross-linkers can be modulated by tailoring reaction conditions as known in the art. In some embodiments, the covalent cross-link can comprise a disulfide bond between one or more tau polypeptide monomers and/or dimers.

In some embodiments, at least a portion of the tau polypeptide monomer and/or dimer population are phosphorylated at the target site (e.g., S396, S404, and/or S199). For example, at least 50% or more (including, e.g., at least 60%, at least 70%, at least 80%, at least 90%, at least 95%, or more) of the tau polypeptide monomeric or dimeric molecules in the population are phosphorylated at the target site(s) (e.g., S396, S404, and/or S199), while the remaining portion, e.g., can be non-phosphorylated at the target site(s). In one embodiment, all of the tau polypeptide monomeric or dimeric molecules in the population are phosphorylated at the target site(s) (e.g., S396, S404, and/or S199).

In some embodiments, the target phosphorylated soluble HMW tau polypeptide of the preparation described herein can further comprise a detectable label. In some embodiments, the detectable label can be fused to the target phosphorylated soluble HMW tau polypeptide as a fusion protein. In these embodiments, the detectable label can be fused to a portion (e.g., at least 30% or more) of the tau polypeptide monomer and/or dimer population. In some embodiments the detectable label can be conjugated to the target phosphorylated soluble HMW tau polypeptide, e.g., via a crosslinking reaction or bioconjugation method known in the art.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

In one aspect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages or numbers may mean±1% of the value being referred to. For example, about 100 means from 99 to 101. As another example, about 50% means from 49.5% to 50.5%.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The term “neurons” as used herein refers to cells that express one or more neuron-specific markers. Examples of such markers can include, but are not limited to, neurofilament, microtubule-associated protein-2, tau protein, neuron-specific Class III β-tubulin, and NeuN. In some embodiments, neurons can include cells that are post-mitotic and express one or more neuron-specific markers.

As used herein, the term “transcriptional repressor” refers to an agent (e.g., protein) that binds to specific sites on DNA and prevents transcription of nearby genes. In some embodiments, the transcriptional repressor is an agent (e.g., protein, peptide, aptamer, and/or a nucleic acid molecule) that binds to specific sites on DNA and prevents transcription of MAPT gene. In some embodiments, the transcriptional repressor can be regulatable.

As used herein, the terms “administering,” or “administration” refer to the placement of an agent (e.g., an antimicrobial agent) into a subject by a method or route which results in at least partial localization of such agents at a desired site, such as a site of infection, such that a desired effect(s) is produced. Examples of administration routes can include, but are not limited to, intracranial administration, intracortical administration, intracerebroventricular administration, and parenteral administration. The phrase “parenteral administration” as used herein refers to modes of administration other than enteral and topical administration, usually by injection. In some embodiments, the administration can comprise catheterization (using a catheter).

As used herein an “expression vector” refers to a DNA molecule, or a clone of such a molecule, which has been modified through human intervention to contain segments of DNA combined and juxtaposed in a manner that would not otherwise exist in nature. DNA constructs can be engineered to include a first DNA segment encoding an acetylation-resistant an anti-phosphorylated soluble HMW tau species antagonist operably linked to additional DNA segments encoding a desired recombinant protein of interest. In addition, an expression vector can comprise additional DNA segments, such as promoters, transcription terminators, enhancers, and other elements. One or more selectable markers can also be included. DNA constructs useful for expressing cloned DNA segments in a variety of prokaryotic and eukaryotic host cells can be prepared from readily available components or purchased from commercial suppliers.

By “cell culture” or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells can be cultured in suspension or while attached to a solid substrate. Fluidized bed bioreactors, hollow fiber bioreactors, roller bottles, shake flasks, or stirred tank bioreactors, with or without microcarriers, can be used.

As used herein, “cell culture medium” is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. “Serum-free” applies to a cell culture medium that does not contain animal sera, such as fetal bovine serum. Various tissue culture media, including defined culture media, are commercially available.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Immunology by Wemer Luttmann, published by Elsevier, 2006. Definitions of common terms in molecular biology can also be found in Benjamin Lewin, Genes X, published by Jones & Bartlett Publishing. 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.). Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss: 5th edition (2005), Animal Cell Culture Methods (Methods in Cell Biology, Vol. 57, Jennie P. Mather and David Barnes editors, Academic Press, 1st edition. 1998) which are all incorporated by reference herein in their entireties.

Embodiments of various aspects described herein can be defined in any of the following numbered paragraphs:
1. A composition comprising phosphorylated soluble high molecular weight (HMW) tau species, wherein the phosphorylated soluble HMW tau species is non-fibrillar and has a molecular weight of at least about 500 kDa, and wherein the composition is substantially free of soluble low molecular weight (LMW) tau species, and wherein the amount of the soluble HMW tau species phosphorylated at amino acid residue serine 422 of tau protein is lower than amount of the soluble HMW tau species phosphorylated at one or more of the following amino acid residues: serine 396, serine 199, and serine 404.
2. The composition of paragraph 1, wherein the phosphorylated soluble HMW tau species has a molecular weight of at least about 669 kDa.
3. The composition of paragraph 1, wherein the phosphorylated soluble HMW tau species has a molecular weight of about 669 kDa to about 1000 kDa.
4. The composition of any of paragraphs 1-3, wherein the phosphorylated soluble HMW tau species is in a form of globular particles.
5. The composition of paragraph 4, wherein the particle size ranges from about 10 nm to about 30 nm.
6. The composition of claim any of paragraph 1-5, wherein the phosphorylated soluble HMW tau species is positive for Alz50 and negative for Thioflavin-S(ThioS).
7. The composition of any of paragraphs 1-6, wherein the phosphorylated soluble HMW tau species is soluble in phosphate-buffered saline and/or cerebrospinal fluid.
8. The composition of any of paragraphs 1-7, wherein the phosphorylated soluble HMW tau species is preferentially taken up by a neuron and axonally transported from the neuron to a synaptically-connected neuron, as compared to neuron uptake and neuron-to-neuron transport of soluble LMW tau species.
9. The composition of paragraph 8, wherein the soluble LMW tau species has a molecular weight of no more than 200 kDa.
10. The composition of any of paragraphs 1-9, further comprising an adjuvant for raising an antibody against the phosphorylated soluble HMW tau species.
11. The composition of paragraph 10, wherein the phosphorylated soluble HMW is covalently conjugated to the adjuvant.
12. The composition of any of paragraphs 1-11, wherein the phosphorylated soluble HMW is covalently conjugated to a carrier peptide.
13. An isolated antibody or antigen-binding portion thereof that specifically binds a soluble HMW tau species phosphorylated at serine 396 and does not bind soluble low molecular weight (LMW) tau species, wherein the phosphorylated soluble HMW tau species is non-fibrillar, has a molecular weight of at least about 500 kDa, and wherein the LMW tau species has a molecular weight of no more than 200 kDa.
14. The isolated antibody or antigen-binding portion thereof of paragraph 13 that specifically binds the phosphorylation site S396.
15. An isolated antibody or antigen-binding portion thereof that specifically binds a soluble HMW tau species phosphorylated at serine 404 and does not bind soluble low molecular weight (LMW) tau species, wherein the phosphorylated soluble HMW tau species is non-fibrillar, has a molecular weight of at least about 500 kDa, and wherein the LMW tau species has a molecular weight of no more than 200 kDa.
16. The isolated antibody or antigen-binding portion thereof of paragraph 15 that specifically binds the phosphorylation site S404.
17. An isolated antibody or antigen-binding portion thereof that specifically binds a soluble HMW tau species phosphorylated at serine 199 and does not bind soluble low molecular weight (LMW) tau species, wherein the phosphorylated soluble HMW tau species is non-fibrillar, has a molecular weight of at least about 500 kDa, and wherein the LMW tau species has a molecular weight of no more than 200 kDa.
18. The isolated antibody or antigen-binding portion thereof of paragraph 17 that specifically binds the phosphorylation site S199.
19. The isolated antibody or antigen-binding portion thereof of any of paragraphs 13-18, which reduces the soluble HMW tau species phosphorylated at S396, S404, or S199 being taken up by a neuron.
20. The isolated antibody or antigen-binding portion thereof of any of paragraphs 13-19, which reduces the soluble HMW tau species phosphorylated at S396, S404, or S199 being axonally transported from a neuron to a synaptically-connected neuron.
21. The isolated antibody or antigen-binding portion thereof of any of paragraphs 13-20, wherein the soluble HMW tau species phosphorylated at $396, S404, or S199 has a molecular weight of at least about 669 kDa.
22. The isolated antibody or antigen-binding portion thereof of paragraphs 21, wherein the soluble HMW tau species phosphorylated at S396, S404, or S199 has a molecular weight of about 669 kDa to about 1000 kDa.
23. The isolated antibody or antigen-binding portion thereof of any of paragraphs 13-22, wherein the soluble HMW tau species phosphorylated at $396, S404, or S199 is in a form of globular particles.
24. The isolated antibody or antigen-binding portion thereof of paragraph 23, wherein the particle size ranges from about 10 nm to about 30 nm.
25. The isolated antibody or antigen-binding portion thereof of any of paragraphs 13-24, wherein the soluble HMW tau species phosphorylated at S396, S404, or S199 is soluble in phosphate-buffered saline and/or cerebrospinal fluid.
26. A method of preventing propagation of pathological tau protein between synaptically-connected neurons comprising selectively reducing the extracellular level of a first phosphorylated soluble HMW tau species in contact with a synaptically-connected neuron, wherein the first phosphorylated soluble HMW tau species is non-fibrillar, has a molecular weight of at least about 500 kDa, and is phosphorylated at least at serine 396, wherein a reduced level of the first phosphorylated soluble HMW tau species results in reduced propagation of pathological tau protein between synaptically-connected neurons.
27. The method of paragraph 26, further comprising selectively reducing the extracellular level of a second phosphorylated soluble HMW tau species in contact with a synaptically-connected neuron, wherein the second phosphorylated soluble HMW tau species is non-fibrillar, has a molecular weight of at least about 500 kDa, and is phosphorylated at least at serine 199 and/or serine 404.
28. The method of paragraph 26 or 27, wherein the extracellular level of a third phosphorylated soluble HMW tau species that is phosphorylated at serine 422 is not substantially reduced during said selective reduction.
29. The method of any of paragraphs 26-28, wherein the extracellular level of soluble LMW tau species is not substantially reduced during said selective reduction.
30. The method of any of paragraphs 26-29, wherein the first and/or second phosphorylated soluble HMW tau species is selectively reduced by contacting the extracellular space or fluid in contact with the synaptically-connected neurons with an antagonist of the first and/or second phosphorylated soluble HMW tau species.
31. The method of paragraph 30, wherein the antagonist of the first and/or second phosphorylated soluble HMW tau species is selected from the group consisting of an antibody, a zinc finger nuclease, a transcriptional repressor, a nucleic acid inhibitor, a small organic molecule, an aptamer, a gene-editing composition, and a combination thereof.
32. A method of reducing tau-associated neurodegeneration in a subject comprising selectively reducing the level of a first phosphorylated soluble HMW tau species in the brain of the subject determined to have, or be at risk for, tau-associated neurodegeneration, wherein the first phosphorylated soluble HMW tau species is non-fibrillar, has a molecular weight of at least about 500 kDa, and is phosphorylated at least at serine 396, wherein a reduced level of the first phosphorylated soluble HMW tau species results in reduced tau-associated neurodegeneration.
33. The method of paragraph 32, further comprising selectively reducing the level of a second phosphorylated soluble HMW tau species in the brain of the subject, wherein the second phosphorylated soluble HMW tau species is non-fibrillar, has a molecular weight of at least about 500 kDa, and is phosphorylated at least at serine 199 and/or serine 404.
34. The method of paragraph 32 or 33, wherein the level of a third phosphorylated soluble HMW tau species that is phosphorylated at S422 is not substantially reduced during the treatment.
35. The method of any of paragraphs 32-34, wherein the level of soluble LMW tau species in the subject is not substantially reduced during the treatment.
36. The method of any of paragraphs 32-35, wherein at least a portion of the first and/or second phosphorylated soluble HMW tau species population is present in brain interstitial fluid of the subject.
37. The method of any of paragraphs 32-36, wherein at least a portion of the first and/or second phosphorylated soluble HMW tau species population is present in cerebrospinal fluid of the subject.
38. The method of any of paragraphs 32-37, wherein the first and/or second phosphorylated soluble HMW tau species is selectively reduced by administering to the brain of the subject an antagonist of the first and/or second soluble HMW tau species.
39. The method of paragraph 38, wherein the antagonist of the first and/or second soluble HMW tau species is selected from the group consisting of an antibody, a zinc finger nuclease, a transcriptional repressor, a nucleic acid inhibitor, a small organic molecule, an aptamer, a gene-editing composition, and a combination thereof.
40. The method of any of paragraphs 32-39, further comprising selecting a subject determined to have soluble HMW tau species present in the brain at a level above a reference level.
41. The method of any of paragraphs 32-40, wherein the tau-associated neurodegeneration is Alzheimer's disease, Parkinson's disease, or frontotemporal dementia.
42. A method of diagnosing tau-associated neurodegeneration comprising
a. fractionating a sample of brain interstitial fluid or cerebrospinal fluid from a subject;
b. detecting a first phosphorylated soluble HMW tau species in the sample such that the presence and amount of the first phosphorylated soluble HMW tau species is determined, wherein the first phosphorylated soluble HMW tau species is non-fibrillar, has a molecular weight of at least about 500 kDa, and is phosphorylated at least at serine 396; and
c. identifying the subject to have, or be at risk for tau-associated neurodegeneration when the level of the first phosphorylated soluble HMW tau species in the sample is the same as or above a reference level; or
   identifying the subject to be less likely to have tau-associated neurodegeneration when the level of the first phosphorylated soluble HMW tau species is below a reference level.
43. The method of paragraph 42, further comprising detecting a second phosphorylated soluble HMW tau species in the sample such that the presence and amount of the second phosphorylated soluble HMW tau species is determined, wherein the second phosphorylated soluble HMW tau species is non-fibrillar, has a molecular weight of at least about 500 kDa, and is phosphorylated at least at serine 199 and/or serine 404.
44. The method of paragraph 42 or 43, further comprising administering to the brain of the subject identified to have, or be at risk for tau-associated neurodegeneration an antagonist of the first and/or second soluble HMW tau species.
45. The method of paragraph 44, wherein the antagonist of the first and/or second soluble HMW tau species is selected from the group consisting of an antibody, a zinc finger nuclease, a transcriptional repressor, a nucleic acid inhibitor, a small organic molecule, an aptamer, a gene-editing composition, and a combination thereof.
46. The method of any of paragraphs 42-45, wherein the sample is substantially free of soluble LMW tau species, wherein the soluble LMW tau species has a molecular weight of no more than 200 kDa.
47. The method of any of paragraphs 42-46, wherein the sample comprises soluble LMW tau species, wherein the soluble LMW tau species has a molecular weight of no more than 200 kDa.
48. The method of paragraph 47, further comprising detecting the amount of the soluble LMW tau species phosphorylated at amino acid residue serine 396 of tau protein in the sample.
49. The method of any of paragraphs 42-48, wherein the subject is identified to have, or be at risk for tau-associated neurodegeneration if a ratio of the first phosphorylated soluble HMW tau species to soluble LMW tau species phosphorylated at least at S396 is the same as or above a reference level ratio; or the subject is identified to be less likely to have tau-associated neurodegeneration if the ratio of the first phosphorylated soluble HMW tau species to soluble LMW tau species phosphorylated at least at S396 is below the reference level ratio.
50. The method of any of paragraphs 42-49, wherein the fractionating comprises size exclusion.
51. The method of any of paragraphs 42-50, wherein the tau-associated neurodegeneration is Alzheimer's disease, Parkinson's disease, or frontotemporal dementia.
52. A method of identifying an agent that is effective to reduce cross-synaptic spread of misfolded tau proteins comprising
a. contacting a first neuron in a first chamber of a neuron culture device with a composition comprising a first phosphorylated soluble HMW tau species, the first phosphorylated soluble HMW tau species being non-fibrillar, having a molecular weight of at least about 500 kDa, and being phosphorylated at least at amino acid residue serine 396 (S396) of tau protein, wherein the first neuron is axonally connected with a second neuron in a second chamber of the neuron culture device, and wherein the second neuron is not contacted with the first phosphorylated soluble HMW tau species;
b. contacting the first neuron from (a) in the first chamber with a candidate agent; and
c. detecting transport of the first phosphorylated soluble HMW tau species from the first neuron to the second neuron, thereby identifying an effective agent for reducing cross-synaptic spread of misfolded tau proteins based on detection of the presence of the first phosphorylated soluble HMW tau species in an axon and/or soma of the second neuron.
53. The method of paragraph 52, wherein the neuron culture device is a microfluidic device.
54. The method of paragraph 53, wherein the microfluidic device comprises a first chamber for placing a first neuron and a second chamber for placing a second neuron, wherein the first chamber and the second chamber are interconnected by at least one microchannel exclusively sized to permit axon growth.
55. A solid support comprising phosphorylated soluble HMW tau polypeptide immobilized thereupon, said solid support substantially lacking LMW tau, and wherein substantially all of the phosphorylated soluble HMW tau species are phosphorylated at one, two, or all of the amino acid residues: serine 396, serine 199, and serine 404.
56. A solid support comprising phosphorylated soluble HMW tau species antagonists immobilized hereon, said solid support substantially lacking LMW tau, and wherein substantially all of the phosphorylated soluble HMW tau species antagonists specifically bind soluble HMW tau species bearing phosphate at serine 396, serine 199, or serine 404.
57. A preparation of S396-phosphorylated soluble HMW tau polypeptide comprising covalent cross-links between one or more tau polypeptide monomers.

### EXAMPLES

The disclosure is further illustrated by the following examples which should not be construed as limiting. The examples are illustrative only, and are not intended to limit, in any manner, any of the aspects described herein. Further, various changes and modifications to the disclosed embodiments, which will be apparent to those of skill in the art, can be made without departing from the spirit and scope of the present invention. The following examples do not in any way limit the invention.

### Example 1. Neuronal Uptake and Propagation of a Rare Phosphorylated High-Molecular-Weight Tau Species Derived from Tau-Transgenic Mouse and Human Alzheimer's Disease Brain

Accumulation and aggregation of microtubule-associated protein tau (1), as intracellular inclusions known as neurofibrillary tangles (NFTs) (insoluble, fibrillar aggregates of tau proteins), is a pathological hallmark of neurodegenerative diseases including Alzheimer's disease (AD) (2, 3). Cognitive deficits in AD are most closely linked with progression of NFTs in a hierarchical pattern, starting in the entorhinal cortex (EC) and marching throughout the brain during disease progression (4, 5). Although the precise mechanisms for this characteristic tau pathology spread remain unknown, previous reports suggest a trans-synaptic transfer of tau proteins between neurons (6-8). By developing the rTgTauEC mouse model of early AD that overexpresses human mutant P301L tau selectively in the EC, it has been previously demonstrated that aggregated tau accumulates in synaptically connected downstream areas such as dentate gyrus (DG), indicating that NFT propagation occurs by cross-synaptic spread of pathologically misfolded tau proteins (9-12). Other previous reports described that pathological forms of tau replicate conformation and spread among cells, thus suggesting that prion-like mechanisms underlie the propagation of tau (13-16). It has been reported that tau can be secreted from intact neurons into the extracellular space in an activity-dependent manner (17-18). However, none of the previous reports identifies a specific extracellular misfolded tau species that can be taken up by neurons, thus contributing to tau pathology spreading. Better understanding of the molecular basis of tau propagation is key to preventing progression from early mild memory impairment to full cognitive deterioration and dementia.

Previous reports showed that cellular tau uptake and trans-cellular propagation occur in various systems in vitro and in vivo; however, whether the tau species involved in neuron-to-neuron transfer is fibrillar or not, and what its specific properties are, is not discussed in any of the previous reports.

In this Example, to identify specific tau species responsible for propagation, the inventors compared the uptake and propagation properties of different tau species derived from brain extracts of tau-transgenic mouse lines rTg4510 (expressing aggregating P301L tau (0N4R)) (19) and rTg21221 (expressing non-aggregating wild-type (WT) human tau (0N4R)) (20), human sporadic AD brain extracts, and recombinant WT full-length human tau (2N4R, 441 aa). The propagating tau species were isolated via differential centrifugation and size exclusion chromatography (SEC), biochemically characterized, and neuronal uptake of each tau species was assessed in mouse primary cortical neurons and in vivo. For all different sources of tau, efficient uptake was only observed for high-molecular-weight (HMW) tau species.

The transfer of tau between neurons was examined using a newly developed microfluidic neuron culture platform, which comprises three distinct chambers that are connected through arrays of thin channels such that the axon growth and formation of synaptic connections are precisely controlled between neurons in different chambers. Furthermore, a unique large-pore (1,000 kDa cut-off) probe in vivo microdialysis (21, 22) was used to investigate the presence of HMW tau species in brain interstitial fluid (ISF) of awake, freely-moving mice. The findings presented herein indicate that PBS-soluble phosphorylated soluble HMW tau species, present in the brain extracellular space, are involved in neuronal uptake and propagation.

### Results

### Identification and Characterization of Tau Species Taken Up by Neurons

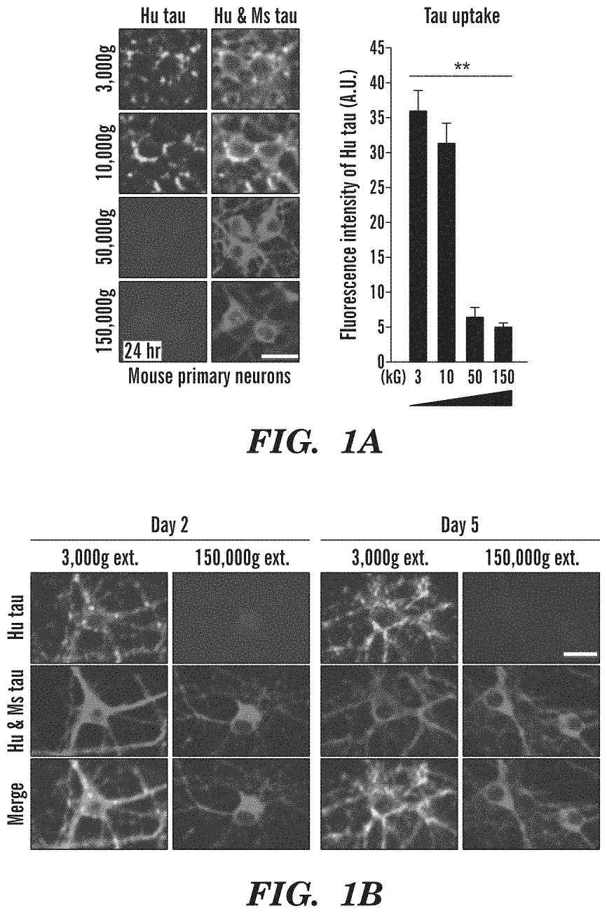

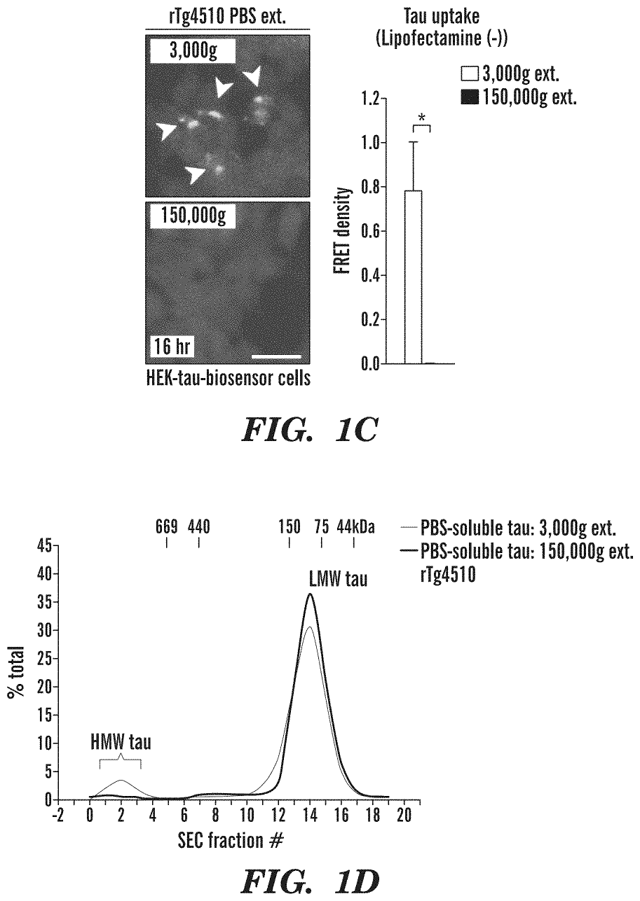

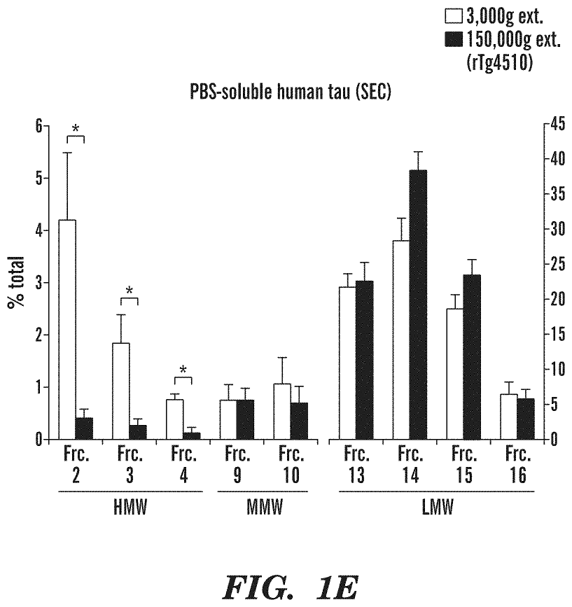

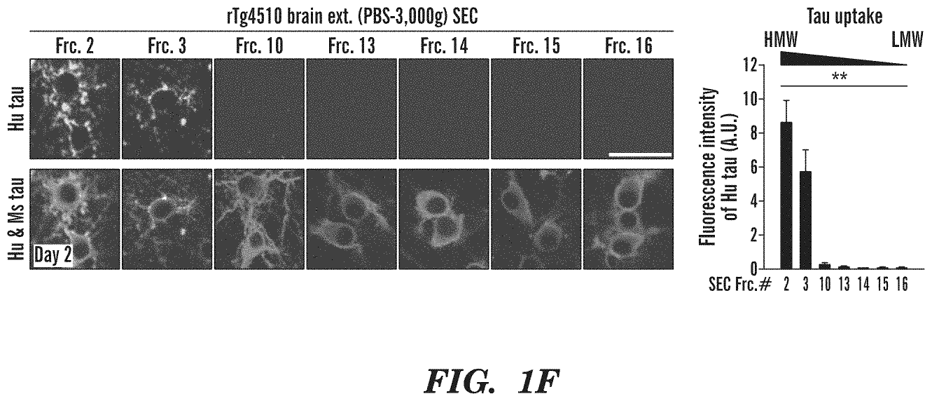

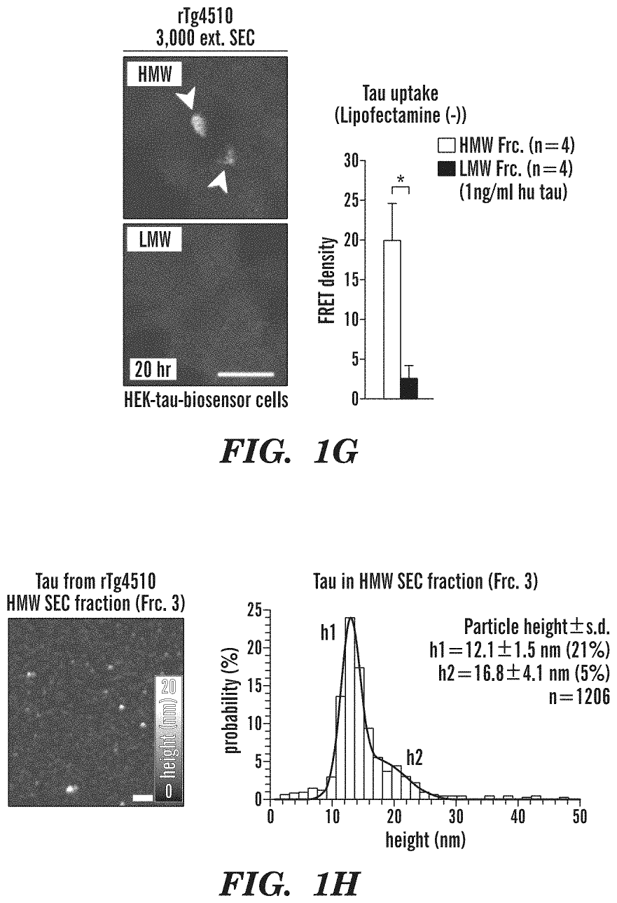

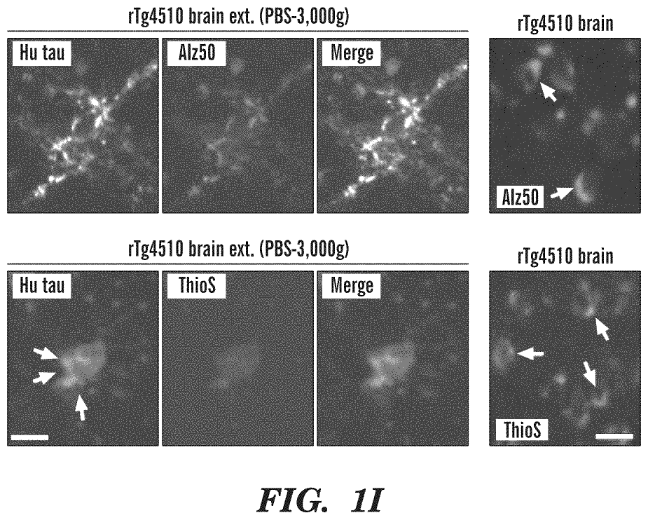

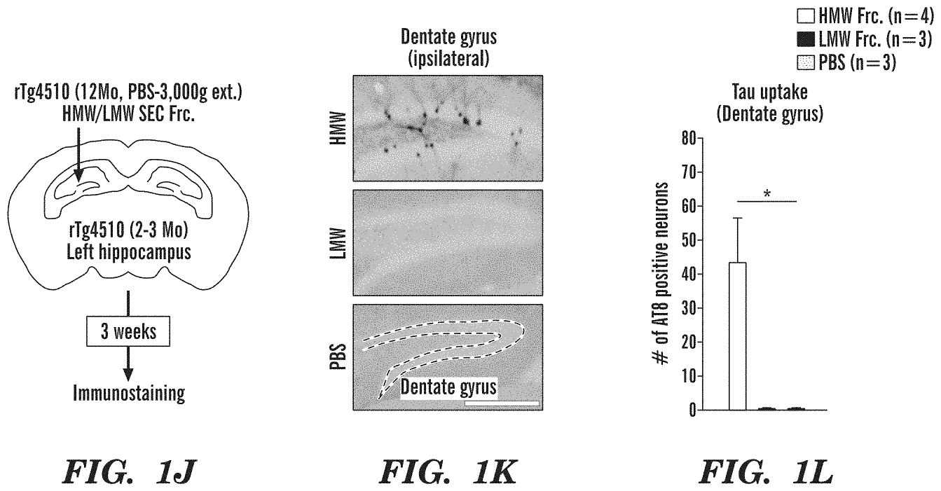

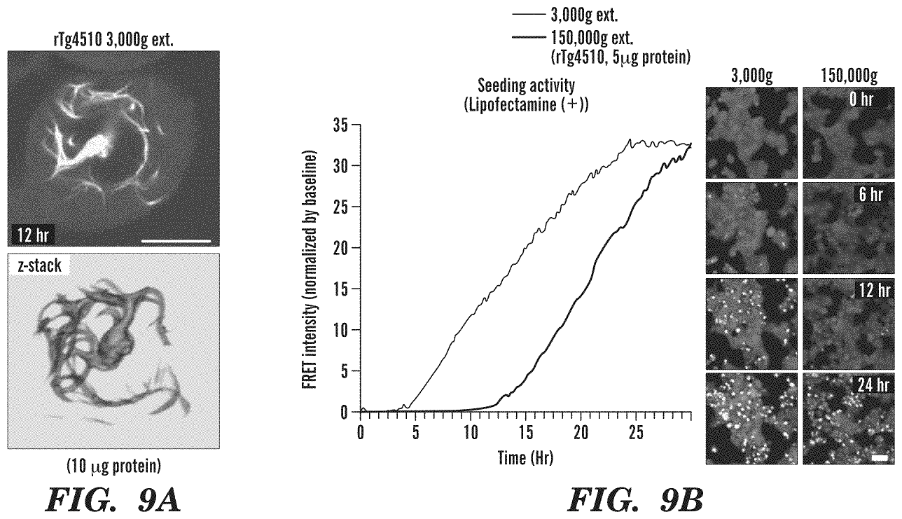

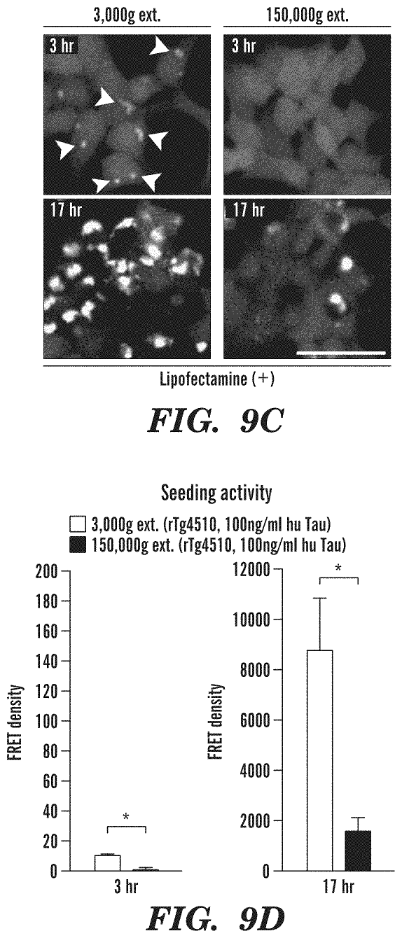

Identification and characterization of tau species taken up by neurons is critical for understanding the mechanism of neuron-to-neuron tau propagation. The molecular weight (MW) of tau species involved in neuronal uptake was first examined. PBS-soluble brain extracts were prepared from rTg4510 mice, which overexpress human mutant P301L tau, by centrifugation either at 3,000 g, 10,000 g, 50,000 g, or 150,000 g, and the supernatant was applied to mouse primary cortical neurons. The uptake of tau was assessed by immunofluorescence labeling of intracellular human tau. After 24 hours, human tau uptake was detected in neurons treated with 3,000 g and 10,000 g brain extracts, which contained HMW proteins. No uptake occurred from 50,000 g and 150,000 g extracts (FIG. 1A) from which HMW tau was depleted by sedimentation. In neurons treated for longer incubation periods, robust tau uptake was observed from 3,000 g extract after 2 and 5 days, however, little uptake occurred from 150,000 g extract even after 5 days of incubation (FIG. 1B). Cellular tau uptake from the 3,000 g extract was also assessed using FRET-based HEK-tau-biosensor cells (FIG. 1C). 3,000 g brain extracts showed significantly higher seeding activity than 150,000 g extracts (FIG. 9A). The seeding activity of 150,000 g extracts eventually (within 24 hours) caught up with that of 3,000 g extracts (FIG. 9B), indicating that uptake is at least one of the key elements in the kinetics of tau uptake and aggregation processes.

The MW size distribution of tau species contained in each brain extract was then assessed by size exclusion chromatography (SEC). The 3,000 g brain extract had a small peak of HMW tau species (SEC Frc.2-4) in addition to a dominant low-molecular-weight (LMW) tau peak (SEC Frc.13-16, 50-150 kDa), while the 150,000 g brain extract from the same rTg4510 mouse brain had only a LMW tau peak and a trace amount of HMW tau species (FIGS. 1D and 1E). The involvement of HMW tau species in neuronal uptake was validated by incubating each SEC-fraction with primary neurons (FIG. 1F). The most extensive tau uptake was observed for HMW fractions (Frc.2, 3). Essentially no detectable uptake was observed from the dramatically more abundant LMW fractions, indicating that HMW tau species were the forms being taken up. Tau uptake assay in HEK-tau-biosensor cells also demonstrated that HMW tau can be taken up by cells more efficiently than LMW tau species (FIG. 1G).

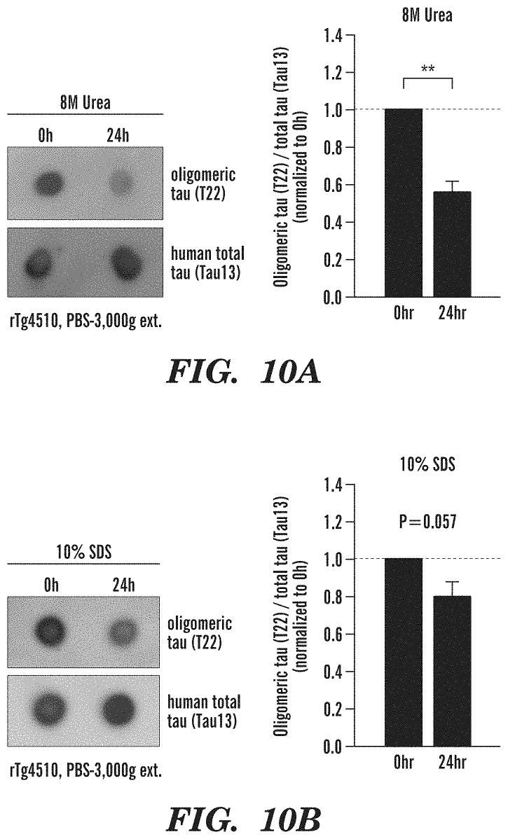

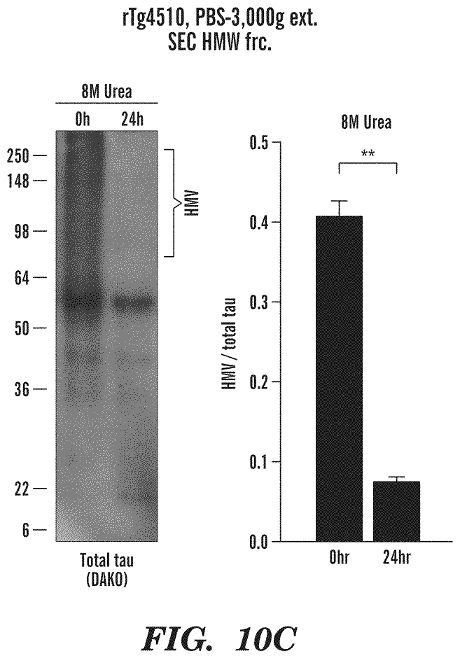

Exposure to 8 M urea reduced the immunoreactivity of the tau oligomer-specific antibody (T22) in the 3,000 g brain extract (dot blot, FIGS. 10A-10B), and the HMW smear of tau in the HMW SEC fraction largely disappeared after urea incubation (SDS-PAGE, FIG. 10C), indicating the existence of a multimeric tau assembly in this fraction. To further characterize HMW tau, tau was immunoprecipitated from HMW SEC fraction and characterized by atomic force microscopy (AFM) (FIG. 1H). HMW SEC fraction (Frc. 3) contained small globular but no fibrillar tau aggregates (FIG. 1H). The size (particle height) distribution revealed particles of 12.1±1.5 (h1) and 16.8±4.1 (h2) nm (height ±s.d., n=1206) (FIG. 1H). In some embodiments, these tau-containing particles can be made exclusively of tau. In some embodiments, these tau-containing particles can contain other constituents such as proteins and lipids.

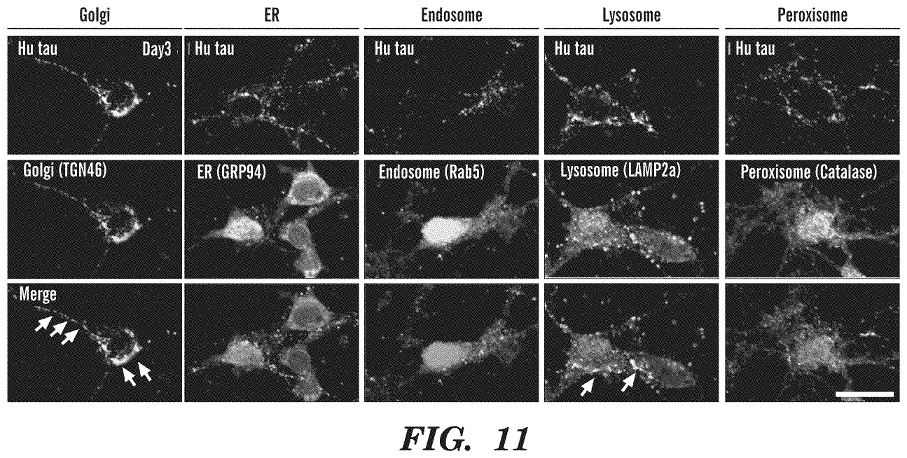

Human tau species observed within primary neurons 2-5 days after exposure to rTg4510 brain extract were Alz50 positive (FIG. 1I, top) but negative for Thioflavin-S(ThioS) staining (FIG. 11, bottom), indicating an early stage of pathological conformation of tau that is taken up. Furthermore, tau species taken up by primary neurons co-localized with subcellular organelle markers such as the Golgi apparatus and the lysosomes at day 3 (FIG. 11).

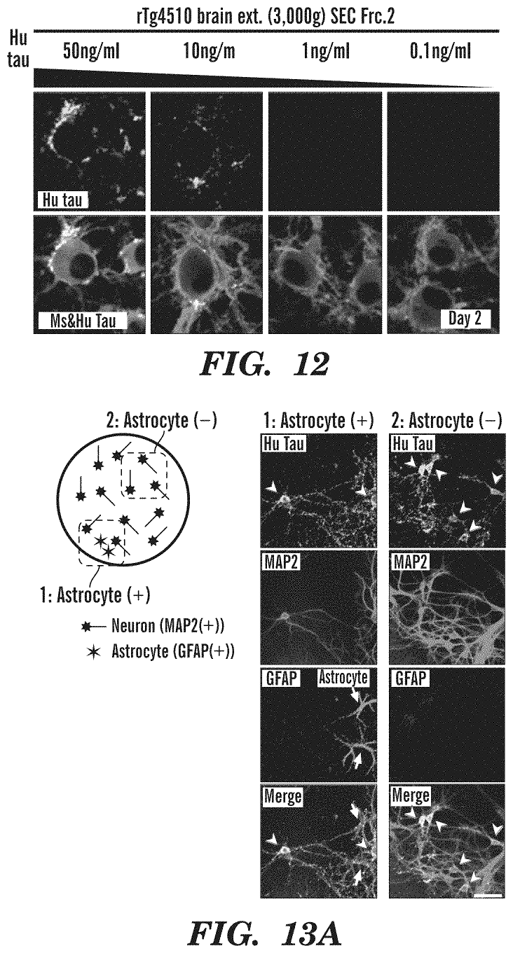

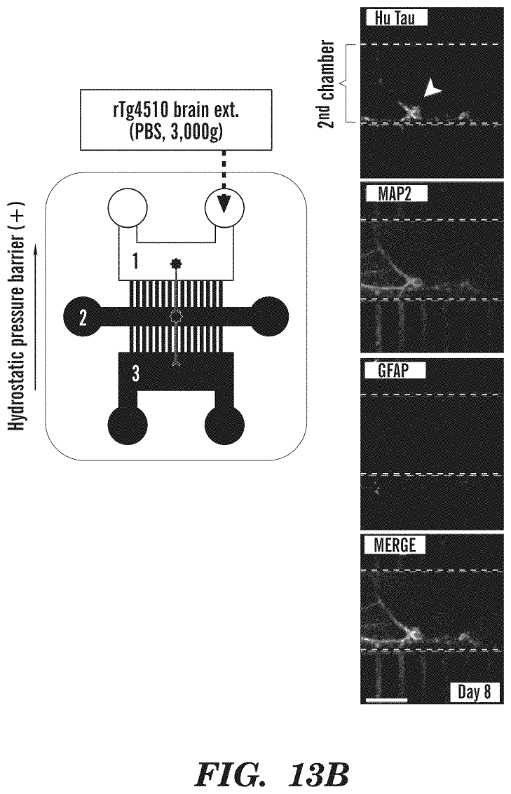

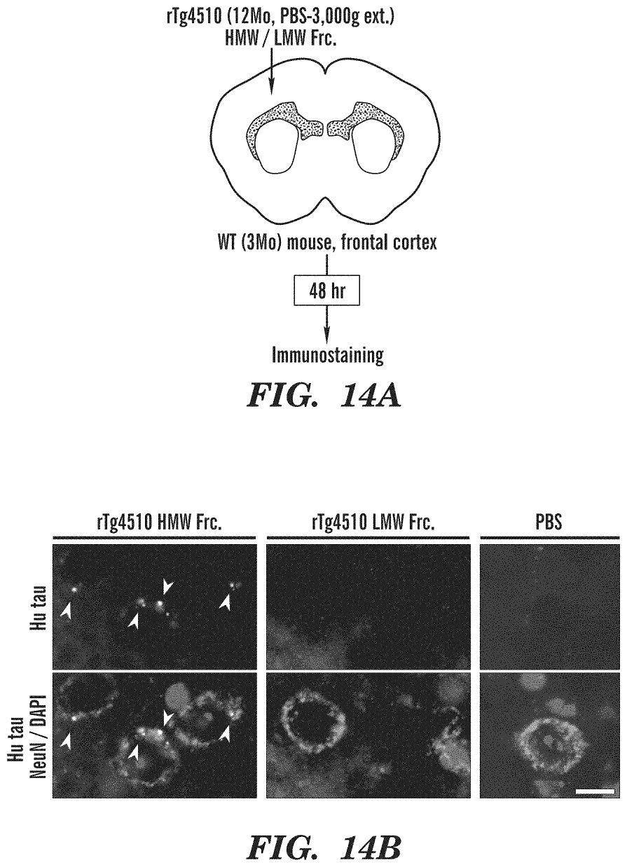

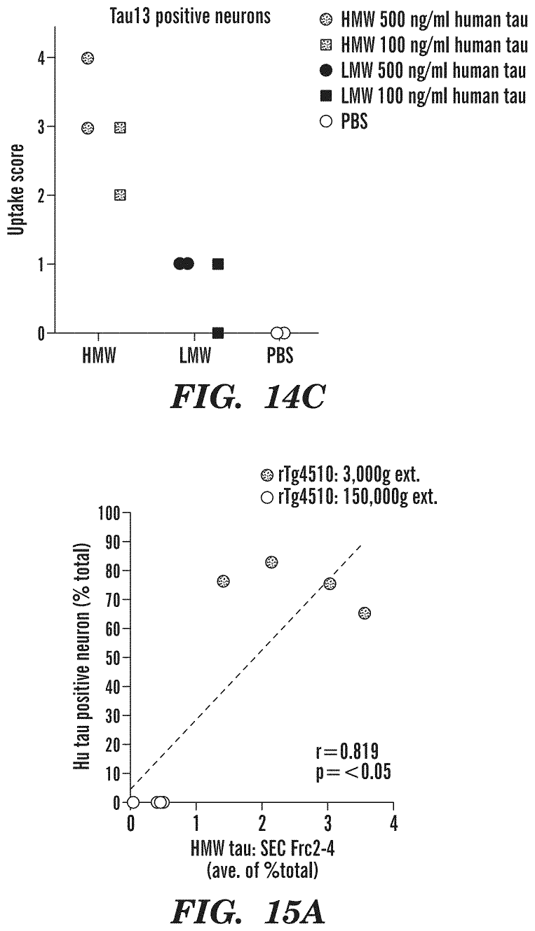

Uptake of human tau occurred in a concentration-dependent manner (FIG. 12). Applying different human tau concentrations, it was found that the minimum concentration of HMW tau from rTg4510 brain extract required for detection of neuronal uptake was 10 ng/ml (FIG. 12), which is lower than the ISF levels of tau in tau-transgenic mouse (approximately 250 ng/ml (25)). Tau uptake in primary neurons occurred in both the presence and absence of GFAP-labeled astrocytes (FIG. 13A). Importantly, neuronal uptake of HMW tau occurred in vivo as well; human tau uptake in neurons was detected in young rTg4510 (pre-tangle stage) (FIGS. 1J-L) and WT (FIGS. 14A-14C) mice injected with the HMW SEC fraction of Tg4510 (12 mo) brain extract, but not in those injected with the LMW fractions.

### Phosphorylated Soluble HMW Tau is Taken Up by Neurons

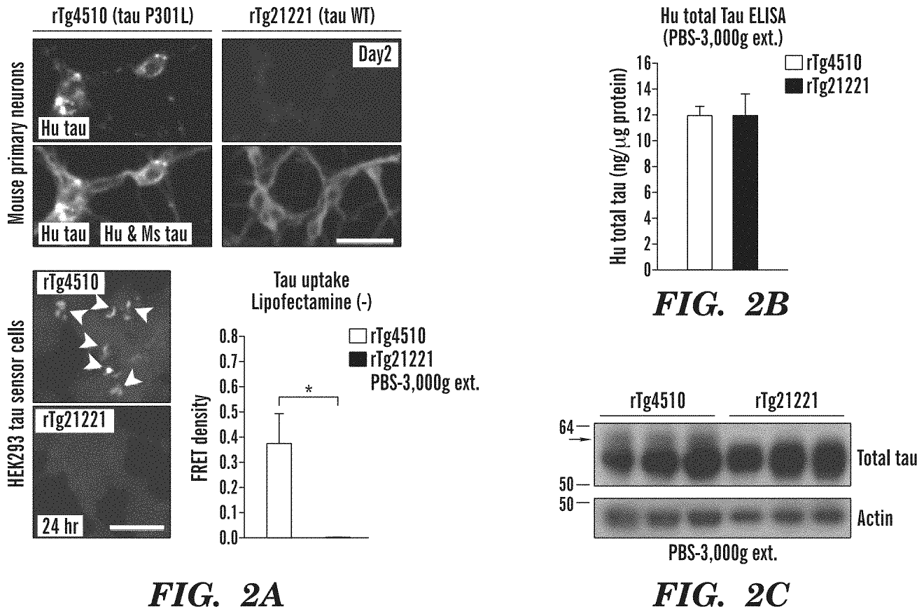

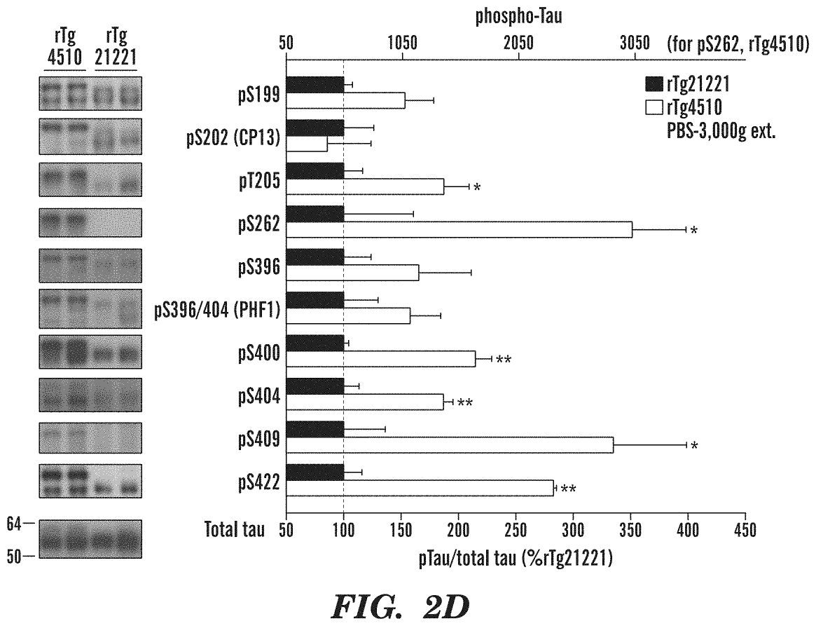

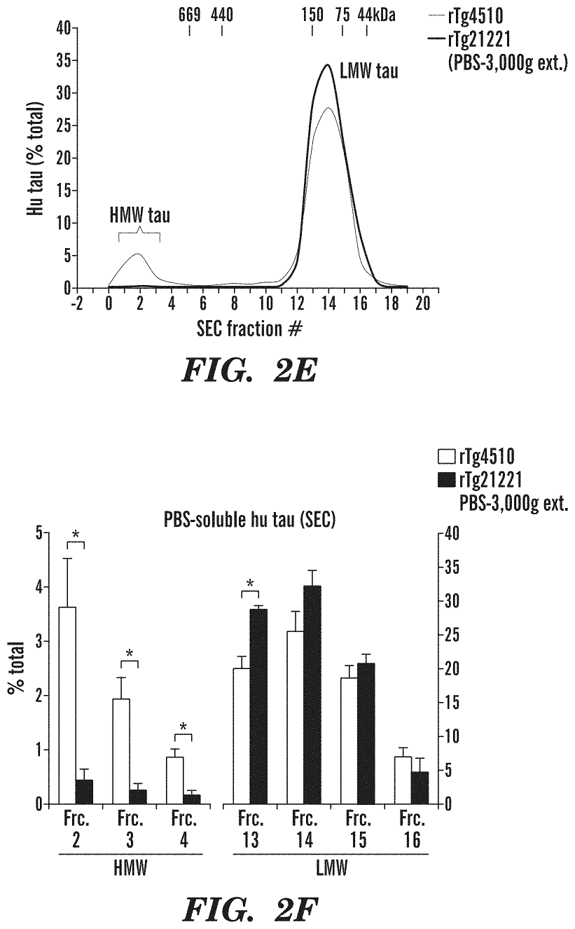

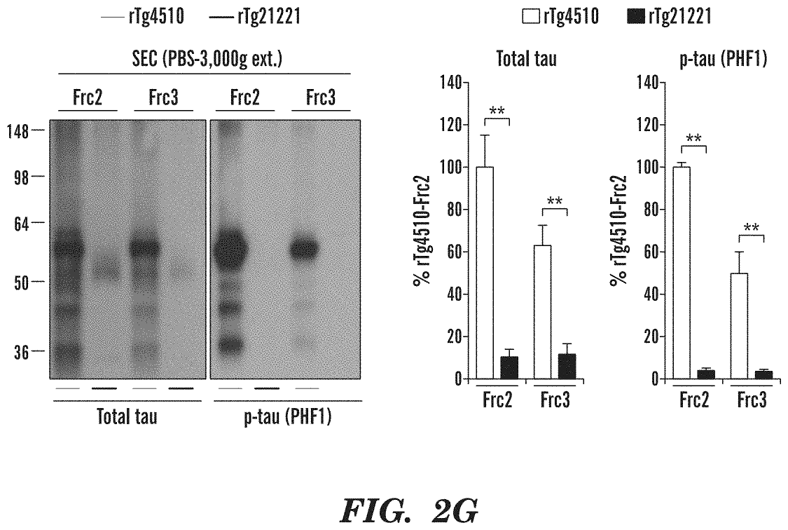

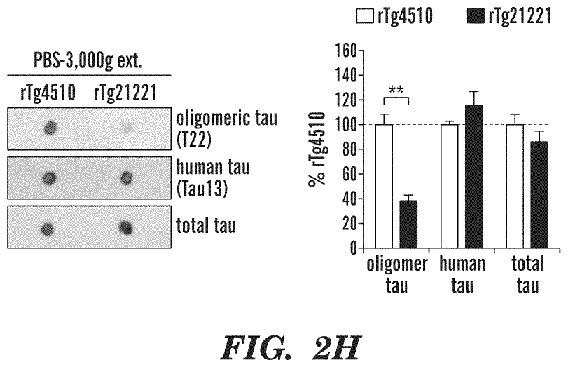

To evaluate the relevance of tau aggregation and phosphorylation for neuronal uptake, 3,000 g brain extracts were prepared from rTg21221 mice and the uptake and biochemical properties were compared to those of rTg4510 homogenate. rTg21221 mice overexpress WT human tau under the same promoter as rTg4510 mice and show phosphorylation but no accumulation of misfolded and aggregated tau species in the brain (20). Unlike the case for the rTg4510 mice, no uptake was observed in primary neurons from rTg21221 brain extracts at day 2 (FIG. 2A, top). Tau uptake assay in HEK-tau-biosensor cells also showed lack of tau uptake from Tg21221 brain extracts (FIG. 2A, bottom). Human tau and total tau levels in PBS-soluble brain extracts were comparable to those seen in rTg4510 brains (FIGS. 2B-2C), although an upward shift of the tau band in western blot (FIG. 2C, arrow) indicated a higher degree of tau phosphorylation in rTg4510 brain.

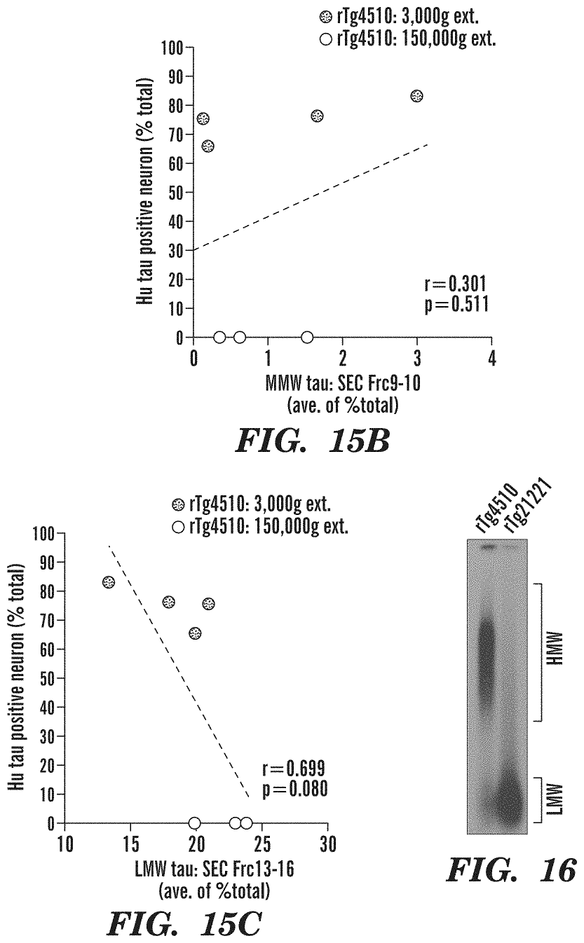

The degree of tau phosphorylation was next compared in rTg4510 and rTg21221 extracts in more detail using 10 different phospho-tau epitope specific antibodies (FIG. 2D). The PBS-extractable tau species from rTg4510 brain had higher levels of phosphorylation compared to the tau species obtained from rTg21221, especially those associated with some specific phosphorylation sites such as pT205, pS262, pS400, pS404, pS409, and pS422. SEC analysis of the MW distribution of tau demonstrated that rTg21221 brain extracts (PBS-soluble, 3,000 g) contained primarily LMW species and very low levels of HMW tau species, whereas rTg4510 brain extract showed both HMW and LMW peaks (FIGS. 2E-2F). The degree of tau uptake into primary neurons correlated significantly with HMW (SEC Frc.2-4, >669 kDa) tau levels, but not with middle molecular weight (MMW) (SEC Frc.9-10, 200-300 kDa) or LMW (SEC Frc. 13-16, 50-150 kDa) tau levels (FIGS. 15A-15C). The differences in HMW tau levels between rTg4510 and rTg21221 brain extracts were also assessed by western blot (SDS-PAGE) analysis of SEC fractions (FIG. 2G) and semi-denaturing detergent agarose gel electrophoresis (SDD-AGE) blot (FIG. 16); HMW tau from rTg4510 brain was highly phosphorylated (FIG. 2G, PHF1). Dot blot analysis demonstrated the presence of oligomeric form of tau in PBS-soluble extracts from rTg4510 brain, although those from rTg21221 brain only had a small amount of tau oligomer assessed with antibody T22 (FIG. 2H). These findings indicate that phosphorylated soluble HMW tau species are taken up by neurons.

Intercellular Propagation of Tau in a Microfluidic Neuron Culture Platform that Models Neuron-to-Neuron Interactions

The transfer of tau between neurons was then assessed using a microfluidic neuron culture platform. The design of this platform includes three distinct chambers forming layering synaptic connections between neurons, which are plated on different chambers and arrays of microgrooves, allowing an exclusive axon growth by sizes (FIG. 3A). Two sets of neurons are plated into the 1st and 2nd chambers (FIG. 3A). The axons from the 1st chamber neurons extend into the 2nd chamber within four days (FIG. 3B, left), and axons from the 2nd chamber neurons extend to the 3rd chamber (FIG. 3B, middle). The resulting two sets of neurons therefore have “in line” synaptic connections at the 2nd chamber (FIG. 3B, right). The 1st chamber neurons were labeled with green fluorescent protein (GFP) and the 2nd chamber neurons with red fluorescent protein (RFP) using a hydrostatic pressure barrier to fluidically isolate neurons in different chambers (26). This showed that the two neuronal populations were connected to each other in the 2nd chamber (FIG. 3C).

Neuron-to-Neuron Transfer of rTg4510 Mouse Brain-Derived Tau Species in the Microfluidic Chamber

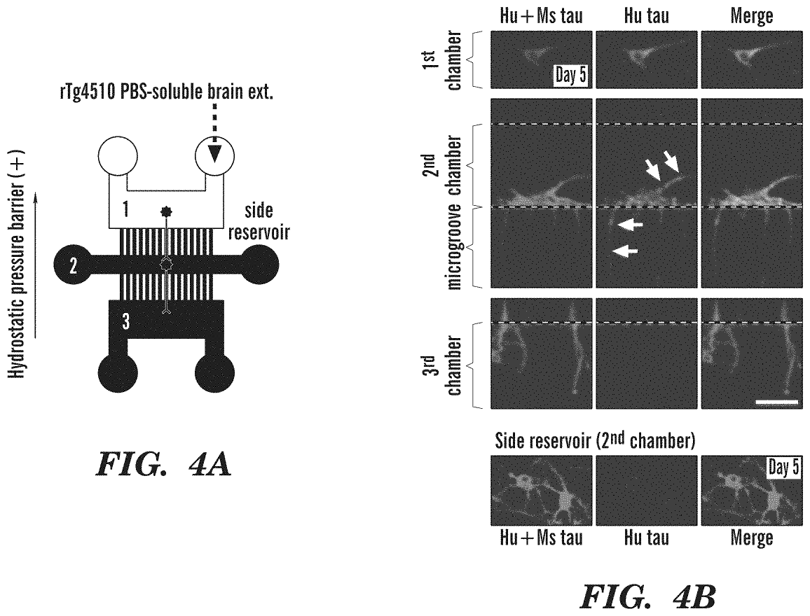

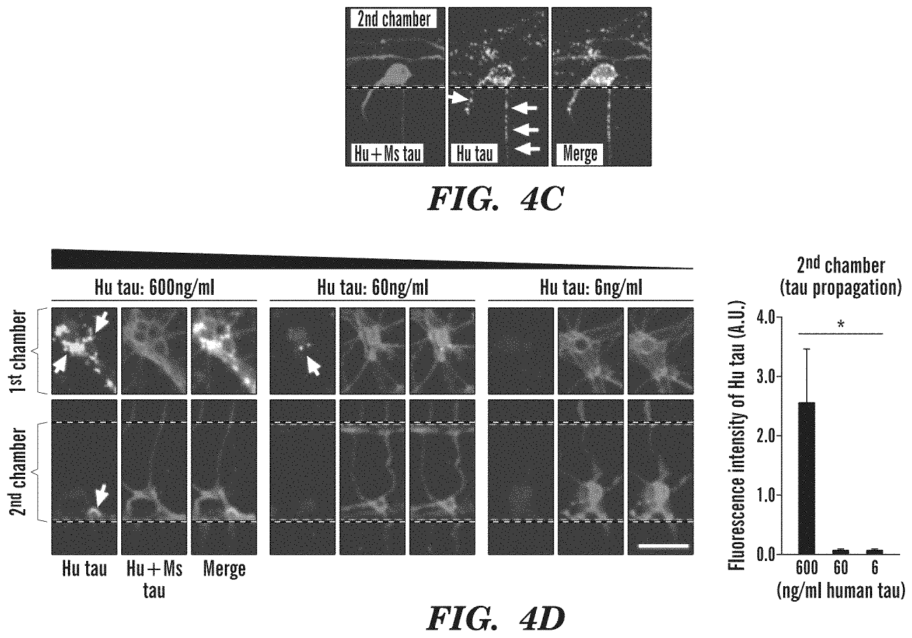

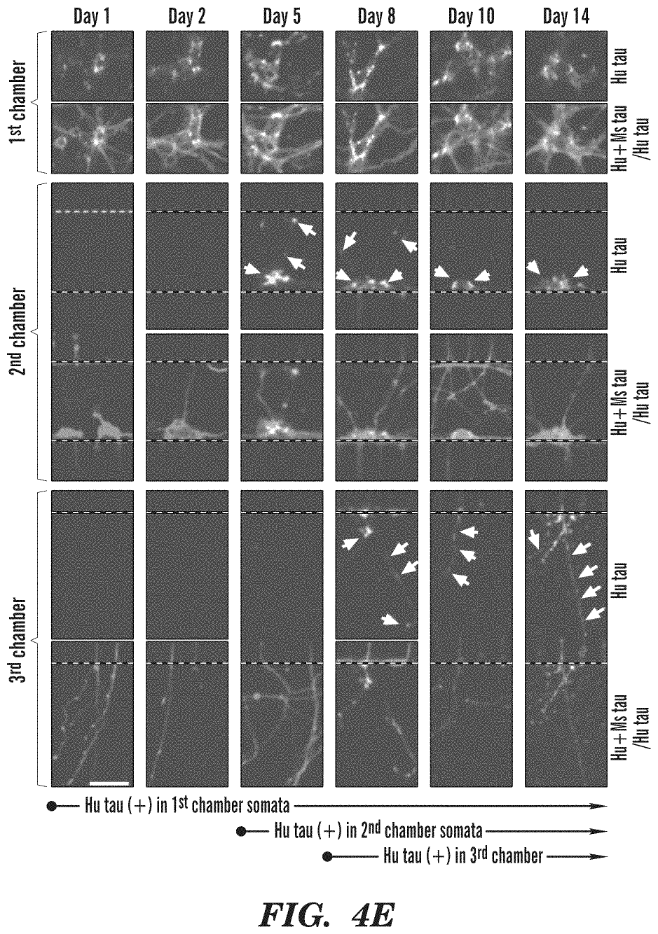

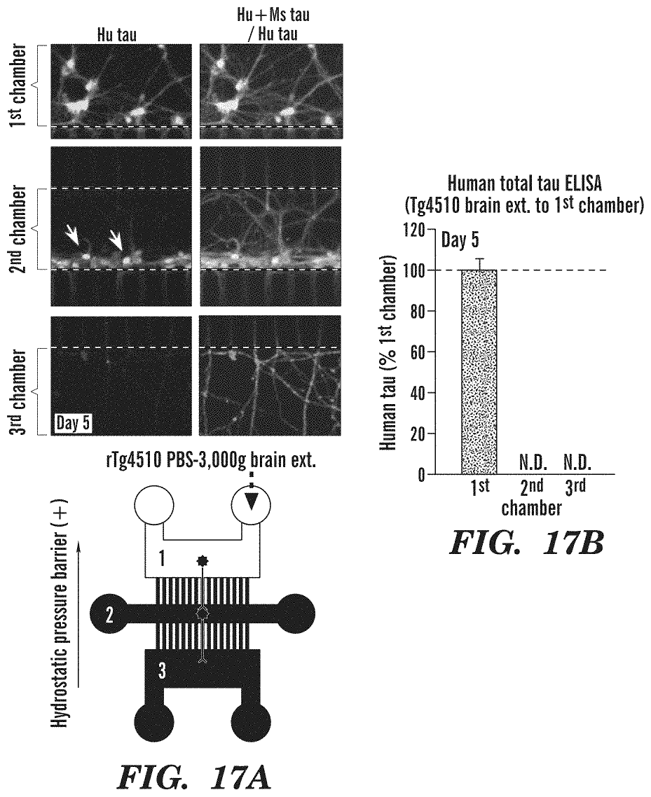

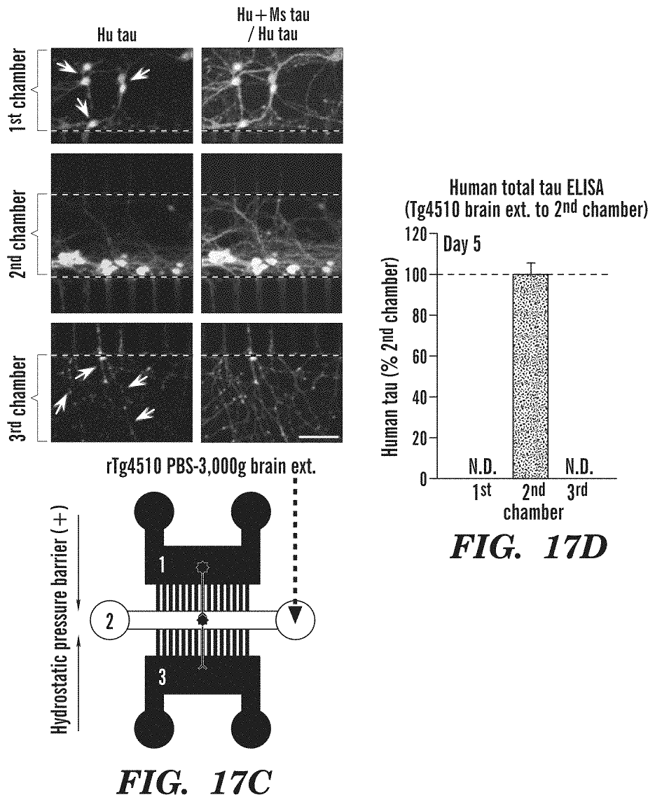

The propagation properties of rTg4510 brain-derived tau species was assessed using the 3-chamber microfluidic neuron chamber. PBS-soluble brain extracts from an rTg4510 mouse (3,000 g, 500 ng/ml human tau) were added to the 1st chamber (FIG. 4A). To assure that only the 1st chamber neurons were exposed to brain extract, the diffusion-driven transport of various tau species was blocked by convective flow in the opposite direction (hydrostatic pressure barrier). After 5 days of incubation, a human tau-specific immunostain revealed positive immunoreactivity in neurons and in axons of the neurons from the 1st chamber, as well as the soma of the neurons in the 2nd chamber (FIG. 4B), indicating that human tau species taken up by the 1st chamber neurons had been transported through their axons and transferred into the 2nd chamber neurons. Neurons that establish little or no axon-dendrite connection with the 1st chamber neurons (in the side reservoir of the 2nd chamber) remained negative for human tau staining (FIG. 4B, bottom), indicating that axonal input from the 1st chamber is necessary for tau transfer. Human tau was also detected in axons and dendrites extending from the human tau positive 2nd chamber neurons (FIG. 4C), indicating further transport of tau species into the 3rd chamber. Retrograde propagation from the 2nd to the 1st chamber also occurred during the same time course of the experiment (FIGS. 17A-17D), which is consistent with previous research in vitro (27) and in vivo (13,15).

The propagation of tau in the microfluidic device was concentration dependent and 500-600 ng/ml of human tau (in the 1st chamber) was needed to detect propagation to the 2nd chamber neurons over the course of a few days (FIGS. 4D-4E). These concentrations are similar to the ISF tau levels in tau-transgenic mice (25). Time-course analysis showed early uptake of human tau in the 1st chamber neurons (as early as day 1), propagation to the 2nd chamber neurons after 5 days, and progression to the 2nd chamber neuron axon terminals in the 3rd chamber after 8 days (FIG. 4E). There was no detectable astrocyte contamination in the 2nd chamber of the microfluidic device (FIG. 13B), indicating that neuron-to-neuron tau transfer can occur in the absence of astrocytes.

### Persistent Axonal Transport and Lifetime of Internalized HMW Tau

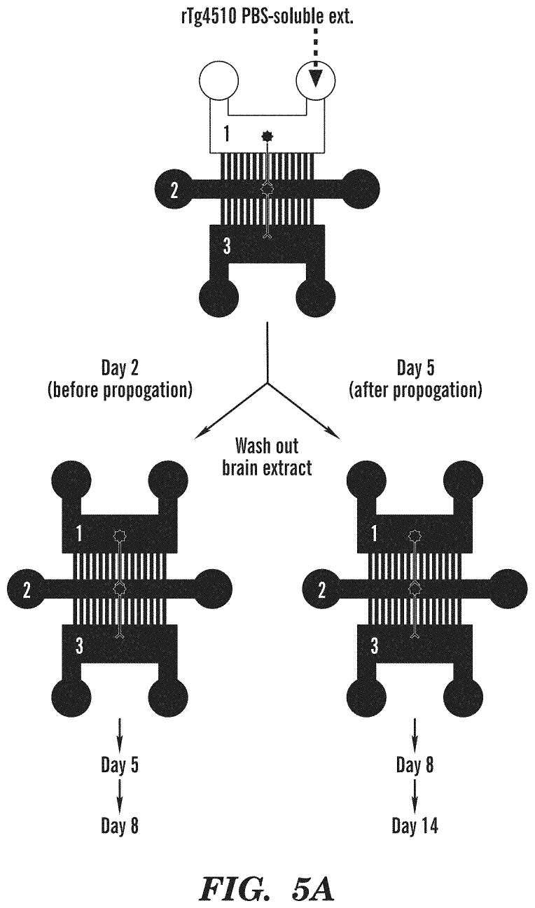

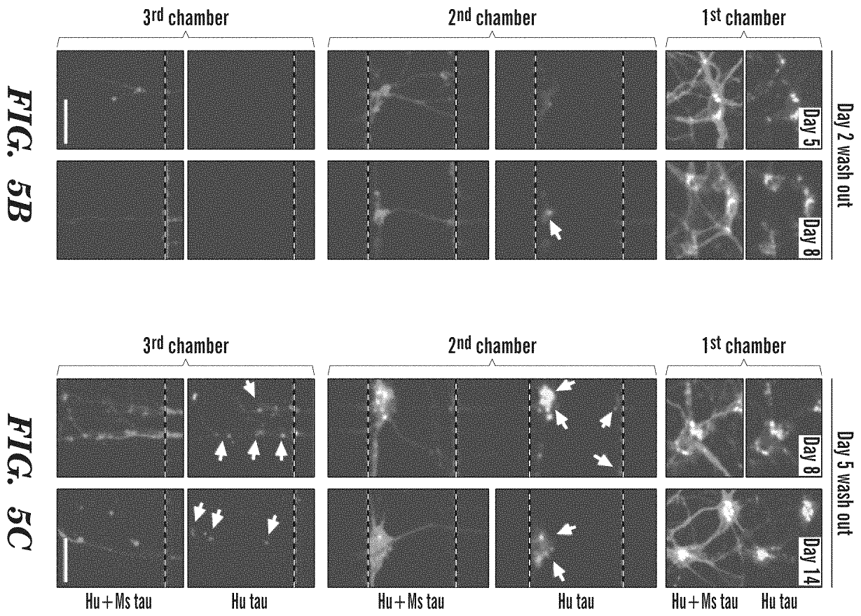

To assess the lifetime of tau in primary neurons after uptake, brain extract from rTg4510 (PBS-3,000 g, 500 ng/ml human tau) was added into the 1st chamber of the microfluidic device and excess tau was removed before (at day 2, FIG. 4E) or after (at day 5, FIG. 4E) tau had propagated to the 2nd chamber neurons, and neurons were further cultured for 6 (day 2-8) or 9 (day 5-14) days, respectively (FIG. 5A). Surprisingly, human tau positive neurons in the 2nd chamber were detected even after removal of brain extract from the 1st chamber prior to propagation (at day 8, 6 days after excess tau removal) (FIG. 5B). Similarly, human tau positive axons were observed in the 3rd chamber at day 8 (3 days after removal of brain extract from the 1st chamber) (FIG. 5C). These findings indicate that once a certain amount of tau was taken up by the neurons, tau could propagate to the next neuron even after removal of extracellular tau species. Tau species taken up by the 1st chamber neurons or propagated to the 2nd chamber neurons could be detected for up to 6 days (day 2-8, FIG. 5B) or 9 days (day 5-14, FIG. 5C) after washing out the human tau from the medium, indicating a slow degradation of HMW tau species in cultured neurons.

Uptake of Phosphorylated Soluble HMW Tau Derived from Human AD Brain

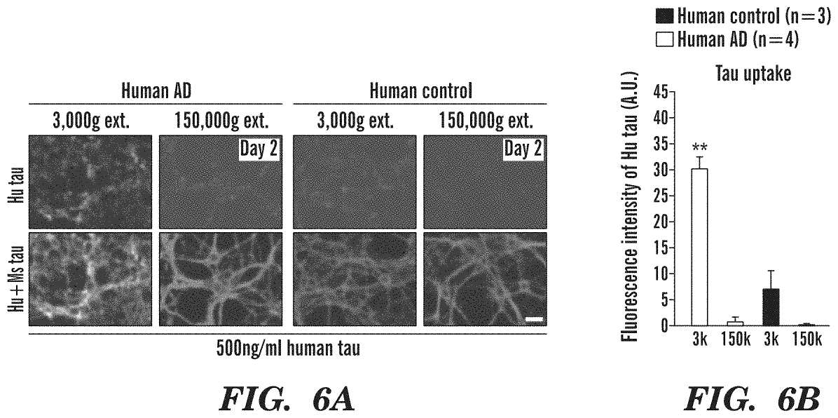

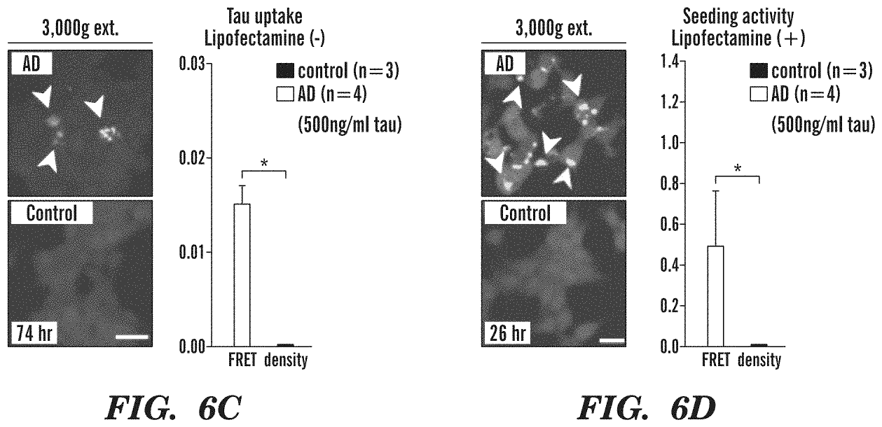

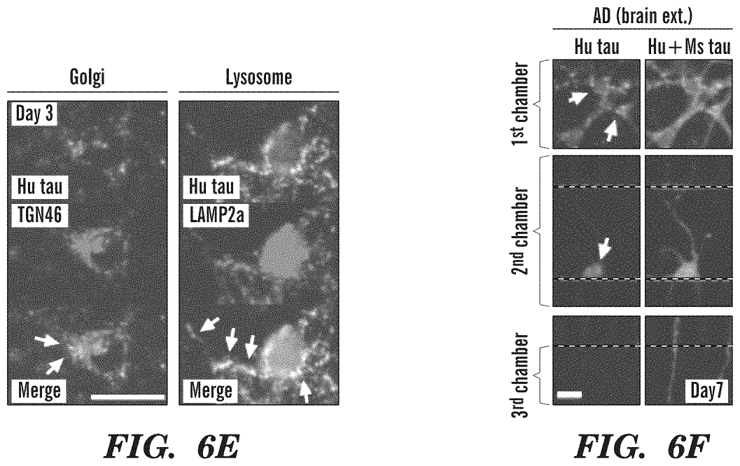

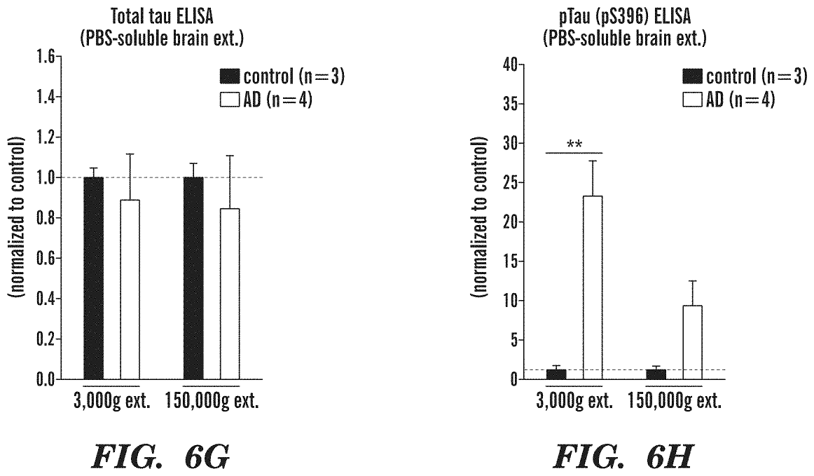

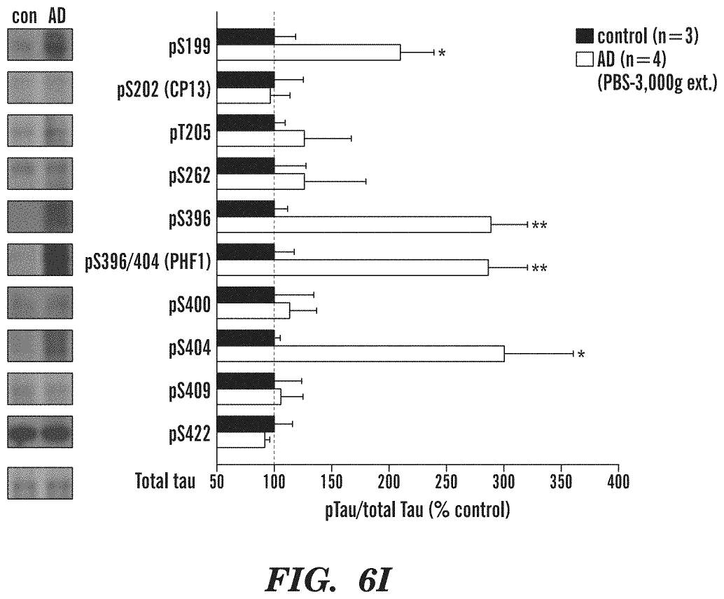

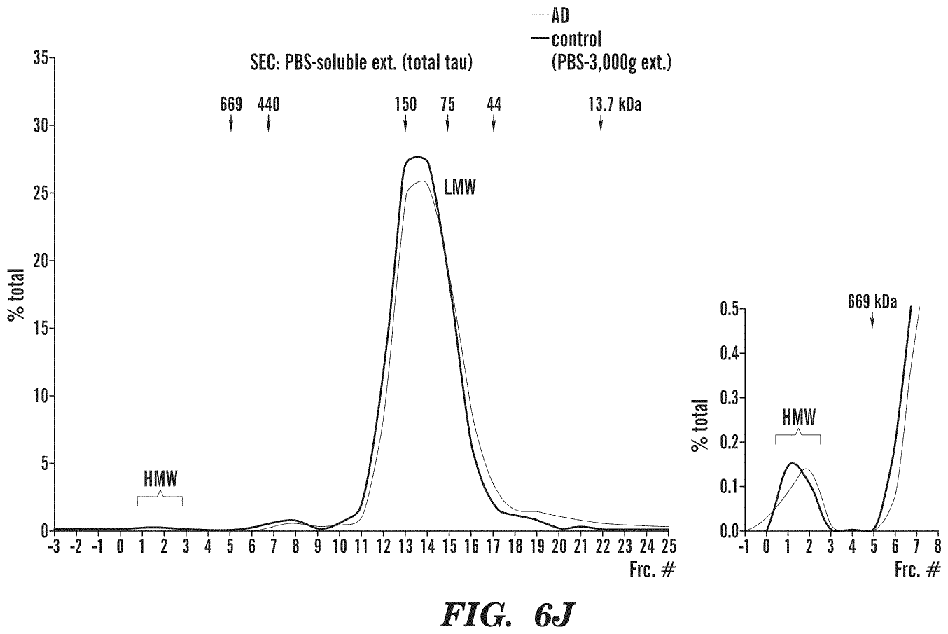

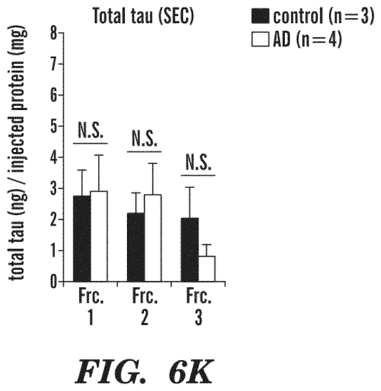

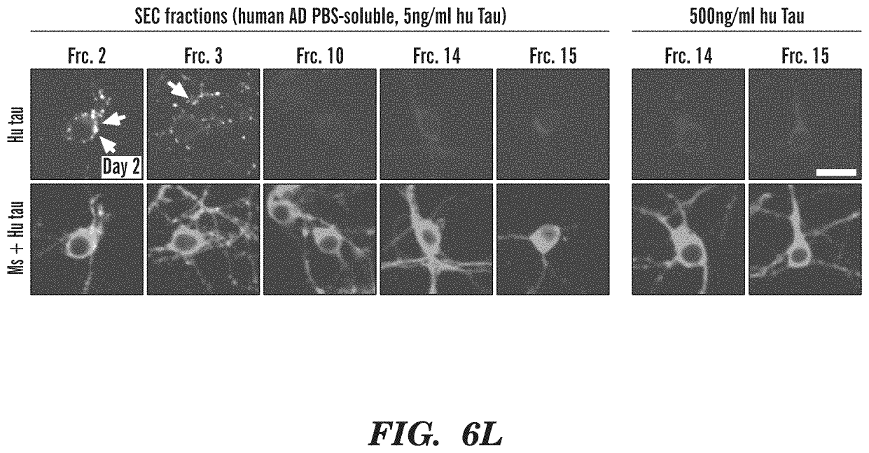

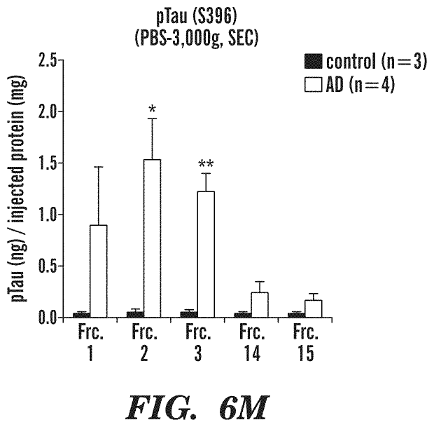

Uptake and propagation properties of tau species derived from human AD and control brain tissues were next examined. Like rTg4510 brain extract, the PBS-soluble extract from human AD brain contained tau species that could be taken up by mouse primary neurons (FIG. 6A). These tau species again were found only in the 3,000 g extract (FIGS. 6A-6B). No uptake was observed from human control brain extracts (FIGS. 6A-6B). Cellular uptake of AD brain-derived tau (3,000 g extract) was also assessed in HEK-tau-biosensor cells (FIG. 6C). 3,000 g extracts from AD brain had higher seeding activity than those from control brain (FIG. 6D). The tau species taken up by neurons co-localized with markers for the Golgi apparatus and the lysosomes (FIG. 6E), indicating the internalization and intracellular processing of tau. The tau species from AD brain extracts also propagated between neurons in the 3-chamber microfluidic device within 7 days (FIG. 6F).

Total tau levels in PBS-soluble extracts from AD and control brains were similar (FIG. 6G). However, the AD brain extract (3,000 g) contained significantly higher levels of phosphorylated tau (FIGS. 6H, 6I, and 6M) when compared to the control brain, especially those associated with some specific phosphorylation sites such as pS199, pS396, and pS404 (FIG. 6I). Surprisingly, both AD and control brain extracts (PBS-3,000 g) had comparable total amounts of HMW tau species on SEC analysis (FIGS. 6J and 6K), despite the clear difference in cellular uptake of tau from the AD and control extracts (FIGS. 6A-6C). The involvement of AD brain-derived HMW tau species in neuronal uptake was assessed by incubating each SEC fraction with primary neurons (FIG. 6L). Little uptake of the lower MW fractions occurred, even when tau was supplied at 100 times higher concentrations (5 vs. 500 ng/ml human tau in the medium).

Phosphorylation levels of tau were then measured in each SEC fraction. The HMW tau species from the AD brain were highly phosphorylated compared to those from control brain (FIG. 6M). Notably, most of the highly phosphorylated tau species from PBS-soluble AD brain extract were detected in the HMW fractions (FIG. 6M). These findings indicate the presence of phosphorylated soluble HMW tau species in PBS-soluble extracts from AD brain tissue and indicate that these phosphorylated forms can be the forms taken up and propagated by neurons.

Phosphorylation of Tau Correlates with Neuronal Uptake

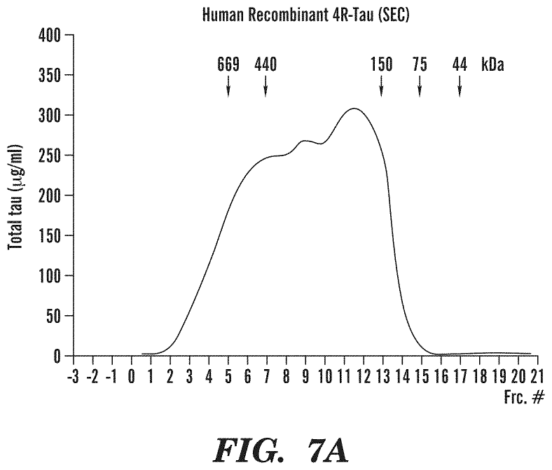

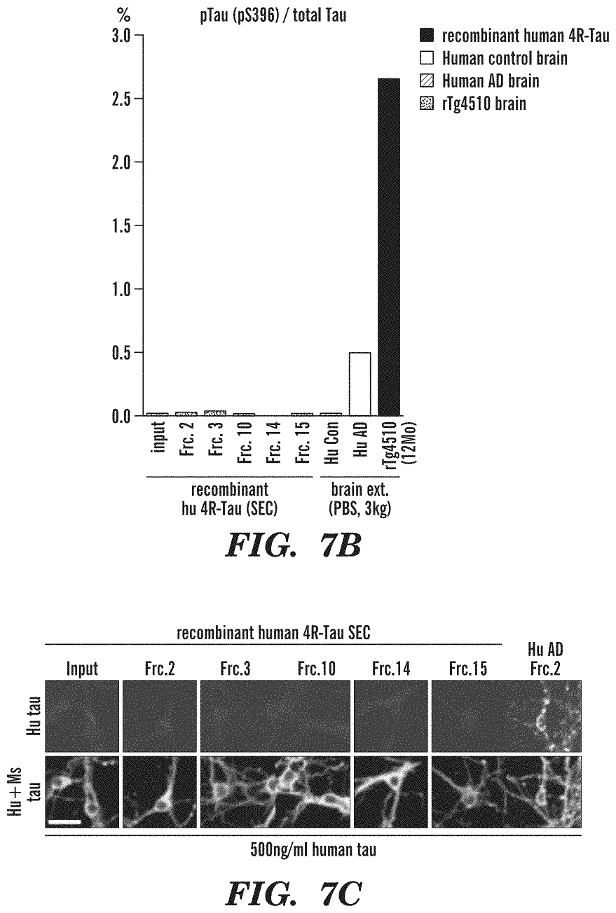

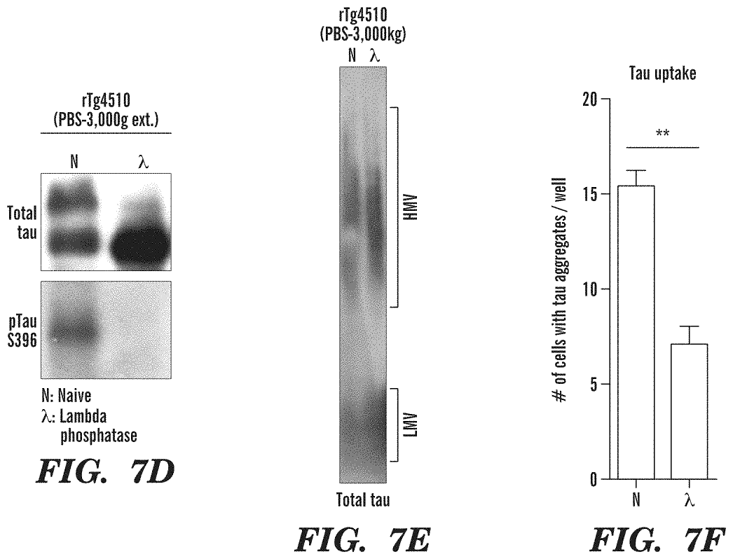

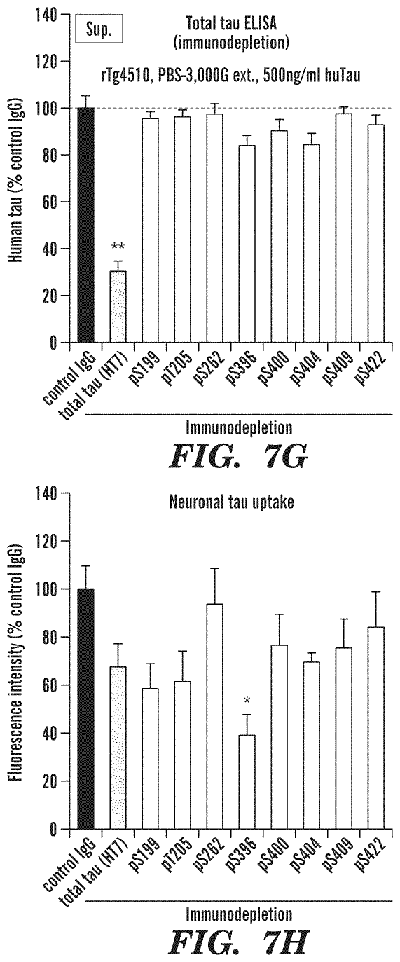

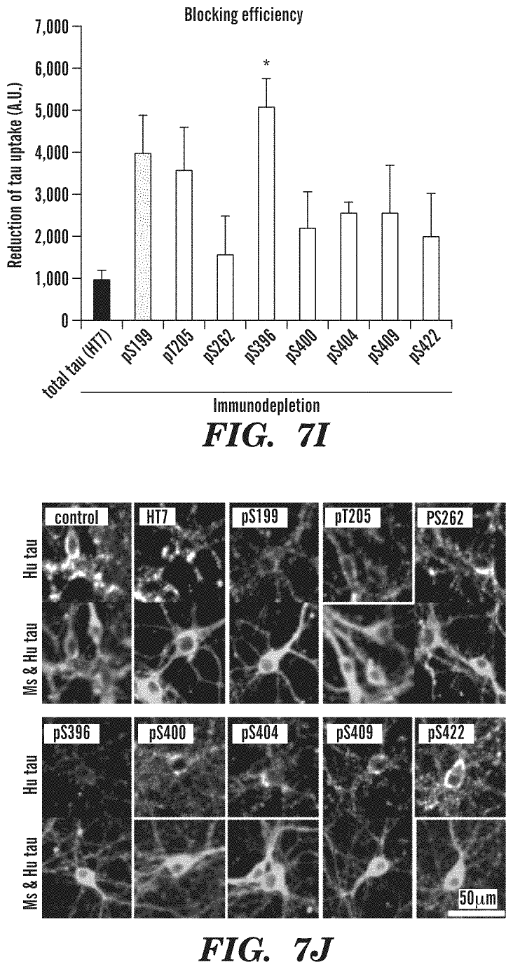

It was sought to determine if tau phosphorylation or, simply, size of tau, was important for cellular uptake by preparing a monomer-dimer-oligomer tau mixture from recombinant human WT full-length tau (441 aa), separated by SEC (FIG. 7A). Each SEC fraction of this non-phosphorylated tau mixture (FIG. 7B) was then incubated with mouse primary neurons. No uptake was observed in primary neurons even from HMW tau fractions (FIG. 7C).

It was next sought to examine the effect of dephosphorylation of tau on cellular uptake. Phosphatase treatment dephosphorylated tau in rTg4510 brain extract (FIG. 7D) without changing HMW tau levels (FIG. 7E), resulting in a significant reduction of cellular uptake of tau (FIG. 7F). Tau uptake from phospho-tau-immunodepleted rTg4510 brain extract in primary neurons was then assessed. Phospho-tau specific antibodies were less efficient at immunodepletion than the total tau antibody (HT7) (FIG. 7G); however, some phospho-tau antibodies (pS199, pT205, and pS396) more efficiently reduced the neuronal tau uptake (FIGS. 7H-7J) than the total tau antibody, indicating that they specifically interacted with a species of tau important for uptake. Taken together, these findings indicate that phosphorylation enhances neuronal uptake.

### Brain Extracellular Tau Species can be Taken Up by Primary Neurons

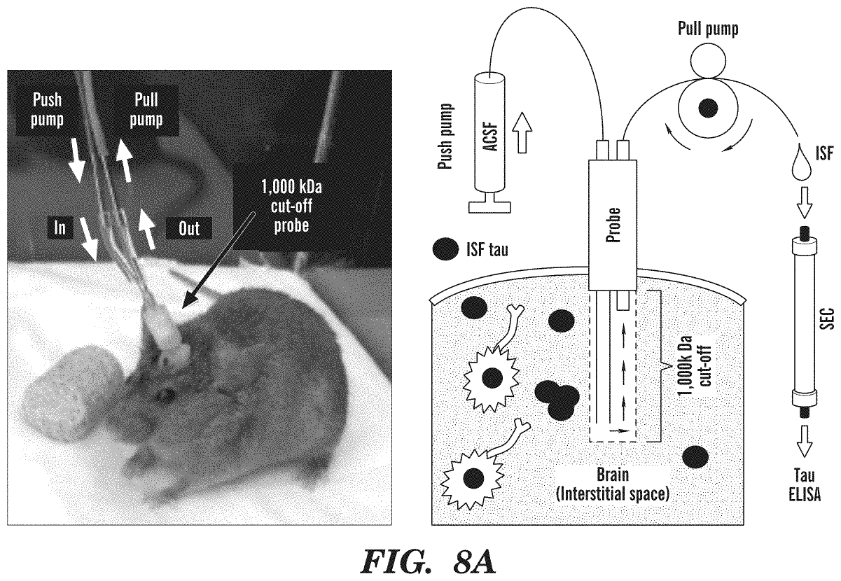

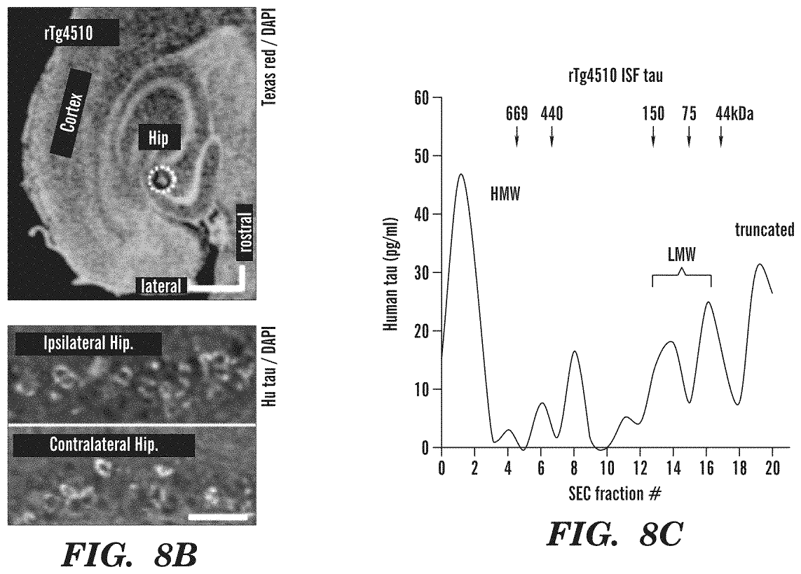

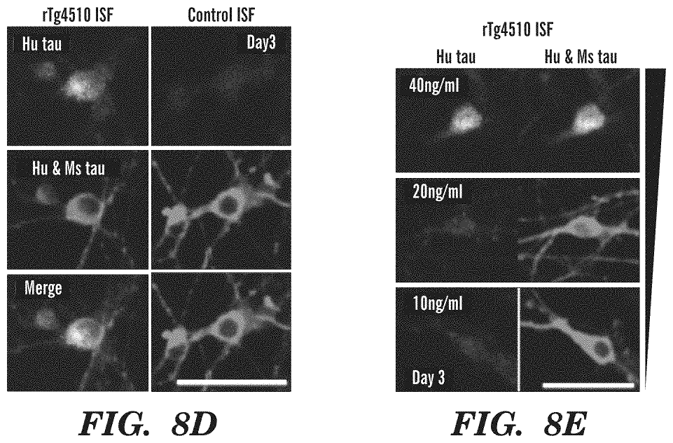

It is known that soluble tau species exist in the cerebrospinal fluid (CSF) and the ISF in the brain (25). It has been described in the International Patent Application No. WO 2015/089375, the content of which is incorporated herein by reference, that soluble HMW tau species, not LMW tau species, are involved in neuronal uptake and propagation between neurons. However, specific phosphorylated forms of high-molecular-weight tau species in the CSF or ISF that are involved in neuronal uptake and propagation are not yet known. Here, the inventors employed a unique large-pore (1,000 kDa cut-off) probe microdialysis technique with push-pull perfusion system that allows consistent collection of HMW molecules from the brain ISF of awake, freely-moving mice (21,22) (FIGS. 8A-8B). SEC fractionation followed by human tau-specific ELISA showed that brain ISF from rTg4510 mouse contained phosphorylated HMW tau species in addition to LMW tau (FIG. 8C). ISF tau from rTg4510 mouse was taken up by primary neurons after 3 days of incubation (FIG. 8D), with 40 ng/ml total human tau being sufficient to detect tau uptake (FIG. 8E). The distribution of tau appeared to be more diffuse in soma compared to tau taken up from brain extracts, which, without wishing to be bound by theory, might be due to the relatively low tau levels in ISF or a different size distribution pattern (FIG. 5C). Combining these data and the immunodepletion data described above, it is shown that secreted HMW tau phosphorylated at S396, S199 and/or S404, present in the ISF of awake behaving animals, can be preferentially taken up by neurons and therefore can account for the higher propagation of tau across neural systems observed in transgenic models.

### Effect of Tau Uptake and Intracellular Aggregation on Cell Viability

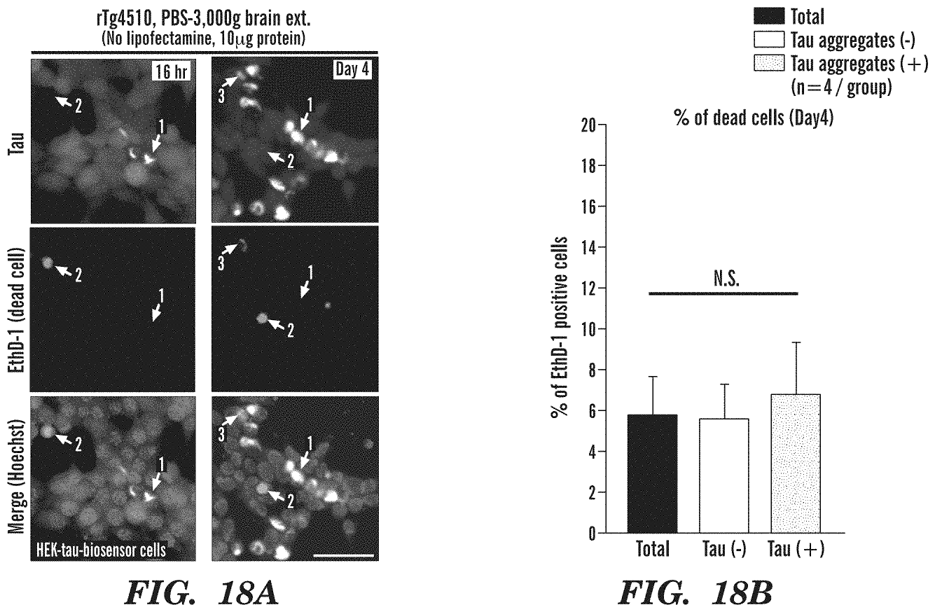

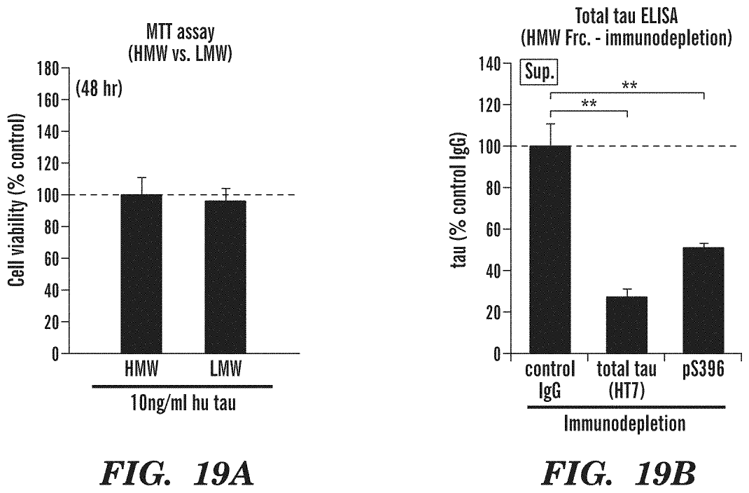

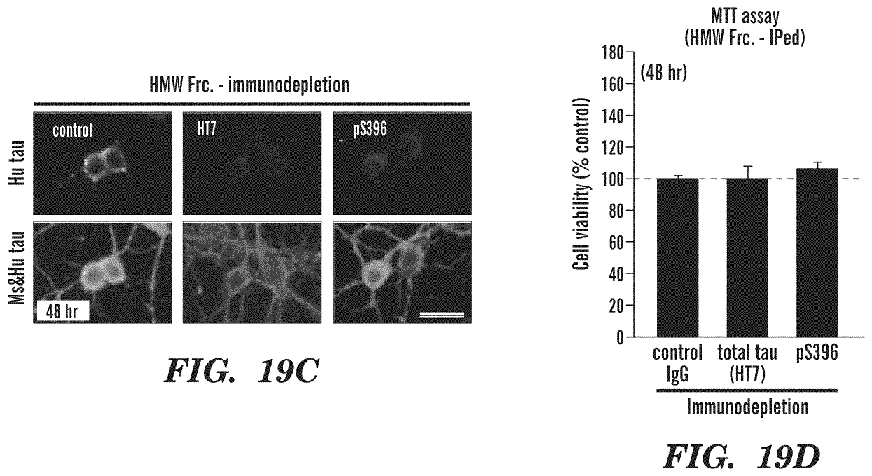

To assess the effect of extracellular tau uptake on cell viability, a cell death assay was performed using ethidium homodimer-1 (EthD-1) staining in HEK-tau-biosensor cells. There was no difference in cell viability between tau-aggregate positive and negative cells for up to 4 days (FIGS. 18A-18B). Additionally, the effect of HMW tau species on neuronal viability was assessed using 3-(4,5-dimethylthiazol-2-yl)-2,5-diphenyl tetrazolium bromide (MTT) assay. No acute toxicity was observed in mouse primary neurons treated with HMW tau at 48 hours (FIGS. 19A-19D). These findings indicate that cellular tau uptake and subsequent intracellular aggregation do not cause acute cell death.

### Discussion

Identifying the tau species that can be transferred between neurons is essential for understanding mechanisms by which misfolded tau propagates in AD and other tauopathies. Here, the uptake and propagation properties of tau from various sources were characterized: brain extracts and ISF from tau-transgenic mice, brain extracts from postmortem AD patients, and recombinant human tau protein. It was discovered that a rare HMW tau species, which accounts for only a small fraction (estimated at <1%) of all soluble tau species in the AD samples, was robustly taken up by neurons, whereas uptake of LMW tau was very inefficient; findings from the microfluidic neuron culture platform indicated that this rare species is uniquely capable of propagating between neurons. Furthermore, tau with similar biochemical characteristics can be identified in the brain ISF of rTg4510 animals obtained while they were awake and behaving, indicating that, without wishing to be bound by theory, it can be a normal product in the brain; this ISF can also donate tau that can be taken up by neurons in culture. Together, these data indicate that (i) a relatively rare, HMW, phosphorylated tau species is released from neurons and found in brain ISF; and (ii) this species can be taken up, axonally transported, secreted, and taken up by synaptically connected neurons and thus “propagated”.

Uptake of tau from mice expressing aggregating P301L tau (rTg4510) depended on tau MW, correlated with the level of phosphorylation. The findings presented herein indicate that oligomerization and pathological phosphorylation increased the uptake efficiency of tau. The HMW tau species had a pathologically misfolded conformation (positive for Alz50 antibody staining) and appeared as non-fibrillar structures in AFM. It was previously reported that synthetic tau fibrils (27-30) or fibrillar tau species extracted from tau-transgenic mouse brain (14, 15) could be taken up by neurons and induce filamentous tau pathology in vitro and in vivo. The findings presented herein indicate instead that soluble, misfolded HMW tau present in the extracellular space likely plays a role in propagation. Preliminary data using extracts from a human G389R tau mutation case with frontotemporal dementia that did not have filamentous tau inclusions nonetheless showed tau uptake into neurons from a PBS-soluble extract. The findings presented herein showed that soluble tau rather than filamentous aggregates support the propagation phenomenon described herein.

The findings presented herein do not entirely exclude the involvement of LMW tau species in uptake and propagation. It may well be possible that the concentration of tau used in this study (500 ng/ml human tau) was too low for uptake of LMW tau species, or that immunostaining was not sensitive enough to detect LMW tau internalized by neurons. Michel et al. (31) reported that extracellular monomeric tau enters SH-SY5Y neuroblastoma cells at concentration as high as 1 μM (approximately 55 μg/ml).

The intracellular accumulation of insoluble tau aggregates has long been considered to be toxic to neurons (32). While it was previously reported that insoluble tau aggregates are not sufficient to impair neuronal function (33), soluble phosphorylated soluble HMW tau species was not yet identified. The present findings from the cell viability assay (FIGS. 18A-18B, and 19A-19D) indicate that tau uptake and subsequent intracellular aggregation do not cause acute cell death. Whether extracellular and taken-up HMW tau have a long-term effect on neuronal function is unclear.

Spread of tau pathological lesions from medial temporal lobe towards temporal and other neocortical regions correlates well with the extent of cognitive impairment (5), and the number of tangles appears to increase in each brain area with increasing duration of disease (3,34). If one mechanism for this spread is trans-synaptic propagation of misfolded tau species, as has been postulated, then understanding the parameters that govern this process is of importance in designing effective strategies to slow progression of cognitive changes in AD. Extracellular tau propagation can be divided into uptake, axonal transport, release, and re-uptake phases, with pathobiological processes leading to enhanced or diminished rates at each step. The data presented herein indicate that uptake of unmodified LMW tau at physiological concentrations is not detected during the time course of the experiments, whereas uptake of a relatively rare HMW phosphorylated species is very efficient, occurring within 24 hours of exposure. Even in vivo, injection of the HMW tau, as opposed to the much more abundant LMW tau species, leads to uptake into neurons both in WT (FIGS. 14A-14C) and pre-tangle stage rTg4510 (FIGS. 1J-1L) mouse brains. At least three lines of the present data indicate that phosphorylation is important: uptake efficiency correlates with extent of phosphorylation (FIGS. 2A-2H and FIGS. 6A-6M); enzymatically dephosphorylating the tau blocks uptake (FIGS. 7D-7f); and some phospho-tau specific antibodies are able to block uptake despite being relatively ineffective in IPing tau from the rTg4510 brain extract (FIGS. 7G-7J). In addition, recombinant nonphosphorylated tau, even when prepared as an HMW oligomer, is not taken up efficiently by neurons (FIGS. 7A-7C).

The HMW tau appears to be quite stable once it is taken up, being detectable days after it is washed off (FIGS. 5A-5C), which might be due to the hyperphosphorylation state of this species (35,36); it undergoes axonal transport, is released, and can be taken up by the next neuron. Thus after initial uptake, at least in the model systems described herein, axonal transport, release into a synapse and trans-synaptic propagation seem to occur relatively rapidly. When applying the findings presented herein to human AD, it indicates that formation, release, and uptake of the HMW phosphorylated forms of tau can be key factors in determining propagation of tau across neural systems. The amount of HMW phospho-tau species accounted for less than 1% of the total PBS-soluble tau in AD brain extracts, and less than 10% even in mutant-tau overexpressing rTg4510 mouse brain extracts, in contrast to the much more abundant LMW tau species. The findings presented herein indicate that targeting HMW tau species can be an effective way of blocking or slowing the tau propagation cascade in AD. Intervention to deplete these specific extracellular tau species can inhibit tau propagation and hence disease progression in tauopathies.

### Example 2. A Unique High-Molecular-Weight (HMW) Tau Species is Involved in Propagation and Accumulates in the Cerebrospinal Fluid of Alzheimer's Disease Patients

Alzheimer's disease (AD) clinical progression has been commonly thought to be driven by the accumulation of tau NFT (insoluble, fibrillar aggregates of tau proteins) progressing through the brain affecting increasing numbers of cortical areas. Previous animal model studies also report that NFT propagation occurs by a cross-synaptic spread of pathological forms of tau. However, the inventors discovered a phosphorylated high-molecular-weight (HMW) tau species present in the brain extracts from tau-tg mice and AD patients, which can be taken up by neurons and synaptically propagated to other neurons. In this example, biological and biochemical properties of brain extracellular tau were examined from various sources including brain interstitial fluid (ISF) and cerebrospinal fluid (CSF) from tau-transgenic mouse model (rTg4510), human postmortem ventricular CSF, and lumbar CSF from AD patients.

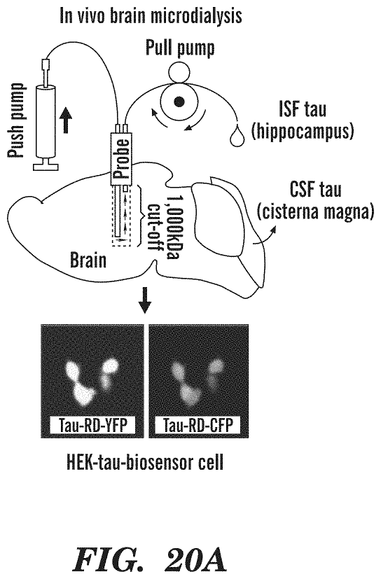

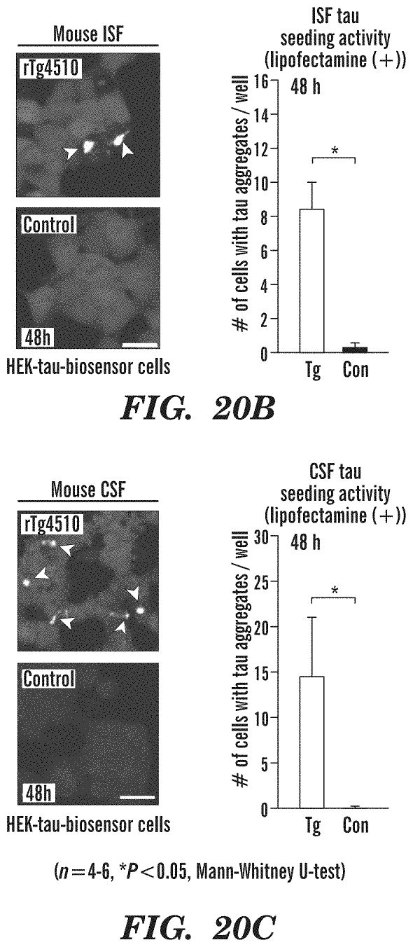

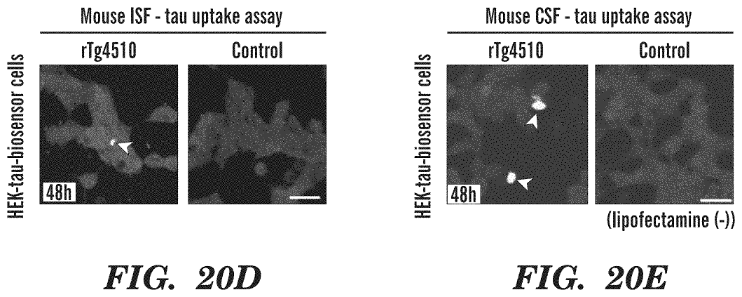

HMW tau molecules were collected from the brain interstitial fluid (ISF) or cerebrospinal fluid (CSF) of tau-transgenic rTg4510 mice and control rTg21221 mice by microdialysis using a probe with a 1000-kDa MW cut-off as described below in the “Exemplary materials and methods” section. Cellular tau uptake and seeding activity of the collected HMW tau molecules were then measured using a FRET-based biosensor HEK293 cell line that stably expresses the human tau repeat domain with the P301S mutation fused with CFP/YFP as described in Holmes et al. (Ref. #23) and also described in the “Exemplary materials and methods” section below. FIGS. 20A-20E show seeding and uptake activity of high-molecular-weight (HMW) extracellular tau derived from brain interstitial fluid (ISF) or cerebrospinal fluid (CSF) of tau-transgenic rTg4510 mice and control rTg21221 mice. FIGS. 20B-20C show brain ISF (FIG. 20B) and CSF (FIG. 20C) derived from tau-transgenic rTg4510 mice had higher seeding activity in vitro that those from the control brain. FIGS. 20D-20E show brain ISF (FIG. 20D) and CSF (FIG. 20E) derived from tau-transgenic rTg4510 mice had higher cellular uptake activity that those from the control brain.

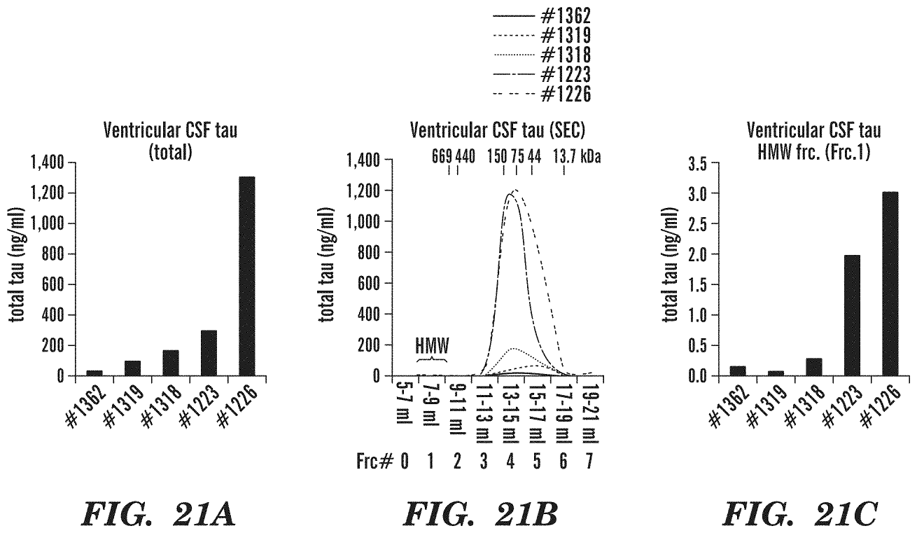

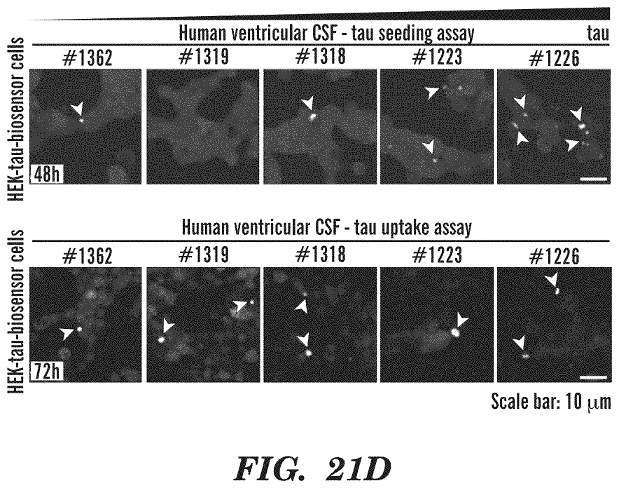

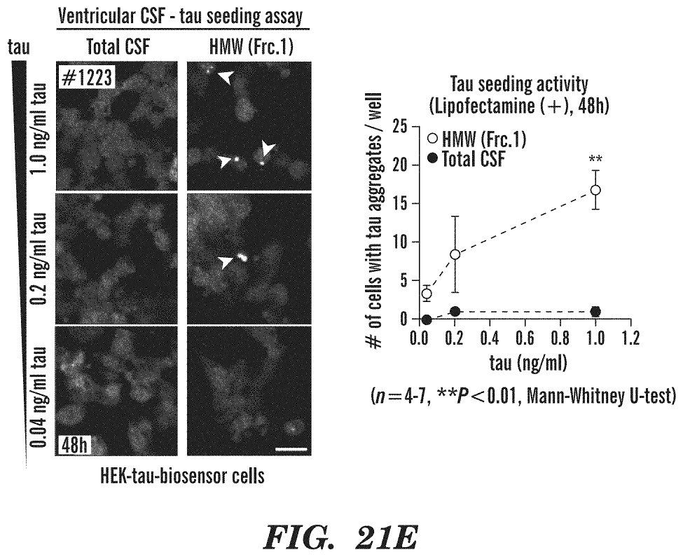

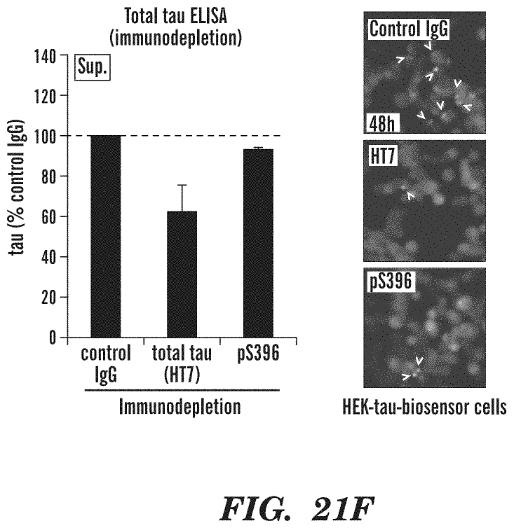

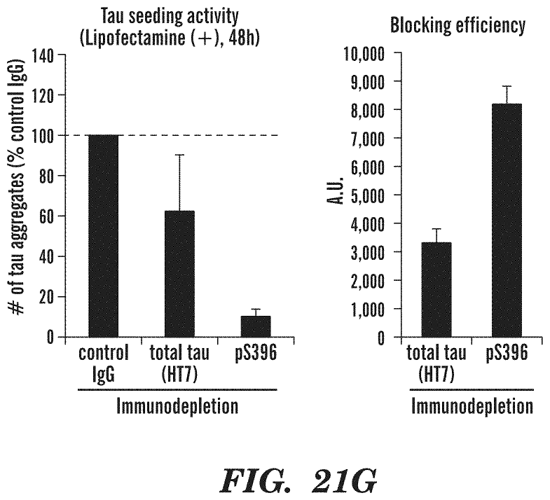

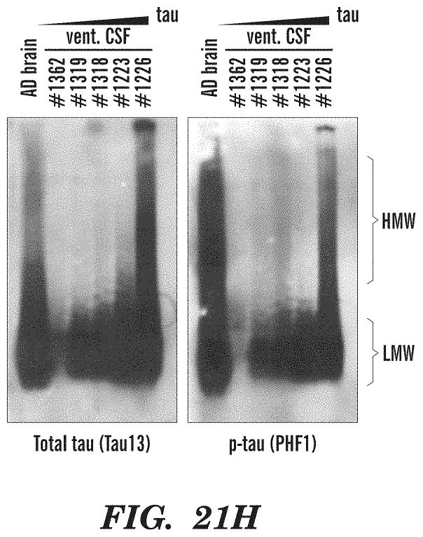

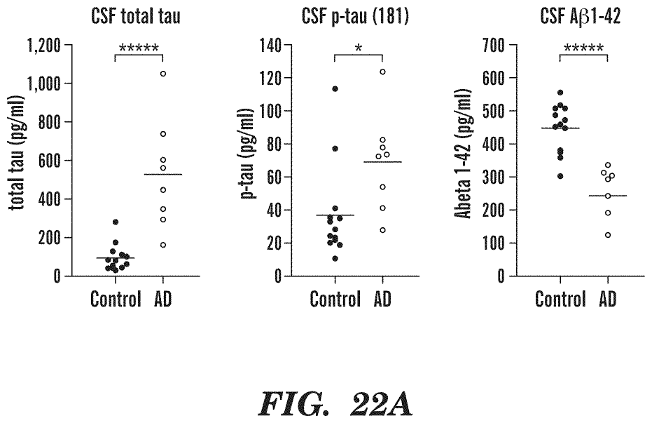

Postmortem ventricular CSF samples were collected from AD patients and subjected to size-exclusion chromatography (SEC) to assess the molecular-weight size distribution of tau present in the samples. FIG. 21A is a graph showing the total tau levels in the ventricular CSF (before fractionation by SEC) of each indicated AD subject, as measured using the human tau-specific ELISA. FIG. 21B is a graph showing molecular-weight size distribution of tau present in the AD human CSF samples assessed by size-exclusion chromatography (SEC). The total tau levels in each fraction were measured using the human tau-specific ELISA. FIG. 21C is a graph showing the levels of HMW tau (fraction 1 from SEC) in the AD human CSF samples as measured using the human tau-specific ELISA.

To determine the tau seeding activity of the ventricular CSF collected from postmortem AD subjects, the human CSF samples was applied to HEK-tau biosensor cells for tau seeding assay as described in Holmes et al. (Ref. #23) and also described in the “Exemplary materials and methods” section below. FIG. 21D is a set of fluorescent images showing seeding activity (upper row) and cellular uptake activity (lower row) of tau derived from the AD human CSF samples as measured using HEK-tau-biosensor cells. FIG. 21E contains a set of fluorescent images and quantitative data comparing seeding activity of tau derived from either AD human total CSF or HMW tau-comprising fractionated CSF in various concentrations, as measured using HEK-tau-biosensor cells. The data show that postmortem ventricular CSF from AD patients contained a rare HMW tau species which had higher seeding activity.

It was next sought to determine if the tau seeding activity of HMW tau species can be inhibited by blocking the HMW tau species with anti-tau antibodies. The AD human ventricular CSF samples were treated with anti-tau antibodies against total tau or against a specific phosphorylation site (e.g., pS395) and then applied to HEK-tau biosensor cells for tau seeding assay as described herein. FIG. 21F shows levels of tau left in the AD human CSF samples after immunodepletion with various indicated antibodies, namely control IgG, anti-total tau antibody (HT7), and anti-pS395 tau antibody. FIG. 21G shows seeding activity (left) and blocking efficiency (as measured by seeding activity) (right) of tau derived from the AD human CSF samples after immunodepletion with the indicated antibodies. The data show that immunodepletion of phospho-tau (e.g., using anti-pS396 tau antibody) reduced seeding activity in vitro.

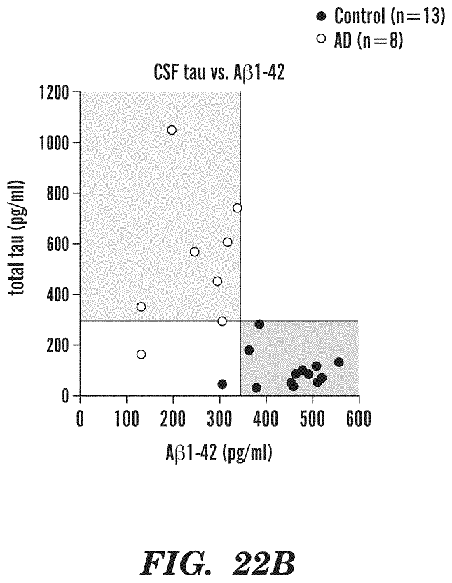

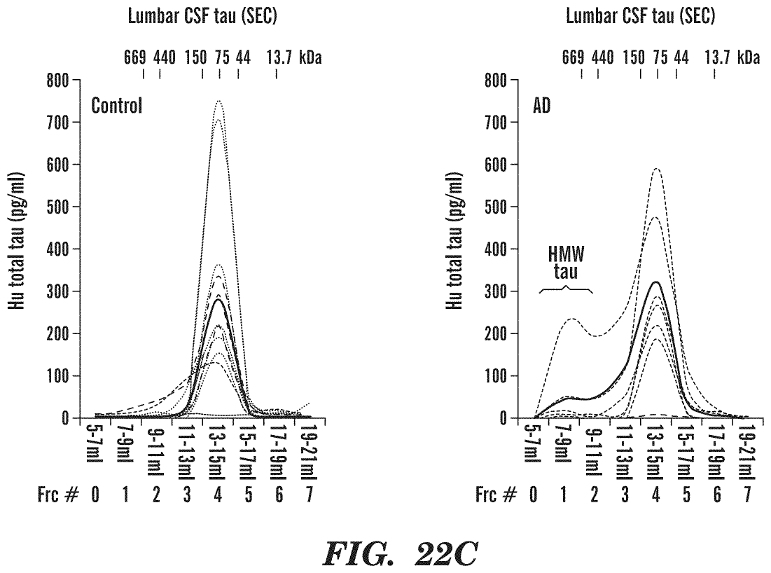

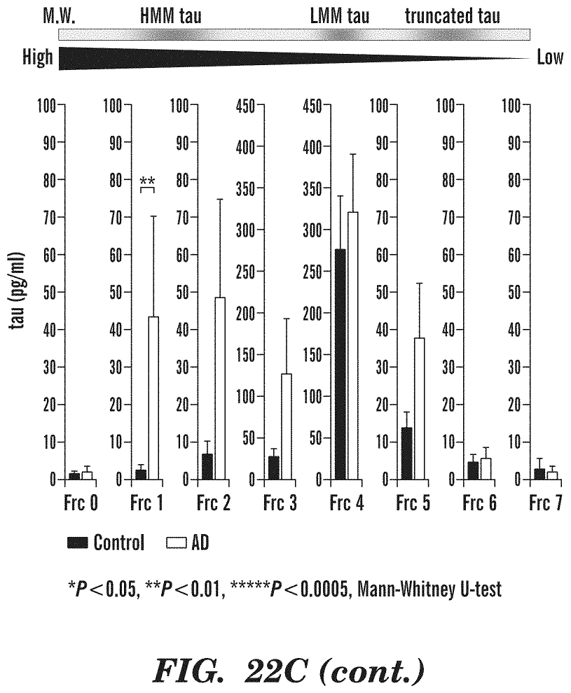

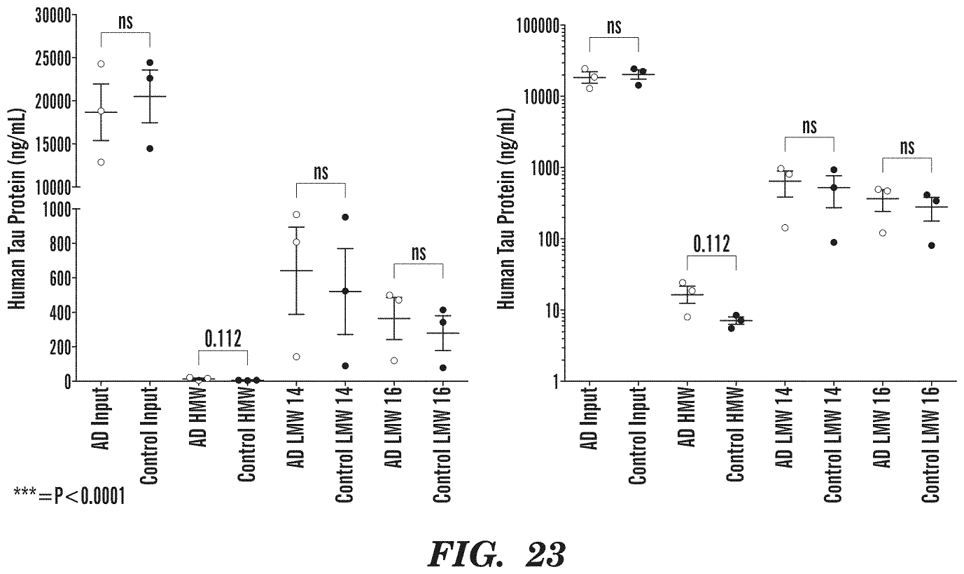

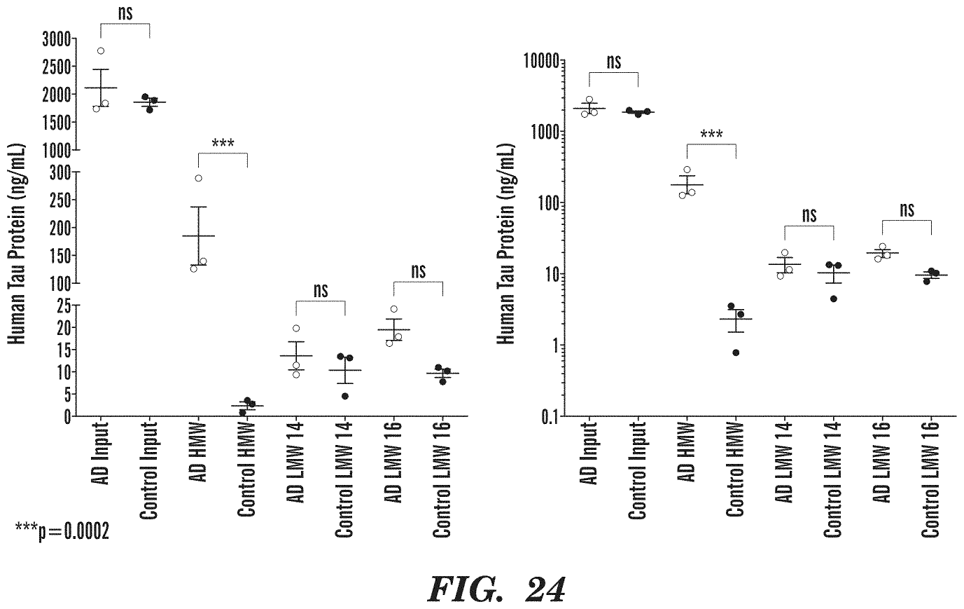

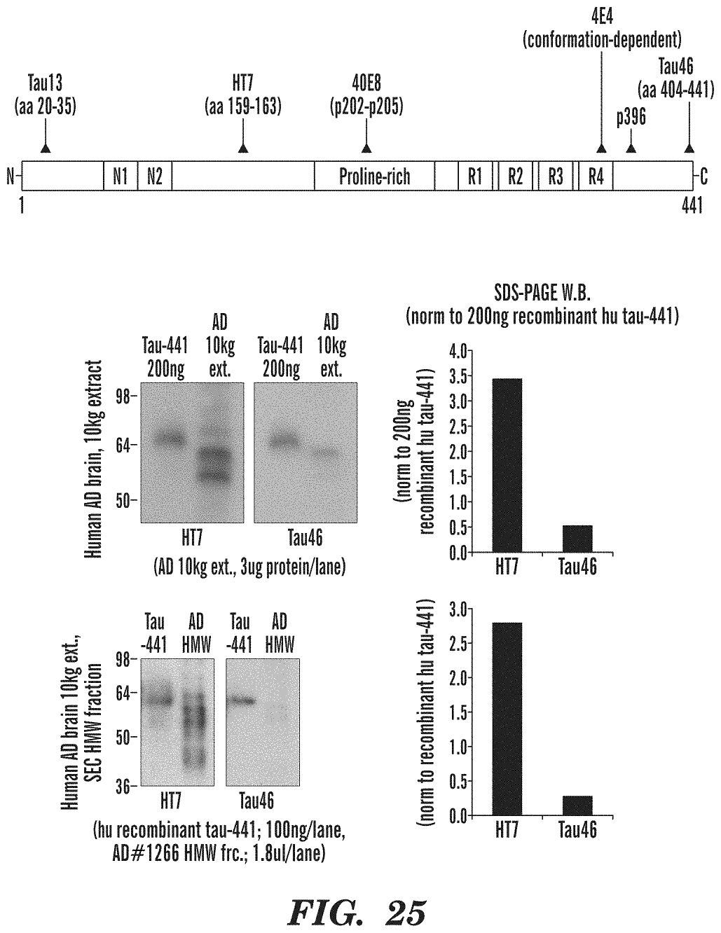

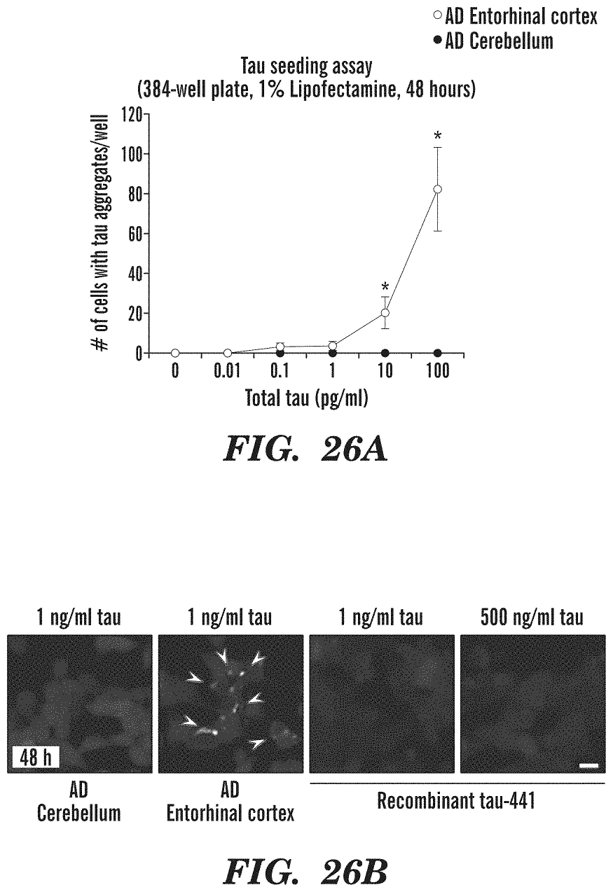

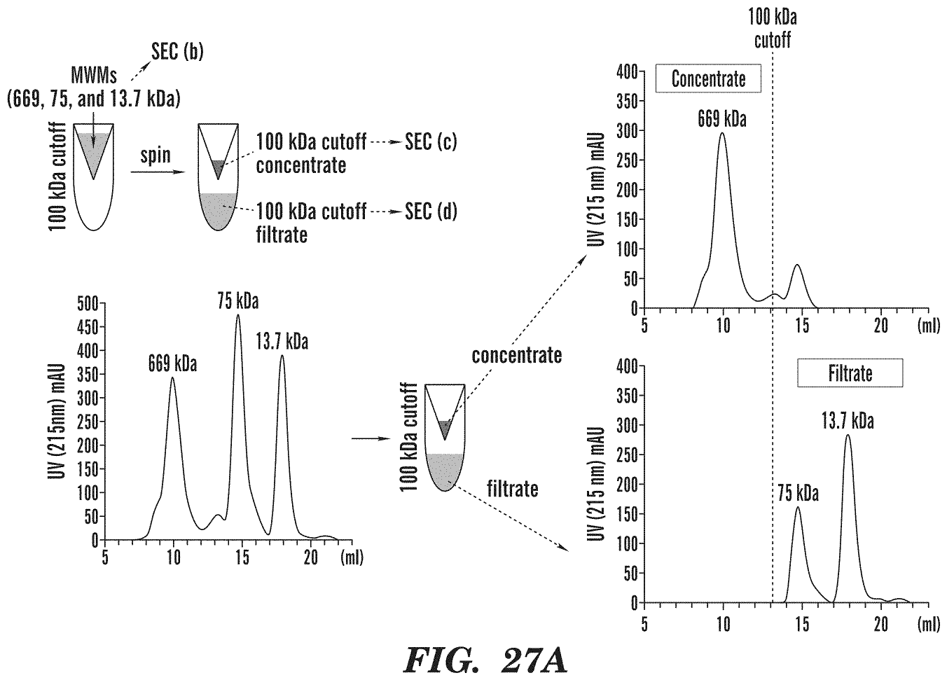

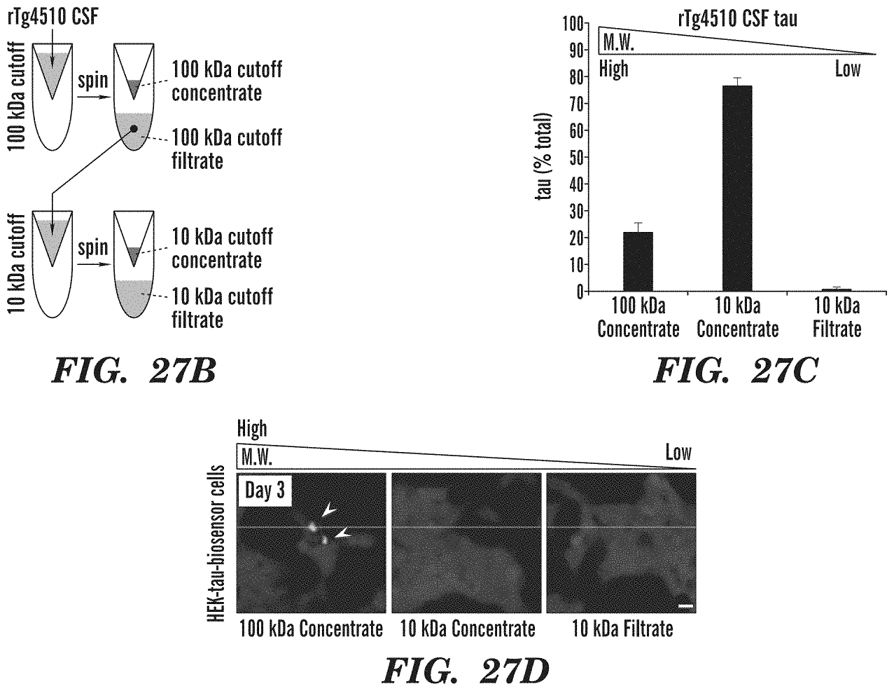

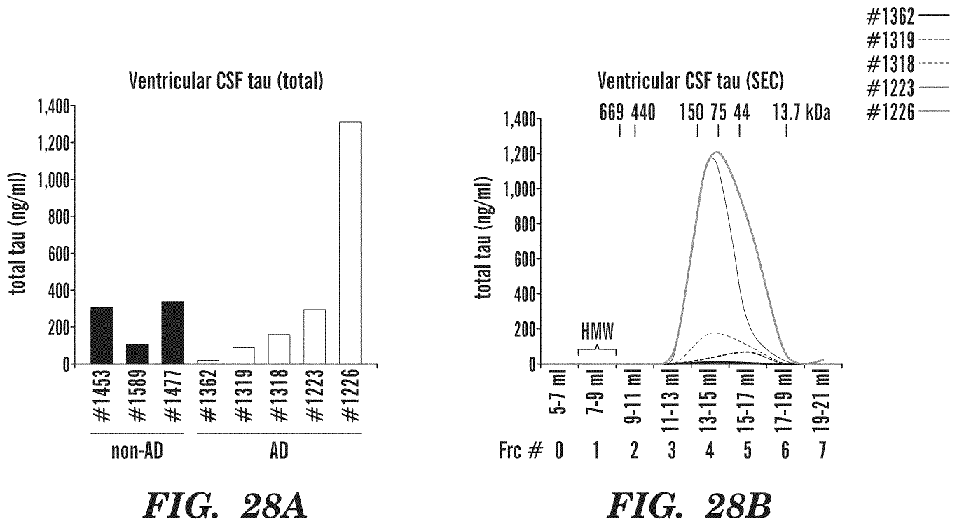

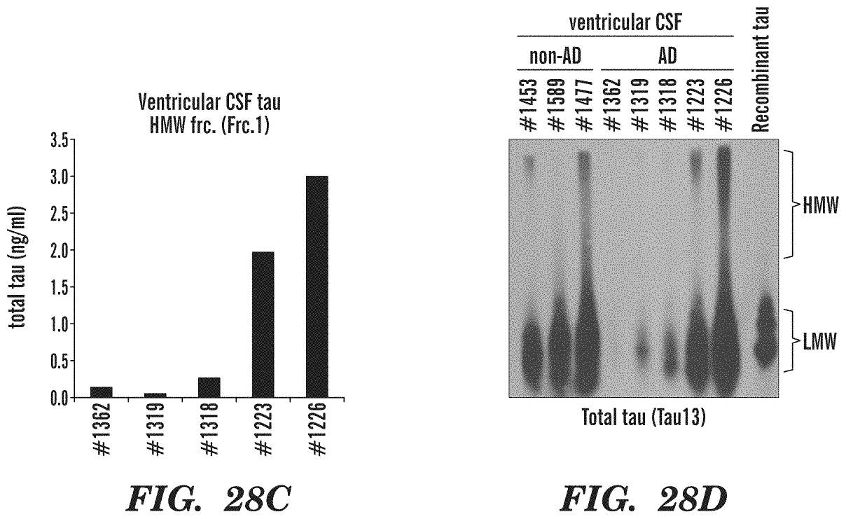

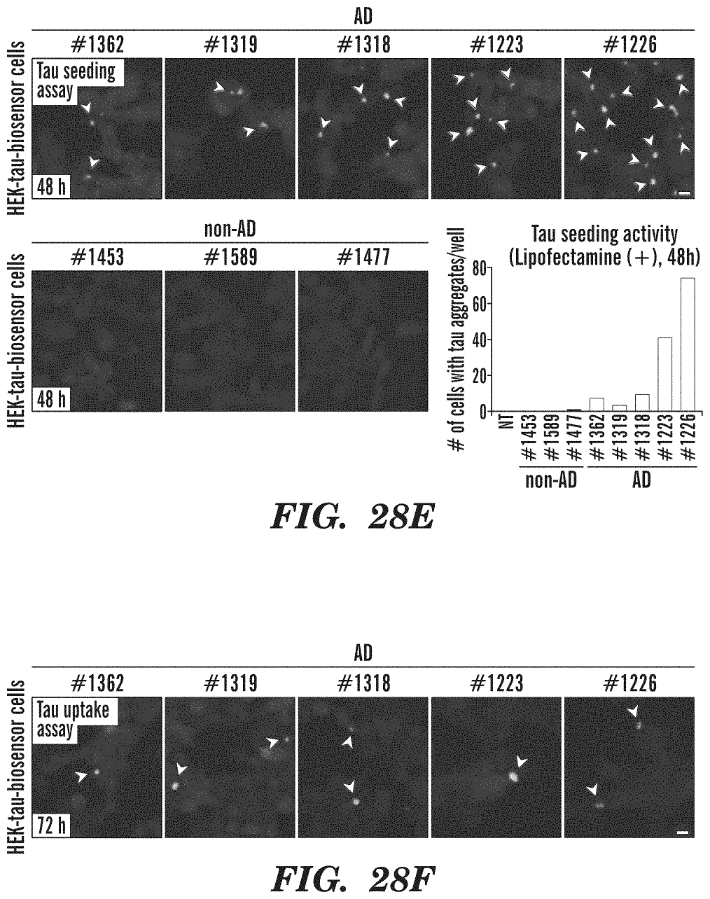

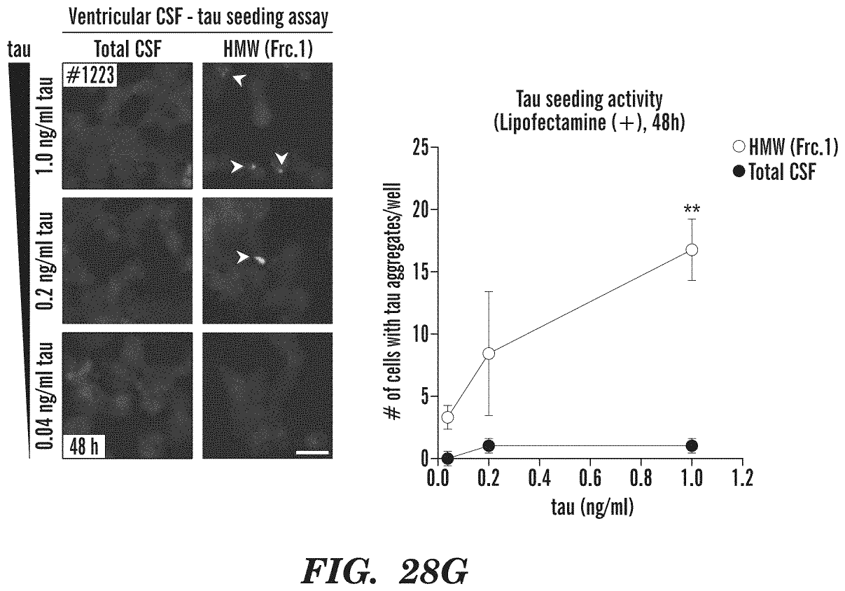

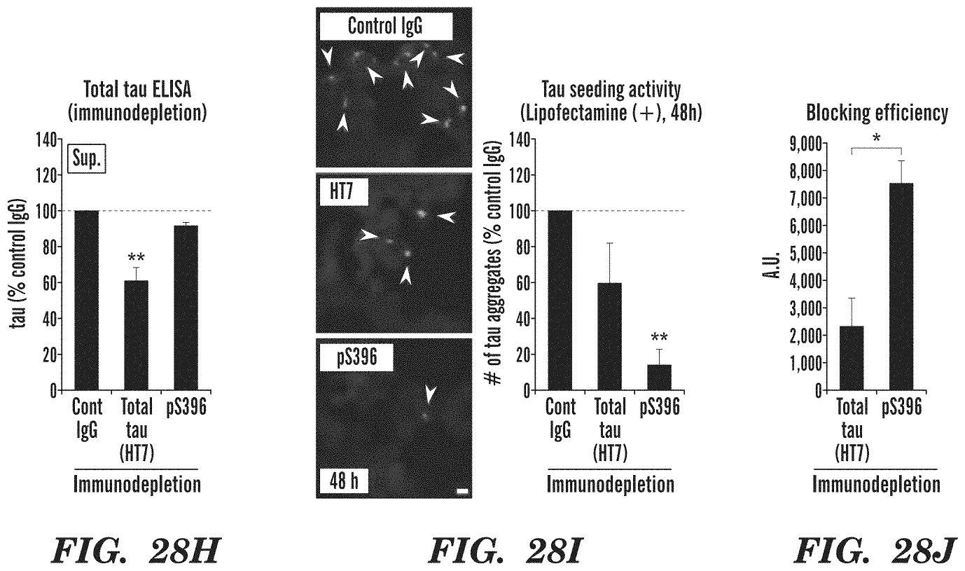

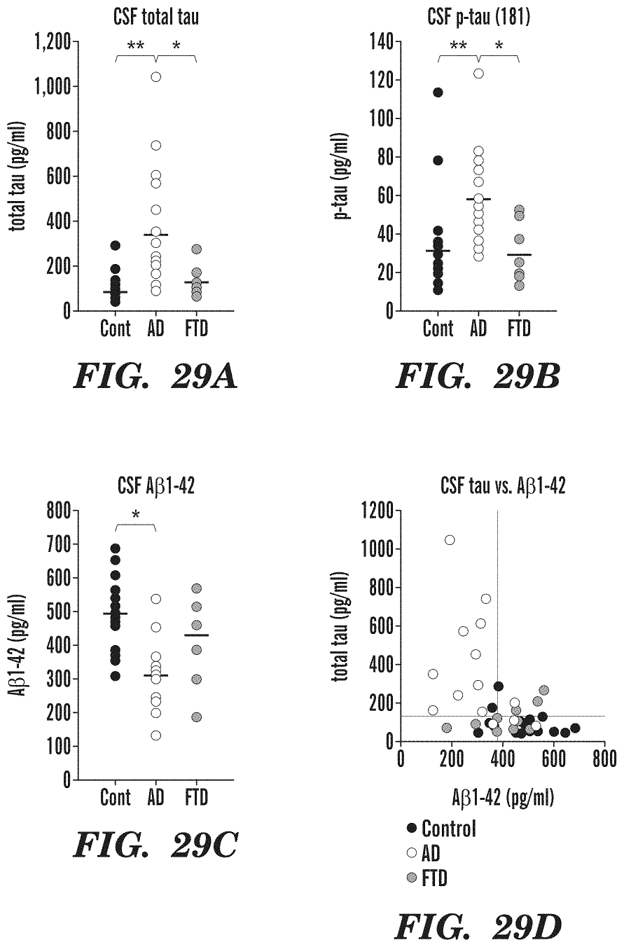

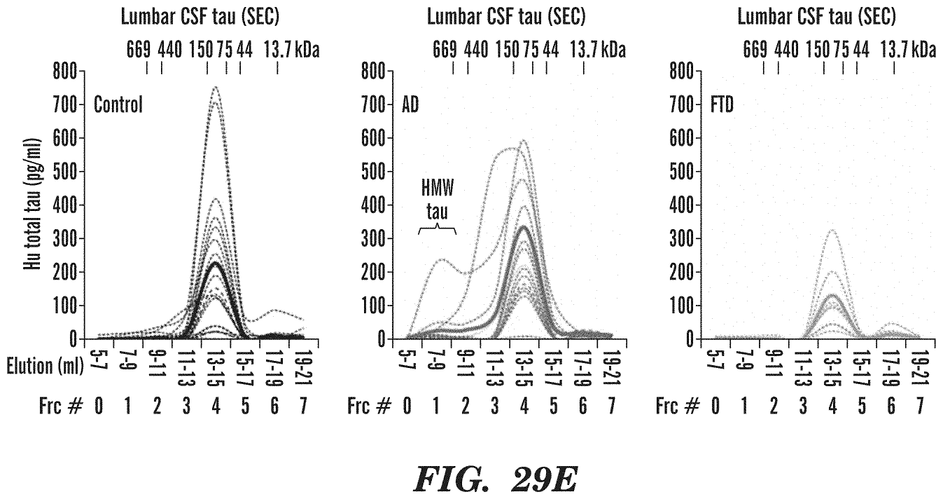

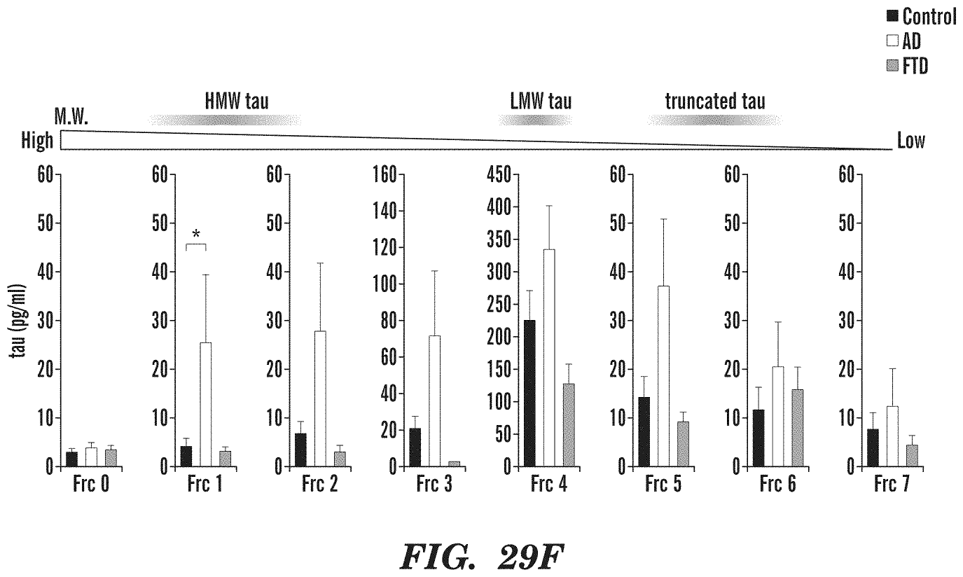

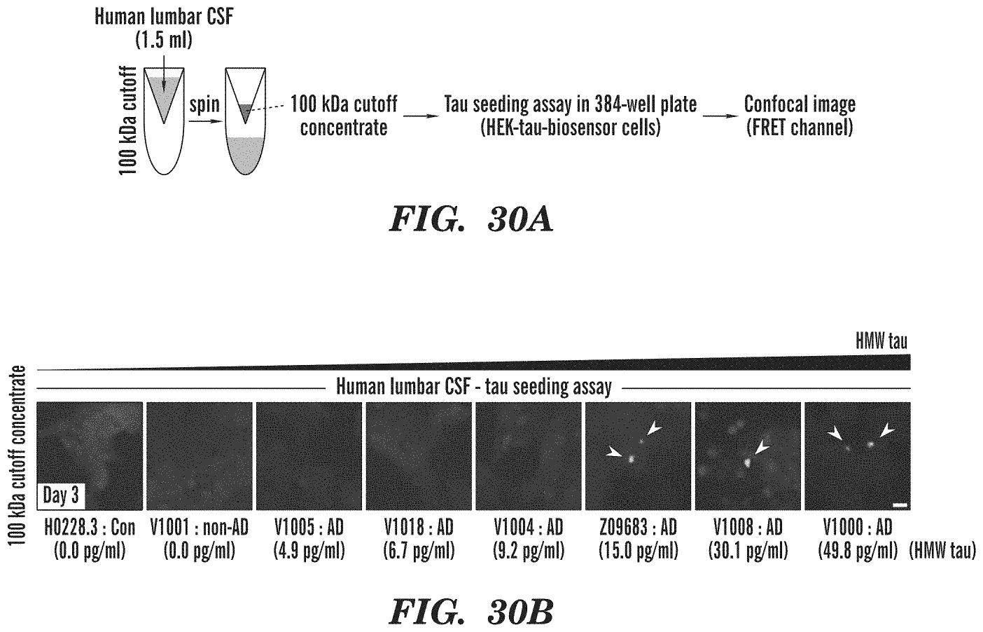

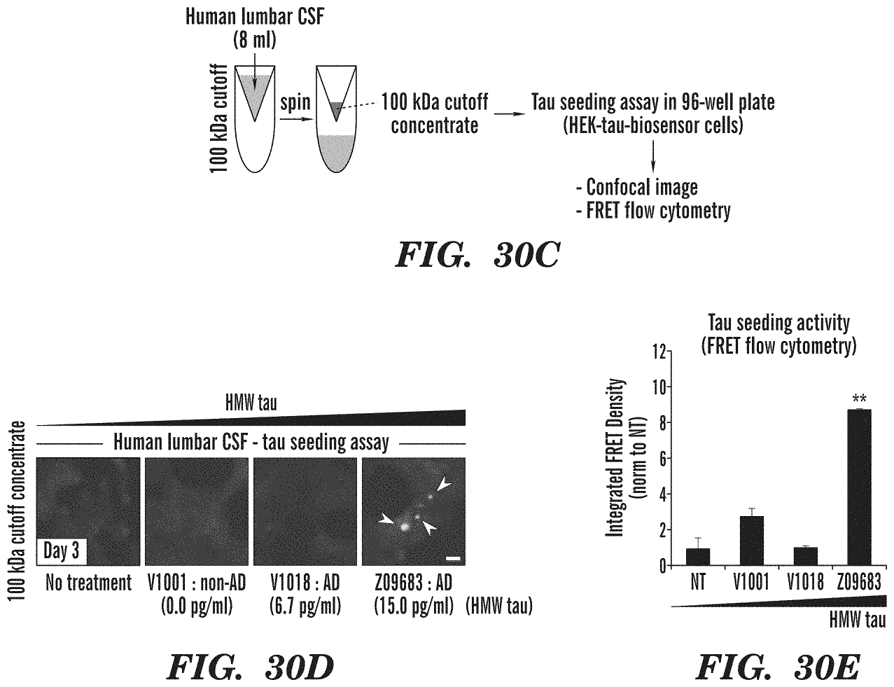

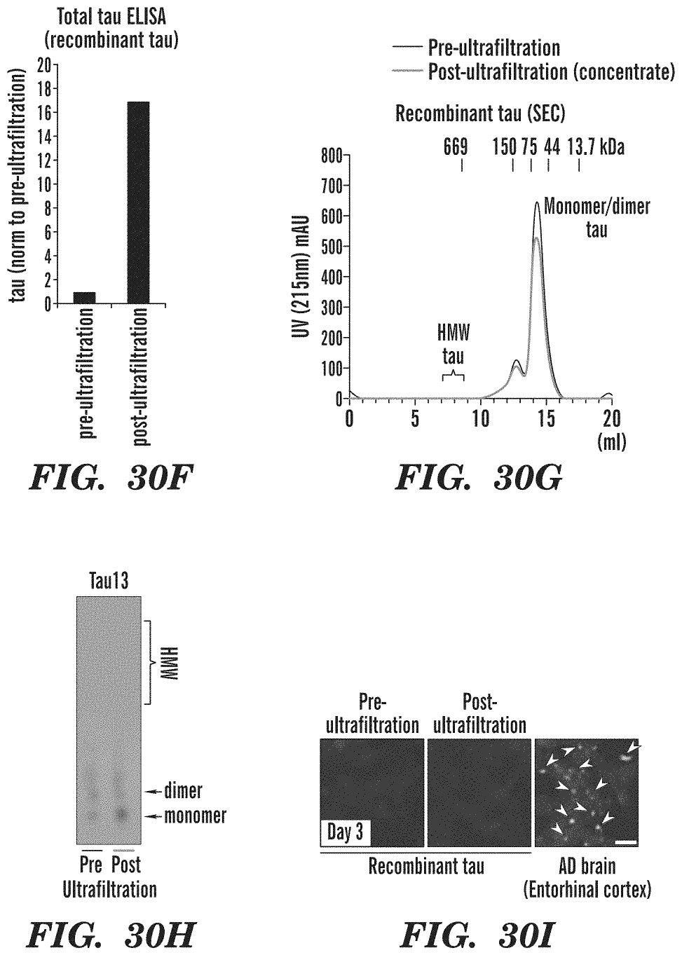

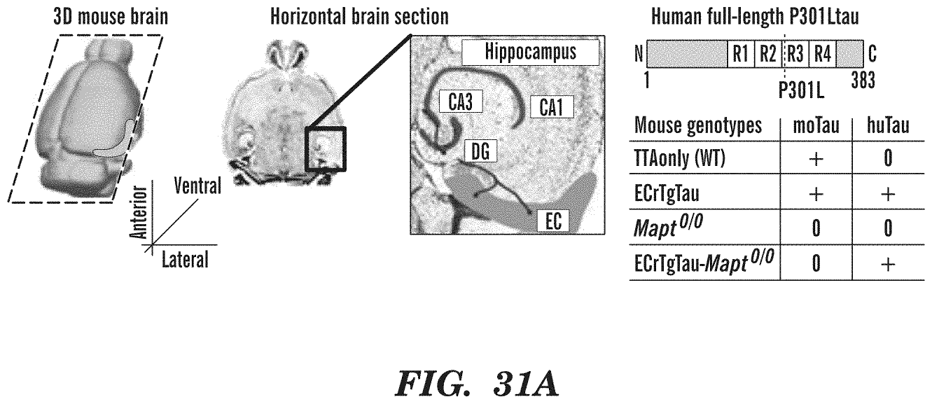

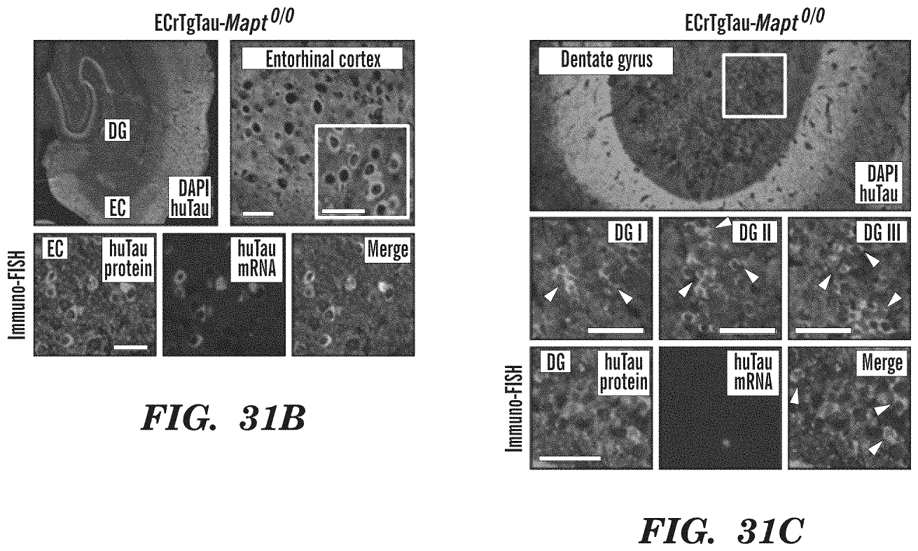

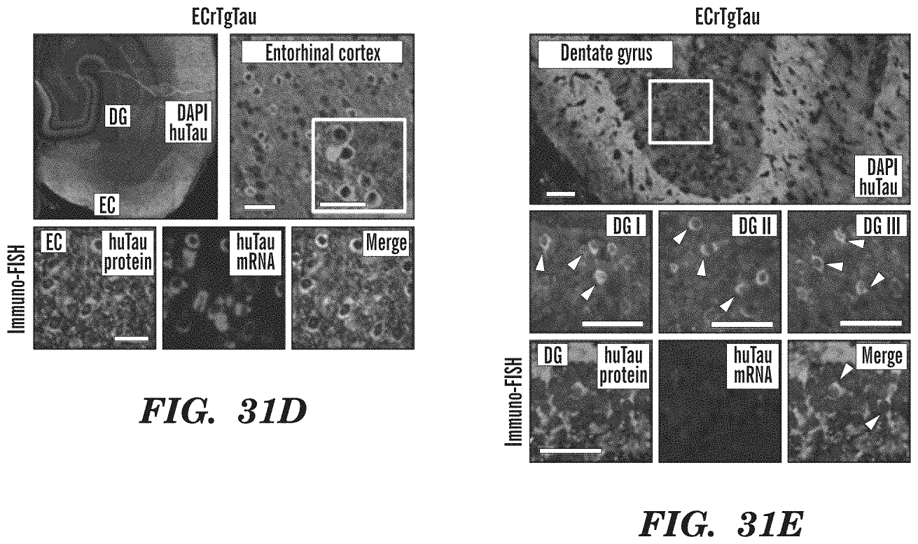

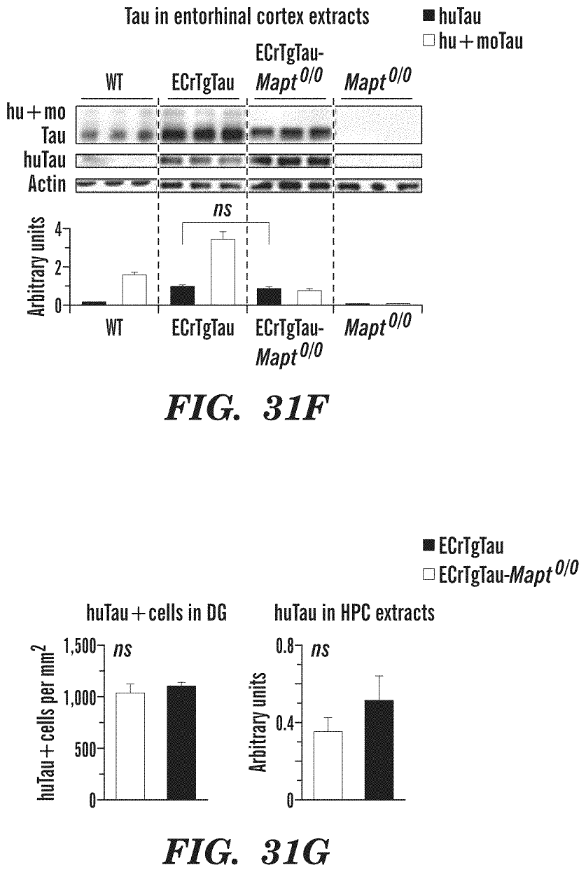

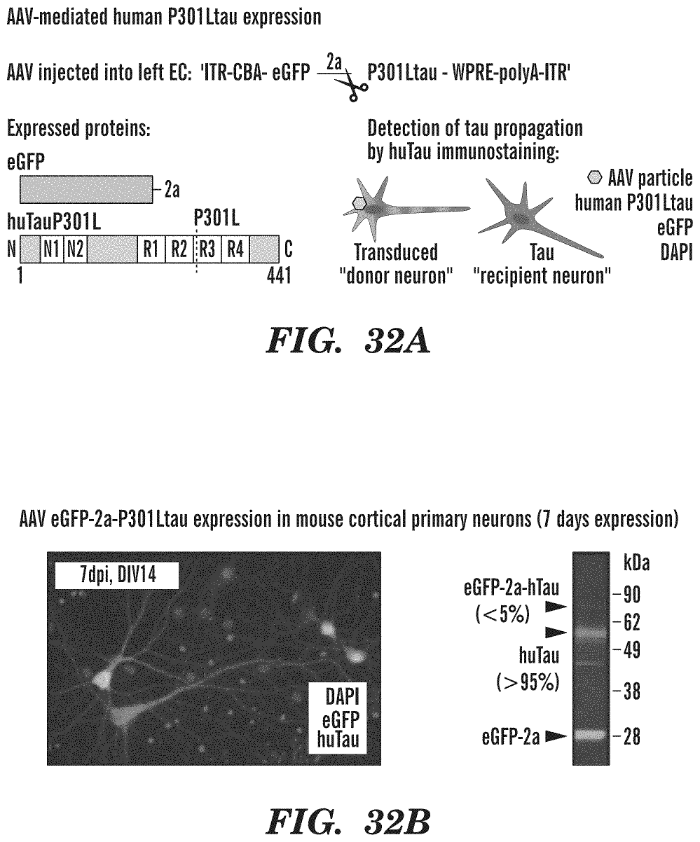

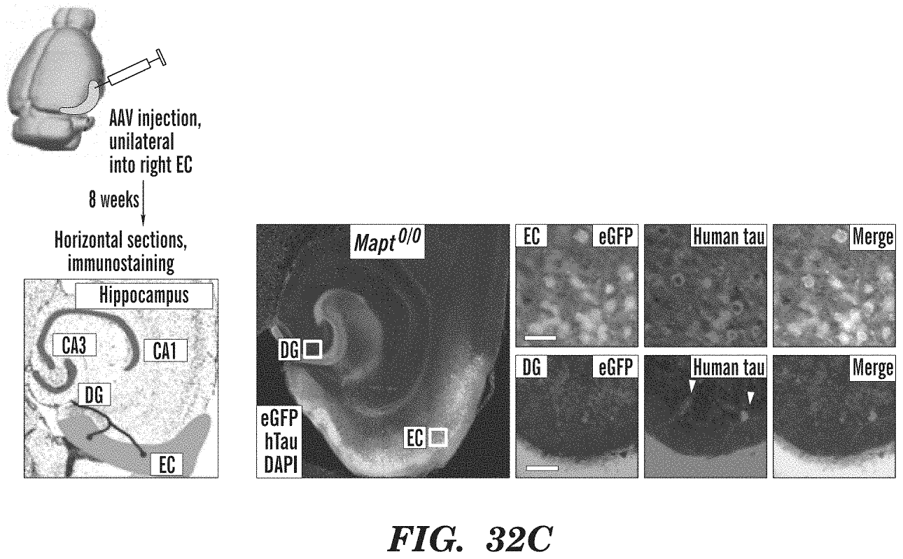

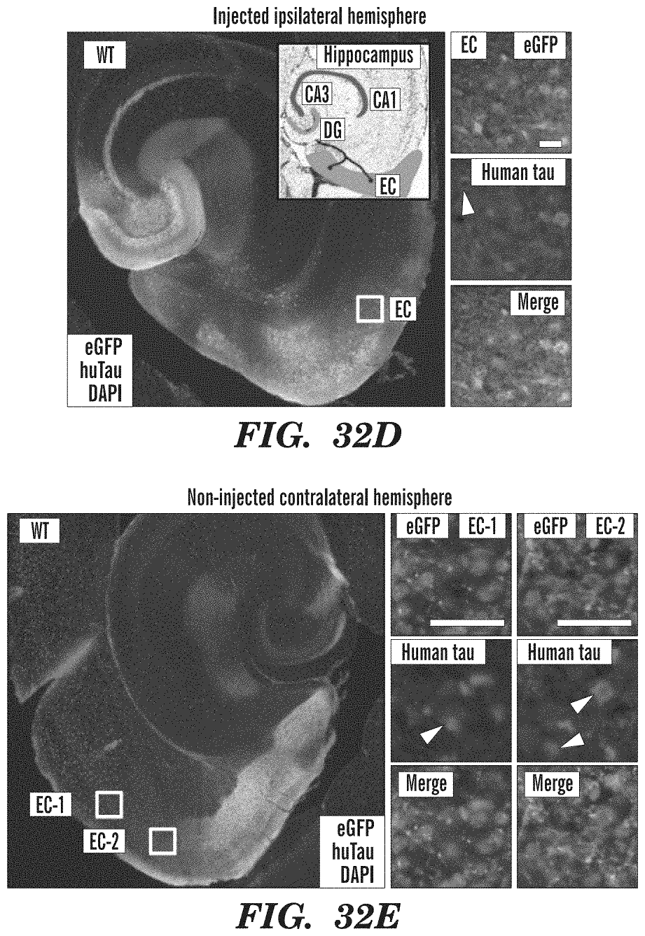

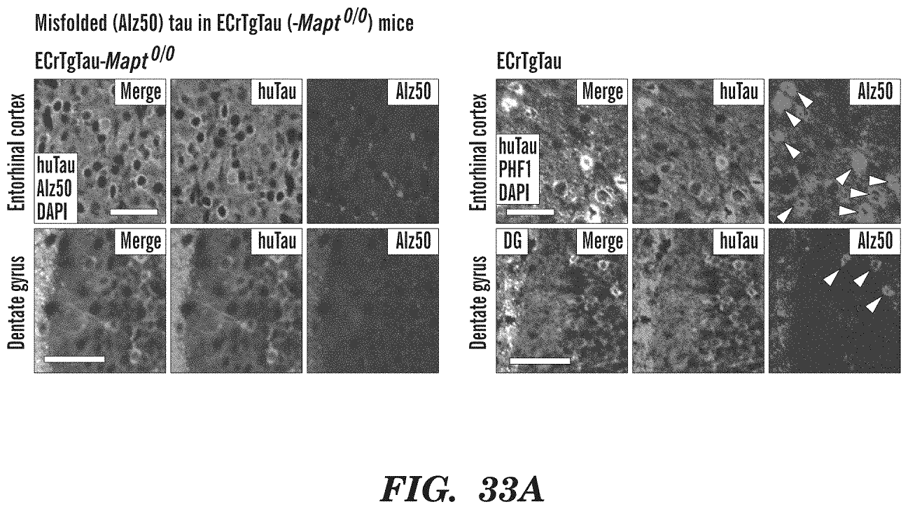

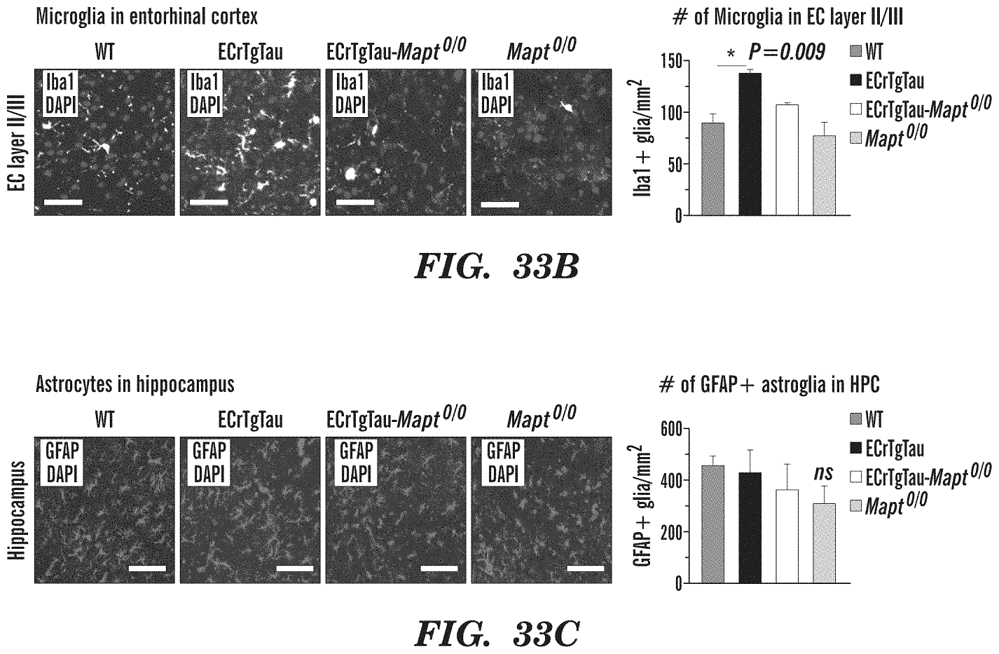

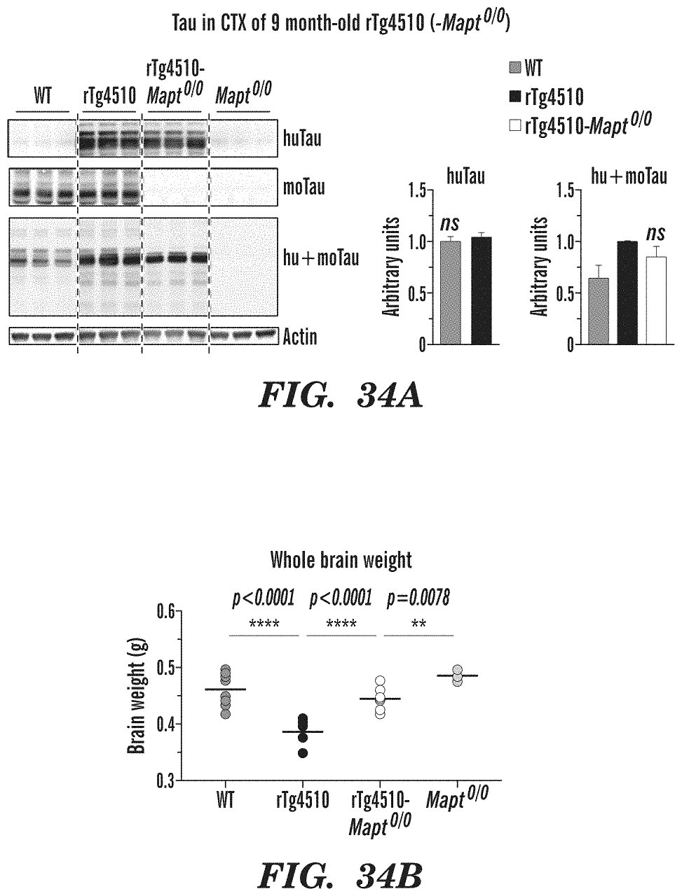

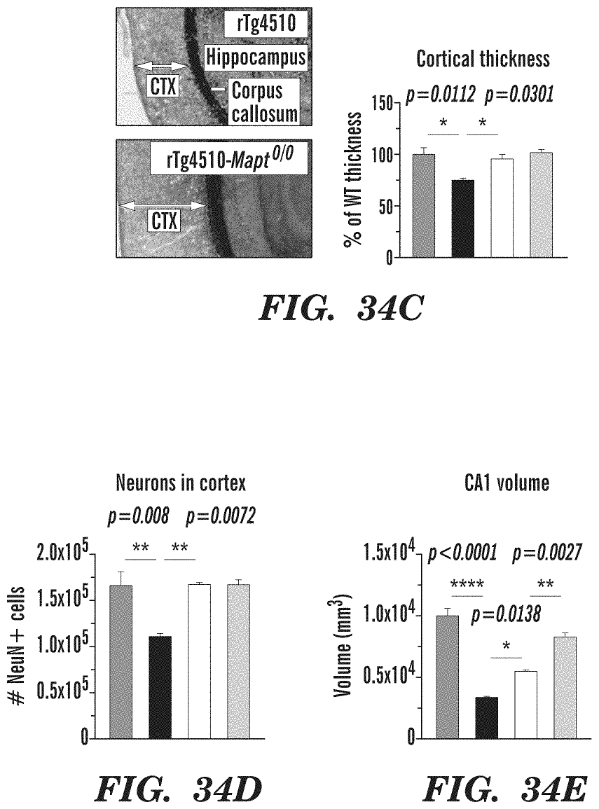

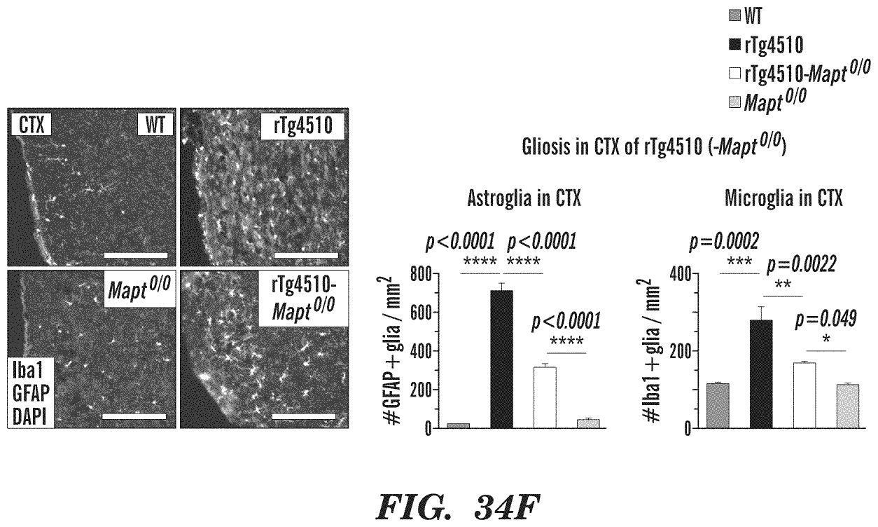

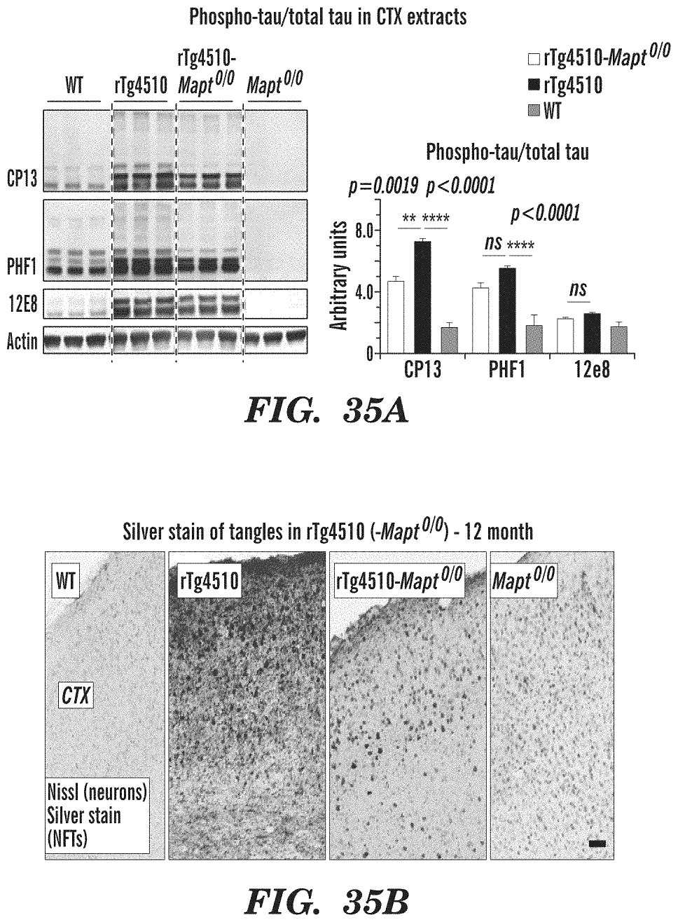

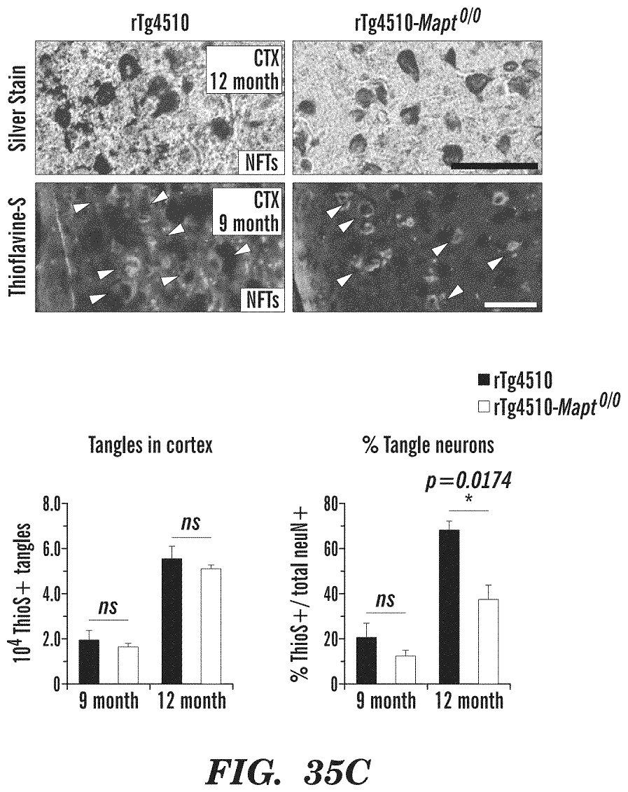

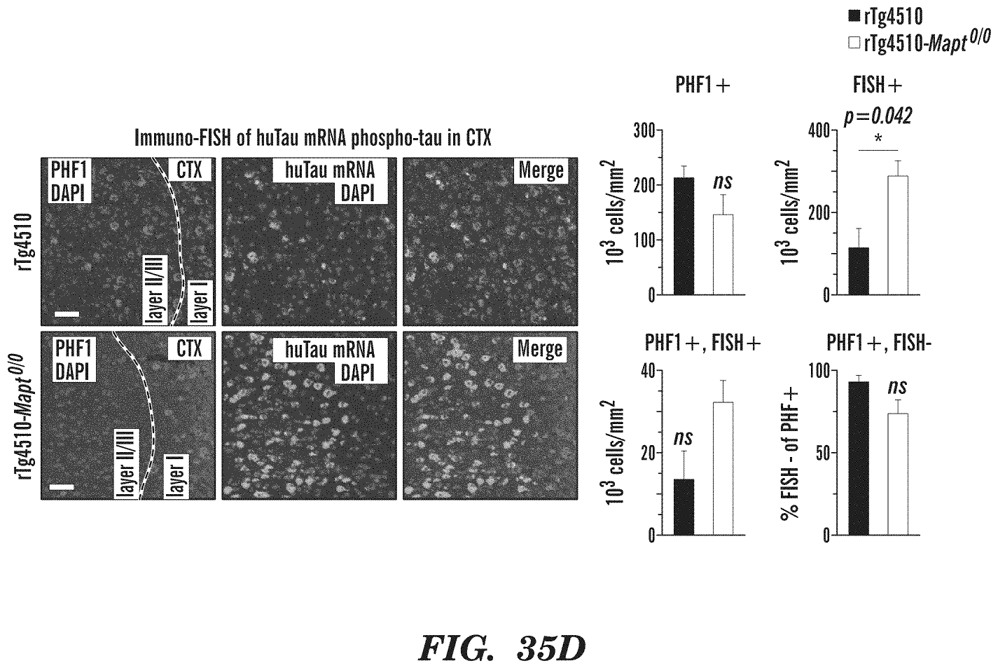

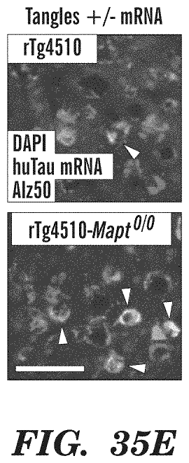

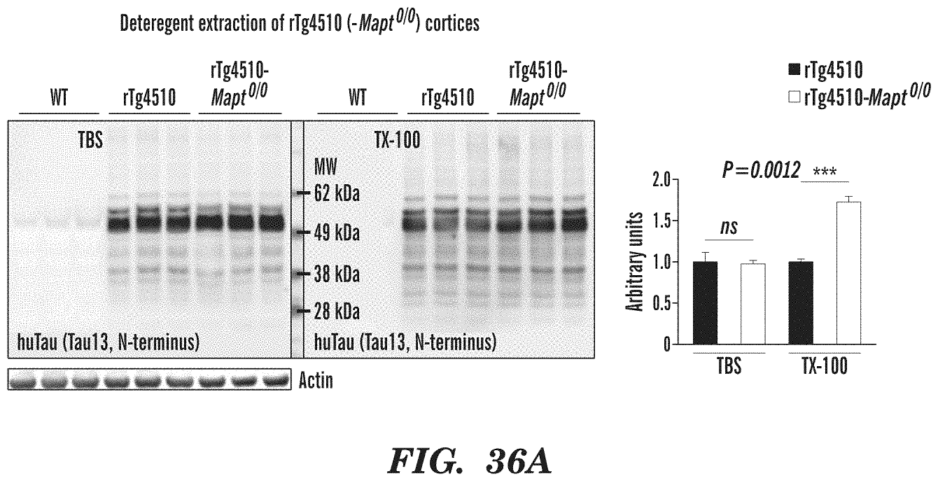

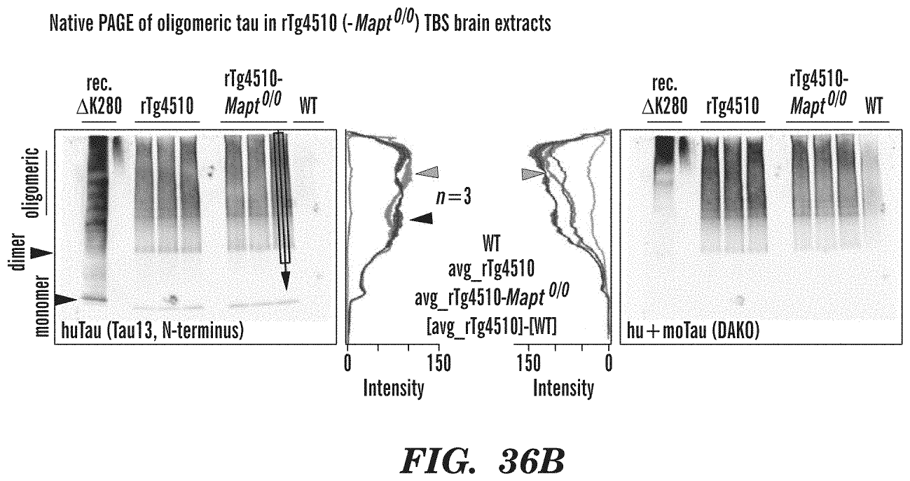

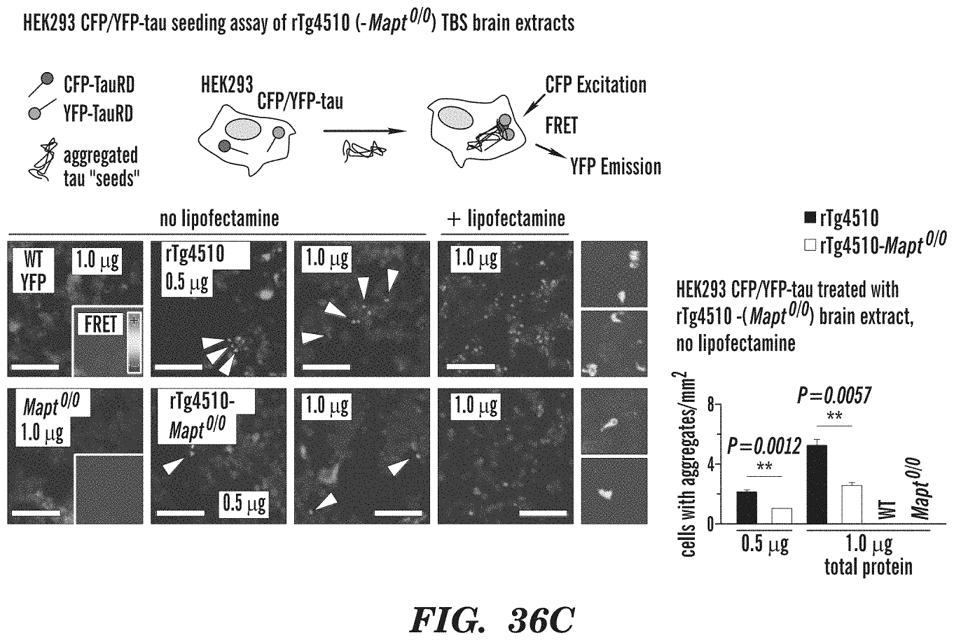

Human lumbar CSF samples were also collected from 8 AD human subjects and 13 control subjects CSF samples to analyze if HMW tau species is present in lumbar CSF. The total tau levels and phosphorylated tau levels were measured in the samples by ELISA using anti-total tau or anti-phospho-tau antibodies. The levels of Aβ1-42 were also measured in the lumbar CSF samples. FIG. 22A is a set of graphs showing total tau levels (left), phospho-tau levels (middle), and Aβ1-42 levels (right) in the lumbar CSF of AD and control human subjects. The levels of total tau and phosphorylated tau species were detected at higher concentrations in AD patients than in control subjects. Aβ1-42 was present at a higher level in the control samples than in the AD patients. FIG. 22B shows the correlation of total tau and Aβ1-42 levels in the lumbar CSF of AD and control human subjects. The AD patients have higher levels of total tau but lower level of Aβ1-42 in their lumbar CSF than in the control patients without AD.

Next, the human lumbar CSF samples from the AD and control patients were fractionated by SEC and the total tau in each faction is measured by human tau-specific ELISA to assess the molecular-weight size distribution of tau present in the samples. FIG. 22C shows total tau levels measured in each indicated fraction of the lumbar CSF collected from control subjects and AD subjects. The left graph shows the data of all 13 control subjects. The middle graph shows the data of all 8 AD subjects. The right graph shows the average total tau levels in each indicated fraction based on the measured data shown in the left and middle graphs. HMW tau species was detected in fraction 1 of the human lumbar CSF samples, and its concentration were substantially higher in AD patients than in control subjects (p<0.01).

The present findings indicate that CSF from AD brain contains a bioactive HMW tau species, which can be used as a biomarker for AD.

### Exemplary Materials and Methods

Animals.

Eleven- to 13-month-old rTg4510, rTg21221, and control animals were used. The rTg4510 (P301L tau) mouse is a well-characterized model of tauopathy, which overexpresses full-length human four-repeat tau (0N4R) with the P301L frontotemporal dementia (FTD) mutation 19. The rTg21221 mouse expresses WTT human tau at levels comparable to rTg4510 mouse and does not show accumulation of tau pathology in the brain (20). Littermate animals with only the activator CK-tTA transgene, which do not overexpress tau, were used as controls. Both male and female mice were used. All experiments were performed under national (United States National Institutes of Health) and institutional (Massachusetts General Hospital Subcommittee for Research Animal Care and the Institutional Animal Care and Use Committee at Harvard Medical School) guidelines.

Human Brain Samples.

Frozen brain tissues from the frontal cortex of four patients with AD, three non-demented control subjects were obtained from the Massachusetts Alzheimer's Disease Research Center Brain Bank. The demographic characteristics of the subjects are shown in Table 2 below. All the study subjects or their next of kin gave informed consent for the brain donation, and the Massachusetts General Hospital Institutional Review Board approved the study protocol. All the AD subjects fulfilled the NIA-Reagan criteria for high likelihood of AD. Cortical gray matter was weighed and processed as described in the following section (Brain extraction).

TABLE 2
Characteristics of the subjects with
AD and controls used in the study.

| | | | Post- | | Braak |
|---|---|---|---|---|---|
| | Age | | mortem- | | stage, |
| Case | at death | | interval | Diag- | CERAD |
| (sample #) | (years) | Sex | (hours) | nosis | score |
| AD (#1762) | 71 | Female | 16 | AD | VI, C |
| AD (#1745) | 85 | Male | 24 | AD | VI, C |
| AD (#1683) | 83 | Male | 10 | AD | VI, C |
| AD (#1497) | 82 | Female | 8 | AD | VI, C |
| Control (#1703) | 73 | Female | 20 | Control | — |
| Cortrol (#1669) | 86 | Male | 10 | Control | — |
| Cortrol (#1506) | 86 | Male | 10 | Control | — |

Cases were matched for age (80.3 ± 3.15 (AD) vs. 81.7 ± 4.33 (control) years, P = 0.796, t(5) = −0.273) and postmortem interval (14.5 ± 3.59 (AD) vs. 13.3 ± 3.33 (control) hours, P = 0.828, t(5) = 0.229).
Mean ± S.E.M., Student's t-test.

Brain Extraction.

Mice were perfused with cold PBS containing protease inhibitors (protease inhibitor mixture; Roche, USA), and the brain was rapidly excised and frozen in liquid nitrogen, then stored at −80° C. before use. Brain tissue was homogenized in 5 volumes (wt/vol) of cold PBS using a Teflon-glass homogenizer. The homogenate was briefly sonicated (Fisher Scientific Sonic Dismembrator Model 100, output 2, 6×1 sec) and centrifuged at 3,000×g for 5 min at 4° C. (3,000 g extract), 10,000×g for 15 min at 4° C. (10,000 g extract), 50,000×g for 30 min at 4° C. (50,000 g extract), or 150,000×g for 30 min at 4° C. (150,000 g extract). The supernatants were collected and stored at −80° C. before use.

Primary Cortical Neuron Culture.

Primary cortical neurons were prepared from cerebral cortices of embryonic day (E) 14-15 CD1 mouse embryos (Charles River Laboratories) as described in Danzer et al. (Ref #37) with modifications. Cortices were dissected out and mechanically dissociated in Neuro-basal (Life Technologies, Inc., USA) medium supplemented with 10% fetal bovine serum, 2 mM Glutamax, 100 U/ml penicillin, and 100 g/ml streptomycin (plating medium), centrifuged at 150 g for 5 min, and resuspended in the same medium. Neurons were plated at a density of 0.6×10⁵ viable cells on a Lab-Tek 8-well chambered coverglass (Nalge Nunc) or microfluidic devices (see below for cell density and protocol) previously coated with poly-D-lysine (50 μg/ml, Sigma) overnight. Cultures were maintained at 37° C. with 5% CO₂ in Neuro-basal medium with 2% (vol/vol) B27 nutrient, 2 mM Glutamax, 100 U/ml penicillin, and 100 g/ml streptomycin (culture medium).

Tau Uptake in Primary Neurons.

Mouse primary neurons (7-8 days in vitro) were incubated with PBS-soluble brain extracts (3,000 g, 10,000 g, 50,000 g, or 150,000 g centrifugation supernatant, or SEC fractions from 3,000 g extract) from mouse (rTg4510 or rTg21221 animals) or human (control or sporadic AD) brain tissues, microdialysate from rTg4510/control mice, or recombinant tau oligomer mixture solution. Neurons were maintained at 37° C. in 5% CO₂ in a humidified incubator. Each sample was diluted with culture medium to obtain the designated human tau concentrations (measured by human tau ELISA). Neurons were washed extensively with PBS, fixed, and immunostained with human tau-specific antibody (Tau13, # MMS-520R, Covance, 1:2000) to detect exogenously applied human tau in mouse primary neurons at the designated time point. For most experiments described in this Example, total (human and mouse) tau antibody (# A0024, DAKO, 1:1000) was used as a neuronal marker. Each sample was filtered through a 0.2-μm membrane filter to remove large aggregates and fibrils before incubation.

Tau Uptake and Seeding Activity Assay in HEK293-Tau-Biosensor Cells.

Stably expressing CFP-/YFP-TauRD(P301S) HEK293 cells (Holmes et al, 2014) (Ref. #23) were plated at 30,000 cells per well in a 96-well PDL-coated plate. The following day, PBS-soluble brain extracts (3,000 g) were applied at designated concentrations of human tau or total proteins in a total of 40 μL Opti-MEM (#11058-021, Life technologies) with (for tau seeding assay) or without (for tau uptake assay) Lipofectamine 2000 transfection reagent (#11668019, Life technologies). Cells were fixed with 4% PFA at designated time points after the extracts were applied and confocal images were obtained via FRET channel (excited with a 458 nm laser and fluorescence was captured with 500-550 nm filter). FRET density, defined as the number of FRET-positive tau aggregates multiplied by the mean fluorescence intensity of FRET-positive tau aggregates and then normalized by the number of cells (DAPI staining), was used for quantification analysis. Each condition was performed at least in triplicate.

In Vivo Tau Uptake Assay in WT Mice.

Stereotactic injections of brain extract were performed as described in Holmes et al. (Ref. #38) with some modifications. WT mice (male, 3 months old, male, C57BL6/J) were injected by using a 30-gauge Hamilton microsyringe in the left frontal cortex (bregma+1.3 mm, 1.5 mm lateral to midline, −1.6 mm relative to bregma) at an infusion rate of 0.2 μL/min. 2.5 μL of HMW (Frc.2-3) or LMW (Frc.13-14) SEC fractions (100 or 500 ng/ml human tau) from rTg4510 brain extract (male, 12 months old, PBS-soluble, 3,000 g) were injected. The same volume of PBS was injected as a negative control. Mice were killed 48 hours after injection and brain sections from frontal cortex were immunostained with human tau specific antibody (Tau13, # MMS-520R, Covance, 1:2000), chicken polyclonal anti-NeuN antibody (# ab134014, Abcam, 1:500), and counterstained with DAPI. Anti-mouse Alexa488 (1:1000) and CY3-labeled anti-chicken IgG (1:1000) secondary antibodies were used to detect human tau and NeuN, respectively (see also Immunostaining of brain sections). Images were acquired on an AxioImager Z1 epifluorescence microscope (Carl Zeiss, Oberkochen, Germany). Images were semi-quantitatively evaluated for human tau staining by a rater who was blinded to the experimental conditions, using a score from 0 (no human tau labeling on NeuN positive neurons) to 4 (maximum human tau labeling on NeuN positive neurons).

In Vivo Tau Uptake Assay in Young rTg4510 Mice.

Hippocampal injections of brain extract were performed as described in Sanders et al. (Ref #13) with some modifications. rTg4510 mice (male, 2 to 3 months old, pre-tangle stage) were injected by using a 30-gauge Hamilton microsyringe in the left hippocampus (bregma −2.5 mm, 2.0 mm lateral to midline, −1.8 mm relative to bregma) at an infusion rate of 0.2 μL/min. 2.5 μL of HMW (Frc.2-3) or LMW (Frc.13-14) SEC fractions (100 ng/ml human tau) from rTg4510 brain extract (male, 12 months old, PBS-soluble, 3,000 g) were injected. The same volume of PBS was injected as a negative control. Mice were killed three weeks after injection and serial coronal brain sections (40 μm) were taken though the entire brain. Sections were incubated with 0.3% hydrogen peroxide for 10 min at R.T., blocked in 3% milk in TBS with 0.25% Triton X-100, and incubated with biotinylated AT8 antibody (ThermoScientific, MN1020B, 1:1000) in 3% milk in TBS with 0.25% Triton X-100 overnight at 4° C. After washing in TBS, sections were developed with nickel-enhanced DAB substrate using the VECTASTAIN Elite ABC Kit (Vector Laboratories). Every seventh section was stained. Images were obtained using an Olympus BX51 microscope mounted with a DP 70 Olympus digital camera. The number of AT8-positive neurons was manually counted by a blinded investigator (seven sections for each mouse).

Atomic Force Microscopy (AFM).

Immunoprecipitation (IP) isolation of tau from rTg4510 brain extract for AFM analysis was performed as described in Lasagna-Reeves et al. (Ref. #39) with some modifications. Tosylactivated magnetic Dynabeads (#14203, Life Technologies) were coated with human tau-specific Tau13 antibody. Beads were washed (0.2 M Tris, 0.1% bovine serum albumin, pH 8.5) and incubated with HMW SEC fraction (Frc.3 from 10,000 g extract, rTg4510) sample for 1 hour at R.T. Beads were washed three times with PBS and eluted using 0.1 M glycine, pH 2.8, and the pH of each eluted fraction was immediately adjusted using 1 M Tris pH 8.0. For AFM imaging, isolated tau fractions were adsorbed onto freshly cleaved muscovite mica and imaged using oscillation mode AFM (Nanoscope m, Di-Veeco, Santa Barbara, Calif.) and Si₃N₄ cantilevers (NPS series, Di-Veeco) in PBS, as described in Wegmann et al. (Ref. #24). For size (AFM heights) distribution histogram of HMW tau oligomers (SEC Frc. 3), 1206 particles from seven randomly-picked images (1.5 μm×1.5 μm) were analyzed (FIG. 1H).

In Vivo Microdialysis.

In vivo microdialysis sampling of brain interstitial fluid tau was performed as described in Takeda et al. (Ref. #21) and Takeda et al. (Ref. #22). The microdialysis probe had a 4 mm shaft with a 3.0 mm, 1000 kDa molecular weight cutoff (MWCO) polyethylene membrane (PEP-4-03, Eicom, Japan). This probe contains a ventilation hole near the top which serves to produce a reservoir of fluid within the probe that is open to the atmosphere. This structure minimizes pressure which would otherwise cause a net flow of perfusate out through the large pore membrane. Before use, the probe was conditioned by briefly dipping it in ethanol, and then washed with an artificial cerebrospinal fluid (aCSF) perfusion buffer (in mM: 122 NaCl, 1.3 CaCl₂), 1.2 MgCl₂, 3.0 KH₂PO₄, 25.0 NaHCO₃) that was filtered through a 0.2 μm pore-size membrane. The preconditioned probe's outlet and inlet were connected to a peristaltic pump (ERP-10, Eicom) and a microsyringe pump (ESP-32, Eicom), respectively, using fluorinated ethylene propylene (FEP) tubing (σ 250 μm i.d.).

Probe implantation was performed as previously in Takeda et al. (Ref. #21), with some modifications. The animals were anesthetized with isoflurane, while a guide cannula (PEG-4, Eicom) was stereotactically implanted in the hippocampus (bregma −3.1 mm, −2.5 mm lateral to midline, −1.0 mm ventral to dura).

Three or four days after the implantation of the guide cannula, the mice were placed in a standard microdialysis cage and a probe was inserted through the guide. After insertion of the probe, in order to obtain stable recordings, the probe and connecting tubes were perfused with aCSF for 180 min at a flow rate of 10 μl/min before sample collection. Samples were collected at a flow rate of 0.5 μl/min.

Tau ELISA.

The concentrations of human tau in the samples (brain extracts, brain ISF samples, and recombinant human tau solution, and SEC-separated samples) were determined by Tau (total) Human ELISA kit (# KHB0041, Life Technologies) and Tau [pS396] Human ELISA kit (# KHB7031, Life Technologies), according to the manufacturer's instructions.

Immunoblot Analysis.

Brain extracts were electrophoresed on Novex Tris-Glycine gels (Life Technologies, Grand Island, N.Y., USA) in Tris-Glycine SDS running buffer for SDS-PAGE (Life Technologies). Gels were transferred to PVDF membranes, and membranes were blocked for 60 min at RT. in 5% (wt/vol) BSA/TBS-T, and then probed with primary antibodies overnight at 4° C. in 2% (wt/vol) BSA/TBS-T. The following primary antibodies were used: mouse monoclonal antibody DA9 (total tau (aa112-129), courtesy of Peter Davies, 1:5000), mouse monoclonal antibody PHF1 (pS396/pS404 tau, courtesy of Peter Davies, 1:5000), mouse monoclonal antibody CP13 (pS202 tau, courtesy of Peter Davies, 1:1000), rabbit polyclonal anti-phospho tau antibodies (pS199 (#44734G), pT205 (#44738G), pS262 (#44750G), pS396 (#44752), pS400 (#44754G), pS404 (#44758G), pS409 (#44760G), and pS422 (#44764G)) from Life Technologies (1:2000 dilution for these antibodies), and mouse monoclonal anti-actin antibody (# A4700, Sigma-Aldrich, 1:2500). After washing three times in PBS-T, blots were incubated with HRP-conjugated goat anti-mouse (#172-1011, Bio-Rad) or anti-rabbit (#172-1019, Bio-Rad) IgG secondary antibodies (1:2000 dilution) for 1 hour at R.T. Immunoreactive proteins were developed using an ECL kit (Westem Lightning, PerkinElmer, USA) and detected on Hyperfilm ECL (GE healthcare, USA). 15 μg protein/lane were loaded, unless indicated otherwise. Scanned images were analyzed using Image J (National Institutes of Health).

Dot blot analysis. For dot blot, brain extracts (0.75 μg protein in 1.5 μl) were spotted directly onto nitrocellulose membranes (#88018, Thermo Scientific). Membranes were blocked for 60 min at R.T. in 5% (wt/vol) BSA/TBS-T, and then probed with primary antibodies for 60 min at R.T. in 2% (wt/vol) BSA/TBS-T. The following primary antibodies were used: rabbit polyclonal tau oligomer-specific antibody T22 (# ABN454, Millipore, 1:1000)40, mouse monoclonal antibody Tau13 (# MMS-520R, Covance, 1:2000), rabbit polyclonal anti-total tau antibody (# ab64193, Abcam, 1:1000). After washing three times in PBS-T, blots were incubated with HRP-conjugated goat anti-mouse (#172-1011, Bio-Rad) or anti-rabbit (#172-1019, Bio-Rad) IgG secondary antibodies (1:2000 dilution) for 60 min at R.T. Immunoreactive proteins were developed using an ECL kit (Western Lightning, PerkinElmer, USA) and detected on Hyperfilm ECL (GE healthcare, USA).

Urea/SDS Treatment.

The rTg4510 brain extracts (12 months old, PBS-3,000 g) were incubated with 8 M urea or 10% SDS (1.0 μg/μl total protein in 8 M urea or 10% SDS) for 24 hours at 37° C. prior to application to the membrane for dot blot analysis. The SEC HMW fractions (Frc.2) from the rTg4510 brain extracts (12 months old, PBS-3,000 g) were incubated with 8 M urea for 24 hours at 37° C. and analyzed by SDS-PAGE (non-reducing condition) using total tau antibody (# A0024, DAKO, 1:1000).

Semi-Denaturing Detergent Agarose Gel Electrophoresis (SDD-AGE).

Semi-denaturing detergent agarose gel electrophoresis (SDD-AGE) was carried out as described in Sanders et al. (Ref #13) with some modifications. Brain extract was thawed on ice. A 1.5% agarose gel was prepared by dissolving agarose in buffer G (20 mM Tris-Base, 200 mM glycine) and then adding 0.02% SDS. A total of 50 μg (for Tg4510 and Tg21221 brain extracts (FIG. 16)) or 25 μg (for lambda phosphatase experiment (FIG. 7E)) of brain extract protein was incubated with 0.02% SDS sample buffer for a total of 7 minutes at room temperature just prior to loading. The SDD-AGE was run in Laemmli buffer (Buffer G with 0.1% SDS) at 30V for 14 hours or until the dye front reached the end of the gel. Protein was then transferred via capillary action to Immoblin P (Millipore) membrane at 4° C. for 16-24 hours. Membranes were blocked in 5% non-fat dry milk (NFDM)/TBS-T for 1 hour and then probed for total tau using rabbit polyclonal anti-tau antibody (# ab64193, Abcam, 1:4000) overnight at 4° C. Membranes were washed three times with TBS-T, probed with goat anti-rabbit IgG-HRP (#172-1019, Bio-Rad, 1:2000) for 1.5 hours at room temperature, and washed three times with TBS-T. Membranes were developed using ECL kit (Western Lightning, PerkinElmer, USA) and detected on Hyperfilm ECL (GE healthcare, USA).

Immunodepletion.

Immunodepletion of tau from rTg4510 brain extracts was performed using Dynabeads Protein G Immunoprecipitation Kit (#10007D, Life Technologies) according to the manufacturer's instructions with some modifications. 0.75 mg of Dynabeads Protein G was incubated with 1 μg of anti-tau antibodies (anti-phospho tau antibodies (see Immunoblot analysis), total tau antibody (HT7, # MN1000, Thermo Scientific), and control IgG) for 10 min with rotation at room temperature. After washing with 200 μl of washing buffer, the Dynabeads-antibody complex was incubated with 300 μl of rTg4510 brain extracts (12 months old, PBS-3,000 g, 500 ng/ml human tau) for 10 min with rotation at room temperature. Dynabeads-antibody-antigen complex was isolated using a magnetic holder and the supernatant was collected for tau uptake assay and ELISA measurement. After washing three times with 100 μl of washing buffer, Dynabeads-antibody-antigen complex was resuspended in 20 μl of elution buffer and incubated for 2 min at room temperature. Dynabeads-antibody complex was isolated suing a magnetic holder and supernatant (immunoprecipitated tau) was collected for tau ELISA measurement.

Lambda Phosphatase Treatment.

25 μg protein of brain extract (12-month-old rTg4510, PBS-3,000 g) was incubated for 1 hour at 30° C. with 400 units of Lambda Protein Phosphatase (NEB) supplemented with IX NEBuffer for protein metalophosphatase (PMP) and 1 mM MnCl₂, immediately followed by 1 hour at 65° C. to inactivate the Lambda Phosphatase enzymatic activity.

Size-Exclusion Chromatography (SEC).

Brain PBS-soluble extracts, ISF microdialysate, oligomer tau (recombinant hTau-441) mixture solution were separated by size-exclusion chromatography (SEC) on single Superdex200 10/300GL columns (#17-5175-01, GE Healthcare) in phosphate buffered saline (# P3813, Sigma-Aldrich, filtered through a 0.2-μm membrane filter), at a flow rate of 0.5 ml/min, with an AKTA purifier 10 (GE Healthcare). Each brain extract was diluted with PBS to contain 6000 ng of human tau in a final volume of 350 μl, which was filtered through a 0.2-μm membrane filter and then loaded onto an SEC column. The individual fractions separated by SEC were analyzed by ELISA (Tau (total) Human ELISA kit, diluted 1:50 in kit buffer). For the ISF sample, 400 μl of microdialysate from rTg4510 mice was loaded onto the column after filtration through a 0.2-μm membrane, and SEC fractions were measured by human tau ELISA. For the oligomer tau mixture solution, 500 μl of sample (hTau-441, 3.35 mg/ml with 2 mM DTT, filtered through a 0.2-μm membrane filter) was loaded onto column and each SEC-fraction was diluted 1:200,000 in kit buffer for human tau ELISA.

Immunocytochemistry.

Primary neurons were washed extensively with PBS (three times) and fixed with 4% paraformaldehyde (PFA) for 15 min. Neurons were washed with PBS, permeabilized with 0.2% Triton X-100 in PBS for 15 min (R.T.), blocked with 5% normal goat serum (NGS) in PBS-T (R.T.), and then incubated with the primary antibodies overnight at 4° C. in 2% NGS/PBS-T. The following primary antibodies were used to detect tau: mouse monoclonal antibody Tau13 (specific for human tau (aa20-35), # MMS-520R, Covance, 1:2000), rabbit polyclonal anti-total tau antibody (recognizes both human and mouse tau, # A0024, DAKO, 1:1000), mouse monoclonal antibody Alz50 (the conformation-specific antibody, courtesy of Peter Davies, Albert Einstein College of Medicine; 1:100). Goat anti-mouse Alexa488 and anti-rabbit Alexa555 secondary antibodies (Life Technologies, 1:1,000) were applied in 2% NGS in PBS-T for 1 hour at R.T. CY3-labeled anti-mouse IgM secondary antibody (Invitrogen, 1:200) was used to detect Alz50. After washing in PBS, coverslips were mounted with aqueous mounting medium (Vectashield). For co-staining with ThioS, neurons were first immunostained with rabbit polyclonal antibody TAUY9 (specific for human tau (aa12-27), # BML-TA3119-0025, Enzo Life Sciences, 1:200) and goat anti-rabbit Alexa555 secondary antibody (Invitrogen, 1:200), and then incubated with 0.025% (wt/vol) ThioS in 50% ethanol for 8 min. ThioS was differentiated in 80% ethanol for 30 s. Neurons were washed with water for 3 min, and coverslips were mounted using mounting medium (Vectashield). For co-staining with microtubule associated protein 2 (MAP2) and glial fibrillary acidic protein (GFAP), chicken polyclonal anti-MAP2 antibody (# ab5392, Abcam, 1:1000) and rabbit polyclonal anti-GFAP antibody (# ab7260, Abcam, 1:1000) were used for primary antibodies, and CY3-labeled anti-chicken IgG (1:1000) and anti-rabbit Alexa350 (1:500) secondary antibodies were used to detect MAP2 and GFAP, respectively.

The subcellular localization of human tau was studied by co-staining with the following primary antibodies: rabbit polyclonal anti-TGN46 antibody (the Golgi apparatus marker, # ab16059, Abcam, 1:200), rabbit polyclonal anti-GRP94 antibody (the endoplasmic reticulum marker, # ab3670, Abcam, 1:100), rabbit polyclonal anti-LAMP2a antibody (the lysosome marker, # ab18528, Abcam, 1:200), rabbit polyclonal anti-Rab5 antibody (the endosome marker, # ab13253, Abcam, 1:200), rabbit polyclonal anti-catalase antibody (the peroxisome marker, # ab1877, Abcam, 1:200). Goat anti-rabbit Alexa555 secondary antibody (Invitrogen, 1:200) was used to detect subcellular markers. Images were acquired using confocal microscope (Zeiss Axiovert 200 inverted microscope, Carl Zeiss).

Immunostaining of Brain Sections.

Mice were euthanized by CO₂ asphyxiation and perfused with PBS. Brains were fixed in 4% PFA for 2 days at 4° C., incubated for 2 days in 30% sucrose in PBS, and then 40-μm-thick sections were cut on a freezing sliding microtome. Sections were permeabilized with 0.2% Triton-X 100 in PBS, blocked in 5% NGS in PBS, and incubated in primary antibody (mouse monoclonal antibody Alz50 (courtesy of Peter Davies, 1:100)) in 2% NGS in PBS overnight at 4° C. CY3-labeled anti-mouse IgM secondary antibody (Invitrogen, 1:200) was applied in 2% NGS in PBS for 1 hour (R.T.). After washing in PBS, brain slices were mounted on microscope slides, and coverslips were mounted using DAPI containing mounting medium (Vectashield). NFTs were stained with 0.025% ThioS in 50% ethanol for 8 min. ThioS was differentiated in 80% ethanol for 30 sec. Sections were washed with water for 3 min, and coverslips were mounted using mounting medium.

Recombinant Human Tau Expression and Preparation of Tau Oligomer Mixture Solution.

Human full-length wild-type tau (2N4R, 441 aa) was expressed in E. coli BL21 DE3 using tau/pET29b plasmid (Adgene). Expression was induced at OD=0.6 by adding 1 mM IPTG for 3.5 hours at 37° C. Tau purification was performed by heat treatment and FPLC Mono S chromatography (Amersham Biosciences) as described in Barghorn et al. (Ref. #41). Cells of 300 ml culture were boiled in 3 ml buffer solution [50 mM MES, pH 6.8, 500 mM NaCl, 1 mM MgCl2, 5 mM DTT] for 20 min. Whole cell lysate was ultracentrifuged at 125,000 g for 45 min, and supernatant was dialyzed (MWCO 20 kDa) against 20 mM MES, pH 6.8, 50 mM NaCl, 2 mM DTT. Protein and tau content was determined by BCA assay kit (Pierce), SEC, and Western Blot. Tau oligomer mixture solution was prepared by incubating recombinant human tau (3.35 mg/ml) with 2 mM DTT for 2 days at 37° C., followed by SEC separation and ELSA measurement of tau (FIG. 7A).

Microfluidic Three-Chamber Devices.

A novel neuron-layering microfluidic platform was designed. The neuron-layering microfluidic platform comprised three distinct chambers connected through microgroove arrays (3×8×600 μm in height, width, and length) using standard soft lithographic techniques (42). The length of the microgrooves was such that no MAP2-positive dendrites entered the adjacent chambers (FIG. 3B). Taylor et al. previously reported that a 450 μm microgrooves are sufficiently long to isolate axon terminals from soma and dendrites (26). The platform was punched on two side reservoirs of each chamber and bonded to a poly-D-lysine (50 μg/ml, Sigma) coated glass-bottom dish (# P50G-1.5-30-F, MatTek Corporation) to enhance neuronal adhesion.

First, primary cortical neurons isolated from E15 mouse embryos were plated into the 1st chamber at an approximate density of 0.6×10⁵ viable cells (in 10 μl plating medium) per device. After 15 min (most neurons in the 1st chamber adhered to the bottom of dish during this period), 0.3×10⁵ viable cells in 2 μl plating medium were loaded into the 2nd chamber via one of the side reservoirs. Microfluidic devices were set at a tilt (approximately an 80 degree angle) in an incubator (37° C. in 5% CO2) immediately after neurons were plated into the 2nd chamber, so that the neurons would settle down to surfaces close to the third chamber by gravity. Therefore, most neurons in the 2nd chamber were plated in a line along a sidewall of the 2nd chamber, which had microgrooves connecting to the 3rd chamber. This protocol was empirically established to allow most of the axons of neurons in the 2nd chamber to extend into the 3rd chamber (FIG. 3B). After 3 hours, the devices were set in a normal position without tilting and maintained at 37° C. in 5% CO₂ in culture medium. The medium was changed every 4-5 days.

For tau uptake and the propagation assay, PBS-soluble extracts from rTg4510 and AD brain tissue were added to the 1st chamber (10μ in total) on 7 or 8 days in vitro. The 2nd and 3rd chambers were filled with 40 μl of media (20 μl in each reservoir). The volume difference between the chambers resulted in continuous convection (“hydrostatic pressure barrier”; 10 μl in the 1st chamber and 40 μl in the 2nd and 3rd chambers), which prevented diffusion of brain extract from the 1st chamber into other chambers. After incubation for the designated periods, neurons were washed, fixed, and immunostained as described above (see “Immunocytochemistry”).

Transfections of GFP and RFP (FIG. 3C) were carried out inside microfluidic devices as follows: Initially, the 1st chamber neurons (7 DIV) were transfected with GFP (0.04 μg DNA+0.1 μl of Lipofectamine 2000 (#11668-019, Life Technologies) in 10 μl of NeuroBasal for 3 hours. Diffusion of DNA from the 1st chamber to the 2nd chamber was prevented by a hydrostatic pressure barrier, as described above. After washing the DNA (GFP)-containing medium from the 1st chamber (washed three times with NeuroBasal), the 2nd chamber neurons were transfected with RFP (0.04 μg DNA+0.1 μl of Lipofectamine 2000 in 10 μl NeuroBasal) for 3 hours. Diffusion of DNA from the 2nd chamber to the 1st and 3rd chambers was prevented by the hydrostatic pressure barrier (40 μl media in the 1st and 3rd chambers). After washing the DNA (RFP)-containing medium from the 2nd chamber (washed three times with NeuroBasal), devices were maintained at 37° C. in 5% CO₂ in culture medium. Expressions of GFP and RFP were examined 2 days later.

Neurons in the microfluidic device were examined using a Zeiss Axiovert 200 inverted microscope (Carl Zeiss) equipped with a Zeiss LSM 510 META (Zeiss, Jena, Germany) confocal scanhead using 488- and 543-nm lasers. All images were acquired using a 25×APO-Plan Neoflu lens or 63×1.2 NA C-APO-Plan Neoflu lens (Carl Zeiss).

Ethidium Homodimer-1 (EthD-1) Staining.

Cell viability assay with EthD-1 staining (# L-3224, Life Technologies) was performed according to the manufacturer's instructions with some modifications. Cells were washed twice with PBS and incubated with EthD-1 (4 μM in PBS) and Hoechst 33342 (# H3570, Life Technologies, 1 μg/ml in PBS) for 20 min at 37° C. in 5% CO₂ in a humidified incubator. Images were acquired using confocal microscope (Zeiss Axiovert 200 inverted microscope, Carl Zeiss).

Mtf Assay.

Neuronal viability was assessed using a 3-(4,5-dimethylthiazol-2-yl)-2,5-diphenyl tetrazolium bromide (MTT) assay kit (TACS MTT Cell Proliferation Assays, #4890-25-K, Trevigen) according to the manufacturer's instructions with some modifications. Mouse primary neurons were incubated with HMW or LMW SEC fractions from rTg4510 brain extracts (12 months old, PBS-3,000 g, 10 ng/ml human tau) in 96-well plate (Corning, #3603) (2.5×104 cells/well in 100 μl) for 48 hours. 10 μl of MTT was added to each well at 48 hours and neurons were incubated for 2 hours in an incubator (37° C. in 5% CO₂). When purple precipitate is visible under the microscope, 100 μl of detergent reagent was added to each well and incubated for 4 hours at room temperature. The absorbance of each well was measured at 600 nm in a microplate reader (Wallac Victor 1420 Multilabel Counter, Perkin Elmer).

Statistical Analysis.

All data were expressed as mean±S.E.M Two-group comparisons were performed by unpaired t-test, unless stated otherwise. Comparison among three or more groups was performed by analysis of variance (ANOVA) and Tukey-Kramer multiple range test, unless stated otherwise. Correlations were analyzed with Pearson correlation analysis. P values less than 0.05 were considered significant.

Subjects:

Study participants were recruited from the Clinical Cores of Massachusetts General Hospital and the University of Washington Alzheimer's Disease Research Center (ADRC). All participants underwent standard neuropsychological evaluation and clinical examination. Informants were also interviewed and a clinical diagnosis was assigned at consensus diagnostic meetings. All control subjects had Mini-Mental State Examination (MMSE) scores of ≥28, and neither subjects nor informants reported changes in social/occupational functioning suggesting a decline in cognitive function. Diagnosis of probable AD was made based on standard criteria of NINCDS-ADRDA 18. Following written informed consent, lumbar punctures were performed between 9 am and 12:30 μm and the CSF was immediately aliquoted into polypropylene storage tubes and placed on dry ice. Specimens were then transferred and stored at −80° C. before use. Subjects were recruited for lumbar punctures at both Massachusetts General Hospital and the University of Washington, and assays were performed at Massachusetts General Hospital. The demographic and clinical characteristics of all study participants are detailed in Tables 4 and 5. Lumbar CSF samples were obtained from fifteen clinically diagnosed AD patients, ten FTD patients, and nineteen non-demented control subjects.

Ventricular CSF: Postmortem ventricular CSF samples were obtained from the Massachusetts Alzheimer's Disease Research Center Brain Bank. The demographic characteristics of the subjects are shown in Table 3. All the study subjects or their next of kin gave informed consent for the brain donation, and the Massachusetts General Hospital Institutional Review Board approved the study protocol. All the AD subjects fulfilled the NIA-Reagan criteria for high likelihood of AD. Ventricular CSF samples were collected by syringe aspiration from the lateral ventricles. Specimens were stored at −80° C. before use. The total tau levels in each sample were determined by tau ELISA (# KHB0041, Life Technologies).

TABLE 3
Characteristics of the subjects used in the ventricular CSF study.

| | | Age | | | |
|---|---|---|---|---|---|
| | | at death | | | |
| Case (sample #) | | (years) | Sex | Diagnosis | Braak stage |
| AD | #1318 | 93 | Male | AD, CAA | VI |
| | #1319 | 56 | Female | AD | VI |
| | #1362 | 79 | Male | AD | VI |
| | #1223 | 78 | Female | AD | V |
| | #1226 | 83 | Male | AD | VI |
| non-AD | #1453 | 66 | Female | FTD | I |
| | #1589 | 66 | Male | ALS | — |
| | #1477 | 92 | Male | Control | II |

Postmortem ventricular CSF samples were obtained from the Massachusetts Alzheimer's Disease Research Center Brain Bank. The demographic characteristics of the subjects are shown in Table 3. To conduct a more detailed biochemical analysis, a large sample volume of human ventricular CSF obtained at autopsy was utilized. Postmortem ventricular CSF samples were obtained from five cases of neuropathologically confirmed AD (Braak stage V-VI) and three non-AD cases (Table 3).

TABLE 4
Demographic data of study participants.

| | AD | Control | FTD | p value |
|---|---|---|---|---|
| Number | 15 | 19 | 10 | |
| Age at LP | 71.1 ± 6.8 | 69.7 ± 7.4 | 63.8 ± 8.0 | P = 0.089 |
| (years), | | | | (Kruskal- |
| mean (SE) | | | | Wallis test) |
| Gender | 10:5 | 8:11 | 8:2 | |
| (male:female) | | | | |
| MMSE, mean | 24.3 ± 4.3^{a} | 29.6 ± 0.6 | 18.3 ± 9.6^{a} | P < 0.0001 |
| (SE) | | | | (Kruskal- |
| | | | | Wallis test) |

Abbreviations:
AD, Alzheimer's disease;
FTD, frontotemporal dementia;
LP, lumbar puncture;
MMSE, Mini-Mental State Examination.
aP < 0.01compared to control (Steel-Dwass multiple comparison test)

TABLE 5
Characteristics of study participants.

| | | Age at LP | Age of onset | Education | | apoE | |
|---|---|---|---|---|---|---|---|
| Diagnosis | Gender | (years) | (years) | (years) | MMSE | genotype | |
| AD | M | 62 | 58 | n.a. | 26 | n.a | MGH |
| AD | M | 83 | 82 | n.a. | 27 | n.a. | MGH |
| AD | M | 72 | 63 | n.a. | 27 | n.a. | MGH |
| AD | F | 74 | 70 | n.a. | 22 | n.a. | MGH |
| AD | F | 67 | 65 | n.a. | 26 | n.a. | MGH |
| AD | M | 77 | 71 | n.a. | n.a. | n.a. | MGH |
| AD | M | 63 | 60 | n.a. | 14 | n.a. | MGH |
| MCI | M | 85 | 82 | n.a. | 29 | n.a. | MGH |
| AD | F | 65 | 57 | 12 | 27 | 3.4 | UW |
| AD | F | 68 | 62 | 16 | 28 | 3.4 | UW |
| AD | F | 65 | 57 | 20 | 21 | 4.4 | UW |
| AD | M | 71 | 68 | 18 | 26 | 4.4 | UW |
| AD | M | 73 | 66 | 20 | 18 | 3.4 | UW |
| AD | M | 73 | 67 | 12 | 22 | 3.3 | UW |
| AD | M | 68 | 63 | 18 | 27 | 3.4 | UW |
| control | F | 59 | — | 16 | 30 | 3.3 | MGH |
| control | F | 57 | — | 16 | 30 | 3.3 | MGH |
| control | M | 79 | — | 16 | 30 | 3.3 | MGH |
| control | F | 81 | — | 16 | 29 | 3.3 | MGH |
| control | M | 78 | — | 16 | 30 | 3.4 | MGH |
| control | M | 77 | — | 16 | 30 | 3.3 | MGH |
| control | F | 66 | — | 16 | 30 | 2.4 | MGH |
| control | F | 80 | — | 16 | 29 | 3.3 | MGH |
| control | F | 61 | — | 16 | 30 | 3.3 | MGH |
| control | M | 60 | — | 16 | 30 | 4.4 | MGH |
| control | F | 71 | — | 16 | 28 | 3.3 | MGH |
| control | F | 68 | — | 16 | 30 | 2.3 | UW |
| control | F | 65 | — | 18 | 30 | 3.4 | UW |
| control | F | 65 | — | 20 | 29 | 3.4 | UW |
| control | F | 70 | — | 20 | 29 | 3.3 | UW |
| control | M | 68 | — | 20 | 30 | 3.3 | UW |
| control | M | 71 | — | 13 | 30 | 3.4 | UW |
| control | M | 74 | — | 18 | 30 | 2.2 | UW |
| control | M | 74 | — | 18 | 29 | 3.4 | UW |

| | | Age at LP | Age of onset | Education | | apoE | | |
|---|---|---|---|---|---|---|---|---|
| Diagnosis | Gender | (years) | (years) | (years) | MMSE | genotype | | |
| FTD | M | 76 | n.a. | 16 | 26 | 2.3 | MGH | UPDRS total score, n.a. |
| FTD | M | 63 | n.a. | 16 | 28 | 3.4 | MGH | UPDRS total score, n.a. |
| FTD | F | 56 | n.a. | 16 | 10 | 3.3 | MGH | UPDRS total score, n.a. |
| FTD | M | 55 | 52 | 14 | 29 | 3.4 | UW | UPDRS total score, 50/108, |
| | | | | | | | | family history of memory problems (+) |
| FTD | M | 67 | 61 | 18 | n.a. | 3.3 | UW | UPDRS total score, 7/108 |
| FTD | M | 70 | 68 | 12 | 4 | 3.4 | UW | UPDRS total score, 17/108 |
| FTD | M | 53 | 52 | 14 | 16 | 3.4 | UW | UPDRS total score, 3/108 |
| FTD | F | 60 | 55 | 16 | 15 | 3.4 | UW | UPDRS total score, 7/108 |
| FTD | M | 74 | 64 | 18 | n.a. | 2.3 | UW | UPDRS total score, n.a. |
| FTD | M | 64 | 52 | 14 | n.a. | 3.3 | UW | UPDRS total score, 13/108, |
| | | | | | | | | family history of memory problems (+) |

Abbreviations: AD, Alzheimer's disease; MCI, mild cognitive impairment; FTD, frontotemporal dementia; LP, lumbar puncture; MMSE, Mini-Mental State Examination, MGH, Massachusetts General hospital; UW, University of Washington.

### REFERENCES

1. Mandelkow, E. M., et al. Tau domains, phosphorylation, and interactions with microtubules. Neurobiology of aging 16, 355-362; discussion 362-353 (1995).
2. Iqbal, K., Liu, F., Gong, C. X. & Grundke-Iqbal, I. Tau in Alzheimer disease and related tauopathies. Current Alzheimer research 7, 656-664 (2010).
3. Braak, H. & Braak, E. Neuropathological staging of Alzheimer-related changes. Acta neuropathologica 82, 239-259 (1991).
4. Hyman, B. T., Van Hoesen, G. W., Damasio, A. R. & Barnes, C. L. Alzheimer's disease: cell-specific pathology isolates the hippocampal formation. Science (New York, N.Y.) 225, 1168-1170 (1984).
5. Serrano-Pozo, A., et al. Examination of the clinicopathologic continuum of Alzheimer disease in the autopsy cohort of the National Alzheimer Coordinating Center. Journal of neuropathology and experimental neurology 72, 1182-1192 (2013).
6. Pooler, A. M., et al. Propagation of tau pathology in Alzheimer's disease: identification of novel therapeutic targets. Alzheimer's research & therapy 5, 49 (2013).
7. Medina, M. & Avila, J. The role of extracellular Tau in the spreading of neurofibrillary pathology. Frontiers in cellular neuroscience 8, 113 (2014).
8. Walker, L. C., Diamond, M. I., Duff, K. E. & Hyman, B. T. Mechanisms of protein seeding in neurodegenerative diseases. JAMA neurology 70, 304-310 (2013).
9. Polydoro, M., et al. Reversal of neurofibrillary tangles and tau-associated phenotype in the rTgTauEC model of early Alzheimer's disease. The Journal of neuroscience: the official journal of the Society for Neuroscience 33, 13300-13311 (2013).
10. de Calignon, A., et al. Propagation of tau pathology in a model of early Alzheimer's disease. Neuron 73, 685-697 (2012).
11. Liu, L., et al. Trans-synaptic spread of tau pathology in vivo. PloS one 7, e31302 (2012).
12. Harris, J. A., et al. Human P301L-mutant tau expression in mouse entorhinal-hippocampal network causes tau aggregation and presynaptic pathology but no cognitive deficits. PloS one 7, e45881 (2012).
13. Sanders, D. W., et al. Distinct tau prion strains propagate in cells and mice and define different tauopathies. Neuron 82, 1271-1288 (2014).
14. Clavaguera, F., et al. Transmission and spreading of tauopathy in transgenic mouse brain. Nature cell biology 11, 909-913 (2009).
15. Ahmed, Z., et al. A novel in vivo model of tau propagation with rapid and progressive neurofibrillary tangle pathology: the pattern of spread is determined by connectivity, not proximity. Acta neuropathologica 127, 667-683 (2014).
16. Goedert, M., Falcon, B., Clavaguera, F. & Tolnay, M. Prion-like mechanisms in the pathogenesis of tauopathies and synucleinopathies. Current neurology and neuroscience reports 14, 495 (2014).
17. Pooler, A. M., Phillips, E. C., Lau, D. H., Noble, W. & Hanger, D. P. Physiological release of endogenous tau is stimulated by neuronal activity. EMBO reports 14, 389-394 (2013).
18. Yamada, K., et al. Neuronal activity regulates extracellular tau in vivo. The Journal of experimental medicine 211, 387-393 (2014).
19. Santacruz, K., et al. Tau suppression in a neurodegenerative mouse model improves memory function. Science (New York, N.Y.) 309, 476-481 (2005).
20. Hoover, B. R., et al. Tau mislocalization to dendritic spines mediates synaptic dysfunction independently of neurodegeneration. Neuron 68, 1067-1081 (2010).
21. Takeda, S., et al. Brain interstitial oligomeric amyloid beta increases with age and is resistant to clearance from brain in a mouse model of Alzheimer's disease. FASEB journal: official publication of the Federation of American Societies for Experimental Biology 27, 3239-3248 (2013).
22. Takeda, S., et al. Novel microdialysis method to assess neuropeptides and large molecules in free-moving mouse. Neuroscience 186, 110-119 (2011).
23. Holmes, B. B., et al. Proteopathic tau seeding predicts tauopathy in vivo. Proceedings of the National Academy of Sciences of the United States of America 111, E4376-4385 (2014).
24. Wegmann, S., et al. Human Tau isoforms assemble into ribbon-like fibrils that display polymorphic structure and stability. The Journal of biological chemistry 285, 27302-27313 (2010).
25. Yamada, K., et al. In vivo microdialysis reveals age-dependent decrease of brain interstitial fluid tau levels in P301S human tau transgenic mice. The Journal of neuroscience: the official journal of the Society for Neuroscience 31, 13110-13117 (2011).
26. Taylor, A. M., et al. A microfluidic culture platform for CNS axonal injury, regeneration and transport. Nature methods 2, 599-605 (2005).
27. Wu, J. W., et al. Small misfolded Tau species are internalized via bulk endocytosis and anterogradely and retrogradely transported in neurons. The Journal of biological chemistry 288, 1856-1870 (2013).
28. Guo, J. L. & Lee, V. M. Seeding of normal Tau by pathological Tau conformers drives pathogenesis of Alzheimer-like tangles. The Journal of biological chemistry 286, 15317-15331 (2011).
29. Frost, B., Jacks, R L. & Diamond, M. I. Propagation of tau misfolding from the outside to the inside of a cell. The Journal of biological chemistry 284, 12845-12852 (2009).
30. Iba, M., et al. Synthetic tau fibrils mediate transmission of neurofibrillary tangles in a transgenic mouse model of Alzheimer's-like tauopathy. The Journal of neuroscience: the official journal of the Society for Neuroscience 33, 1024-1037 (2013).
31. Michel, C. H., et al. Extracellular monomeric tau protein is sufficient to initiate the spread of tau protein pathology. The Journal of biological chemistry 289, 956-967 (2014).
32. Ghoshal, N., et al. Tau conformational changes correspond to impairments of episodic memory in mild cognitive impairment and Alzheimer's disease. Experimental neurology 177, 475-493 (2002).
33. Kuchibhotla, K. V., et al. Neurofibrillary tangle-bearing neurons are functionally integrated in cortical circuits in vivo. Proceedings of the National Academy of Sciences of the United States of America 111, 510-514 (2014).
34. Gomez-Isla, T., et al. Neuronal loss correlates with but exceeds neurofibrillary tangles in Alzheimer's disease. Annals of neurology 41, 17-24 (1997).
35. Rodriguez-Martin, T., et al. Tau phosphorylation affects its axonal transport and degradation. Neurobiology of aging 34, 2146-2157 (2013).
36. Johnson, G. V. Tau phosphorylation and proteolysis: insights and perspectives. Journal of Alzheimer's disease: JAD 9, 243-250 (2006).
37. Danzer, K. M., et al. Heat-shock protein 70 modulates toxic extracellular alpha-synuclein oligomers and rescues trans-synaptic toxicity. FASEB journal: official publication of the Federation of American Societies for Experimental Biology 25, 326-336 (2011).
38. Holmes, B. B., et al. Heparan sulfate proteoglycans mediate internalization and propagation of specific proteopathic seeds. Proceedings of the National Academy of Sciences of the United States of America 110, E3138-3147 (2013).
39. Lasagna-Reeves, C. A., et al. Alzheimer brain-derived tau oligomers propagate pathology from endogenous tau. Scientific reports 2, 700 (2012).
40. Lasagna-Reeves, C. A., et al. Identification of oligomers at early stages of tau aggregation in Alzheimer's disease. FASEB journal: official publication of the Federation of American Societies for Experimental Biology 26, 1946-1959 (2012).
41. Barghorn, S., Biernat, J. & Mandelkow, E. Purification of recombinant tau protein and preparation of Alzheimer-paired helical filaments in vitro. Methods in molecular biology (Clifton, N.J.) 299, 35-51 (2005).
42. Cho, H., Hamza, B., Wong, E. A. & Irimia, D. On-demand, competing gradient arrays for neutrophil chemotaxis. Lab on a chip 14, 972-978 (2014).
43. Takeda, S. et al. Neuronal uptake and propagation of a rare phosphorylated high-molecular-weight tau derived from Alzheimer's disease brain. Nat Commun. October 13; 6:8490 (2015).
44. Takeda, S. et al. Seed-competent HMW tau species accumulates in the cerebrospinal fluid of Alzheimer's disease mouse model and human patients. Ann Neurol. 2016 Jun. 28.
45. Wegmann, S. et al. Removing endogenous tau does not prevent tau propagation yet reduces its neurotoxicity. EMBO J. 2015 Dec. 14; 34(24):3028-41.

To the extent not already indicated, it will be understood by those of ordinary skill in the art that any one of the various embodiments herein described and illustrated can be further modified to incorporate features shown in any of the other embodiments disclosed herein. Thus, other embodiments are within the scope and spirit of the invention. Further, while the description above refers to the invention, the description may include more than one invention.

All patents and other publications identified herein are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### SEQUENCE LISTING

Amino Acid Sequence of Full-Length Tau (2n4r)-MAPT Isoform 2 (NP_005091) MAEPRQEFEV MEDHAGTYGL GDRKDQGGYT MHQDQEGDTD AGLKESPLQT PTEDGSEEPG SETSDAKSTP TAEDVTAPLV DEGAPGKQAA AQPHTEIPEG TTAEEAGIGD TPSLEDEAAG HVTQARMVSK SKDGTGSDDK KAKGADGKTK IATPRGAAPP GQKGQANATR IPAKTPPAPK TPPSSGEPPK SGDRSGYSSP GSPGTPGSRS RTPSLPTPPT REPKKVAVVR TPPKSPSSAK SRLQTAPVPM PDLKNVKSKI GSTENLKHQP GGGKVQIINK KLDLSNVQSK CGSKDNIKHV PGGGSVQIVY KPVDLSKVTS KCGSLGNIHH KPGGGQVEVK SEKLDFKDRV QSKIGSLDNI THVPGGGNKK IETHKLTFRE NAKAKTDHGA EIVYKSPVVS GDTSPRHLSN VSSTGSIDMV DSPQLATLAD EVSASLAKQG L (SEQ ID NO: 1)

1.-12. (canceled)

13. An isolated antibody or antigen-binding portion thereof that specifically binds;
(a) a soluble high molecular weight (HMW) tau species phosphorylated at serine 396;
(b) a soluble HMW tau species phosphorylated at serine 404; or
(c) a soluble HMW tau species phosphorylated at serine 199; and
does not bind soluble low molecular weight (LMW) tau species;
wherein the phosphorylated soluble HMW tau species is non-fibrillar, has a molecular weight of at least about 500 kDa, and wherein the LMW tau species has a molecular weight of no more than 200 kDa.

14. The isolated antibody or antigen-binding portion thereof of claim 13 (a), (b) or (c), that specifically binds the phosphorylation site S396, S404, or S199, respectively.

15.-18. (canceled)

19. The isolated antibody or antigen-binding portion thereof of claim 13, which reduces the soluble HMW tau species phosphorylated at S396, S404, or S199 being taken up by a neuron.

20. The isolated antibody or antigen-binding portion thereof of claim 13, which reduces the soluble HMW tau species phosphorylated at S396, S404, or S199 being axonally transported from a neuron to a synaptically-connected neuron.

21. The isolated antibody or antigen-binding portion thereof of claim 13, wherein the soluble HMW tau species phosphorylated at S396, S404, or S199 has a molecular weight of at least about 669 kDa.

22. (canceled)

23. The isolated antibody or antigen-binding portion thereof of claim 13, wherein the soluble HMW tau species phosphorylated at S396, S404, or S199 is in a form of globular particles.

24. The isolated antibody or antigen-binding portion thereof of claim 23, wherein the particle size ranges from about 10 nm to about 30 nm.

25. (canceled)

26. A method of preventing propagation of pathological tau protein between synaptically-connected neurons comprising selectively reducing the extracellular level of a first phosphorylated soluble HMW tau species in contact with a synaptically-connected neuron, wherein the first phosphorylated soluble HMW tau species is non-fibrillar, has a molecular weight of at least about 500 kDa, and is phosphorylated at least at serine 396, wherein a reduced level of the first phosphorylated soluble HMW tau species results in reduced propagation of pathological tau protein between synaptically-connected neurons.

27. The method of claim 26, further comprising selectively reducing the extracellular level of a second phosphorylated soluble HMW tau species in contact with a synaptically-connected neuron, wherein the second phosphorylated soluble HMW tau species is non-fibrillar, has a molecular weight of at least about 500 kDa, and is phosphorylated at least at serine 199 and/or serine 404.

28. The method of claim 26, wherein the extracellular level of a third phosphorylated soluble HMW tau species that is phosphorylated at serine 422 is not substantially reduced during said selective reduction.

29. The method of claim 26, wherein the extracellular level of soluble LMW tau species is not substantially reduced during said selective reduction.

30. The method of claim 26, wherein the first and/or second phosphorylated soluble HMW tau species is selectively reduced by contacting the extracellular space or fluid in contact with the synaptically-connected neurons with an antagonist of the first and/or second phosphorylated soluble HMW tau species.

31. The method of claim 30, wherein the antagonist of the first and/or second phosphorylated soluble HMW tau species is selected from the group consisting of an antibody, a zinc finger nuclease, a transcriptional repressor, a nucleic acid inhibitor, a small organic molecule, an aptamer, a gene-editing composition, and a combination thereof.

32. A method of reducing tau-associated neurodegeneration in a subject comprising selectively reducing the level of a first phosphorylated soluble HMW tau species in the brain of the subject determined to have, or be at risk for, tau-associated neurodegeneration, wherein the first phosphorylated soluble HMW tau species is non-fibrillar, has a molecular weight of at least about 500 kDa, and is phosphorylated at least at serine 396, wherein a reduced level of the first phosphorylated soluble HMW tau species results in reduced tau-associated neurodegeneration.

33. The method of claim 32, further comprising selectively reducing the level of a second phosphorylated soluble HMW tau species in the brain of the subject, wherein the second phosphorylated soluble HMW tau species is non-fibrillar, has a molecular weight of at least about 500 kDa, and is phosphorylated at least at serine 199 and/or serine 404.

34. The method of claim 32, wherein the level of a third phosphorylated soluble HMW tau species that is phosphorylated at S422 is not substantially reduced during the treatment.

35. The method of claim 32, wherein the level of soluble LMW tau species in the subject is not substantially reduced during the treatment.

36. (canceled)

37. (canceled)

38. The method of claim 32, wherein the first and/or second phosphorylated soluble HMW tau species is selectively reduced by administering to the brain of the subject an antagonist of the first and/or second soluble HMW tau species.

39. The method of claim 38, wherein the antagonist of the first and/or second soluble HMW tau species is selected from the group consisting of an antibody, a zinc finger nuclease, a transcriptional repressor, a nucleic acid inhibitor, a small organic molecule, an aptamer, a gene-editing composition, and a combination thereof.

40. The method of claim 32, further comprising selecting a subject determined to have soluble HMW tau species present in the brain at a level above a reference level.

41.-57. (canceled)


SEQUENCE LISTING (1 sequence)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL